SEQUENCE LISTING (41 sequences)

SEQ ID NO: 1 - DNA, 440 bp, Unknown
  Description of Unknown Lachnospiraceae sequence
cctacggggggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtaggtggtatggcaagtcagaggtgaaaacccagggcttaaccttgggattgcctttgaaactgtcagactagagtgcaggaggggtaagtggaattcctagtgtagcggtgaaatgcgtagatattaggaggaacaccagtggcgaaggcggcttactggactgtaactgacactgaggctcgaaagcgtggggagcaaacaggattagatacccgagtagtc

SEQ ID NO: 2 - DNA, 442 bp, Unknown
  Description of Unknown Firmicutes sequence
cctacggggggctgcagtggggaatattgggcaatggaggaaactctgacccagcaacgccgcgtggaggaagaaggttttcggatcgtaaactcctgtccttggagacgagtagaagacggtatccaaggaggaagccccggctaactacgtgccagcagccgcggtaatacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaacaggattagataccccagtagtc

SEQ ID NO: 3 - DNA, 441 bp, Butyricicoccus sp.
cctacggggggctgcagtggggaatattgcgcaatgggggaaaccctgacgcagcaacgccgcgtgattgaagaaggccttcgggttgtaaagatctttaatcagggacgaaacatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagatacccctgtagtc

SEQ ID NO: 4 - DNA, 440 bp, Unknown
  Description of Unknown Lachnospiraceae sequence
cctacgggtggctgcagtggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggtgaggcaagtctgaagtgaaatgccggggctcaaccccggaactgctttggaaactgtcgtactagagtgtcggaggggtaagcggaattcctagtgtagcggtgaaatgcgtagatattaggaggaacaccagtggcgaaggcggcttgctggactgtaactgacactgaggctcgaaagcgtggggagcaaacaggattagatacccttgtagtc

SEQ ID NO: 5 - DNA, 441 bp, Unknown
  Description of Unknown Clostridiales sequence
cctacggggggcagcagtcgggaatattgcgcaatggaggaaactctgacgcagtgacgccgcgtataggaagaaggttttcggattgtaaactattgtcgttagggaagatacaagacagtacctaaggaggaagctccggctaactacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcgactttctggacaataactgacgttgaggcacgaaagtgtggggagcaaacaggattagataccccagtagtc

SEQ ID NO: 6 - DNA, 443 bp, Unknown
  Description of Unknown Ruminococcaceae sequence
cctacggggggctgcagtggggaatattgggcaatgggcgaaagcctgacccagcaacgccgcgtgaaggaagaaggtcttcggattgtaaacttcttttatgagggacgaaggaagtgacggtacctcatgaataagccacggctaactacgtgccagcagccgcggtaatacgtaggtggcaagcgttgtccggatttactgggtgtaaagggcgcgtaggcgggatggcaagtcagatgtgaaatccatgggctcaacccatgaactgcatttgaaactgtcgttcttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacaggattagatacccctgtagtc

SEQ ID NO: 7 - DNA, 440 bp, Unknown
  Description of Unknown Ruminococcaceae sequence
cctacggggggctgcagtgggggatattgcacaatgggggaaaccctgatgcagcaacgccgcgtgagggaagaaggttttcggattgtaaacctctgtcctcagggaagataatgacggtacctgaggaggaagctccggctaactacgtgccagcagccgcggtaatacgtagggagcaagcgttgtccggatttactgggtgtaaagggtgcgtaggcgggatatcaagtcagacgtgaaatccatcggcttaactgatgaactgcgtttgaaactggtattcttgagtgagtcagaggcaggcggaattcccggtgtagcggtgaaatgcgtagagatcgggaggaacaccagtggcgaaggcggcctgctggggcttaactgacgctgaggcacgaaagcgtggggagcaaacaggattagatacccgagtagtc

SEQ ID NO: 8 - DNA, 443 bp, Unknown
  Description of Unknown Firmicutes sequence
cctacggggggctgcagtggggaatattgggcaatggagggaactctgacccagcaatgccgcgtgagtgaagaaggttttcggattgtaaaactctttaagcagggacgaagaaagtgacggtacctgcagaataagcatcggctaactacgtgccagcagccgcggtaatacgtaggatgcaagcgttatccggaatgactgggcgtaaagggtgcgtaggcggtaaatcaagttggcagcgtaattccggggcttaactccggaactactgccaaaactggtgaactagagtgtgtcaggggtaagtggaattcctagtgtagcggtggaatgcgtagatattaggaggaacaccggaggcgaaagcgacttactggggcacaactgacgctgaggcacgaaagcgtggggagcaaacaggattagataccccggtagtc

SEQ ID NO: 9 - DNA, 440 bp, Unknown
  Description of Unknown Ruminococcaceae sequence
cctacgggaggcagcagtgggggatattgcacaatggaggaaactctgatgcagcaacgccgcgtgagggaagaaggatttcggtttgtaaacctctgtcttcggtgacgaaaatgacggtagccgaggaggaagctccggctaactacgtgccagcagccgcggtaatacgtagggagcaagcgttgtccggaattactgggtgtaaagggtgcgtaggtgggactgcaagtcaggtgtgaaaacggtcggctcaaccgatcgcctgcacttgaaactgtggttcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccccggtagtc

SEQ ID NO: 10 - DNA, 440 bp, Unknown
  Description of Unknown Lachnospiraceae sequence
cctacggggggctgcagtggggaatattgcacaatgggggaaaccctgatgcagcgacgccgcgtgagcgaagaagtatttcggtatgtaaagctctatcagcagggaagataatgacggtacctgactaagaagccccggctaaatacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcggcctactgggcaccaactgacgctgaggctcgaaagtgtgggtagcaaacaggattagataccctagtagtc

SEQ ID NO: 11 - protein, 4 aa, Unknown
  Description of Unknown metabolite sequence
ALWG

SEQ ID NO: 12 - protein, 4 aa, Unknown
  Description of Unknown metabolite sequence
FGGS

SEQ ID NO: 13 - protein, 4 aa, Unknown
  Description of Unknown metabolite sequence
QMPS

SEQ ID NO: 14 - protein, 4 aa, Unknown
  Description of Unknown metabolite sequence
ANCG

SEQ ID NO: 15 - protein, 4 aa, Unknown
  Description of Unknown metabolite sequence
DMDP

SEQ ID NO: 16 - protein, 4 aa, Unknown
  Description of Unknown metabolite sequence
AKFC

SEQ ID NO: 17 - protein, 4 aa, Unknown
  Description of Unknown metabolite sequence
CFFQ

SEQ ID NO: 18 - protein, 4 aa, Unknown
  Description of Unknown metabolite sequence
CPPY

SEQ ID NO: 19 - protein, 4 aa, Unknown
  Description of Unknown metabolite sequence
MMTW

SEQ ID NO: 20 - protein, 4 aa, Unknown
  Description of Unknown metabolite sequence
CEEE

SEQ ID NO: 21 - protein, 4 aa, Unknown
  Description of Unknown metabolite sequence
EIIF

SEQ ID NO: 22 - protein, 4 aa, Unknown
  Description of Unknown metabolite sequence
EAQS

SEQ ID NO: 23 - protein, 4 aa, Unknown
  Description of Unknown metabolite sequence
LSSY

SEQ ID NO: 24 - protein, 4 aa, Unknown
  Description of Unknown metabolite sequence
ALWP

SEQ ID NO: 25 - protein, 4 aa, Unknown
  Description of Unknown metabolite sequence
IKCG

SEQ ID NO: 26 - protein, 4 aa, Unknown
  Description of Unknown metabolite sequence
HMVV

SEQ ID NO: 27 - protein, 4 aa, Unknown
  Description of Unknown metabolite sequence
RKFV

SEQ ID NO: 28 - protein, 4 aa, Unknown
  Description of Unknown metabolite sequence
KFFF

SEQ ID NO: 29 - protein, 4 aa, Unknown
  Description of Unknown metabolite sequence
RLPR

SEQ ID NO: 30 - protein, 4 aa, Unknown
  Description of Unknown metabolite sequence
QFFF

SEQ ID NO: 31 - protein, 4 aa, Unknown
  Description of Unknown metabolite sequence
EEGY

SEQ ID NO: 32 - protein, 4 aa, Unknown
  Description of Unknown metabolite sequence
LSSY

SEQ ID NO: 33 - protein, 4 aa, Unknown
  Description of Unknown metabolite sequence
RLVC

SEQ ID NO: 34 - protein, 4 aa, Unknown
  Description of Unknown metabolite sequence
RKWV

SEQ ID NO: 35 - protein, 4 aa, Unknown
  Description of Unknown metabolite sequence
HMVV

SEQ ID NO: 36 - protein, 4 aa, Unknown
  Description of Unknown metabolite sequence
ALWP

SEQ ID NO: 37 - protein, 4 aa, Unknown
  Description of Unknown metabolite sequence
ILFW

SEQ ID NO: 38 - protein, 4 aa, Unknown
  Description of Unknown metabolite sequence
NKVP

SEQ ID NO: 39 - protein, 4 aa, Unknown
  Description of Unknown metabolite sequence
RVPY

SEQ ID NO: 40 - DNA, 50 bp, Artificial Sequence
  Description of Artificial Sequence Synthetic primer
  modified_base (42)..(42): a, c, t, or g
tcgtcggcagcgtcagatgtgtataagagacagcctacgggnggcwgcag

SEQ ID NO: 41 - DNA, 55 bp, Artificial Sequence
  Description of Artificial Sequence Synthetic primer
gtctcgtgggctcggagatgtgtataagagacaggactachvgggtatctaatcc

(19) United States
(12) Patent Application Publication
(10) Pub. No.: US 2022/0128556 A1
(43) Pub. Date: Apr. 28, 2022

(54) METHODS OF DIAGNOSING DISEASE

(71) Applicant: 4D Pharma Cork Limited, Cork (IE)

(72) Inventors: PAUL O'TOOLE, Cork (IE); Fergus SHANAHAN, Cork (IE); Ian JEFFERY, Cork (IE); Eileen O'HERLIHY, Cork (IE); Anubhav DAS, Cork (IE)

(21) Appl. No.: 17/491,563

(22) Filed: Oct. 1, 2021

Related U.S. Application Data
(63) Continuation of application No. PCT/EP2020/059459, filed on Apr. 2, 2020.

(30) Foreign Application Priority Data
Apr. 3, 2019 (EP) 19167114.8
Apr. 3, 2019 (EP) 19167118.9
Jun. 24, 2019 (GB) 1909052.1
Oct. 18, 2019 (GB) 1915143.0
Oct. 18, 2019 (GB) 1915156.2

(51) Int. Cl. G01N 33/569; G01N 33/68

(52) U.S. Cl. CPC G01N 33/56911; G01N 33/6893

(57) ABSTRACT

The application provides new and improved methods for diagnosing IBS.

### CROSS-REFERENCE

This application is a continuation of International Application No. PCT/EP2020/059459, filed Apr. 2, 2020, which claims the benefit of European Application No. 19167114.8, filed Apr. 3, 2019, European Application No. 19167118.9, filed Apr. 3, 2019, Great Britain Application No. 1909052.1, filed Jun. 24, 2019, Great Britain Application No. 1915143.0, filed Oct. 18, 2019, and Great Britain Application No. 1915156.2, filed Oct. 18, 2019, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 27, 2021, is named 56686-702_301_SL.txt and is 13,273 bytes in size.

### TECHNICAL FIELD

This invention is in the field of diagnosis and in particular the diagnosis of irritable bowel syndrome (IBS).

### BACKGROUND

Irritable bowel syndrome (IBS) is a common condition that affects the digestive system. Results from global epidemiological studies have shown that IBS is present in 3% to 30% of a population, with no common trend across different countries (1). Symptoms include cramps, bloating, diarrhoea and constipation and occur over a long time period, generally years. Disorders such as anxiety, major depression, and chronic fatigue syndrome are common among people with IBS. There is no known cure for IBS and treatment is generally carried out to improve symptoms. Treatment may include dietary changes, medication, probiotics, and/or counselling. Dietary measures that are commonly suggested as treatments include increasing soluble fiber intake, a gluten-free diet, or a short-term diet low in fermentable oligosaccharides, disaccharides, monosaccharides, and polyols (FODMAPs). The medication loperamide is used to help with diarrhea while laxatives are be used to help with constipation. Antidepressants may improve overall symptoms and pain. Like most chronic non-communicable disorders, IBS appears to be heterogeneous (2). It ranges in severity from nuisance bowel disturbance to social disablement, accompanied by marked symptomatic heterogeneity (3). Although frequently considered a disorder of the brain-gut axis (4,5), it is unclear if IBS begins in the gut or in the brain or both. The occurrence of post-infectious IBS (6) suggests that a proportion of cases are initiated in the end-organ, albeit with susceptibility risk factors, some of which may be psychosocial. Advances in microbiome science, with emerging evidence for a modifying influence by the microbiota on neurodevelopment and perhaps on behaviour, have broadened the concept of the mind/body link to encompass the microbiota-gut-brain axis (7).

However, progress in understanding and treating IBS has been limited by the absence of reliable biomarkers and IBS is still defined by symptoms. Currently, gastrointestinal (GI) diseases such as IBS are standardised using the Rome criteria. Diagnosis of IBS using the Rome Criteria is based on whether the patient has symptoms which are associated with IBS. These criteria were established by a group of experts in functional gastrointestinal disorders, known as the Rome Consensus Commission, in order to develop and provide guidance in research. They have been updated in five separate editions, to make them more relevant outside of research, and useful in improving clinical trials (1,8). However, results from one study (1) have shown that the prevalence of IBS is dependent on which edition of the Rome criteria is applied; the later editions exhibited a lower prevalence of IBS amongst populations.

Other criteria used to diagnose IBS include the WONCA criteria, involving the exclusion of other organic diseases, and DSM (Diagnostic and Statistical Manual for Mental Disorders). Here, the analysis included before diagnosis is minimal, with specialist examination occurring only as an exception (1). Investigations have been carried out into gut microbiota alterations in patients with IBS compared to control (non-IBS) groups (9,10,11,12). Interaction of the microbiome with diet, antibiotics and enteric infections, all of which may be involved in IBS, is consistent with the hypothesis that microbiome alterations could activate or perpetuate pathophysiological mechanisms in the syndrome (13,14). Biomarkers have been found to be associated with IBS, which has provided more flexibility for defining subpopulations of IBS that are not based on clinical symptoms (1). However, robust microbiome signatures or biomarkers that separate IBS patients from controls and that help inform therapies are lacking, though signatures have been suggested for IBS severity (12). Furthermore, most microbiota studies to date have employed 16S rRNA profiling, and did not analyse bacterial metabolites.

The Rome criteria are also used to classify IBS subtypes. Currently, IBS subtypes are defined by the Rome criteria (15). These subtypes are IBS-C, IBS-D and IBS-M. IBS-C is IBS with predominant constipation where stool types 1 and 2 (according to the Bristol stool chart) are present more than 25% of the time and stool types 6 and 7 are present less than 25% of the time. IBS-D is IBS with predominant diarrhoea where stool types 1 and 2 are present less than 25% of the time and stool types 6 and 7 more than 25% of the time. IBS-M is IBS where there is a mixture of IBS-C and IBS-D with stool types 1, 2, 6 and 7 present more than 25% of the time, and is known as IBS-mixed type. While these classifications can establish predominance of constipation over diarrhoea and diarrhoea over constipation, they are not very useful for long term treatment of IBS given the heterogenic nature of the disease and the tendency of patients to move from one subtype classification to another within a given time period (16). The current approach has significant limitations including failure to inform treatment of patients who alternate between subtypes sometimes within days (17). More understanding is required for this disease and like other gut related illness a change in gut microbiota can be signatory of a change in disease pattern (18). Furthermore, the forms of diarrhoea or constipation can be diverse. Pharmaceutical agents designed to tackle polar opposite symptoms have the potential for severe unwanted adverse effects if prescribed for a patient who has been misclassified (19). What is of interest are alterations in the microbiome of patients with IBS and what correlation if any there is with the symptoms of IBS. However, IBS subtypes (IBS-C, IBS-D, IBS-M) are not useful for distinguishing between the different microbiomes of patients diagnosed with IBS according to the Rome criteria.

There is a requirement for further and improved methods for diagnosing bowel disorders such as IBS, including the diagnosis of the various IBS subtypes.

### SUMMARY OF THE INVENTION

The inventors have developed new and improved methods for diagnosing IBS. A comprehensive and detailed analysis of the microbiome, the metabolome and gene pathways in patients and control (non-IBS) individuals has allowed new indicators of disease to be identified. The invention therefore provides a method of diagnosing IBS in a patient comprising detecting: a bacterial strain of a taxa associated with IBS; a microbial gene involved in a pathway associated with IBS; and/or a metabolite associated with IBS. The inventors have also developed new and improved methods for stratification of patients with IBS. The invention therefore provides a method of classification of a patient with IBS to a subgroup based on the microbiome, comprising detecting: a bacterial strain of a taxa associated with an IBS subgroup and/or a metabolite associated with an IBS subgroup.

### BRIEF DESCRIPTION OF THE FIGURES

FIGS. 1A-1D. Microbiota compositional analysis of Control and IBS groups. (FIG. 1A) Principal Co-Ordinate Analysis (PCoA) of microbiota beta diversity showing significant difference between Control and IBS groups. PCoA performed using Spearman distance at 16S genus level (p-value=0.001; Control: n=63, IBS n=78). (FIG. 1B) Predictive taxa for IBS determined by Random Forest machine learning on shotgun dataset (Control: n=59; IBS n=80). (FIG. 1C) PCoA of the microbiota composition showing no significant difference between IBS clinical subtypes. PCoA performed using Spearman distance at 16S OTU level (p-value=0.976; IBS-C: n=29, IBS-D: n=20, IBS-M: n=29). (FIG. 1D) Shotgun genus profile of Control and IBS groups (Control: n=58, IBS: n=78). P-values for data/tests presented in panels A and C were calculated using Permutational MANOVA (R function/package:adonis/vegan)

FIG. 2. PCoA of microbiota diversity shows significant difference between Control and IBS groups. PCoA performed using Spearman distance at shotgun genus level (p-value=0.001; Control: n=58, IBS n=78).

FIGS. 3A-3C. Microbiota diversity of IBS and Control groups. (FIG. 3A). The diversity (Observed richness) of the IBS group was significantly different from the Control group based on Wilcoxon rank sum test (pvalue=9.215e-08, Control: n=63, IBS: n=78). (FIG. 3B) The diversity (observed richness) of the IBS clinical sub-types were significantly different from the Control group based on Kruskal-Wallis (p-value=1.28e-06, Control: n=63; IBS-C: n=29; IBS-D: n=20; IBS-M: n=29). (FIG. 3C) The diversity (Shannon index) of the Control was significantly different from the IBS group using differences based on Wilcoxon (p-value=0.00032, Control: n=63, IBS: n=78).

FIGS. 4A-4C. Comparison of Control and IBS urine and fecal metabolomes. (FIG. 4A) PCoA of urine volatile organic compounds (FAIMS) metabolomes. Adonis p-value=0.001; (Control: n=65; IBS: n=80). (FIG. 4B) PCoA of urine MS metabolomics using Spearman distance. Adonis p-value=0.001; (Control: n=63; IBS: n=80). (FIG. 4C) PCoA of fecal MS metabolomics using Spearman distance. Adonis p-value=0.001; (Control: n=63; IBS: n=80). P-values we calculated using Permutational MANOVA (R function/package:adonis/vegan)

FIG. 5. PCoA of FAIMS urine metabolomics using Spearman distance shows a significant difference between Control and IBS clinical sub-types (Adonis p-value=0.001; Control: n=63; IBS-C: n=29; IBS-D: n=20; IBS-M: n=29).

FIGS. 6A-6B. Urine metabolomic Receiver operating characteristic (ROC) curves to distinguish IBS from Control status. (FIG. 6A) ROC curve analysis using 10-Fold cross-validation on urine LC/GC-MS metabolomics (Control: n=61; IBS: n=78 where 85% (52/61 of the control group and 95% (74/78) of the IBS group were correctly predicted. (FIG. 6B) ROC curve analysis using 10-Fold cross-validation on urine FAIMS metabolomics (Control: n=63; IBS: n=78 where 70% (44/63 of the control group and 83% (65/78) of the IBS group were correctly predicted.

FIG. 7. PCoA of fecal metabolomics using Spearman distance shows no significant difference between the IBS clinical sub-types (p-value=0.202; IBS-C: n=29; IBS-D: n=20; IBS-M: n=29).

FIG. 8. Between class analysis (BCA) showing two microbiota-IBS clusters when compared to the Control group (Control: n=63, IBS Cluster I: n=35, IBS Cluster II: n=43).

FIG. 9. Core workflow of an alternative machine learning pipeline. N represents number of features returned by Least Absolute Shrinkage and Selection Operator (LASSO).

FIG. 10. Principal Coordinate analysis of co-abundant genes in metagenomics samples shows a significant split between IBS (80 samples) and Controls (59 samples). Significance of the split was determined using PMANOVA (p<0.001).

FIG. 11. Heatmap of microbiome OTU data with hierarchical clustering using Canberra distance and ward linkage.

FIG. 12. Alpha diversity (observed species) of the healthy controls and the three IBS subgroups (IBS-1, IBS-2, IBS-3). Observed species (richness) is defined as the count of unique OTU's within a sample. Significance was determined using ANOVA.

FIG. 13. PCoA of Canberra distances of healthy controls and the three IBS subgroups (IBS-1, IBS-2, IBS-3) at the genus level for samples sequenced using 16S.

FIG. 14. PCoA of Canberra distances of healthy controls and the three IBS subgroups (IBS-1, IBS-2, IBS-3) at the species level for shotgun sequenced samples.

FIG. 15. PCoA of Canberra distances of healthy controls and the three IBS subgroups (IBS-1, IBS-2, IBS-3) for the fecal metabolomics samples.

FIG. 16. PCoA of Canberra distances of healthy controls and the three IBS subgroups (IBS-1, IBS-2, IBS-3) for the urine metabolomics samples.

FIG. 17. Microbiota compositional analysis of Control and IBS groups. PCoA of the metagenomic species analysis (co-abundant genes, CAGs) showing a significant difference between Control and IBS groups. (Control: n=59; IBS n=80). P-values for data/tests presented were calculated using Permutational MANOVA (R function/package:adonis/vegan)

### DISCLOSURE OF THE INVENTION

Bacterial Taxa as Predictive Features of IBS

The inventors have identified bacterial taxa that are predictive of IBS, as demonstrated in the examples. Accordingly, the invention provides methods for diagnosing IBS comprising detecting the presence of certain bacterial taxa. As detailed below, the bacterial taxa used in the invention may be defined with reference to 16S rRNA gene sequences, or the invention may use Linnaean taxonomy. Bacteria of either category of taxa may be detected using Clade-specific bacterial genes, 16S sequences, transcriptomics, metabolomics, or a combination of such techniques. Preferably, these methods comprise detecting bacteria (i.e. one or more bacterial strains) in a fecal sample from a patient. Alternatively, the bacteria may be detected from an oral sample, such as a swab. Generally, detecting a bacterial taxa associated with IBS in the methods of the invention comprises measuring the relative abundance of the bacteria in a sample, for example relative to a corresponding sample from a control (non-IBS) individual or relative to a reference value. In one embodiment, the present invention provides a method for diagnosing IBS, comprising detecting bacterial species which may include one or more of the following genera: Actinomyces, Oscillibacter, Paraprevotella, Lachnospiraceae, Erysipelotrichaceae and Coprococcus. In one embodiment, the present invention provides a method for diagnosing IBS, comprising detecting a bacterial strain belonging to a genus selected from the group consisting of: Escherichia, Clostridium, Streptococcus, Parabacteroides, Turicibacter, Eubacterium, Bacteroides, Klebsiella, Pseudoflavonifractor, and Enterococcus. In a particular embodiment, the bacterial species is of the genus Actinomyces. In a particular embodiment, the bacterial species is of the genus Oscillibacter. In a particular embodiment, the bacterial species is of the genus Paraprevotella. In a particular embodiment, the bacterial species is of the genus Lachnospiraceae. In a particular embodiment, the bacterial species is of the genus Erysipelotrichaceae. In a particular embodiment, the bacterial species is of the genus Coprococcus. In a particular embodiment, the bacterial species is of the genus Escherichia. In a particular embodiment, the bacterial species is of the genus Clostridium. In a particular embodiment, the bacterial species is of the genus Streptococcus. In a particular embodiment, the bacterial species is of the genus Parabacteroides. In a particular embodiment, the bacterial species is of the genus Turicibacter. In a particular embodiment, the bacterial species is of the genus Eubacterium. In a particular embodiment, the bacterial species is of the genus Bacteroides. In a particular embodiment, the bacterial species is of the genus Klebsiella. In a particular embodiment, the bacterial species is of the genus Pseudoflavonifractor. In a particular embodiment, the bacterial species is of the genus Enterococcus. In preferred embodiments, the method of the invention comprises detecting bacteria (i.e. one or more bacterial strains) of more than one of the genera listed in Table 1, such as detecting bacteria of Actinomyces, Oscillibacter, Paraprevotella, Lachnospiraceae, Erysipelotrichaceae and Coprococcus. In certain embodiments, the bacteria (i.e. one or more bacterial strains) may be detected using Clade-specific bacterial genes, 16S sequences, transcriptomics or metabolomics. In any such embodiments, detecting the bacteria comprises measuring the relative abundance of the bacteria in a sample, for example relative to a corresponding sample from a control (non-IBS) individual or relative to a reference value. The examples demonstrate that such methods are particularly effective.

In one embodiment, the present invention provides a method for diagnosing IBS, comprising detecting one or more bacterial species selected from the following: Ruminococcus gnavus, Coprococcus catus, Bamesiella intestinihominis, Anaerotruncus colihominis, Eubacterium eligens, Clostridium symbiosum, Roseburia inulinivorans, Paraprevotella clara, Ruminococcus lactaris, Clostridium citroniae, Clostridium leptum, Ruminococcus bromii, Bacteroides thetaiotaomicron, Eubacterium biforme, Bifidobacterium adolescentis, Parabacteroides distasonis, Dialister invisus, Bacteroides faecis, Butyrivibrio crossotus, Clostridium nexile, Bacteroides cellulosilyticus, Pseudoflavonifractor capillosus, Streptococcus anginosus, Streptococcus sanguinis, Desulfovibrio desulfuricans and/or Clostridium ramosum. In certain embodiments, the method of the invention comprises detecting two or more species from the above list, such as at least 5, 10, 15, 20 or all of the species. In one embodiment, the present invention provides a method for diagnosing IBS, comprising detecting one or more bacterial strains that may be selected from the list consisting of Lachnospiraceae bacterium_3_1_46FAA, Lachnospiraceae bacterium_7_1_58FAA, Lachnospiraceae bacterium_1_4_56FAA, Lachnospiraceae bacterium_2_1_58FAA, Coprococcus sp_ART55_1, Alistipes sp_AP11 and/or Bacteroides sp_1_1_6, or corresponding strains, such as strains with a 16S rRNA gene sequence that is at least 95%, 96%, 97%, 98%, 99%, 99.5% or 99.9% identical to the 16S gene rRNA sequence of the reference bacterium. In certain embodiments, the method of the invention comprises detecting two or more bacteria from the above list, such as at least 3, 4, 5 or all of the bacteria. In any such embodiments, detecting the bacteria comprises measuring the relative abundance of the bacteria in a sample, for example relative to a corresponding sample from a control (non-IBS) individual or relative to a reference value. In certain embodiments, the bacteria (i.e. one or more bacterial strains) may be detected using Clade-specific bacterial genes, 16S sequences, transcriptomics or metabolomics.

In one embodiment, the present invention provides a method for diagnosing IBS, comprising detecting one or more bacterial species selected from the following: Prevotella buccalis, Butyricicoccus pullicaecorum, Granulicatella elegans, Pseudoflavonifractor capillosus, Clostridium ramosum, Streptococcus sanguinis, Clostridium citroniae, Desulfovibrio desulfuricans, Haemophilus pittmaniae, Paraprevotella clara, Streptococcus anginosus, Anaerotruncus colihominis, Clostridium symbiosum, Mitsuokella multacida, Clostridium nexile, Lactobacillus fermentum, Eubacterium biforme, Clostridium leptum, Bacteroides pectinophilus, Coprococcus catus, Eubacterium eligens, Roseburia inulinivorans, Bacteroides faecis, Bamesiella intestinihominis, Bacteroides thetaiotaomicron, Ruminococcus bromii, Ruminococcus gnavus, Ruminococcus lactaris, Parabacteroides distasonis, Butyrivibrio crossotus, Bacteroides cellulosilyticus, Bifidobacterium adolescentis, and/or Dialister invisus. In certain embodiments, the method of the invention comprises detecting two or more species from the above list, such as at least 5, 10, 15, 20 or all of the species. In one embodiment, the present invention provides a method for diagnosing IBS, comprising detecting one or more bacterial strains that may be selected from the list consisting of Lachnospiraceae bacterium_2_1_58FAA, Lachnospiraceae bacterium_7_1_58FAA, Lachnospiraceae bacterium_1_4_56FAA, Lachnospiraceae bacterium_3_1_46FAA, Alistipes sp_AP11, Bacteroides_sp_1_1_6, and/or Coprococcus_sp_ART55_1, or corresponding strains, such as strains with a 16S rRNA gene sequence that is at least 95%, 96%, 97%, 98%, 99%, 99.5% or 99.9% identical to the 16S gene rRNA sequence of the reference bacterium. In certain embodiments, the method of the invention comprises detecting two or more bacteria from the above list, such as at least 3 or 4 or all of the bacteria. In any such embodiments, detecting the bacteria (i.e. one or more bacterial strains) comprises measuring the relative abundance of the bacteria in a sample, for example relative to a corresponding sample from a control (non-IBS) individual or relative to a reference value. In certain embodiments, the bacteria (i.e. one or more bacterial strains) may be detected using clade-specific bacterial genes, 16S sequences, transcriptomics or metabolomics.

In one embodiment, the present invention provides a method for diagnosing IBS, comprising detecting one or more bacterial strains belonging to an operational taxonomic unit (OTU) associated with IBS. As known in the art, an operational taxonomic unit (OTU) is an operational definition used to classify groups of closely related individuals. As used herein, an “OTU” is a group of organisms which are grouped by DNA sequence similarity of a specific taxonomic marker gene (49). In some embodiments, the specific taxanomic marker gene is the 16S rRNA gene. In some embodiments, the Ribosomal Database Project (RDP) taxonomic classifier is used to assign taxonomy to representative OTU sequences. For example, the sequence information in Table 12 can be used to classify whether bacteria (i.e. one or more bacterial strains) belong to the OTUs listed in Table 11. Bacteria having at least 97% sequence identity to the sequences in Table 12 belong to the corresponding OTUs in Table 11. In preferred embodiments, the OTU is selected from tables 1, 11 and/or 12. In any such embodiments, detecting the bacteria (i.e. one or more bacterial strains) comprises measuring the relative abundance of the bacteria in a sample, for example relative to a corresponding sample from a control (non-IBS) individual or relative to a reference value.

In certain embodiments, the bacterial species belongs to a sequence-based taxon. In preferred embodiments, the sequence-based taxon is selected from tables 1-3.

In one embodiment, a bacterial species or strain predictive of IBS is more abundant in patients suffering from IBS. In a particular embodiment, the method of the invention comprises measuring the abundance of a bacterial species or strain, wherein increased abundance is associated with IBS, and wherein the strain or species is selected from: Ruminococcus gnavus, Lachnospiraceae bacterium_3_1_46FAA, Lachnospiraceae bacterium_7_1_58FAA, Anaerotruncus colihominis, Lachnospiraceae bacterium_1_4_56FAA, Clostridium symbiosum, Clostridium citroniae, Lachnospiraceae bacterium_2_1_58FAA, Clostridium nexile, and/or Clostridium ramosum, In one embodiment, the present invention provides a method for diagnosing IBS, comprising detecting one or more bacterial species or strains which is more abundant in patients suffering from IBS. In certain embodiments, the method of the invention comprises detecting two or more species or strains from the above list, such as at least 5, 10, 15, 20 or all of the species.

In one embodiment, the bacterial species predictive of IBS is significantly more abundant in patients suffering from IBS. In a preferred embodiment, the bacterial species predictive of IBS that is significantly more abundant in patients suffering from IBS is Ruminococcus gnavus and/or Lachnospiraceae spp.

In one embodiment, a bacterial species or strain predictive of IBS is less abundant in patients suffering from IBS. In a particular embodiment, the method of the invention comprises measuring the abundance of a bacterial species or strain, wherein decreased abundance is associated with IBS, and wherein the strain or species is selected from: Coprococcus catus, Barnesiella intestinihominis, Eubacterium eligens, Paraprevotella clara, Ruminococcus lactaris, Eubacterium biforme, and/or Coprococcus sp_ART55_1. In one embodiment, the present invention provides a method for diagnosing IBS, comprising detecting one or more bacterial species or strains which are less abundant in patients suffering from IBS.

In one embodiment, the bacterial species predictive of IBS is significantly less abundant in patients suffering from IBS. In a preferred embodiment, the bacterial species predictive of IBS that is significantly less abundant in patients suffering from IBS is Barnesiella intestinihominis and/or Coprococcus catus.

In a particular embodiment, the present invention provides a method for diagnosing IBS, comprising detecting bacterial taxa which are predictive of IBS selected from table 2. In certain embodiments, the bacterial taxa predictive of IBS are significantly more abundant in patients suffering from IBS, for example as shown in tables 2 and/or 3. In other embodiments, the bacterial taxa predictive of IBS is significantly less abundant in patients suffering from IBS, for example as shown in tables 2 and/or 3.

In one embodiment, a bacterial species or strain predictive of IBS is differentially abundant in patients suffering from IBS. In a particular embodiment, the method of the invention comprises measuring the abundance of a bacterial species, wherein differential abundance is associated with IBS, and wherein the species is selected from: Ruminococcus gnavus, Clostridium bolteae, Anaerotruncus colihominis, Flavonifractor plautii, Clostridium clostridioforme, Clostridium hathewayi, Clostridium symbiosum, Ruminococcus torques, Alistipes senegalensis, Prevotella copri, Eggerthella lenta, Clostridium asparagiforme, Barnesiella intestinihominis, Clostridium citroniae, Eubacterium eligens, Clostridium ramosum, Coprococcus catus, Eubacterium biforme, Ruminococcus lactaris, Bacteroides massiliensis, Haemophilus parainfluenzae, Clostridium nexile, Clostridium innocuum, Bacteroides Xylanisolvens, Oxalobacter formigenes, Alistipes putredinis, Paraprevotella clara and/or Odoribacter splanchnicus. In a particular embodiment, the method of the invention comprises measuring the abundance of a bacterial strain, wherein differential abundance is associated with IBS, and wherein the strain is selected from: Clostridiales bacterium 1 7 47FAA, Lachnospiraceae bacterium 1 4 56FA, Lachnospiraceae bacterium 51 57FAA, Lachnospiraceae bacterium 3 1 46FAA, Lachnospiraceae bacterium 7 1 58FAA, Coprococcus sp ART55 1, Lachnospiraceae bacterium 3 1 57FAA CT1, Lachnospiraceae bacterium 2 1 58FAA and/or Eubacterium sp 3 1 31. In certain embodiments, the bacteria (i.e. one or more bacterial strains) may be detected using Clade-specific bacterial genes, 16S sequences, transcriptomics or metabolomics.

In one embodiment, a bacterial species or strain predictive of IBS is differentially abundant in patients suffering from IBS. In a particular embodiment, the method of the invention comprises measuring the abundance of a bacterial species, wherein differential abundance is associated with IBS, and wherein the species is selected from: Escherichia coli, Streptococcus aginosus, Parabacteroides johnsonii, Streptococcus gordonii, Clostridium boltae, Turicibacter sanguinis, Paraprevotella Xylamphila, Streptococcus mutans, Bacteroides plebeius, Clostridium clostridioforme, Klebsiella pneumoniae, Clostridium hathewayi, Bacteroides fragilis, Prevotella disiens, Clostridium leptum, Pseudoflavonifractor capillosus, Bacteroides intestinalis, Enterococcus faecalis, Streptococcus infantis, Alistipes shahii, Clostridium asparagiforme, Clostridium symbiosum and/or Streptococcus sanguinis. In a particular embodiment, the method of the invention comprises measuring the abundance of a bacterial strain, wherein differential abundance is associated with IBS, and wherein the strain is selected from: Clostridiales bacterium 1 7 47FAA, Eubacterium sp 3 1 31, Lachnospiraceae bacterium 5 1 57FAA, Clostridiaceae bacterium JC118 and/or Lachnospiraceae bacterium 1 4 56FA. In certain embodiments, the bacteria (i.e. one or more bacterial strains) may be detected using Clade-specific bacterial genes, 16S sequences, transcriptomics or metabolomics.

In one embodiment, the fecal microbiota alpha diversity of patients with IBS is reduced. In one embodiment, the intra-individual microbiota diversity of patients with IBS is reduced. In one embodiment, the fecal microbiota alpha diversity of patients with IBS is significantly lower than non-IBS patients. In one embodiment, the intra-individual microbiota diversity of patients with IBS is significantly lower than non-IBS patients. In a further embodiment, the microbiota alpha diversity is not significantly different between IBS clinical subtypes.

In one embodiment, the present invention provides a method for diagnosing IBS, comprising detecting one or more bacterial strains belonging to an operational taxonomic unit (OTU) associated with IBS. In preferred embodiments, the OTU is selected from table 11. In one embodiment, the OTU associated with IBS is classified as belonging to the Firmicutes phylum. In a particular embodiment, the OTU associated with IBS is classified as belonging to the Clostridia class. In a particular embodiment, the OTU associated with IBS is classified as belonging to the Clostridiales order. In a particular embodiment, the OTU associated with IBS is classified as belonging to the Clostridiales Lachnospiraceae family or the Ruminococcaceae family. In a particular embodiment, the OTU associated with IBS is classified as belonging to the Butyricicoccus genus.

In one embodiment, the present invention provides a method for diagnosing IBS, comprising detecting bacterial strains belonging to one or more OTUs listed in Table 11. The sequences in Table 12 can be used to classify bacteria as belonging to the OTUs listed in Table 11. Bacteria (i.e. one or more bacterial strains) having at least 97% sequence identity to the sequences in Table 12 belong to the corresponding OTUs in Table 11. The alignment is across the length of the sequence. In both Metaphlan2 and HUMAnN2 runs, alignment for species composition is done using bowtie 2. Bowtie2 is run with “very-sensitive argument” and the alignment performed is “Global alignment”.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 1. In certain such embodiments, the bacteria is classified as belonging to the Lachnospiraceae family.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 2. In certain such embodiments, the bacteria is classified as belonging to the Firmicutes phylum.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 3. In certain such embodiments, the bacteria is classified as belonging to the Butyricicoccus genus.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 4. In certain such embodiments, the bacteria is classified as belonging to the Lachnospiraceae family.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 5. In certain such embodiments, the bacteria is classified as belonging to the Clostridiales order.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 6. In certain such embodiments, the bacteria is classified as belonging to the Ruminococcaceae family.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 7. In certain such embodiments, the bacteria is classified as belonging to the Ruminococcaceae family.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 8. In certain such embodiments, the bacteria is classified as belonging to the Firmicutes phylum.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No: 9. In certain such embodiments, the bacteria is classified as belonging to the Ruminococcaceae family.

In one embodiment, the present invention provides a method for diagnosing IBS, comprising detecting bacteria (i.e. one or more bacterial strains) having a 16S rRNA gene sequence at least 97% (e.g. 98%, 99%, 99.5% or 100%) identical to SEQ ID No:10. In certain such embodiments, the bacteria is classified as belonging to the Lachnospiraceae family.

In preferred embodiments, the invention provides a method for diagnosing IBS, comprising detecting different bacteria (i.e. one or more bacterial strains) having 16S rRNA gene sequences at least 97% (e.g. 98%, 99%, 99.5% or 100%) identical to two or more of SEQ ID No:1-10, such as 5, 8, or all of SEQ ID No:1-10.

Alteration of Pathways as a Predictor of IBS

The inventors have identified that certain pathways are over or underrepresented in the genomes of the microbiota of patients suffering from IBS. Therefore, the invention provides methods for diagnosing IBS based on the presence or abundance of genes, pathways, or bacteria carrying such genes. Methods of diagnosis comprising detecting genes involved in one or more of the pathways identified herein may be particularly useful for use with different populations of patients because different patient populations may have different microbiome populations.

In one embodiment, the present invention provides a method for diagnosing IBS, comprising detecting microbial genes involved in one or more of the pathways selected from the list in table 4. In certain embodiments, the presence, or increased abundance relative to a control (non-IBS) individual, of genes involved in a pathway recited in Table 4 is associated with IBS. In a preferred embodiment, the method comprises detecting genes involved in amino acid biosynthesis/degradation pathways. The data show that these pathways are significantly more abundant in patients with IBS. In a preferred embodiment, the method comprises detecting genes involved in starch degradation V pathway. The data show that such genes are significantly more abundant in patients with IBS. In another embodiment, genes that are significantly more abundant in patients with IBS are associated with Lachnospiraceae and Ruminococcus species. In certain embodiments, the method of the invention comprises detecting genes involved in at least 2, 5, 10, 15, 20 or 30 of the pathways in table 4. In any such embodiments, detecting the genes comprises measuring the relative abundance of the genes, or bacteria carrying the genes in a sample, for example relative to a corresponding sample from a control (non-IBS) individual or relative to a reference value. In certain embodiments, the presence of the microbial genes is detected by detecting metabolites in the sample. In certain embodiments, the presence of the microbial genes is detected by detecting a taxa of bacteria know to carry the microbial genes.

In other embodiments, the absence or decreased abundance relative to a control (non-IBS) individual of genes involved in a pathway are associated with IBS, for example as shown in table 4. In a preferred embodiment, genes involved in galactose degradation, sulfate reduction, sulfate assimilation and cysteine biosynthesis pathways are detected. The data show that these pathways are significantly less abundant in patients with IBS. In a particular embodiment, pathways indicative of sulphur metabolism are less abundant in patients with IBS. In any such embodiments, detecting the genes comprises measuring the relative abundance of the genes, or bacteria carrying the genes in a sample, for example relative to a corresponding sample from a control (non-IBS) individual or relative to a reference value.

In certain embodiments, methods comprising detecting the presence or absence or relative abundance of genes involved in a pathway comprise detecting nucleic acid sequences in a sample from the patient. Additionally or alternatively, the methods comprise detecting bacterial species known to carry the genes of the relevant pathway.

In one embodiment, the present invention provides a method for diagnosing IBS, comprising detecting the differential abundance of one or more pathways predictive of IBS relative to control (non-IBS) individuals. In a particular embodiment, the adenosine ribonucleotide de novo biosynthesis functional pathway is differentially abundant in IBS relative to control (non-IBS) individuals. In a preferred embodiment, the adenosine ribonucleotide de novo biosynthesis functional pathway is more abundant in IBS patients relative to control (non-IBS) individuals.

Alteration of Metabolomes as a Predictor of IBS

The inventors have identified metabolites that are associated with IBS and the invention provides methods for diagnosing IBS that comprise detecting such metabolites. Methods of diagnosis comprising detecting metabolites identified herein may be particularly useful for use with different populations of patients because different patient populations may have different microbiome populations, but there may be more uniformity in terms of detectable metabolites. Generally, detecting a metabolite associated with IBS in the methods of the invention comprises measuring the concentration of the metabolite in a sample or measuring changes in the concentration of a metabolite and optionally comparing the concentration to a corresponding sample from a control (non-IBS) individual or relative to a reference value. In some embodiments, detecting a metabolite associated with IBS in the methods of the invention comprises measuring the concentration of a precursor of the metabolite and optionally comparing the concentration to a corresponding sample from a control (non-IBS) individual or relative to a reference value. In some embodiments, detecting a metabolite associated with IBS in the methods of the invention comprises measuring the concentration of a breakdown product of the metabolite and optionally comparing the concentration to a corresponding sample from a control (non-IBS) individual or relative to a reference value. In certain embodiments, the method comprises detecting a bacterial taxa known to produce a metabolite predictive of IBS.

Alteration of Urine Metabolomes as a Predictor of Ibs

In one embodiment, the present invention provides a method for diagnosing IBS, comprising detecting urine metabolites which may include one or more of the following: A 80987, Ala-Leu-Trp-Gly, Medicagenic acid 3-O-b-D-glucuronide and/or (−)-Epigallocatechin sulfate. In one embodiment, the present invention provides a method for diagnosing IBS, comprising detecting one or more urine metabolites selected from the list in table 5. In any such embodiments, detecting the metabolite comprises measuring the concentration of the metabolite in a sample, for example relative to a corresponding sample from a control (non-IBS) individual or relative to a reference value. In other embodiments, detecting the metabolite comprises measuring the concentration of the metabolite in a sample, and normalising the concentration relative to urine creatinine levels in each sample. In some embodiments, the method comprises detecting a precursor or breakdown product of the above metabolites. In one embodiment, machine learning is applied to urine metabolome data to diagnose IBS.

In a particular embodiment, the method comprises detecting adenosine, such as measuring the concentration of adenosine in a sample. The examples demonstrate that adenosine is more abundant in IBS patients relative to control (non-IBS) individuals. Thus, a level of adenosine that is increased relative to a healthy control is indicative of IBS.

In one embodiment, the present invention provides a method for diagnosing IBS, comprising detecting one or more urine metabolites that are differentially abundant in patients suffering from IBS compared to a healthy control (i.e. from one or more subjects who does not suffer from IBS). In one embodiment, the one or more urine metabolites that are differentially abundant in patients suffering from IBS are: N-Undecanoylglycine, Gamma-glutamyl-Cysteine, Alloathyriol, Trp-Ala-Pro, A 80987, Medicagenic acid 3-O-b-D-glucuronide, Ala-Leu-Trp-Gly, Butoctamide hydrogen succinate, (−)-Epicatechin sulfate, 1,4,5-Trimethyl-naphtalene, Tricetin 3′-methyl ether 7,5′-diglucuronide, Torasemide, (−)-Epigallocatechin sulfate, Dodecanedioylcarnitine, 1,6,7-Trimethylnaphthalene, Tetrahydrodipicolinate, Sumiki's acid, Silicic acid, Delphinidin 3-(6″-O-4-malyl-glucosyl)-5-glucoside, L-Arginine, Leucyl-Methionine, Phe-Gly-Gly-Ser, Gin-Met-Pro-Ser, Creatinine, Ala-Asn-Cys-Gly, 2-hydroxy-2-(hydroxymethyl)-2H-pyran-3(6H)-one, Thiethylperazine, 5-((2-iodoacetamido)ethyl)-1-aminonapthalene sulfate, dCTP, Isoleucyl-Proline, 3,4-Methylenesebacic acid, Dimethylallylpyrophosphate/Isopentenyl pyrophosphate, (4-Hydroxybenzoyl)choline, Diazoxide, 3,5-Di-O-galloyl-1,4-galactarolactone, 2-Hydroxypyridine, Decanoylcarnitine, Asp-Met-Asp-Pro, 3-Methyldioxyindole, (1S,3R,4S)-3,4-Dihydroxycyclohexane-1-carboxylate, Ala-Lys-Phe-Cys, 3-Indolehydracrylic acid, [FA (18:0)] N-(9Z-octadecenoyl)-taurine, Ferulic acid 4-sulfate, Urea, N-Carboxyacetyl-D-phenylalanine, 4-Methoxyphenylethanol sulfate, UDP-4-dehydro-6-deoxy-D-glucose, Linalyl formate, Demethyloleuropein, 5′-Guanosyl-methylene-triphosphate, Allyl nonanoate, 2-Phenylethyl octanoate, beta-Cellobiose, D-Galactopyranosyl-(1->3)-D-galactopyranosyl-(1->3)-L-arabinose, Cys-Phe-Phe-Gln, Hippuric acid, Cys-Pro-Pro-Tyr, Met-Met-Thr-Trp, methylphosphonate, 3′-Sialyllactosamine, 2,4,6-Octatriynoic acid, Delphinidin 3-O-3″,6″-O-dimalonylglucoside, L-Valine, Met-Met-Cys, Cysteinyl-Cysteine, (all-E)-1,8,10-Heptadecatriene-4,6-diyne-3,12-diol, L-Lysine, Pivaloylcarnitine, Lenticin, Phenol glucuronide, Tyrosyl-Cysteine, Osmundalin, Tetrahydroaldosterone-3-glucuronide, N-Methylpyridinium, L-prolyl-L-proline, Glutarylcarnitine, [FA (15:4)] 6,8,10,12-pentadecatetraenal, Methyl bisnorbiotinyl ketone, Acetoin, LysoPC(18:2(9Z,12Z)), Hexyl 2-furoate, N-carbamoyl-L-glutamate, L-Homoserine, L-Asparagine, Tiglylcarnitine, Thymine, 3-hydroxypyridine, Menadiol disuccinate, 9-Decenoylcarnitine, Pyrocatechol sulfate, sedoheptulose anhydride, (+)-gamma-Hydroxy-L-homoarginine, Thioridazine, Cys-Glu-Glu-Glu, Marmesin rutinoside, L-Serine, L-Urobilinogen, Isobutyrylglycine, S-Adenosylhomocysteine, 2,3-dioctanoylglyceramide, 3-Methoxy-4-hydroxyphenylglycol glucuronide, sulfoethylcysteine, Hydroxyphenylacetylglycine, Pyrroline hydroxycarboxylic acid, 1-(alpha-Methyl-4-(2-methylpropyl)benzeneacetate)-beta-D-Glucopyranuronic acid, 2-Methylbutylacetate, N1-Methyl-4-pyridone-3-carboxamide, Cortolone-3-glucuronide, Asn-Cys-Gly, N6,N6,N6-Trimethyl-L-lysine, Benzylamine, 5-Hydroxy-L-tryptophan, Armillaric acid, Leucine/Isoleucine, 2-Butylbenzothiazole, D-Sedoheptulose 7-phosphate, [Fv Dimethoxy,methyl(9:1)] (2S)-5,7-Dimethoxy-3′,4′-methylenedioxyflavanone, Oxoadipic acid, Thr-Cys-Cys, Creatine, Hydroxybutyrylcarnitine, 5′-Dehydroadenosine, Phe-Thr-Val, dUDP, L-Glutamine and/or Kaempferol 3-(2″,3″-diacetyl-4″-p-coumaroylrhamnoside). In one embodiment, the present invention provides a method for diagnosing IBS, comprising detecting one or more urine metabolites predictive of IBS. In one embodiment, the urine metabolite predictive of IBS is selected from: N-Undecanoylglycine, Gamma-glutamyl-Cysteine, Alloathyriol, Trp-Ala-Pro, A 80987, Medicagenic acid 3-O-b-D-glucuronide, Ala-Leu-Trp-Gly, Butoctamide hydrogen succinate, (−)-Epicatechin sulfate, 1,4,5-Trimethyl-naphtalene, Tricetin 3′-methyl ether 7,5′-diglucuronide, Torasemide, (−)-Epigallocatechin sulfate, Dodecanedioylcarnitine, 1,6,7-Trimethylnaphthalene, Tetrahydrodipicolinate, Sumiki's acid, Silicic acid, Delphinidin 3-(6″-O-4-malyl-glucosyl)-5-glucoside, L-Arginine, Leucyl-Methionine, Phe-Gly-Gly-Ser, Gin-Met-Pro-Ser, Creatinine, Ala-Asn-Cys-Gly, 2-hydroxy-2-(hydroxymethyl)-2H-pyran-3(6H)-one, Thiethylperazine, 5-((2-iodoacetamido)ethyl)-1-aminonapthalene sulfate, dCTP, Isoleucyl-Proline, 3,4-Methylenesebacic acid, Dimethylallylpyrophosphate/Isopentenyl pyrophosphate, (4-Hydroxybenzoyl)choline, Diazoxide, 3,5-Di-O-galloyl-1,4-galactarolactone, 2-Hydroxypyridine, Decanoylcarnitine, Asp-Met-Asp-Pro, 3-Methyldioxyindole, (1S,3R,4S)-3,4-Dihydroxycyclohexane-1-carboxylate, Ala-Lys-Phe-Cys, 3-Indolehydracrylic acid, [FA (18:0)] N-(9Z-octadecenoyl)-taurine, Ferulic acid 4-sulfate, Urea, N-Carboxyacetyl-D-phenylalanine, 4-Methoxyphenylethanol sulfate, UDP-4-dehydro-6-deoxy-D-glucose, Linalyl formate, Demethyloleuropein, 5′-Guanosyl-methylene-triphosphate, Allyl nonanoate, 2-Phenylethyl octanoate, beta-Cellobiose, D-Galactopyranosyl-(1->3)-D-galactopyranosyl-(1->3)-L-arabinose, Cys-Phe-Phe-Gln, Hippuric acid, Cys-Pro-Pro-Tyr, Met-Met-Thr-Trp, methylphosphonate, 3′-Sialyllactosamine, 2,4,6-Octatriynoic acid, Delphinidin 3-O-3″,6″-0-dimalonylglucoside, L-Valine, Met-Met-Cys, Cysteinyl-Cysteine, (all-E)-1,8,10-Heptadecatriene-4,6-diyne-3,12-diol, L-Lysine, Pivaloylcarnitine, Lenticin, Phenol glucuronide, Tyrosyl-Cysteine, Osmundalin, Tetrahydroaldosterone-3-glucuronide, N-Methylpyridinium, L-prolyl-L-proline, Glutarylcarnitine, [FA (15:4)] 6,8,10,12-pentadecatetraenal, Methyl bisnorbiotinyl ketone, Acetoin, LysoPC(18:2(9Z,12Z)), Hexyl 2-furoate, N-carbamoyl-L-glutamate, L-Homoserine, L-Asparagine, Tiglylcarnitine, Thymine, 3-hydroxypyridine, Menadiol disuccinate, 9-Decenoylcarnitine, Pyrocatechol sulfate, sedoheptulose anhydride, (+)-gamma-Hydroxy-L-homoarginine, Thioridazine, Cys-Glu-Glu-Glu, Marmesin rutinoside, L-Serine, L-Urobilinogen, Isobutyrylglycine, S-Adenosylhomocysteine, 2,3-dioctanoylglyceramide, 3-Methoxy-4-hydroxyphenylglycol glucuronide, sulfoethylcysteine, Hydroxyphenylacetylglycine, Pyrroline hydroxycarboxylic acid, 1-(alpha-Methyl-4-(2-methylpropyl)benzeneacetate)-beta-D-Glucopyranuronic acid, 2-Methylbutylacetate, N1-Methyl-4-pyridone-3-carboxamide, Cortolone-3-glucuronide, Asn-Cys-Gly, N6,N6,N6-Trimethyl-L-lysine, Benzylamine, 5-Hydroxy-L-tryptophan, Armillaric acid, Leucine/Isoleucine, 2-Butylbenzothiazole, D-Sedoheptulose 7-phosphate, [Fv Dimethoxy,methyl(9:1)] (2S)-5,7-Dimethoxy-3′,4′-methylenedioxyflavanone, Oxoadipic acid, Thr-Cys-Cys, Creatine, Hydroxybutyrylcarnitine, 5′-Dehydroadenosine, Phe-Thr-Val, dUDP, L-Glutamine and/or Kaempferol 3-(2″,3″-diacetyl-4″-p-coumaroylrhamnoside).. In one embodiment, the present invention provides a method for diagnosing IBS, comprising detecting differential abundance of one or more urine metabolites selected from the list in table 6. In certain embodiments, the method of the invention comprises detecting 2, 5, 10, 15 or 20 or all of the metabolites from table 6. In any such embodiments, detecting the metabolite comprises measuring the concentration of the metabolite in a sample, for example the concentration relative to a corresponding sample from a control (non-IBS) individual or relative to a reference value. In some embodiments, detecting the metabolite comprises measuring the concentration of the metabolite in a sample, and normalising the concentration relative to urine creatinine levels in each sample. In some embodiments, the method comprises detecting a precursor or breakdown product of the above metabolites.

In certain embodiments, the abundance of urine metabolites is significantly increased in patients with IBS, for example as shown in table 6. In one embodiment, the method comprises detecting metabolites involved in fatty acid oxidation and/or fatty acid metabolism, which are significantly more abundant in patients with IBS. In a preferred embodiment, N-Undecanoylglycine is detected, which is significantly more abundant in patients with IBS. In another preferred embodiment, Decanoylcarnitine is detected, which is significantly more abundant in patients with IBS.

In one embodiment, a urine metabolite predictive of IBS is more abundant in patients suffering from IBS compared to a healthy control. In one embodiment, the present invention provides a method for diagnosing IBS, comprising detecting one or more urine metabolites that have been found to be predictive that a patient is suffering from IBS. In one embodiment, the present invention provides a method for diagnosing IBS, comprising detecting one or more urine metabolites that are more abundant in patients suffering from IBS compared to a healthy control (i.e. from one or more subjects who does not suffer from IBS). In certain embodiments, the abundance of urine metabolites is increased in patients with IBS, for example as shown in table 6 and/or table 21b. In one embodiment, the one or more urine metabolites that are more abundant in patients suffering from IBS are: A 80987, Medicagenic acid 3-O-b-D-glucuronide, N-Undecanoylglycine, Ala-Leu-Trp-Gly, Gamma-glutamyl-Cysteine, Butoctamide hydrogen succinate, (−)-Epicatechin sulfate, 1,4,5-Trimethyl-naphtalene, Trp-Ala-Pro, Dodecanedioylcarnitine, 1,6,7-Trimethylnaphthalene, Sumiki's acid, Phe-Gly-Gly-Ser, 2-hydroxy-2-(hydroxymethyl)-2H-pyran-3(6H)-one, 5-((2-iodoacetamido)ethyl)-1-aminonapthalene sulfate, Thiethylperazine, dCTP, Dimethylallylpyrophosphate/Isopentenyl pyrophosphate, Asp-Met-Asp-Pro, 3,5-Di-O-galloyl-1,4-galactarolactone, Decanoylcarnitine, [FA (18:0)] N-(9Z-octadecenoyl)-taurine, UDP-4-dehydro-6-deoxy-D-glucose, Delphinidin 3-O-3″,6″-O-dimalonylglucoside, Osmundalin and/or Cysteinyl-Cysteine. In a preferred embodiment, one or more urine metabolites selected from: A 80987, Medicagenic acid 3-O-b-D-glucuronide, N-Undecanoylglycine, Ala-Leu-Trp-Gly, and/or Gamma-glutamyl-Cysteine are detected, which are more abundant in patients with IBS compared to healthy controls. In one embodiment, the present invention provides a method for diagnosing IBS, comprising detecting an increase in abundance of one or more urine metabolites selected from the list in table 6 and/or table 21b. In certain embodiments, the method of the invention comprises detecting 2, 5, 10, 15 or 20 or all of the metabolites from table 6 and/or table 21b. In any such embodiments, detecting the metabolite comprises measuring the concentration of the metabolite in a sample, for example the concentration relative to a corresponding sample from a control (non-IBS) individual or relative to a reference value. In some embodiments, detecting the metabolite comprises measuring the concentration of the metabolite in a sample, and normalising the concentration relative to urine creatinine levels in each sample. In some embodiments, the method comprises detecting a precursor or breakdown product of the above metabolites. In a preferred embodiment, epicatechin sulfate is detected, which is more abundant in patients with IBS. In a preferred embodiment, medicagenic acid 3-O-b-D-glucuronide is detected, which is more abundant in patients with IBS.

In certain embodiments, the abundance of urine metabolites is significantly decreased in patients with IBS, for example as shown in table 6. In one embodiment, the method comprises detecting metabolites involved in the biosynthesis of nitric oxide, which are significantly less abundant in patients with IBS. In one embodiment amino acids are significantly less abundant in patients with IBS, for example L-arginine.

In one embodiment, a urine metabolite predictive of IBS is less abundant in patients suffering from IBS compared to a healthy control. In one embodiment, the present invention provides a method for diagnosing IBS, comprising detecting one or more urine metabolites that have been found to be predictive that a patient is not suffering from IBS, i.e. that the patient is a healthy control. In one embodiment, the present invention provides a method for diagnosing IBS, comprising detecting one or more urine metabolites that are less abundant in patients suffering from IBS compared to a healthy control (i.e. from one or more subjects who does not suffer from IBS). In one embodiment, the present invention provides a method for diagnosing IBS, comprising detecting one or more urine metabolites that are more abundant in healthy controls (i.e. from one or more subjects who does not suffer from IBS) compared to patients suffering from IBS. In certain embodiments, the abundance of urine metabolites is decreased in patients with IBS, for example as shown in table 6 and/or table 21a. In one embodiment, the one or more urine metabolites that are less abundant in patients suffering from IBS are: Tricetin 3′-methyl ether 7,5′-diglucuronide, Alloathyriol, Torasemide, (−)-Epigallocatechin sulfate, Tetrahydrodipicolinate, Silicic acid, Delphinidin 3-(6″-O-4-malyl-glucosyl)-5-glucoside, Creatinine, L-Arginine, Leucyl-Methionine, Gln-Met-Pro-Ser, Ala-Asn-Cys-Gly, Isoleucyl-Proline, 3,4-Methylenesebacic acid, (4-Hydroxybenzoyl)choline, Diazoxide, (1S,3R,4S)-3,4-Dihydroxycyclohexane-1-carboxylate, 2-Hydroxypyridine, Ala-Lys-Phe-Cys, 3-Methyldioxyindole, N-Carboxyacetyl-D-phenylalanine, Urea, Ferulic acid 4-sulfate, 3-Indolehydracrylic acid, Demethyloleuropein, 5′-Guanosyl-methylene-triphosphate, Linalyl formate, 4-Methoxyphenylethanol sulfate, Allyl nonanoate, D-Galactopyranosyl-(1->3)-D-galactopyranosyl-(1->3)-L-arabinose, Met-Met-Thr-Trp, Cys-Pro-Pro-Tyr, methylphosphonate, 2-Phenylethyl octanoate, Hippuric acid, Glutarylcarnitine and/or Cys-Phe-Phe-Gln. In a preferred embodiment, one or more urine metabolites selected from: Tricetin 3′-methyl ether 7,5′-diglucuronide, Alloathyriol, Torasemide, (−)-Epigallocatechin sulfate and/or Tetrahydrodipicolinate are detected, which are less abundant in patients with IBS compared to healthy controls. In one embodiment, the present invention provides a method for diagnosing IBS, comprising detecting a decrease in abundance of one or more urine metabolites selected from the list in table 6 and/or table 21a. In certain embodiments, the method of the invention comprises detecting 2, 5, 10, 15 or 20 or all of the metabolites from table 6 and/or table 21a. In any such embodiments, detecting the metabolite comprises measuring the concentration of the metabolite in a sample, for example the concentration relative to a corresponding sample from a control (non-IBS) individual or relative to a reference value. In some embodiments, detecting the metabolite comprises measuring the concentration of the metabolite in a sample, and normalising the concentration relative to urine creatinine levels in each sample. In some embodiments, the method comprises detecting a precursor or breakdown product of the above metabolites.

In one embodiment, the present invention provides a method for diagnosing IBS, comprising detecting one or more urine metabolites that are differentially abundant in patients suffering from IBS compared to a healthy control (i.e. from one or more subjects who does not suffer from IBS). In a preferred embodiment, the one or more urine metabolites that are differentially abundant in patients suffering from IBS are sulfate, glucuronide, carnitine, glycine and glutamine conjugates. In one embodiment, the method comprises detecting metabolites involved in phase 2 metabolism, which are is upregulated in patients with IBS. In any such embodiments, detecting the metabolite comprises measuring the concentration of the metabolite in a sample, for example the concentration relative to a corresponding sample from a control (non-IBS) individual or relative to a reference value. In other embodiments, detecting the metabolite comprises measuring the concentration of the metabolite in a sample, and normalising the concentration relative to urine creatinine levels in each sample.

Alteration of Fecal Metabolomes as a Predictor of IBS

In one embodiment, the present invention provides a method for diagnosing IBS, comprising detecting one or more fecal metabolites selected from: 3-deoxy-D-galactose, Tyrosine, I-Urobilin, Adenosine, Glu-Ile-Ile-Phe, 3,6-Dimethoxy-19-norpregna-1,3,5,7,9-pentaen-20-one, 2-Phenylpropionate, MG(20:3(8Z,11Z,14Z)/0:0/0:0), 1,2,3-Tris(1-ethoxyethoxy)propane, Staphyloxanthin, Hexoses, 20-hydroxy-E4-neuroprostane, Nonyl acetate, 3-Feruloyl-1,5-quinolactone, trans-2-Heptenal, Pyridoxamine, L-Arginine, Dodecanedioic acid, Ursodeoxycholic acid, 1-(Malonylamino)cyclopropanecarboxylic acid, Cortisone, 9,10,13-Trihydroxystearic acid, Glu-Ala-Gln-Ser, Quasiprotopanaxatriol, N-Methylindolo[3,2-b]-5alpha-cholest-2-ene, PG(20:0/22:1(11Z)), (−)-Epigallocatechin, 2-Methyl-3-ketovaleric acid, Secoeremopetasitolide B, PC(20:1(11Z)/P-16:0), Glu-Asp-Asp, N5-acetyl-N5-hydroxy-L-ornithine acid, Silicic acid, (1xi,3xi)-1,2,3,4-Tetrahydro-1-methyl-beta-carboline-3-carboxylic acid, PS(36:5), Chorismate, Isoamyl isovalerate, PA(0-36:4), PE(P-28:0) and/or gamma-Glutamyl-S-methylcysteinyl-beta-alanine. In certain embodiments, the method of the invention comprises detecting at least 2, 5, 10, 15 or 20 or all of these metabolites. In any such embodiments, detecting the metabolite comprises measuring the concentration of the metabolite in a sample, for example the concentration relative to a corresponding sample from a control (non-IBS) individual or relative to a reference value.

In one embodiment, the invention provides a method for diagnosing IBS, comprising detecting one or more fecal metabolites selected from: L-Phenylalanine, Adenosine, MG(20:3(8Z,11Z,14Z)/0:0/0:0), L-Alanine, 3,6-Dimethoxy-19-norpregna-1,3,5,7,9-pentaen-20-one, Glu-Ile-Ile-Phe, Glu-Ala-Gln-Ser, 2,4,8-Eicosatrienoic acid isobutylamide, Piperidine, Staphyloxanthin, beta-Carotinal, Hexoses, Ile-Arg-Ile, 11-Deoxocucurbitacin I, 1-(Malonylamino)cyclopropanecarboxylic acid, PG(37:2), [PR] gamma-Carotene/beta,psi-Carotene, 20-hydroxy-E4-neuroprostane, Ethylphenyl acetate, Dodecanedioic acid, Ile-Lys-Cys-Gly, Tuberoside, D-galactal, 3,6-Dihydro-4-(4-methyl-3-pentenyl)-1,2-dithiin, demethylmenaquinone-6, L-Arginine, PC(o-16:1(9Z)/14:1(9Z)), Mesobilirubinogen, Traumatic acid, alpha-Tocopherol succinate, 3-Methylcrotonylglycine, (S)-(E)-8-(3,6-Dimethyl-2-heptenyl)-4′,5,7-trihydroxyflavanone, xi-7-Hydroxyhexadecanedioic acid, beta-Pinene, Leu-Ser-Ser-Tyr, Orotic acid, Heptane-1-thiol, Glu-Asp-Asp, LysoPE(18:2(9Z,12Z)/0:0), LysoPE(22:0/0:0), Creatine, Inosine, SM(d32:2), Arg-Leu-Val-Cys, PS(0-18:0/15:0), Pyridoxamine, N-Heptanoylglycine, Hematoporphyrin IX, 3beta,5beta-Ketotriol, 2-Phenylpropionate, trans-2-Heptenal, LysoPC(0:0/18:0), Linoleoyl ethanolamide, LysoPE(24:0/0:0), 2-Methyl-3-hydroxyvaleric acid, Quasiprotopanaxatriol, N-oleoyl isoleucine, (−)-(E)-1-(4-Hydroxyphenyl)-7-phenyl-6-hepten-3-ol, [FA hydroxy(4:0)] N-(3S-hydroxy-butanoyl)-homoserine lactone, Riboflavin cyclic-4′,5′-phosphate, Arg-Lys-Trp-Val, PC(20:1(11Z)/P-16:0), 3,5-Dihydroxybenzoic acid, Tyrosine, 2,3-Epoxymenaquinone, His-Met-Val-Val, PI(41:2), Phenol, 3,3′-Dithiobis[2-methylfuran], Ala-Leu-Trp-Pro, 1,2,3-Tris(1-ethoxyethoxy)propane, Vanilpyruvic acid, 2-Hydroxy-3-carboxy-6-oxo-7-methylocta-2,4-dienoate, Secoeremopetasitolide B, 2-O-Benzoyl-D-glucose, Ile-Leu-Phe-Trp, (R)-lipoic acid, PA(20:4(5Z,8Z,11Z,14Z)e/2:0), PE(P-16:0e/0:0), Benzyl isobutyrate, Hexyl 2-furoate, Trp-Ala-Ser, LysoPC(15:0), 4-Hydroxycrotonic acid, 3-Feruloyl-1,5-quinolactone, Furfuryl octanoate, PC(22:2(13Z,16Z)/15:0), (−)-1-Methylpropyl 1-propenyl disulphide, PC (36:6), Leucyl-Glycine, CE(16:2), Triterpenoid, Violaxanthin, [FA hydroxy(17:0)] heptadecanoic acid, 2-Hydroxyundecanoate, Chorismate, delta-Dodecalactone, 3-O-Protocatechuoylceanothic acid, PG(16:1(9Z)/16:1(9Z)), p-Cresol sulfate, Quercetin 3′-sulfate, PS(26:0)), Ala-Leu-Phe-Trp, L-Glutamic acid 5-phosphate, N,2,3-Trimethyl-2-(1-methylethyl)butanamide, Isoamyl isovalerate, n-Dodecane, PC(14:1(9Z)/14:1(9Z)), Lucyoside Q, Endomorphin-1, 3-Hydroxy-10′-apo-b,y-carotenal, Pyrroline hydroxycarboxylic acid, S-Propyl 1-propanesulfinothioate, N-Methylindolo[3,2-b]-5alpha-cholest-2-ene, Tocopheronic acid, 1-(2,4,6-Trimethoxyphenyl)-1,3-butanedione, Homogentisic acid, LysoPE(18:1(9Z)/0:0), N-stearoyl valine, trans-Carvone oxide, 1,1′-Thiobis-1-propanethiol, 2-(Ethylsulfonylmethyl)phenyl methylcarbamate, menaquinone-4, Benzeneacetamide-4-O-sulphate, N5-acetyl-N5-hydroxy-L-ornithine, Succinic acid, Asn-Lys-Val-Pro, LysoPC(14:1(9Z)), Phenol glucuronide, 2-methyl-Butanoic acid, 2-methylbutyl ester, 3-O-Caffeoyl-1-O-methylquinic acid, [FA hydroxy(24:0)] 3-hydroxy-tetracosanoic acid, N-(2-hydroxyhexadecanoyl)-sphinganine-1-phospho-(1′-myo-inositol), gamma-Dodecalactone, PA(22:1(11Z)/0:0), Butyl butyrate, TG(20:5(5Z,8Z,11Z,14Z,17Z)/18:1(9Z)/22:5(7Z,10Z,13Z,16Z,19Z))[iso6], Clausarinol, 4-Methyl-2-pentanone, Trigoneline, Arg-Val-Pro-Tyr, 2,3-Methylenesuccinic acid, Serinyl-Threonine, Lycoperoside D, Geraniol, 1-18:2-lysophosphatidylglycerol, omega-6-Hexadecalactone, Ambrettolide, gamma-Glutamyl-S-methylcysteinyl-beta-alanine, FA oxo(22:0), D-Ribose, LysoPC(17:0), PA(0-36:4), C19 Sphingosine-1-phosphate, 4-Hydroxy-5-(dihydroxyphenyl)-valeric acid-O-methyl-O-sulphate, PE(14:1(9Z)/14:0), Citronellyl tiglate, Ethyl methylphenylglycidate (isomer 1), N-Acetyl-leu-leu-tyr and/or PS(O-34:3). In certain embodiments, the method of the invention comprises detecting at least 2, 5, 10, 15 or 20 or all of these metabolites. In any such embodiments, detecting the metabolite comprises measuring the concentration of the metabolite in a sample, for example the concentration relative to a corresponding sample from a control (non-IBS) individual or relative to a reference value.

In a preferred embodiment, method comprises detecting the fecal metabolite L-tyrosine. In a preferred embodiment, the method comprises detecting L-arginine. In a preferred embodiment, method comprises detecting the bile acid ursodeoxycholic acid (UDCA). In a preferred embodiment, the method comprises detecting bile pigment lurobilin. In a preferred embodiment, the method comprises detecting dodecanedioic acid. In a preferred embodiment, the method comprises detecting L-Phenylalanine. In a preferred embodiment, the method comprises detecting L-Phenylalanine. In a preferred embodiment, the method comprises detecting Adenosine. In a preferred embodiment, the method comprises detecting MG(20:3(8Z,11Z,14Z)/0:0/0:0). In a preferred embodiment, the method comprises detecting L-Alanine. In a preferred embodiment, the method comprises detecting 3,6-Dimethoxy-19-norpregna-1,3,5,7,9-pentaen-20-one.

In one embodiment, the present invention provides a method for diagnosing IBS, comprising detecting one or more fecal metabolites selected from the list in table 7. In one embodiment, the present invention provides a method for diagnosing IBS, comprising detecting one or more fecal metabolites selected from the list in table 13. In any such embodiments, detecting the metabolite comprises measuring the concentration of the metabolite in a sample, for example the concentration relative to a corresponding sample from a control (non-IBS) individual or relative to a reference value. In one embodiment, machine learning is applied to fecal metabolome data to diagnose IBS.

In one embodiment, the present invention provides a method for diagnosing IBS, comprising detecting one or more fecal metabolites that are differentially abundant in patients suffering from IBS. In one embodiment, the one or more fecal metabolites that are differentially abundant in patients suffering from IBS are: 2-Phenylpropionate, 3-Buten-1-amine, Adenosine, I-Urobilin, 2,3-Epoxymenaquinone, [FA (22:5)] 4,7,10,13,16-Docosapentaynoic acid, 3,6-Dimethoxy-19-norpregna-1,3,5,7,9-pentaen-20-one, Cucurbitacin S, N-Heptanoylglycine, 11-Deoxocucurbitacin I, Staphyloxanthin, Piperidine, Leu-Ser-Ser-Tyr, L-Urobilin, L-Phenylalanine, Ala-Leu-Trp-Pro, 3-Feruloyl-1,5-quinolactone, PG(P-16:0/14:0), 3-deoxy-D-galactose, MG(20:3(8Z,11Z,14Z)/0:0/0:0), Mesobilirubinogen, L-Alanine, Tyrosine, PG(O-30:1), beta-Pinene, 2,4,8-Eicosatrienoic acid isobutylamide, Glutarylglycine, [PR] gamma-Carotene/beta,psi-Carotene, Neuromedin B (1-3), Heptane-1-thiol, Violaxanthin, Isolimonene, Ile-Lys-Cys-Gly, His-Met-Val-Val, Allyl caprylate, Hydroxyprolyl-Tryptophan, Dodecanedioic acid, 2-O-Benzoyl-D-glucose, 2-Ethylsuberic acid, D-Urobilin, 20-hydroxy-E4-neuroprostane, PG(O-31:1), Anigorufone, Nonyl acetate, L-Arginine, PG(P-32:1), Glu-Ala-Gln-Ser, PG(31:0), Cucurbitacin I, Arg-Lys-Phe-Val, Genipinic acid, Hexoses, Lys-Phe-Phe-Phe, PI(41:2), D-galactal, Traumatic acid, Adenine, PC(22:2(13Z,16Z)/15:0), 2-Phenylethyl beta-D-glucopyranoside, PG(37:2), Glycerol tributanoate, Arg-Leu-Pro-Arg, 2-O-p-Coumaroyl-D-glucose, 3,4-Dihydroxyphenyllactic acid methyl ester, PG(P-28:0), PG(34:0), L-Lysine, Ribitol, LysoPE(18:2(9Z,12Z)/0:0), PA(20:4(5Z,8Z,11Z,14Z)e/2:0), 5-Dehydroshikimate, Threoninyl-Isoleucine, L-Methionine, PS(26:0)), alpha-Pinene, Fenchene, Glu-Ile-Ile-Phe, Gln-Phe-Phe-Phe, Ursodeoxycholic acid, PC(34:2), 3,17-Androstanediol glucuronide, Pyridoxamine, [ST hydrox] (25R)-3alpha,7alpha-dihydroxy-5beta-cholestan-27-oyl taurine, PA(42:2), [FA (16:0)] 2-bromo-hexadecanal, 3,6-Dihydro-4-(4-methyl-3-pentenyl)-1,2-dithiin, 3-Methylcrotonylglycine xi-7-Hydroxyhexadecanedioic acid, Camphene, 2-Hydroxy-3-carboxy-6-oxo-7-methylocta-2,4-dienoate, 7C-aglycone, 1-(3-Aminopropyl)-4-aminobutanal, Benzyl isobutyrate, (S)-(E)-8-(3,6-Dimethyl-2-heptenyl)-4′,5,7-trihydroxyflavanone, 1,3-di-(5Z,8Z,11Z,14Z,17Z-eicosapentaenoyl)-2-hydroxy-glycerol (d5), SM(d18:0/18:0), L-Homoserine, 17beta-(Acetylthio)estra-1,3,5(10)-trien-3-ol acetate, [ST (2:0)] 5beta-Chola-3,11-dien-24-oic Acid, PG(33:2), PE(22:4(7Z,10Z,13Z,16Z)/P-16:0), Protoporphyrinogen IX, alpha-Tocopherol succinate, Methyl (9Z)-6′-oxo-6,5′-diapo-6-carotenoate, PG(16:1(9Z)/16:1(9Z)), PC(o-22:1(13Z)/20:4(8Z,11Z,14Z,17Z)), PG(31:2), alpha-phellandrene, [PS (12:0/13:0)] 1-dodecanoyl-2-tridecanoyl-sn-glycero-3-phosphoserine (ammonium salt), Glu-Asp-Asp, PG(33:1), PA(0-20:0/22:6(4Z,7Z,10Z,13Z,16Z,19Z)), [FA oxo(19:0)] 18-oxo-nonadecanoic acid, PG(16:1(9Z)/18:0), Leu-Val, demethylmenaquinone-6, PC(o-16:1(9Z)/14:1(9Z)), PG(P-32:0), (24E)-3beta,15alpha,22S-Triacetoxylanosta-7,9(11),24-trien-26-oic acid, PA(33:5), LysoPC(0:0/18:0), Ile-Arg-Ile, Lauryl acetate, Glu-Glu-Gly-Tyr, 3-(Methylthio)-1-propanol, (−)-(E)-1-(4-Hydroxyphenyl)-7-phenyl-6-hepten-3-ol, Dimethyl benzyl carbinyl butyrate and/or Methyl 2,3-dihydro-3,5-dihydroxy-2-oxo-3-indoleacetic acid. In one embodiment, the present invention provides a method for diagnosing IBS, comprising detecting differential abundance of one or more fecal metabolites selected from the list in table 8. In certain embodiments, the method of the invention comprises detecting at least 2, 5, 10, 15 or 20 or all of these metabolites. In some embodiments, the method comprises detecting a precursor or breakdown product of the above metabolites.

In certain embodiments, the abundance of metabolites is significantly increased in patients with IBS, for example as shown in table 8. In one embodiment, bile acids are significantly more abundant in patients with IBS. In a particular embodiment, [ST hydroxy] (25R)-3alpha,7alpha-dihydroxy-5beta-cholestan-27-oyl taurine is detected or is measured. It is significantly more abundant in patients with IBS. In a particular embodiment, [ST (2:0)] 5beta-Chola-3,11-dien-24-oic acid is detected or is measured. It is significantly more abundant in patients with IBS. In a particular embodiment, UDCA is detected or is measured, it is significantly more abundant in patients with IBS. In another embodiment, amino acids are significantly more abundant in patients with IBS. for example tyrosine and/or lysine. In particular embodiments, the method of the invention comprises detecting or quantifying the levels of tyrosine or lysine in a sample and diagnosing IBS. In certain embodiments, the abundance of metabolites is significantly decreased in patients with IBS, for example as shown in table 8.

In one embodiment, the present invention provides a method for diagnosing IBS, comprising detecting one or more fecal metabolites that are differentially abundant in patients suffering from IBS compared to a healthy control (i.e. from one or more subjects who does not suffer from IBS). In a preferred embodiment, the one or more fecal metabolites that are differentially abundant in patients suffering from IBS are sulfate, glucuronide, carnitine, glycine and glutamine conjugates. In one embodiment, the method comprises detecting metabolites involved in phase 2 metabolism, which are is upregulated in patients with IBS. In any such embodiments, detecting the metabolite comprises measuring the concentration of the metabolite in a sample, for example the concentration relative to a corresponding sample from a control (non-IBS) individual or relative to a reference value.

In one embodiment, the present invention provides a method for diagnosing IBS-D (IBS associated with diarrhoea), comprising detecting one or more fecal metabolites that are differentially abundant in patients suffering from IBS-D. In one embodiment, bile acids are differentially abundant in patients with IBS-D. In one embodiment, total bile acid, secondary bile acids, sulphated bile acids, UDCA and/or conjugated bile acids are differentially abundant in patients with IBS-D. In a particular embodiment, total bile acid is differentially abundant in patients with IBS-D. In a particular embodiment, secondary bile acids are differentially abundant in patients with IBS-D. In a particular embodiment, sulphated bile acids are differentially abundant in patients with IBS-D. In a particular embodiment, UDCA is differentially abundant in patients with IBS-D. In a particular embodiment, conjugated bile acids are differentially abundant in patients with IBS-D. In any such embodiments, detecting the metabolite comprises measuring the concentration of the metabolite in a sample, for example the concentration relative to a corresponding sample from a control (non-IBS) individual or relative to a reference value.

Methods of Detecting Urine Metabolites

GC/LC-MS

Metabolites may be detected by any suitable method known in the art. In one embodiment, urine metabolites that are differentially abundant in patients suffering from IBS compared to a healthy control (i.e. from one or more subjects who does not suffer from IBS) are detected using GC/LC-MS.

In a particular embodiment, GC/LC-MS is preferably used for detecting urine metabolites that are predictive of IBS. For urine metabolomics, the values of metabolites may be normalized with reference to urine creatinine levels in each sample.

FAIMS (High Field Asymmetric Waveform Ion Mobility Spectrometry)

In one embodiment, urine metabolites that are differentially abundant in patients suffering from IBS are detected using FAIMS. In a particular embodiment, FAIMS is preferably used for detecting urine metabolites that are predictive of IBS. For urine metabolomics, the values of metabolites may be normalized with reference to urine creatinine levels in each sample.

Ion mobility spectrometry (IMS) is a well-known technique for analysing ion separation in the gaseous phase based on differences in ion mobilities under the influence of an electric field. Field Asymmetric Ion Mobility Spectrometry (FAIMS) is a specific example of an IMS technique that uses a high voltage asymmetric waveform at radio frequency combined with a static compensation voltage applied between two electrodes to separate ions at atmospheric pressure. Different ions pass through the electric fields to a detector at different compensation voltages. Thus, by varying the compensation voltage, a FAIMS analyser can detect the presence of different ions in the sample. The FAIMS instrument benefits from small size and lack of pumping requirements, allowing for portability as a standalone instrument. FAIMS is described in more detail in reference (20).

The FAIMS output consists of two modes: a positive mode (for positively charged ions) and a negative mode (for negatively charged ions). Each of these modes is made up of 51 dispersion fields (DFs), totaling 102 DFs taking both modes into account. Each DF is applied to the testing sample following the principle of linear sweep voltammetry, i.e. the compensation voltage is varied from a starting value to an end value, separated by 512 equally spaced voltages. The ion current value at each of the equally spaced voltages is measured. Each pair of compensation voltage and measured ion current can be referred to as a data point. Across all dispersion fields for both the positive and negative modes, there are 52224 data points.

Previous applications of FAIMS have used the method to study gastrointestinal toxicity, bile acid diarrhoea, and colorectal cancer. For example, PCT application WO 2016/038377 describes a method for diagnosing coeliac disease or bile acid diarrhoea by analysing the concentration of a signature compound in a body sample from a test subject using FAIMS and comparing this concentration with a reference for the concentration of the signature compound in an individual who does not suffer from the disease. An increase in the concentration of the signature compound in the body sample from the test subject compared to the reference suggests that the subject is suffering from the disease being screened for, or has a pre-disposition thereto, or provides a negative prognosis of the subject's condition.

In use, the FAIMS analyser is operated by running the device with air (no sample) and water, to clean the analyser. A urine sample is then introduced to obtain the signals. The FAIMS analyser is operated with water and then with air again before the next test sample is run. The signals from all of the dispersion fields are then aligned using crosscorrelation.

In some embodiments, the method of diagnosing IBS of the present invention is a computer-implemented method. In a preferred embodiment, the computer-implemented method is a method for analysing a FAIMS profile of a urine sample to determine the presence or absence of IBS and/or classify the urine sample into an IBS subset is provided. The method comprises:
obtaining signals corresponding to the FAIMS profile of the urine sample, air, and water;
pre-processing the obtained signals by performing one or more of: smoothing the signals, trimming off baseline noise from the signals, and aligning the signals in regions of interest;
extracting a plurality of features from the pre-processed signal; and
applying a trained classifier using the extracted features to determine the presence or absence of IBS and/or classify the urine sample into an IBS subset.

Advantageously, by applying signal smoothing to the received signals, the raw signal strength is retained while reducing the ‘noise’ in the signal. By trimming the signal, noise is reduced, improving the quality of the output and reducing technical artefacts between runs caused by crosscontamination and carry-over signals.

Overall, the method retains more features for analysis compared to the prior art method, which, in the context of a diagnostic application, improves the capability to distinguish between populations and stratify subgroups within a population.

Preferably, pre-processing the obtained signals comprises all three steps of smoothing the signals, trimming off baseline noise from the signals, and aligning the signals in regions of interest.

Obtaining the FAIMS signal may comprise analysing the biological sample with a FAIMS system to produce a signal corresponding to the FAIMS profile of the biological sample.

Preferably, the signal smoothing is performed using a Savitzky-Golay filter, as described in Anal. Chem., 36(8), 1964, Savitzky A., Golay M J E. “Smoothing and Differentiation of Data by Simplified Least Squares Procedures”, pages 1627-1639 (21). Using a Savitzky-Golay filter is advantageous because it keeps the peak signal values intact, which can improve the accuracy of the classification. The signal smoothing may be applied to the dispersion fields of both positive and negative modes of the signal.

The signal trimming may be performed using an optimised baseline cut-off. The signal alignment may be performed using cross correlation.

Selection of features from the signals may be performed using a linear regression model, for example LASSO. LASSO is described in more detail in Journal of the Royal Statistical Society, Series B, 58(1), 1996, R. Tibshirani, “Regression Shrinkage and Selection via the Lasso”, pages 267-288 (22).

The trained classifier is preferably a support vector machine. Alternatively, the classifier may be a random forest. In a preferred embodiment, the classifier is a random forest.

Integrative Analysis of Diet, Microbiome and Metabolome in IBS Patients

In certain embodiments, the invention provides a method of diagnosing IBS comprising one or more of i) detecting a bacterial species, for example as discussed above, ii) detecting genes involved in one or more of the pathways, for example as discussed above, iii) detecting metabolites, for example as discussed above. In any such embodiments, detecting the bacteria, gene or metabolite comprises measuring the abundance or concentration of said marker in a sample, for example the relative to a corresponding sample from a control (non-IBS) individual or relative to a reference value.

In one embodiment, the invention provides a method of diagnosing IBS, comprising detecting the depletion of a bacterial species. In one embodiment, the depleted bacterial species is one or more of the following: Paraprevotella species, Bacteroides species, Barnesiella intestinihominis, Eubacterium eligens, Ruminococcus lactaris, Eubacterium biforme, Desulfovibrio desulfuricans, Coprococcus species and Eubacterium species. In certain embodiments, the method of the invention comprises detecting one or more of Paraprevotella species, Bacteroides species, Barnesiella intesfinihominis, Eubacterium eligens, Ruminococcus lactaris, Eubacterium biforme, Desulfovibrio desulfuricans, Coprococcus species and Eubacterium species.

In one embodiment, the invention provides a method of diagnosing IBS, comprising detecting the differential utilisation of dietary components. In a particular embodiment, the invention provides a method of diagnosing IBS, comprising detecting the differential utilisation of a high protein diet.

In one embodiment, the invention provides a method of diagnosing IBS, comprising detecting higher levels of peptides and amino acids. In another embodiment, the invention provides a method of diagnosing IBS, comprising detecting increased levels of L-alanine, L-lysine, L-methionine, L-phenylalanine and/or tyrosine.

In one embodiment, the invention provides a method of diagnosing IBS, comprising detecting increased levels of bile acids. In a particular embodiment, the invention provides a method of diagnosing IBS, comprising detecting increased levels of UDCA, sulfolithocholylglycine and [ST hydrox](25R)-3alpha,7alpha-dihydroxy-5beta-cholestan-27-oyl taurine and/or Iurobilin.

In one embodiment, the invention provides a method of diagnosing IBS, comprising detecting increased levels of metabolites. In another embodiment, the invention provides a method of diagnosing IBS, comprising detecting increased levels of allantoin, cis-4-decenedioic acid, decanoylcarnitine and/or dodecanedioylcarnitine.

Diagnostic Methods

The inventors have developed new and improved methods for diagnosing IBS.

In preferred embodiments, the methods of the invention are for use in diagnosing a patient resident in Europe, such as Northern Europe, preferably Ireland or a patient that has a European, Northern European or Irish diet. The examples demonstrate that the methods of the invention are particular effective for such patients.

In certain embodiments of any aspect of the invention, the abundance of bacteria, genes or metabolites is assessed relative to control (non-IBS) individuals. In preferred embodiments, the abundance of urine metabolites is assessed relative to control (non-IBS) individuals. Such reference values may be generated using any technique established in the art.

In certain embodiments of any aspect of the invention, comparison to a corresponding sample from a control (non-IBS) individual is a comparison to a corresponding sample from a healthy individual.

Preferably the method of diagnosing IBS has a sensitivity of greater than 40% (e.g. greater than 45%, 50% or 52%, e.g. 53% or 58%) and a specificity of greater than 90% (e.g. greater than 93% or 95%, e.g. 96%).

In certain embodiments, the method of diagnosis is a method of monitoring the course of treatment for IBS.

In certain embodiments, the step of detecting the presence or abundance of bacteria, such as in a fecal sample, comprises a nucleic acid based quantification methodology, for example 16S rRNA gene amplicon sequencing. Methods for qualitative and quantitative determination of bacteria in a sample using 16S rRNA gene amplicon sequencing are described in the literature and will be known to a person skilled in the art. Other techniques may involve PCR, rtPCR, qPCR, high throughput sequencing, metatranscriptomic sequencing, or 16S rRNA analysis.

In alternative aspects of any embodiment of the invention, the invention provides a method for diagnosing the risk of developing IBS.

In any embodiment of the invention, modulated abundance of a bacterial strain, species, metabolite or gene pathway is indicative of IBS. In preferred embodiments, the abundance of the bacterial strain, species or OTU as a proportion of the total microbiota in the sample is measured to determine the relative abundance of the strain, species or OTU. In preferred embodiments, the concentration of a metabolite is measured, in particular a urine metabolite. In preferred embodiments, the abundance of bacterial strains carrying a gene pathway of interest as a proportion of the total microbiota in the sample is measured to determine the relative abundance of the strains, or concentrations of gene sequences are measured. Then, in such preferred embodiments, the relative abundance of the bacterium or OTU or the concentration of the metabolite or gene sequence in the sample is compared with the relative abundance or concentration in the same sample from a control (non-IBS) individual. A difference in relative abundance of the bacterium or OTU in the sample, e.g. a decrease or an increase, compared to the reference is a modulated relative abundance. As explained herein, detection of modulated abundance can also be performed in an absolute manner by comparing sample abundance values with absolute reference values. Therefore, the invention provides a method of determining IBS status in an individual comprising the step of assaying a biological sample from the individual for a relative abundance of one or more IBS-associated bacteria and/or a modulated concentration of a metabolite or gene pathway, wherein a modulated relative abundance of the bacteria or modulated concentration of a metabolite or gene pathway is indicative of IBS. Similarly, the invention provides a method of determining whether an individual has an increased risk of having IBS comprising the step of assaying a biological sample from the individual for a relative abundance of one or more IBS-associated oral bacteria or IBS-associated metabolites or gene pathways, wherein modulated relative abundance or concentration is indicative of an increased risk.

In any embodiment of the invention, detecting a bacteria may comprise detecting “modulated relative abundance”. As used herein, the term “modulated relative abundance” as applied to a bacterium or OTU in a sample from an individual should be understood to mean a difference in relative abundance of the bacterium or OTU in the sample compared with the relative abundance in the same sample from a control (non-IBS) individual (hereafter “reference relative abundance”). In one embodiment, the bacterium or OTU exhibits increased relative abundance compared to the reference relative abundance. In one embodiment, the bacterium or OTU exhibits decreased relative abundance compared to the reference relative abundance. Detection of modulated abundance can also be performed in an absolute manner by comparing sample abundance values with absolute reference values. In one embodiment, the reference abundance values are obtained from age and/or sex matched individuals. In one embodiment, the reference abundance values are obtained from individuals from the same population as the sample (i.e. Celtic origin, North African origin, Middle Eastern origin). Method of isolating bacteria from oral and fecal sample are routine in the art and are further described below, as are methods for detecting abundance of bacteria. Any suitable method may be employed for isolating specific species or genera of bacteria, which methods will be apparent to a person skilled in the art. Any suitable method of detecting bacterial abundance may be employed, including agar plate quantification assays, fluorimetric sample quantification, qPCR, 16S rRNA gene amplicon sequencing, and dye-based metabolite depletion or metabolite production assays.

Stratifying Patients

In certain embodiments, the methods of the invention are for use in stratifying patients according to the type of IBS that they are suffering from. In particular, in certain embodiments, the methods of the invention are for diagnosing a patient suffering from IBS as having a normal-like microbiota (i.e. a microbiota composition similar to the microbiota composition of a person without IBS), or an altered microbiota (i.e. a microbiota dissimilar to the microbiota of a person without IBS) (see Jeffery I B, O'Toole P W, Ohman L, Claesson M J, Deane J, Quigley E M, Simren M. 2012. “An irritable bowel syndrome subtype defined by species-specific alterations in fecal microbiota.” Gut 61:997-1006 (23)). Patients suffering from IBS with a normal-like microbiota may benefit from different treatments compared to patients with an altered microbiota, so the methods of the invention may result in more appropriate treatment strategies and better outcomes for patients. Therefore, in certain embodiments, the methods of the invention comprise developing and/or recommending a treatment plan for a patient based on their microbiota. IBS patients with normal-like microbiota may benefit from treatments known to ameliorate anxiety or depression. IBS patients with an altered microbiota may benefit from treatments able to instigate beneficial changes in the microbiota and/or address dysbiosis, such as live biotherapeutic products, in particular compositions comprising Blautia hydrogenotrophica (as described in WO2018109461). IBS patients with an altered microbiota may also benefit from diet adjustments, such as a FODMAP (fermentable oligo-, di-, monosaccharides and polyols) diet. Compositions comprising Blautia hydrogenotrophica are also effective for treating visceral hypersensitivity (as described in WO2017148596), which patients with normal-like microbiota may experience, so such compositions will also be useful for treating such patients.

In certain embodiments, the invention provides a method for stratifying patients suffering from IBS into subgroups based on their microbiome and/or metabolome. In a particular embodiment, the method of the invention comprises detecting one or more bacterial strains belonging to at least one genus selected from the group consisting of: Anaerostipes, Anaerotruncus, Anaerofilum, Bacteroides, Blaufia, Eggerthella, Streptococcus, Gordonibacter, Holdemania, Ruminococcus, Veilonella, Akkermansia, Alistipes, Bamesiella, Butyricicoccus, Butyricimonas, Clostridium, Coprococcus, Faecalibacterium, Haemophilus, Howardella, Methanobrevibacter, Oscillobacter, Prevotella, Pseudoflavonifractor, Roseburia, Slackia, Sporobacter and Victivallis. In a particular embodiment, the method of the invention comprises detecting bacterial species which may belong to Clostridium clusters IV, XI or XVIII. In a particular embodiment, the method of the invention comprises detecting bacterial strains which may include one or more of the following species: Anaerostipes hadrus, Bacteroides ovatus, Bacteroides thetaiotaomicron, Clostridium asparagiforme, Clostridium boltaea, Clostridium hathewayi, Clostridium symbiosum, Coprococcus comes, Ruminococcus gnavus, Streptococcus salivarus, Ruminococcus torques, Alistipes senegalensis, Eubacterium eligens, Eubacterium siraeum, Faecalibacterium prausnitzii, Roseburia hominis, Haemophilus parainfluenzae, Ruminococcus callidus, Veilonella parvula and Coprococcus sp. ART55/1. In a particular embodiment, the method of the invention comprises detecting one or more of the following bacterial strains: Lachnospiracaea bacterium 3 1 46FAA, Lachnospiracaea bacterium 5 1 63FAA, Lachnospiracaea bacterium 7 1 58FAA and Lachnospiracaea bacterium 8 1 57FAA. In a particular embodiment, the method of the invention comprises detecting bacterial taxa selected from tables 17, 18, 19 and/or 20. In certain embodiments, the method of the invention comprises detecting a metabolite associated with an IBS subgroup. In certain embodiments, the metabolite is detected in a fecal sample. In certain embodiments, the metabolite is detected in a urine sample.

In certain embodiments, the invention provides a method of assessing whether a patient suffering from IBS would benefit from a treatment able to instigate beneficial changes in the microbiota and/or address dysbiosis, such as a live biotherapeutic product. In a particular embodiment, the method of the invention comprises detecting one or more bacterial strains belonging to at least one genus selected from the group consisting of: Anaerostipes, Anaerotruncus, Anaerofilum, Bacteroides, Blaufia, Eggerthella, Streptococcus, Gordonibacter, Holdemania, Ruminococcus, Veilonella, Akkermansia, Alistipes, Bamesiella, Butyricicoccus, Butyricimonas, Clostridium, Coprococcus, Faecalibacterium, Haemophilus, Howardella, Methanobrevibacter, Oscillobacter, Prevotella, Pseudoflavonifractor, Roseburia, Slackia, Sporobacter and Victivallis. In a particular embodiment, the method of the invention comprises detecting bacterial species which may belong to Clostridium clusters IV, XI or XVIII. In a particular embodiment, the method of the invention comprises detecting bacterial strains which may include one or more of the following species: Anaerostipes hadrus, Bacteroides ovatus, Bacteroides thetaiotaomicron, Clostridium asparagiforme, Clostridium boltaea, Clostridium hathewayi, Clostridium symbiosum, Coprococcus comes, Ruminococcus gnavus, Streptococcus salivarus, Ruminococcus torques, Alistipes senegalensis, Eubacterium eligens, Eubacterium siraeum, Faecalibacterium prausnitzii, Roseburia hominis, Haemophilus parainfluenzae, Ruminococcus callidus, Veilonella parvula and Coprococcus sp. ART55/1. In a particular embodiment, the method of the invention comprises detecting one or more of the following bacterial strains: Lachnospiracaea bacterium 3 1 46FAA, Lachnospiracaea bacterium 5 1 63FAA, Lachnospiracaea bacterium 7 1 58FAA and Lachnospiracaea bacterium 8 1 57FAA. In a particular embodiment, the method of the invention comprises detecting bacterial taxa selected from tables 17, 18, 19 and/or 20. In certain embodiments, the method of the invention comprises detecting a metabolite associated with an IBS subgroup. In certain embodiments, the metabolite is detected in a fecal sample. In certain embodiments, the metabolite is detected in a urine sample.

In certain embodiments, the method of the invention comprises identifying a subgroup which is characterised by an altered microbiome and/or metabolome relative to healthy control subjects. In certain embodiments, the method of the invention comprises identifying a subgroup which is characterised by a microbiome and/or metabolome similar to healthy control subjects. In certain embodiments, the methods of the invention are for use in classifying of a patient suffering from IBS into a subgroup based on their microbiome. In certain embodiments, the methods of the invention are for use in determining whether a patient suffering from IBS would benefit from a treatment able to instigate beneficial changes in the microbiota and/or address dysbiosis, such as live biotherapeutic products. In certain embodiments, it may be deemed that a patient suffering from IBS would benefit from a treatment able to instigate beneficial changes in the microbiota and/or address dysbiosis, such as live biotherapeutic products, if said patient is classified as belonging to a subgroup characterised by an altered microbiome and/or metabolome relative to healthy control subjects. In certain embodiments, it may be deemed that a patient suffering from IBS would not benefit from a treatment able to instigate changes in the microbiota and/or address dysbiosis, such as live biotherapeutic products, if said patient is classified as belonging to a subgroup characterised by similar microbiome and/or metabolome to healthy control subjects.

Kits

The invention also provides kits comprising reagents for performing the methods of the invention, such as kits containing reagents for detecting one or more, such as two or more of the bacterial species, genes or metabolites set out above. As such, provided are kits that find use in practicing the subject methods of diagnosing IBS, as mentioned above. The kit may be configured to collect a biological sample, for example a urine sample or a fecal sample. In a preferred embodiment, the kit is configured to collect a urine sample. The individual may be suspected of having IBS. The individual may be suspected of being at increased risk of having IBS. A kit can comprise a sealable container configured to receive the biological sample. A kit can comprise polynucleotide primers. The polynucleotide primers may be configured for amplifying a 16S rRNA polynucleotide sequence from at least one IBS-associated bacterium to form an amplified 16S rRNA polynucleotide sequence. A kit may comprise a detecting reagent for detecting the amplified 16S rRNA sequence. A kit may comprise instructions for use.

### EXAMPLES

### Summary

Background & Aims: Diagnosis and stratification of irritable bowel syndrome (IBS) is based on symptoms and other disease exclusion. Whether the pathogenesis begins centrally and/or at the end organ is unclear. Some patients have an alteration in their microbiota. Therefore, microbiome and metabolomic profiling was conducted to identify biomarkers for the condition.

To work toward an evidence-based stratification of patients with IBS, a metagenomic study of fecal samples was performed, along with metabolomic analyses of urine and faeces in patients with IBS (according to the Rome IV criteria) in comparison with controls. Microbiome and metabolomic signatures are evident in IBS but these are independent of the traditional clinical symptom-based subsets of IBS (IBS-D vs IBS-C, IBS-alternating or mixed).

Methods: 80 patients with IBS (Rome IV) and 65 non-IBS controls were enrolled.

Anthropometric, medical and dietary information were collected with fecal and urine samples for microbiome and metabolomic analyses. Shotgun and 16S rRNA amplicon sequencing were performed on feces, and urine and fecal metabolites were analysed by gas chromatography (GC)—and liquid chromatography (LC) mass spectrometry (MS).

Results: Differential connections between diet and the microbiome with alterations of the metabolome were evident in IBS. Microbiota composition and predicted microbiome function in patients with IBS differed significantly from those of controls, but these were independent of IBS-symptom subtypes. Fecal metabolomic profiles also differed significantly between IBS patients and controls and were discriminatory for the condition. The urine metabolome contained an array of predictive metabolites but was mainly dominated by dietary and medication-related metabolites.

Conclusion: Despite clinical heterogeneity, IBS can be identified by species-, metagenomics and fecal metabolomic-signatures which are independent of symptom-based subtypes of IBS. These findings are useful for diagnosing IBS and for developing precision therapeutics for IBS.

### Example 1—Microbiota Profiling of Ibs Patients and Controls

Materials and Methods

Subject recruitment: Eighty patients aged 16-70 years with IBS meeting the Rome IV criteria were recruited at Cork University Hospital. Clinical subtyping of the patients (15) was as follows: IBS with constipation (IBS-C), mixed IBS (IBS-M) or IBS with diarrhea (IBS-D). Sixty-five controls of the same age range and of the same ethnicity and geographic region were recruited. Descriptive statistics for the study population are presented in Table 10.

Exclusion criteria included the use of antibiotics within 6 weeks prior to study enrolment, other chronic illnesses including gastrointestinal diseases, severe psychiatric disease, abdominal surgery other than hernia repair or appendectomy. Standard-of-care blood analysis was carried out on all participants if recent results were not available, and all subjects were tested by serology to exclude coeliac disease. The inclusion/exclusion criteria for the control population were the same as for the IBS population with the exception of having to fulfil the Rome IV criteria for IBS. Gastrointestinal (GI) symptom history, psychological symptoms, diet, medical history and medication data were collected on each participant (both IBS and controls) and using the following questionnaires: Bristol Stool Score (BSS), Hospital Anxiety and Depression Scale (HADS) (24); Food Frequency Questionnaire (FFQ) (25). Ethical approval for the study was granted by the Cork Research Ethics Committee (protocol number: 4DC001) before commencing the study and all participants provided written informed consent to take part.

Sample collection: Fecal and urine samples were collected from all participants for microbiome and metabolomics profiling. Subjects collected a freshly voided fecal sample at home using a collection kit and brought the sample to the clinic that day, when a fresh urine sample was collected. Samples were kept at 4° C. until brought to the laboratory for storage at −80° C. which was within a few hours of the sample collection.

Microbiome profiling and metagenomics-16S amplicon sequencing: Genomic DNA was extracted and amplified from frozen fecal samples (0.25 g) using the method described by Brown et al. (26). The modifications from the methods described by Brown et al. (26) included bead beating tubes consisting of 0.5 g of 0.1 mm zirconia beads and 4×3.5 mm glass beads. Fecal samples were homogenised via bead beating for 3×60 s cycles and cooled on ice between each cycle. Genomic DNA was visualised on 0.8% agarose gel and quantified using the SimpliNano Spectrometer (Biochrom™, US). The PCR master mix used 2× Phusion Taq High-Fidelity Mix (Thermo Scientific, Ireland) and 15 ng of DNA. The resulting PCR products were purified, quantified and equimolar amounts of each amplicon were then pooled before being sent for sequencing to the commercial supplier (GATC Biotech AG, Konstanz, Germany) on the MiSeq (2×250 bp) chemistry platforms. Sequencing was performed by GATC Biotech, Germany on an Illumina MiSeq instrument using a 2×250 bp paired end sequencing run.

Microbiome profiling and metagenomics—16S amplicon sequencing: Using the Qiagen DNeasy Blood & Tissue Kit and following the manufacturer's instructions, microbial DNA was extracted from 0.25 g of each of 144 frozen fecal samples (IBS: n=80 and control (n=64). No fecal sample was available for one control subject. The 16S rRNA gene amplicons preparation and sequencing was carried out using the 16S Sequencing Library Preparation Nextera protocol developed by Illumina (San Diego, Calif., USA). 15 ng of each of the DNA fecal extracts was amplified using PCR and primers targeting the V3-V4 variable region of the 16S rRNA gene using the following gene-specific primers:

16S Amplicon PCR Forward Primer
(S-D-Bact-0341-b-S-17) = 5′
(SEQ ID NO: 40)
TCGTCGGCAGCGTCAGATGTGTATAAGAGACAGCCTACGGGNGGCWGCAG
16S Amplicon PCR Reverse Primer
(S-D-Bact-0785-a-A-21) = 5′
(SEQ ID NO: 41)
GTCTCGTGGGCTCGGAGATGTGTATAAGAGACAGGACTACHVGGGTATCT
AATCC

The amplicon size was 531 bp. The products were purified and forward and reverse barcodes were attached by a second round of adapter PCR.

Microbiome profiling and metagenomics—Shotgun sequencing: For shotgun sequencing, 1 μg (concentration>5 ng/μL) of high molecular weight DNA for each sample was sent to GATC Biotech, Germany for sequencing on Illumina HiSeq platform (HiSeq 2500) using 2×250 bp paired-end chemistry. This returned 2,714,158,144 raw reads (2,612,201,598 processed reads) of which 45.6% were mapped to an average of 222,945 gene families per sample with a mean count value of 8,924,302±2,569,353 per sample.

Bioinformatics analysis (16S amplicon sequencing): Miseq 16S sequencing data was returned for 144 subjects. Data generated for 3 samples (2 IBS and 1 control) were removed as the number of reads returned from sequencing was too low for analysis, leaving 141 samples (control: n=63, IBS n=78). Raw amplicon sequence data were merged and the reads trimmed using the flash methodology (27). The USEARCH pipeline was used to generate the OTU table (28). The UPARSE algorithm was used to cluster the sequences into OTUs at 97% similarity (29). UCHIME chimera removal algorithm was used with Chimeraslayer to remove chimeric sequences (30). The Ribosomal Database Project (RDP) taxonomic classifier was used to assign taxonomy to the representative OTU sequences (28) and microbiota compositional (abundance and diversity) information was generated.

Bioinformatics analysis (Shotgun metagenomic sequencing): For shotgun metagenomics, 6 control samples were not sequenced due to data not passing QC or no sample available (control: n=59; IBS n=80). The number of raw read pairs obtained after sequencing, varied from 5,247,013 to 21,280,723 (Mean=9,763,159±2,408,048). Reads were processed in accordance with the Standard Operating Procedure of Human Microbiome Project (HMP) Consortium (31). Metagenomic composition and functional profiles were generated using HUMAnN2 pipeline (32). For each sample, multiple profiles were obtained, including: microbial composition profiles from clade-specific gene information (using MetaPhlAn2), Gene family abundance, pathways stratified per organism, total pathway coverage and abundance.

Machine learning: An in-house machine learning pipeline was applied to each datatype (16S, shotgun, and urine and fecal MS metabolomics) using a twostep approach applying the Least Absolute Shrinkage and Selection Operator (LASSO) feature selection followed by Random Forest

(RF) modelling (33). The models were implemented using R software version 3.4.0, using package glmnet version 2.0-10 for LASSO feature selection, and R package randomForest version 4.6-12. (34).

Each variable consisted of data from 78 IBS patients IBS and 64 controls. First, feature selection was performed using the LASSO algorithm to improve accuracy and interpretability of models by efficiently selecting the relevant features. This process was tuned by parameter lambda, which was optimized for each dataset using a grid search. The training data was filtered to include only the features selected by the LASSO algorithm, and RF was then used for modelling whereby 1500 trees were built. Both LASSO feature selection and RF modelling were performed using 10-fold cross validation (CV) repeated 10 times (10-fold, 10 repeats, R package caret version 6.0-76.), which generated an internal 10-fold prediction yielding an optimal model that predicts the IBS or Control classification of samples. This 10-fold cross-validation procedure was repeated ten times and the average area under the curve (AUC), sensitivity and specificity were reported.

Results

Microbiome Differs Between IBS and Controls but not Across IBS Clinical Subtypes

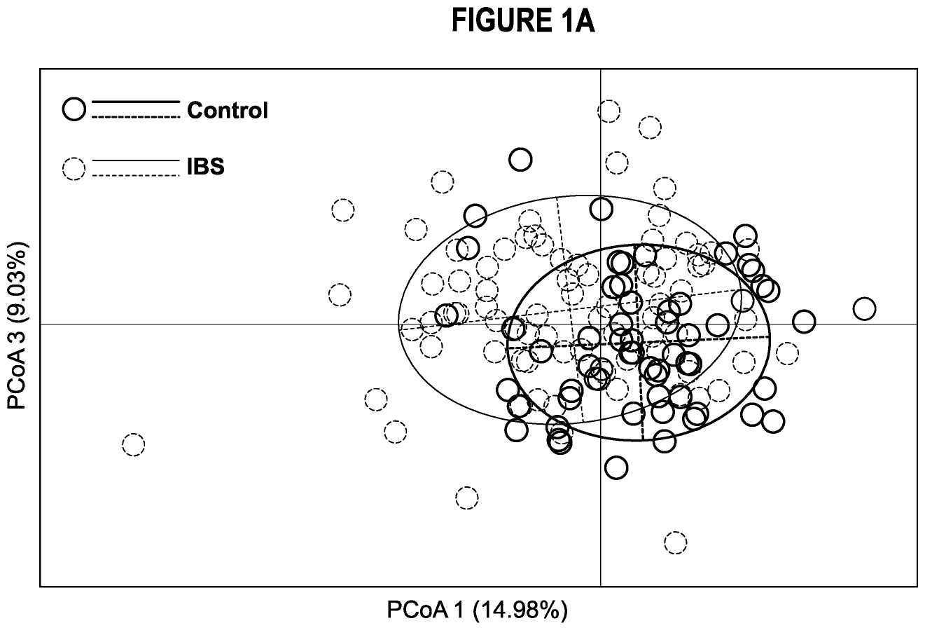

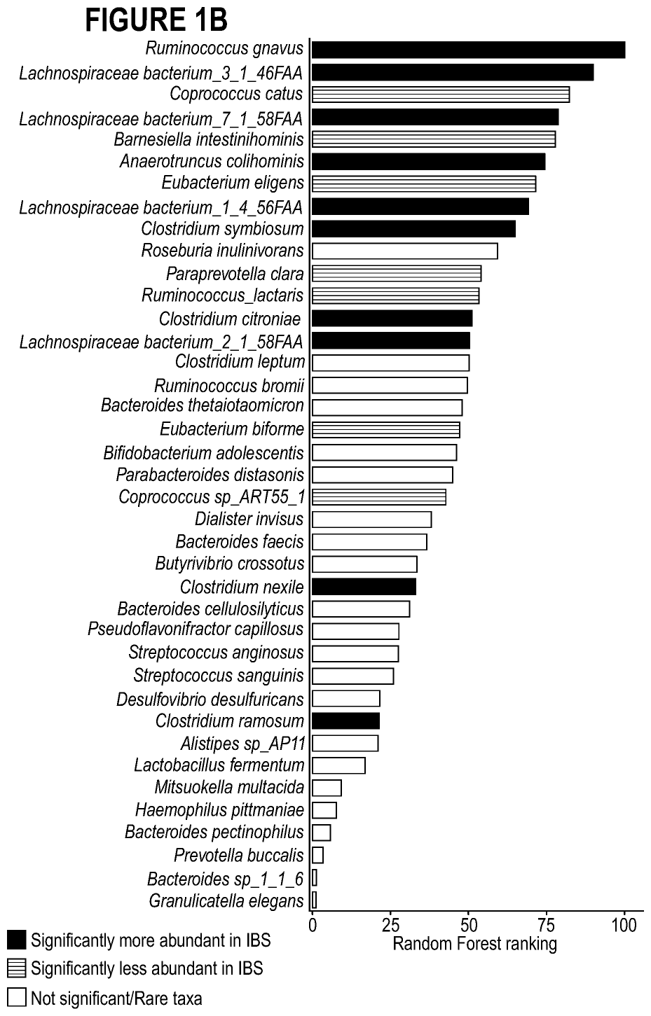

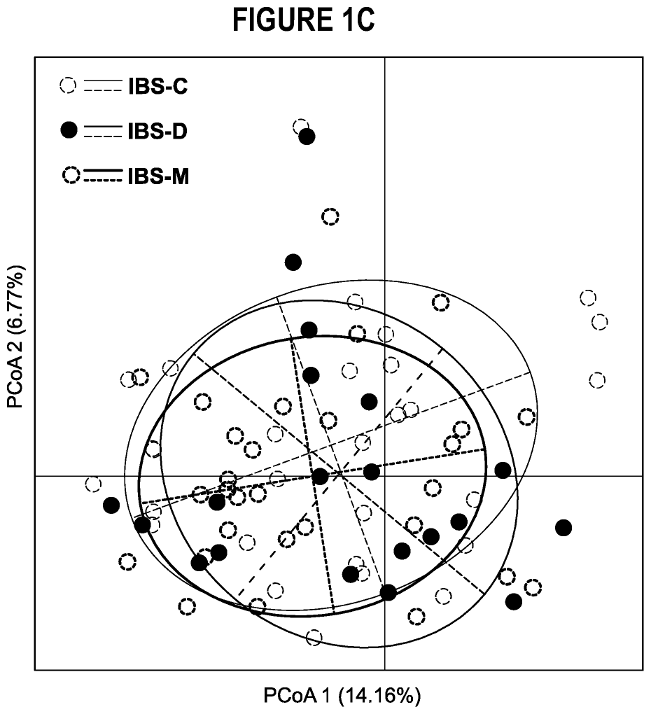

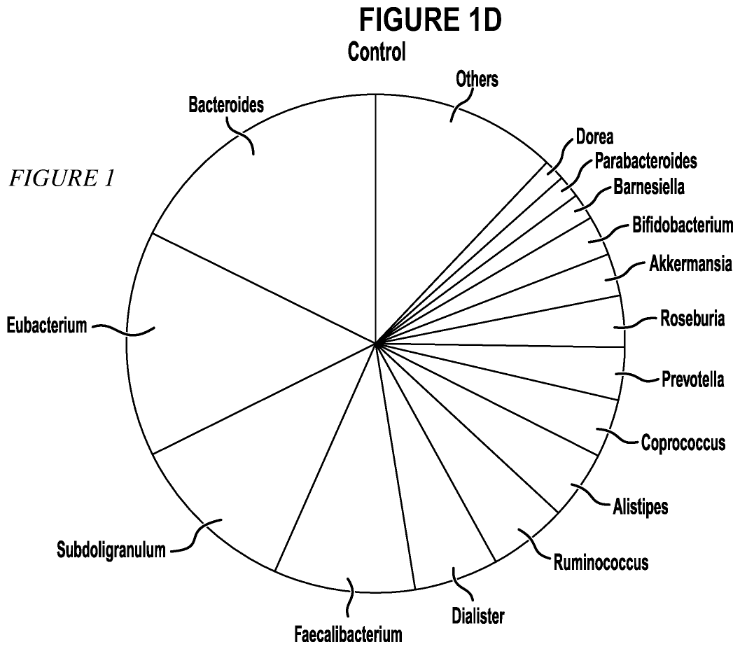

Microbiota profiling by 16S rRNA amplicon sequencing and Principal Co-Ordinate Analysis (PCoA) of the microbiota composition data confirmed that the microbiota of subjects with IBS was distinct from that of controls (FIG. 1a), albeit with some degree of overlap.

Machine learning was used to identify bacterial taxa predictive of IBS and control groups (FIG. 1b). These taxa belonged to the Ruminococcaceae, Lachnospiraceae and Bacteroides families/genera.

Machine learning (based on shotgun data) identified 6 genera predictive of IBS which included Lachnospiraceae, Oscillibacter and Coprococcus with an Area under the Curve (AUC) of 0.835 (sensitivity: 0.815 and specificity: 0.704; Table 1).

At the species level, 40 predictive features (AUC of 0.878; sensitivity: 0.894, specificity: 0.687; Table 2) were identified which included Ruminococcus gnavus and Lachnospiraceae spp which were significantly more abundant in IBS, while Barnesiella intestinihominis and Coprococcus catus were among taxa significantly less abundant in IBS based on pairwise comparison (Table 3). These alterations are consistent with previous studies (10-12), where the taxa that were significantly differentially abundant belonged to the Ruminococcaceae, Lachnospiraceae and Bacteroidetes families/genera.

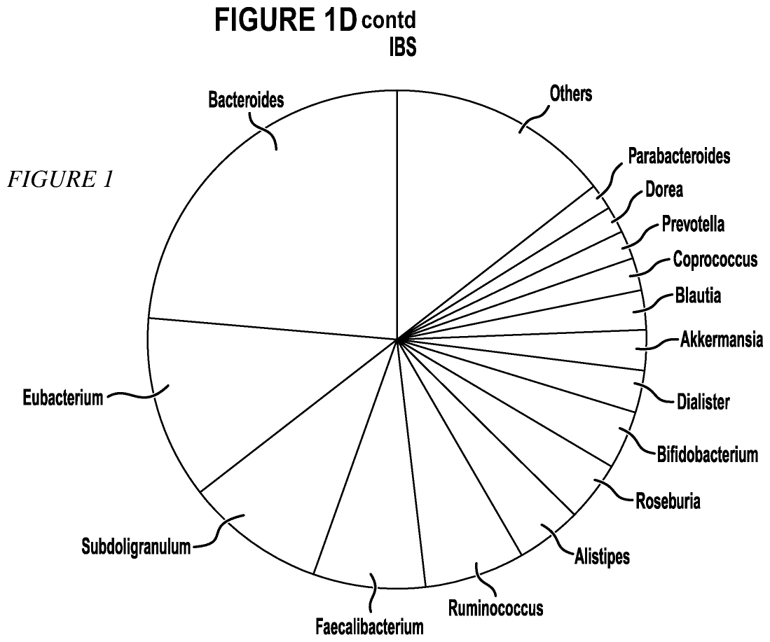

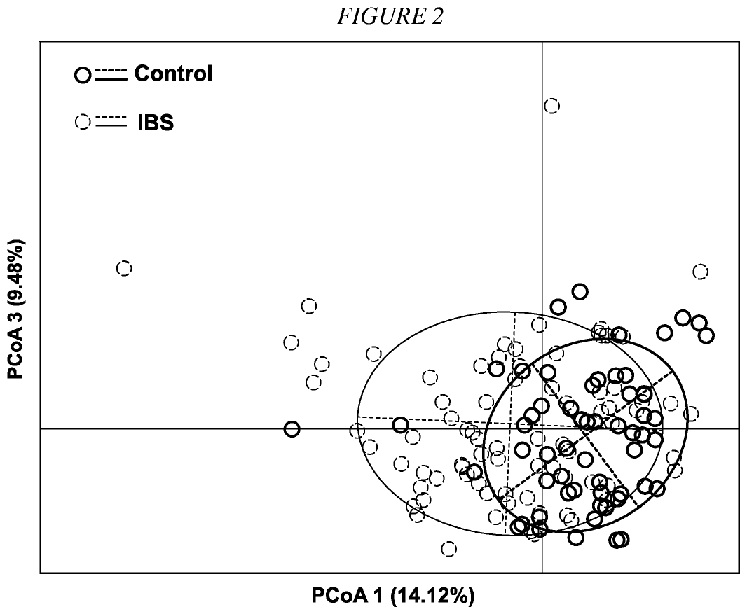

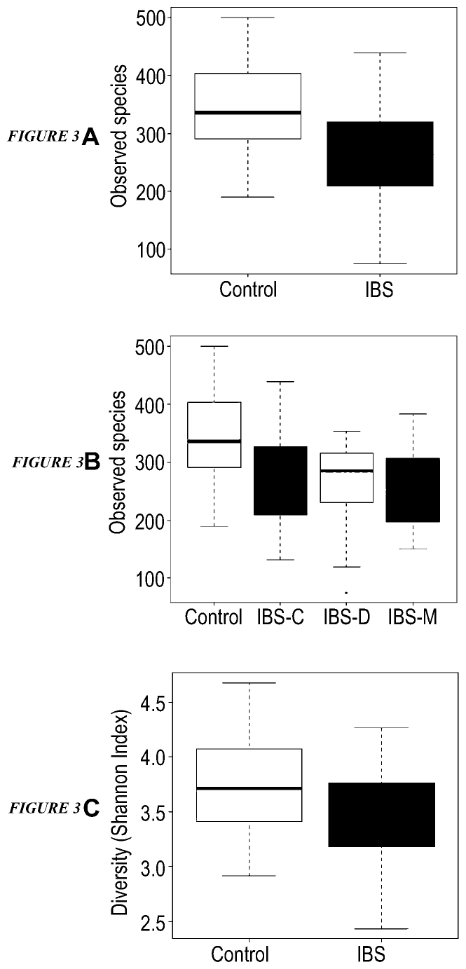

Clinical subtypes of IBS did not separate in a PCoA of microbiota beta diversity derived from 16S profiling data (FIG. 1c). Metagenomic shotgun sequencing corroborated 16S profiling in separating IBS subjects from controls (FIG. 2). Moreover, the microbiota composition at genus and species level (as assigned using shotgun sequence data) underscored the microbiota composition differences between IBS and controls. (FIG. 1d). Pairwise comparison of the annotated metagenome dataset identified 232 shotgun pathways stratified per organism that were significantly more abundant in the IBS group compared to the controls (Table 4). These notably included a number of amino acid biosynthesis/degradation pathways whose altered activity may be relevant to IBS pathophysiology (35).

Other pathways that were less abundant in the metagenome of subjects with IBS included galactose degradation, sulfate reduction, sulfate assimilation and cysteine biosynthesis, collectively indicative of a reduced sulphur metabolism in IBS. The genes encoding 12 pathways were more abundant in IBS subjects including those for starch degradation V. Of a total of 232 functional pathways that were significantly more abundant in the IBS group, 113 were associated with the Lachnospiraceae family or the Ruminococcus species.

Discussion

A species-level microbiome signature for IBS was identified that included some broad taxonomic groups (lower abundance of Bacteroides species, elevated levels of Lachnospiraceae and Ruminococcus spp.) as well as a list of 32 taxa whose collected abundance values could discriminate between IBS and controls. The ability to distinguish the microbiota of subjects with IBS from controls is superior to that of an earlier study based on a supervised split (10), or one which could not distinguish between control and IBS microbiota (12), but which also reported no statistical difference in the phenotypes of the IBS subjects and controls for rates of anxiety, depression, stool frequency and Bristol stool form. The relatively mild disease symptoms of this IBS cohort (12) may have confounded identifying a microbiome signature. Supporting this, in a recent study of the gut microbiome in IBS and IBD, microbiome alterations were significantly associated with a physician diagnosed IBS group but were of fewer and of lower significance in the self-diagnosed IBS subgroup (36).

### Example 2—Urine Metabolome Profiling of Ibs Patients and Controls

Materials and Methods

Subject recruitment and sample collection were carried out as described in Example 1.

Urine FAIMS: FAIMS analysis was performed using a protocol modified from that of Arasaradnam et al. (37) and described below. Any other appropriate method known in the art for detecting metabolites may be used in the methods of the invention. Frozen (−80° C.) urine samples were thawed overnight at 4° C., 5 mL of each urine sample was aliquoted into a 20 mL glass vial and placed into an ATLAS sampler (Owlstone, UK) attached to the Lonestar FAIMS instrument (Owlstone, UK). The sample was heated to 40° C. and sequentially run three times.

Each sample run had a flow rate over the sample of 500 mL/min of clean dry air.

Further make-up air was added to create a total flow rate of 2.5 L/min. The FAIMS was scanned from 0 to 99% dispersion field in 51 steps, +6 V to −6 V compensation voltage in 512 steps and both positive and negative ions were detected to produce an untargeted volatile organic compound (VOC) profile for each sample. The signals for each sample at each DF were smoothed using the Savitzky-Golay filter (window size=9, degree=3). The signals were trimmed based on an optimized cut-off of 0.007 for positive mode and −0.007 for negative mode outputs, to obtain the region of interest, and reduce the baseline noise. Signals were aligned to the trimmed signals at each DF, using cross-correlation, using the mean signal as reference to make them comparable. Since the initial DFs of the FAIMS signal, and higher DFs were non-informative, signals corresponding to 17th DF till 42nd DF of both, positive, and negative modes were considered. These pre-processing steps were performed using customized programs developed in Python, v. 2.7.11, with relevant packages (Scipy v-1.1, and Numpy v-1.15.2). To further reduce the complexity, and to retain informative data, kurtosis normality tests were performed on each feature vector and features with raw p-value >0.1, were considered, and final profile was generated for various statistical analyses.

Bioinformatics analysis of urine metabolome data (FAIMS): Each urine sample analysed using FAIMS yielded a profile with ca. 52,224 data points. A pooled profile containing these data points for each sample was generated for pre-processing, to reduce the noise, size, and complexity of the data.

Urine GC/LC MS: 5 mL samples of frozen urine were sent on dry ice to Metabolomic Discoveries (now Metabolon), Potsdam, Germany. Untargeted metabolomics analysis was performed using liquid chromatography (LC) and Solid Phase Microextraction (SPME) gas chromatography (GC) and metabolites were identified using electrospray ionization mass spectrometry (ESI-MS). Short chain fatty acids (SCFA) analysis was also performed by LC-tandem mass spectrometry.

For urine metabolomics, the values of metabolites were normalized with reference to urine creatinine levels in each sample.

Bioinformatics analysis of urine metabolome data (MS): Urine MS metabolomics data was returned for all IBS subjects (n=80) and all but 2 controls (n=63) as these did not pass QC or no sample was available. A total of 2,887 metabolites were returned from untargeted urine metabolomics analysis, of which 594 were identified. Only the identified features with peak values normalized by creatinine levels in urine (mg/dl) were considered for further analysis.

Machine learning: An in-house machine learning pipeline was applied to each datatype (in this example, urine MS metabolomics) using a twostep approach applying the Least Absolute Shrinkage and Selection Operator (LASSO) feature selection followed by Random Forest (RF) modelling (38), as described in Example 1. The models were implemented using R software version 3.4.0, using package glmnet version 2.0-10 for LASSO feature selection, and RF package randomForest version 4.6-12. (34). The ability of urine FAIMS metabolomics to differentiate between health classes was tested using support vector machines (SVM), with a linear kernel, using python 2.7 and Scikit-Learn (v 0.19.2) (39). Features of FAIMS profile were selected using kurtosis normality test. These features were centered and scaled. The samples were split into training and test set, for 10 fold cross validation. Class weights were balanced. Other parameters were set to default. No supervised feature selection was used.

Results

Altered Urine Metabolomes in IBS

Metabolomic analysis was extended to all subjects, focusing initially on urine as a non-invasive test sample. Two methods were compared: High field asymmetric waveform ion mobility spectrometry (FAIMS) analysis for volatile organics, and both GC- and LC-MS.

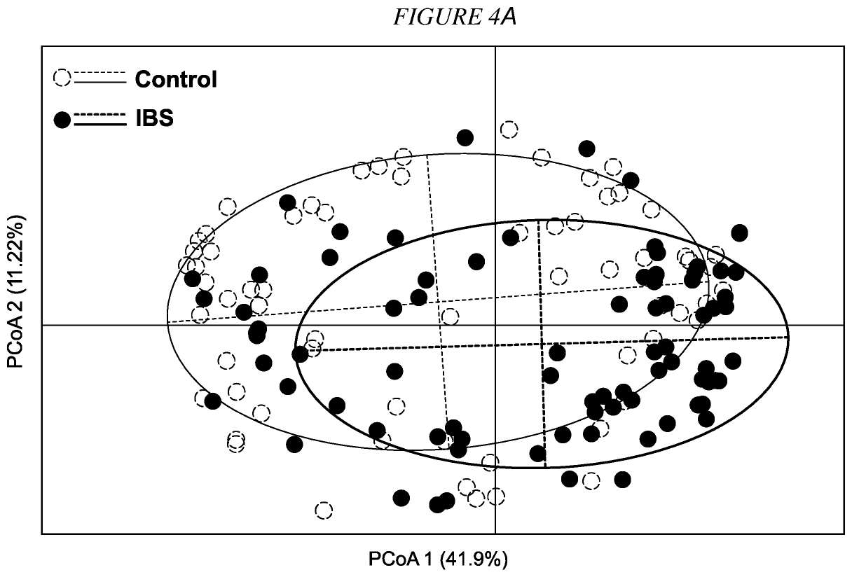

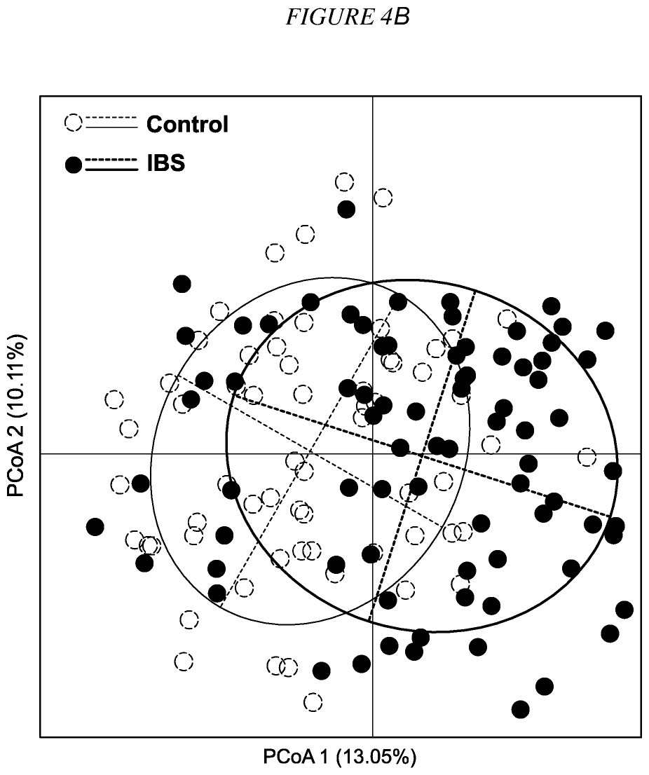

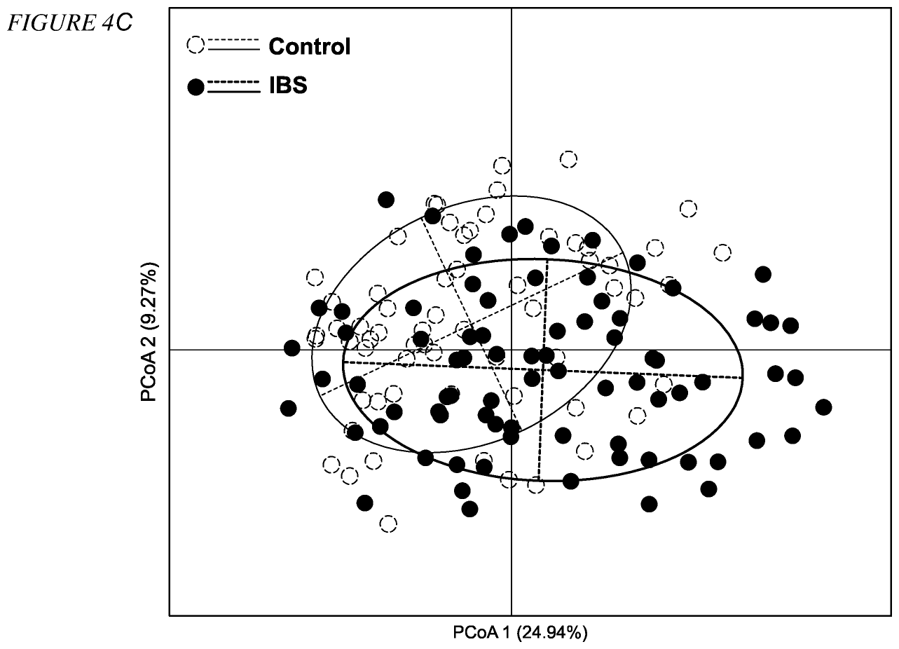

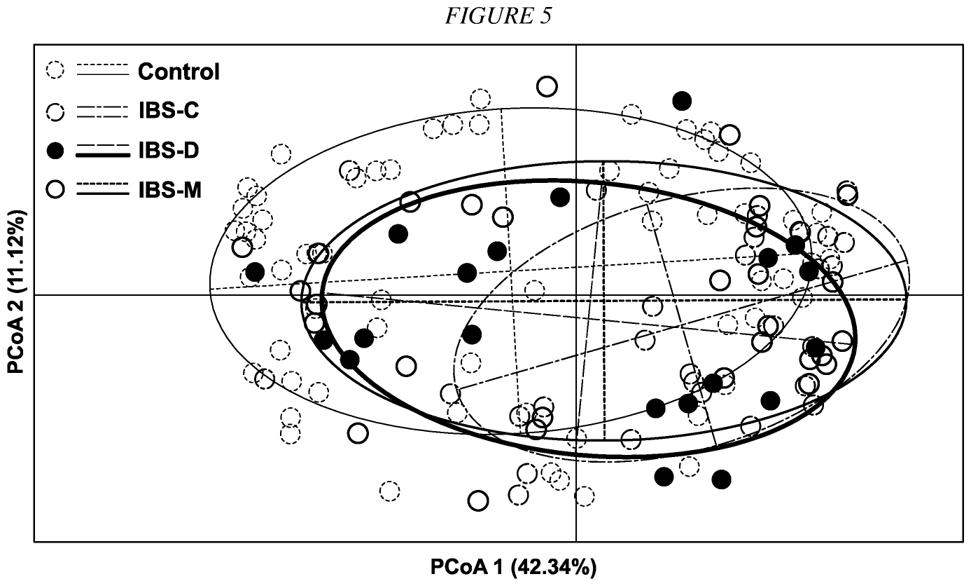

The FAIMS technique did not identify discriminatory metabolites directly, but separated samples/subjects by characteristic plumes of ionized metabolites. In unsupervised analysis, FAIMS readily identified urine samples from controls and IBS (FIG. 4a) but could not distinguish between IBS clinical subtypes (FIG. 5).

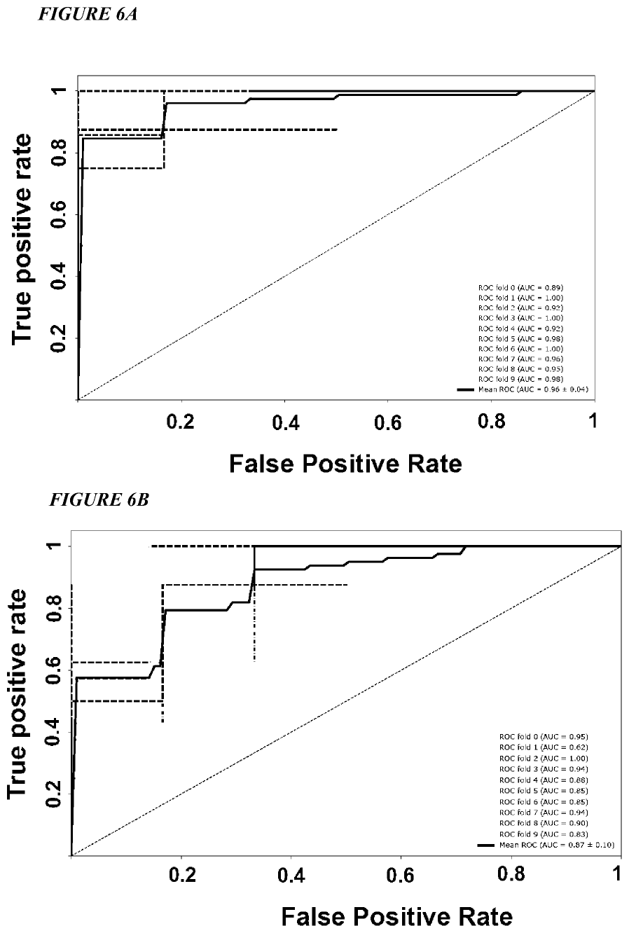

GC/LC-MS analysis of the urine metabolome also separated IBS patients from controls (FIG. 4b) and with greater accuracy than FAIMS (FIGS. 6a and 6b).

Machine learning identified four urine metabolomics features predictive of IBS (AUC 0.999; sensitivity: 0.988, specificity: 1.000) which were reflective of dietary components (Table 5). Pairwise comparison of control and IBS urine metabolomes identified 127 differentially abundant features (Table 6). 89 urine metabolites were significantly less abundant in IBS subjects including a number of amino acids such as L-arginine, a precursor for the biosynthesis of nitric oxide which is associated both with mucosal defence as well as IBS pathophysiology (40). Another 38 metabolites were present at significantly higher levels in IBS including an acylgylcine (N-undecanoylglycine) and an acylcarnitine (decanoylcarnitine). Elevated levels of metabolites from these groups are associated with altered fatty acid oxidation/metabolism and disease (41,42,43).

Discussion

Urine metabolomics was highly discriminatory for IBS. The machine learning model showed that the compounds identified were predominantly diet- or medication-associated.

### Example 3—Fecal Metabolome Profiling of Ibs Patients and Controls

Materials and Methods

Subject recruitment and sample collection were carried out as described in Example 1.

Fecal GC/LC MS: 1 g samples of frozen feces were sent on dry ice to Metabolomic Discoveries (now Metabolon), Potsdam, Germany. For LC-MS, the samples were dried and resuspended to a final concentration of 10 mg per 400 μL before analysis. GC-MS and SCFA analysis were performed using wet samples. Untargeted metabolomics and SCFA analysis was carried out as described previously for urine MS metabolomics.

Bioinformatics analysis of fecal metabolome data: Fecal MS metabolomics data was returned for all IBS subjects (n=80) and all but 2 controls (n=63) as these did not pass QC or no sample was available. 2,933 metabolites were returned from untargeted fecal metabolomics analysis carried out by the service provider of which 753 were identified. Metabolites identified using LC-MS were not normalized, since the fecal samples were already normalized with dry weight (10 mg per 400 μL) during sample preparation. Metabolites identified using GC-MS were normalized with corresponding sample wet weights. Only the identified metabolites were considered for further analyses. Machine learning analysis was carried out as described previously for the urine metabolome. Summary statistics for all datasets were generated using the Wilcoxon rank sum test with q-value adjustment for multiple testing.

Machine learning: An in-house machine learning pipeline was applied to each datatype (in this example, fecal MS metabolomics) using a twostep approach applying the Least Absolute Shrinkage and Selection Operator (LASSO) feature selection followed by Random Forest (RF) modelling (38), as described in Example 1. The models were implemented using R software version 3.4.0, using package glmnet version 2.0-10 for LASSO feature selection, and RF package randomForest version 4.6-12. (39).

Results

Altered Fecal Metabolomes in IBS

Analysis of the Fecal Metabolome by GC/LC-MS Separated IBS Patients from Controls

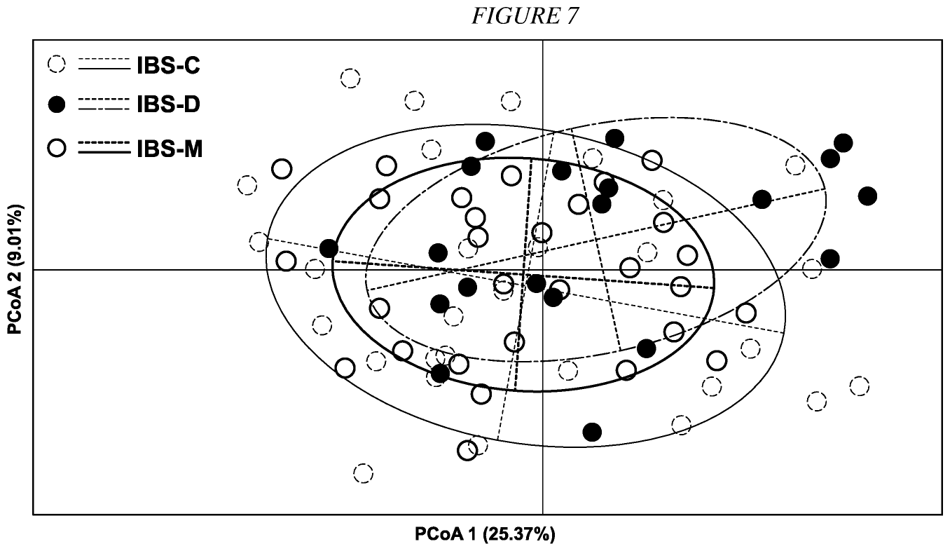

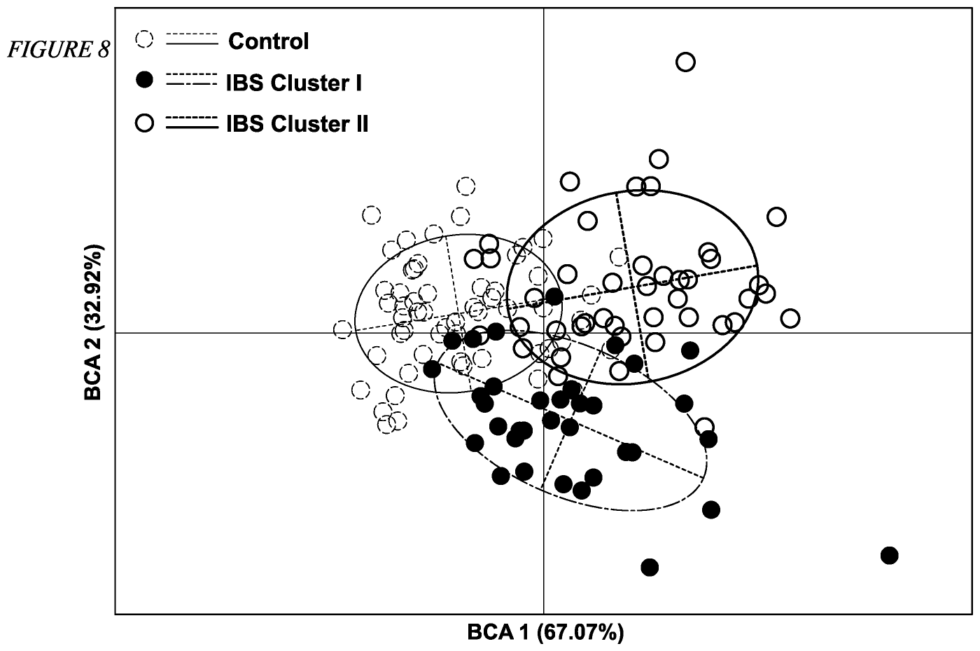

(FIG. 4c) but no difference was observed between the clinical IBS subtypes (FIG. 7). Machine learning applied to this dataset identified 40 fecal metabolites predictive of IBS (AUC:0.862, sensitivity: 0.821 and specificity: 0.647; Table 7) which included the amino acids L-tyrosine, and L-arginine; the bile acid UDCA; a bile pigment Iurobilin and dodecanedioic acid, an indicator of fatty acid oxidation defects (44).

Machine learning applied to the shotgun species dataset produced a marginally better prediction model for IBS than the fecal metabolomic model (AUC 0.878, sensitivity 0.894 and specificity 0.687) based on 40 predictive species (Table 2). The adenosine ribonucleotide de novo biosynthesis functional pathway was significantly more abundant in 11 of the 32 predictive species which resonates with adenosine being the fourth highest ranked predictive metabolite for IBS.

Pairwise comparison analysis of metabolites identified 128 significantly differential abundant features including 77 which were significantly depleted in IBS (Table 8). 51 fecal metabolites were significantly more abundant including tyrosine and lysine and three Bile Acids (BAs):[ ST hydroxy] (25R)-3alpha,7alpha-dihydroxy-5beta-cholestan-27-oyl taurine; [ST (2:0)] 5beta-Chola-3,11-dien-24-oic acid, and UDCA, which is one of the predictive metabolites for IBS.. BAs affect water absorption in intestine, and can lead to diarrhea (45).

The level of bile acid metabolites in the subgroups was analysed and a significant difference was observed in the IBS-D subtype for most bile acid categories (Total BAs, secondary BAs, sulphated BAs, UDCA and conjugated BAs) when compared to the control subjects as shown in Table 9a. These differences were associated with an altered functional potential, reflected by the ursodeoxycholate biosynthesis and glycocholate metabolism pathway gene abundances correlating with the secondary BAs, UDCA and total BA levels (Table 9b). Primary BAs and taurine:glycine conjugated BAs were not significantly different across the groups. Similar findings (in a smaller IBS/control cohort) were reported by Dior and colleagues (46) for secondary BAs, sulphated BAs and UDCA and taurine:glycine conjugated BAs.

Thus the differences in fecal microbiome composition and predicted function in IBS patients and controls are mirrored by differences in the measured metabolome in the two sample types.

Discussion

Here it is shown that the microbiome of patients with IBS is distinct from that of controls and this is reflected in fecal metabolome profiles. However, metagenome and metabolome configurations do not distinguish the so-called clinical subtypes of IBS (IBS-C, -D, -M).

The fecal metabolome correlated well with taxonomic and functional data for the microbiota.

### Example 4—Fecal Metabolome Profiling of Ibs Patients and Controls with an Alternative Machine Learning Pipeline

Materials and Methods

Subject recruitment and sample collection were carried out as described in Example 1.

Fecal GC/LC MS: 1 g samples of frozen feces were sent on dry ice to Metabolomic Discoveries (now Metabolon), Potsdam, Germany. For LC-MS, the samples were dried and resuspended to a final concentration of 10 mg per 400 μL before analysis. GC-MS and SCFA analysis were performed using wet samples. Untargeted metabolomics and SCFA analysis was carried out as described previously for urine MS metabolomics.

Bioinformatics analysis of fecal metabolome data: Fecal MS metabolomics data was returned for all IBS subjects (n=80) and all but 2 controls (n=63) as these did not pass QC or no sample was available. 2,933 metabolites were returned from untargeted fecal metabolomics analysis carried out by the service provider of which 753 were identified. Metabolites identified using LC-MS were not normalized, since the fecal samples were already normalized with dry weight (10 mg per 400 μL) during sample preparation. Metabolites identified using GC-MS were normalized with corresponding sample wet weights. Only the identified metabolites were considered for further analyses. Machine learning analysis was carried out as described previously for the urine metabolome. Summary statistics for all datasets were generated using the Wilcoxon rank sum test with q-value adjustment for multiple testing.

Machine learning: An in-house machine learning pipeline was applied to the fecal metabolomic data. The machine learning pipeline used in this example is similar to the machine learning pipeline used in Examples 1 to 3, but comprised additional optimization and validation steps, using a two step approach within a ten-fold cross-validation. Within each validation fold Least Absolute Shrinkage and Selection Operator (LASSO) feature selection was carried out followed by Random Forest (RF) modelling and an optimised model was validated against the cross validation test data which is external to the cross-validation training subset.

The classified fecal metabolome sample profiles were log₁₀ transformed before they were analysed in the machine learning pipeline. The transformed profiles were then used to classify the samples as IBS (80 samples) or Control (63 samples). The classified samples were then analysed in the machine learning pipeline.

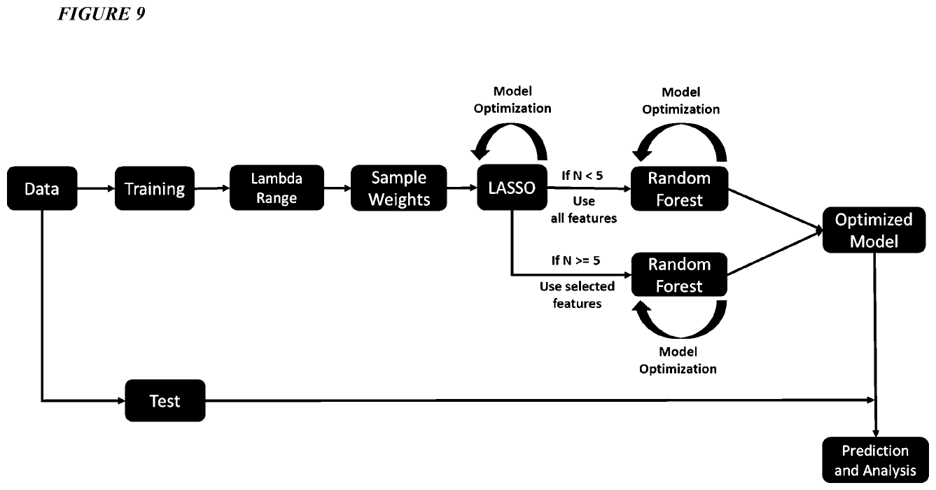

FIG. 9 shows the machine learning pipeline used in this example. The classified fecal metabolome sample profiles were first split into a training set and a test set. The training set was then used to generate an optimal lambda (λ) range for use by the LASSO algorithm. The optimal lambda (λ) range was generated using the previously described cross-validated LASSO and using the glmnet package (version 2.0-18). Pre-determination of an optimal lambda (λ) range reduces the computational time to run the pipeline and removes the need for a user to specify the ranges manually

After determination of the lambda (λ) range, the samples were assigned weights based on their class probabilities. The weights assigned to the training samples in this step were used in all subsequent applicable steps.

A LASSO algorithm substantially as described in Examples 1 to 3 was then applied to the weighted training samples. In this example, the LASSO algorithm used the previously calculated optimal lambda (λ) range, and used the Caret (version 6.0-84 in this example) and glmnet (version 2.0-18 in this example) packages, The ROC AUC (receiver operating characteristic, area under curve) metric was calculated using 10-fold internal cross validation, repeated 10 times. The feature coefficients identified by the optimized LASSO algorithm were extracted and features with non-zero coefficients were selected for further analysis. In FIG. 9, N refers to the number of features returned by the LASSO algorithm. If the number of features selected by LASSO was fewer than 5, then all of the features (pre-LASSO) were used to generate the random forest, i.e. the LASSO filtering was ignored by the random forest generator. If the number of features selected by LASSO was greater than or equal to 5, then only those features selected by LASSO were used for generation of the random forest (downstream classifier generation); otherwise all the features are considered for the classifier generation step.

Following feature selection using LASSO, an optimized random forest classifier (with 1500 trees) was generated using the selected features, or all of the features, as determined by N. This optimised random forest classifier can be used to predict the external test fold. Random forest generation was performed using Caret (version 6.0-84) and internal cross validation, by tuning the ‘mtry’ parameter to maximise the ROC AUC metric. For tuning, if the number of selected features is greater than or equal to 5, mtry ranges from 1 to the square root of the number of selected features or else the range is from 1 to 6. The optimized random forest classifier was then applied to the test set and the performance of the classifier was calculated via the AUC, sensitivity, and specificity metrics.

Both LASSO feature selection and RF modelling were performed within a 10-fold cross validation (CV), which generated an internal 10-fold prediction model that predicts the IBS or control classification of samples. This 10-fold cross-validation procedure was repeated ten times and the average AUC, sensitivity and specificity are reported. The optimized model is then used to predict the cross-validation test subset, and final classifier performance metrics are calculated from across the ten folds of the cross-validation (AUC, Sensitivity and Specificity).

Results

Fecal Metabolome is Predictive of IBS

The optimized random forest classifier was investigated for its predictive ability to classify samples as IBS or Control. External validation was 10-fold cross validation. Internal validation was 10-fold cross validation, repeated 10 times.

The performance summary and feature details are shown in Table 13. Features selected by LASSO having coefficients less than zero are associated with IBS, while positive coefficients are associated with Controls. Overall, for 10 folds, the mean ROC AUC was 0.686 (±0.132). Sensitivity, and specificity were 0.737 (±0.181), and 0.476 (±0.122), respectively. Accuracy was observed to be 0.622±0.095.

The classification threshold was also optimized to achieve maximum sensitivity and specificity using pROC package (version 1.15.0) and Youden J score. The obtained optimized values for Sensitivity and Specificity were 0.55, and 0.794, respectively. Thresholds were also optimized such that specificity >=0.9. The optimized values thus obtained for Sensitivity and Specificity were 0.288, and 0.905, respectively, at a threshold equal to 0.689.

The analysis identified 158 metabolites predictive of IBS, which are listed in Table 13. Metabolites with the highest RF feature importance included L-Phenylalanine, Adenosine and MG(20:3(8Z,11Z,14Z)/0:0/0:0). Increased levels of phenylethylamine, which is involved in the key metabolism pathway of phenylalanine, were found in fecal extracts of IBS mice compared with healthy control mice (47), indicating a connection between fecal phenylalanine levels and IBS, which is consistent with the present findings. Other metabolites which were predictive of IBS included the amino acids Lalanine, L-arginine, tyrosine and inosine previously reported as a biomarker of IBS (along with adenosine). The identified metabolites also included dodecanedioic acid, which, as discussed in Example 3, is an indicator of fatty acid oxidation defects (32).

Discussion

Here it is shown that the fecal metabolome profile of patients with IBS is distinct from that of controls. This observation is consistent with the results obtained using a different machine learning pipeline, as described in Example 3.

### Example 5—Co-Abundance Analysis of Gene Families with the Alternative Machine Learning Pipeline

Materials and Methods

Subject recruitment and sample collection were carried out as described in Example 1.

Co-abundance clustering: Clusters of co-abundant genes (CAGs) representing metagenomically-defined species variables were identified using gene family abundances. The generation of the gene family abundances is described in detail in Example 1, but for completeness is also detailed below.

Microbiome profiling and metagenomics: Genomic DNA was extracted and amplified from frozen fecal samples (0.25 g) using the method described by Brown et al. (26).

Microbiome profiling and metagenomics—Shotgun sequencing: Genomic DNA was extracted as described above. For shotgun sequencing, 1 μg (concentration>5 ng/μL) of high molecular weight DNA for each sample was sent to GATC Biotech, Germany for sequencing on Illumina HiSeq platform (HiSeq 2500) using 2×250 bp paired-end chemistry. This returned 2,714,158,144 raw reads (2,612,201,598 processed reads) of which 45.6% were mapped to an average of 222,945 gene families per sample with a mean count value of 8,924,302±2,569,353 per sample.

Bioinformatics analysis (16S amplicon sequencing): Miseq 16S sequencing data was returned for 144 subjects. Data generated for 3 samples (2 IBS and 1 control) were removed as the number of reads returned from sequencing was too low for analysis, leaving 141 samples (control: n=63, IBS n=78). Raw amplicon sequence data were merged and the reads trimmed using the flash methodology (27). The USEARCH pipeline was used to generate the OTU table (28). The UPARSE algorithm was used to cluster the sequences into OTUs at 97% similarity (29). UCHIME chimera removal algorithm was used with Chimeraslayer to remove chimeric sequences (30). The Ribosomal Database Project (RDP) taxonomic classifier was used to assign taxonomy to the representative OTU sequences (28) and microbiota compositional (abundance and diversity) information was generated.

Bioinformatics analysis (Shotgun metagenomic sequencing): For shotgun metagenomics, 6 control samples were not sequenced due to data not passing QC or no sample available (control: n=59; IBS n=80). The number of raw read pairs obtained after sequencing, varied from 5,247,013 to 21,280,723 (Mean=9,763,159±2,408,048). Reads were processed in accordance with the Standard Operating Procedure of Human Microbiome Project (HMP) Consortium (31). Metagenomic composition and functional profiles were generated using HUMAnN2 pipeline (32). For each sample, multiple profiles were obtained, including: microbial composition profiles from clade-specific gene information (using MetaPhlAn2), Gene family abundance, Pathway coverage and abundance.

After clusters of co-abundant genes representing metagenomically-defined species variables were identified from the gene family abundances, using the HUMAnN2 pipeline, a co-abundance analysis of the gene families was performed using a modified canopy clustering algorithm (Nielsen et al., 2014) (48). The canopy clustering algorithm was run with default parameters for 139 samples (IBS (80 samples) or Controls (59 samples)) using the relative abundance of 1,706,571 gene families (UniRef90 database) stratified by species using the HUMAnN2 methodology (Franzosa et al., 2018) (32).

The resulting gene family clusters were filtered to keep those where at least 90% of the cluster signal originated from more than three samples and contained more than two gene families. This was in order to remove clusters driven by outliers or with too few values, as recommended by Nielsen et al, 2014 (48). The clusters remaining after filtering were termed co-abundant groups or CAGs.

Abundance Indices of CAGs: The abundance indices of the CAGs were generated by Singular Value Decomposition (SVD) as implemented in Principal Component Analysis (PCA) using the dudi.pca command with default parameters (ade4 package in R. R version 3.5.1). The first principal component was extracted as the index and directionality was corrected by the index being compared to the median CAG gene abundance using the spearman correlation of all values within a CAG. CAGs returning a negative correlation were corrected by inverting the principal component values for that CAG. The principal component values were then scaled by subtracting the minimum value for a CAG from each CAG value.

Assignment of Taxonomy to CAGs: As each CAG is composed of multiple gene families, taxonomy was assigned to a CAG by reporting the most common genera and species associated with the gene families in the CAGs, along with the percentage of the CAG that they composed. For CAGs where a genus or species represented greater than 60% of the gene families, a taxonomy was assigned.

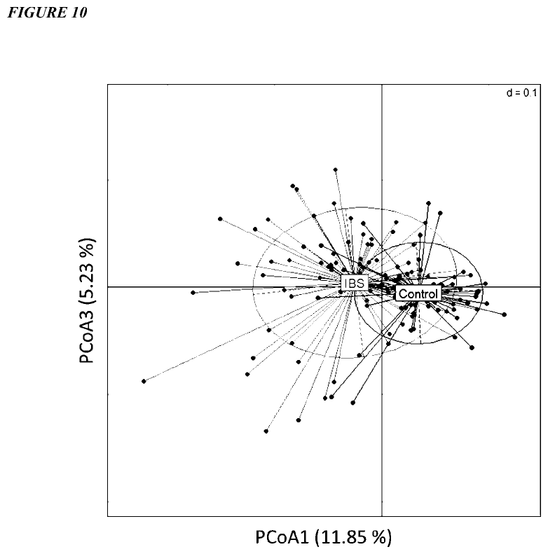

CAG results: After filtering for a minimum of 3 gene families per CAG, the strain level information (as represented by CAGs) within the shotgun dataset consisted of a total of 955 CAGs. The CAGs had a mean of 41.09 and maximum of 3,174 gene families. The distribution of CAGs across samples was sparse, with the mean number of CAGs per sample at 31.86 (3.34% of all 955 CAGs) and the max number of CAGs observed in any sample at 80 (8.38% of CAGs). The CAG cluster profile obtained was used to calculate inter-sample correlation distance based on Kendall correlation. Principal coordinate analysis based on this Beta-diversity metric showed a significant split between IBS and Controls (FIG. 2, PMANOVA p-value <0.001, vegan library), as seen in FIG. 10. No significant split was observed between the IBS subtypes (PMANOVA p-value=0.919).

Machine learning: The in-house machine learning pipeline described in Example 4 was applied to the CAG profiles, following preliminary multivariate analysis.

Results

CAG Cluster Profiles are Predictive of IBS (IBS v Control)

An informative way to reduce the complexity of metagenomic data while increasing biological signal is to assemble the reads into Co-abundant Gene groups or CAGs, representing strain-level variables and commonly referred to as metagenomic species. The optimized random forest classifier, generated using the CAG cluster profiles as input data, was investigated for its predictive ability to classify samples as IBS or Control. External validation was 10 fold CV, while internal validations for optimization, were 10 fold CV repeated 10 times.

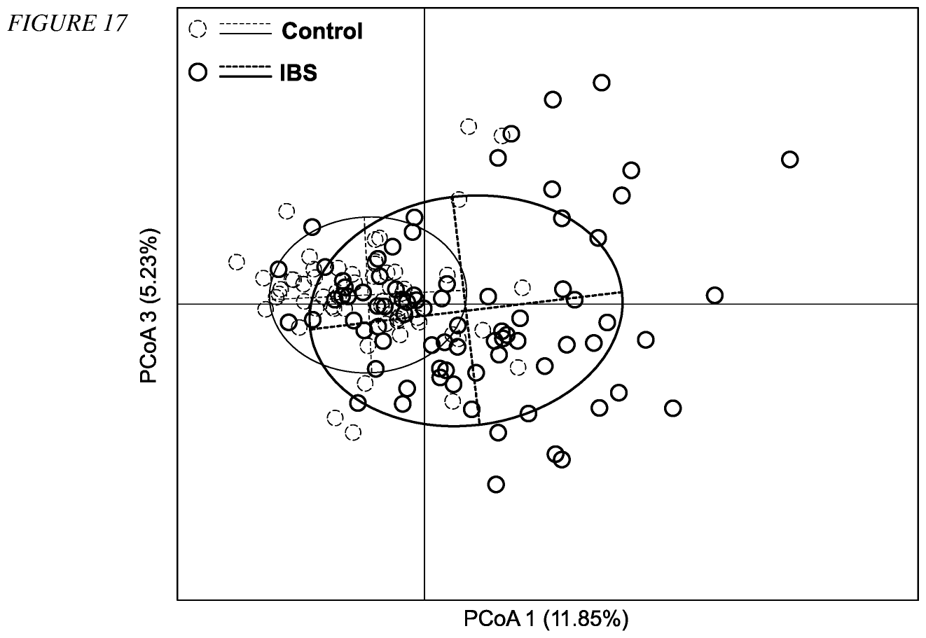

Analysis of these strain-level variables significantly differentiated IBS from controls, as shown in FIG. 17.

The performance summary, and feature details are described in table 14. Features selected by LASSO having coefficients less than zero are associated with IBS while positive coefficients are associated with Controls.

Machine learning applied to the metagenomic species (CAGs) dataset produced prediction model for IBS based on 136 predictive features (Table 14). Overall, for 10 folds, the mean ROC AUC was 0.814 (±0.134). Sensitivity, and specificity were 0.875(±0.102), and 0.497 (±0217), respectively. Accuracy was observed to be 0.713±0.134.

The classification threshold was optimized to achieve maximum sensitivity and specificity using pROC package and Youden J score. The obtained optimized values for Sensitivity and Specificity were 0.75, and 0.797, respectively. Thresholds were also optimized such that specificity was equal to or greater than (>=) 0.9. The optimized values thus obtained for Sensitivity and Specificity were 0.3875, and 0.915, respectively, at a threshold equal to 0.791.

Therefore, the analysis identified 136 CAGs predictive of IBS (table 14). Taxonomic assignment of the CAGs was sparse, with the majority of features unclassified, but assigned features were broadly consistent with the species-level analysis. The CAGs to which taxonomy was assigned include those associated with the genera Escherichia, Clostridium and Streptococcus, amongst others. At the species level, predictive CAGs included those associated with Escherichia coli, Streptococcus anginosus, Parabacteroides johnsonii, Streptococcus gordonii, Clostridium bolteae, Turicibacter sanguinis and Paraprevotella xylamphila, amongst others. A number of CAGs associated with individual strains were also identified, including Clostridiales bacterium 1_7_47 FAA, Eubacterium sp 3_1_31, Lachnospiraceae bacterium 5_1_57 FAA and Clostridiaceae bacterium JC118.

Discussion

Here it is shown that the microbiome of patients with IBS is distinct from that of controls, and that machine learning can be applied to co-abundance clustering of genes to reliably detect IBS.

A strain-level microbiome signature for IBS comprising 136 metagenomic species was identified. The separation between the microbiota of IBS and controls by unsupervised analysis exceeds that of earlier reports (10, 12). The limitations of 16S amplicon datasets and the relatively mild disease symptoms may account for failure to identify a microbiome signature in one report (12). Moreover, microbiome alterations were significantly associated with physician-diagnosed IBS, but were less significant in self-reported Rome criteria IBS (36).

### Example 6—Stratification of Ibs Subtypes Using Unsupervised Learning

Background

The current approach to stratification of patients into clinical subtypes based on predominant symptoms has significant limitations. This Example uses microbiome profiling to stratify IBS patients into subgroups.

Materials and Methods

Subject recuitment: A total of 142 samples were used for the analyses. Patients were recruited through gastroenterology clinics at Cork University Hospital, advertisements in the hospital, GP practices and shopping centres and emails to university staff. 80 patients were selected with IBS satisfying the Rome III/IV criteria and agreed inclusion/exclusion criteria and 65 healthy control. Not all samples were used for each analysis due to differing availability of sample specific datasets (Table 15). For example, sequencing data from 3 samples were of too poor quality to include with data from the remaining 142 samples and so were removed from the analyses.

Microbiome profiling: The samples were sequenced using 16S rRNA amplicon sequencing as described in Example 1. The resulting table showed abundance measures for each taxa across all 142 samples. If OTUs were present in 30% or less of samples they were filtered from the table.

Machine learning: Unsupervised learning was used to group the samples. A heatmap of the microbiome OTU table was generated along with hierarchical clustering applied using the Ward2 dendrogram and the Canberra distance measure.

Results

Descriptive Analysis of Samples

Of 142 samples that were analysed, 64 samples were healthy controls with the remaining 78 samples being IBS. Out of the 78, a group of 29 was diagnosed as the IBS-C subtype, a group of 20 was diagnosed as the IBS-D subtype and a group of 29 was diagnosed as the IBS-M subtype.

Identification of Subtypes

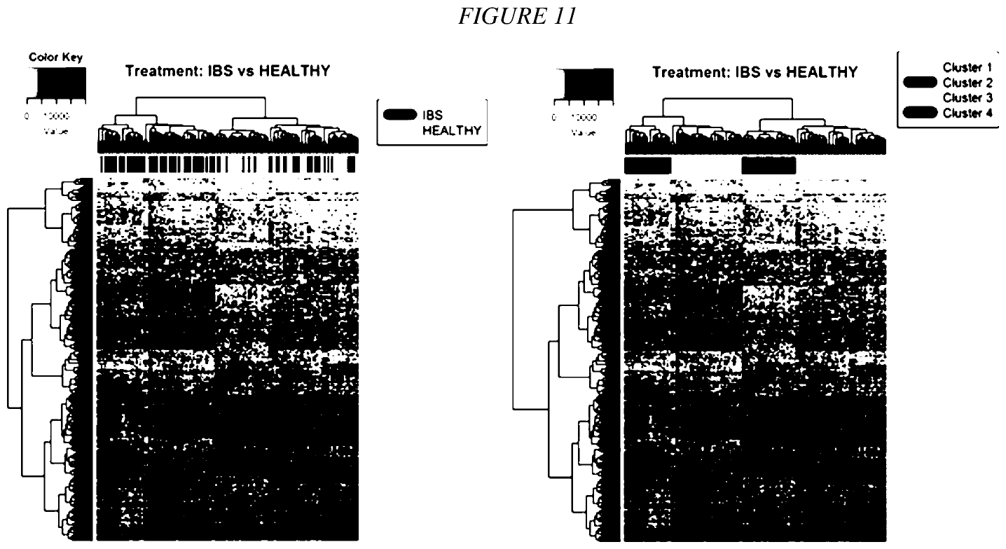

The hierarchical clustering identified 4 clusters (FIG. 11). The four clusters showed an uneven distribution of IBS and healthy controls. This altered beta diversity between healthy and IBS and within IBS provided the basis for the identification of three IBS subgroups (IBS-1, IBS-2, IBS-3). IBS-1 and IBS-2 subgroups relate to clusters 1 and 2 respectively with the IBS samples that co-cluster with healthy controls (clusters 3 and 4) being grouped into the IBS-3 subgroup. All healthy control samples are considered as a separate group in Examples 7-9.

Discussion

Here it is shown that hierarchical clustering applied to microbiome data may be used to define phenotypically distinct subgroups within the IBS population.

### Example 7—Microbiome Profiling and Differential Abundance Analysis (Genus Level) of Ibs Subgroups

Materials and Methods

Subjects: The same subjects were studied as in Example 6. The number of samples analysed in this Example is shown in Table 15.

Analysis of alpha diversity: The same OTU data was used as in Example 6. Observed species (richness) is a measure of diversity defined as the count of unique OTU's within a sample. Statistical analysis was performed using ANOVA.

Analysis of beta diversity: Principal Component Analysis with Canberra distance was used to analyse the differences in diversity of 16S data across the three IBS subgroups. Statistical analysis was performed using Pairwise Permutational MANOVA (adonis function, vegan library in R). The following six pairwise comparisons were made:

1. IBS-1 subgroup vs Healthy (significant).

2. IBS-1 subgroup vs IBS-2 subgroup (significant).

3. IBS-1 subgroup vs IBS-3 subgroup (significant).

4. IBS-2 subgroup vs IBS-3 subgroup (significant).

5. IBS-2 subgroup vs Healthy (significant).

6. IBS-3 subgroup vs Healthy (not significant).

Differential abundance analysis: Statistical analysis was carried out using the DESeq2 pipeline (R library: DESeQ2). Differentially abundant taxa at the genus level were identified for the above six pairwise comparisons.

Results

Differences in Alpha Diversity Across Subgroups

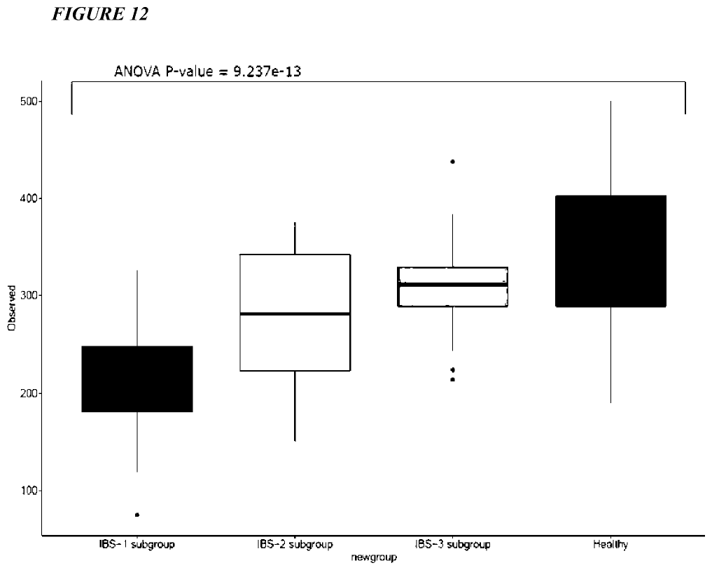

Applying the subgroup stratification of Example 1 to the OTU table and analysing the alpha diversity using the observed species metric within each of the groups revealed significant differences between all 4 groups, as shown in FIG. 12.

Principal Coordinate Analysis of Beta Diversity of 16S Data

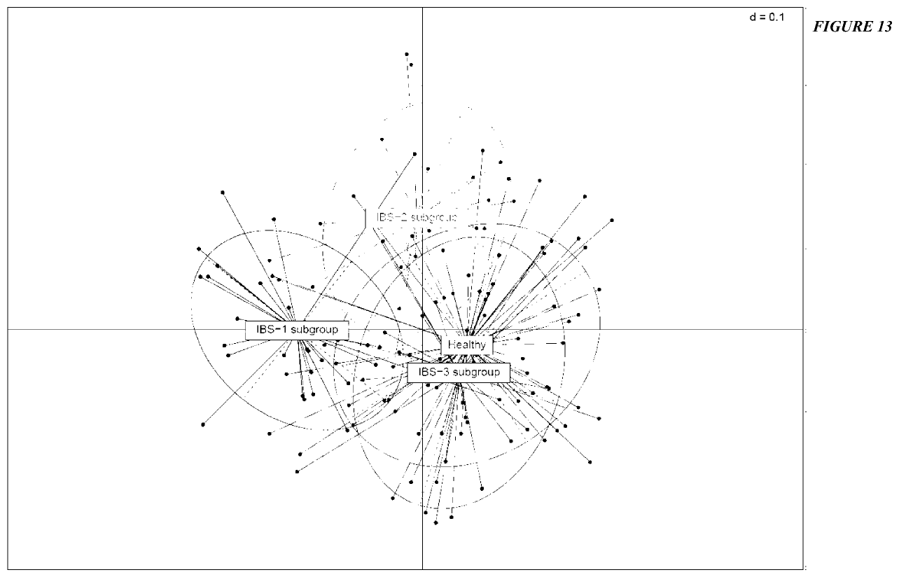

An analysis of the beta diversity using Principal Coordinate Analysis with Canberra distance at genus level across the three IBS subgroups, the results of which are shown in FIG. 13, replicated the distinct separation of the groups as observed in the clustering analysis (Example 1). Pairwise Permutational MANOVA testing of all groups indicated that 5 of the 6 pairwise comparisons were significantly different with the IBS-3 subgroup versus Healthy being not significant indicating a lack of a distinct split between the healthy group and IBS-3 subgroup.

The results show that the IBS-3 subgroup can be claimed to have a normal-like microbiota composition as evidenced by its lack of separation from the healthy controls.

The results of Principal Coordinate Analysis for Examples 7-9 are summarised in Table 16.

Differential Abundance Analysis—Genus Level

The differentially abundant genera identified in this study are shown in Table 17. For the comparison of the IBS-1 subgroup to Healthy groups there were in total 23 significant taxa where 6 were increased in abundance (adjusted p-value <0.05). With the IBS-2 subgroup vs Healthy groups there was 13 significant taxa where 6 were increased in abundance (adjusted p-value <0.05) and IBS-3 subgroup group when compared to the healthy group identified only 1 significant taxa (adjusted p-value <0.05) which was increased in abundance (Table 17). Notably, it was observed that Blautia and Eggertella were increased in both altered IBS groups (IBS-1 and IBS-2 subgroups). Butyricoccus, Copproccus and Prevotella were decreased in both altered IBS groups. Veillonella was the only genus to be increased in the Normal-like IBS group (IBS-3 subgroup).

The IBS-1 and IBS-2 subgroups were also compared to the normal-like IBS-3 subgroup. The results are shown in Table 18. As expected the genus level changes in the IBS-1 and IBS-2 subgroups to IBS-3 subgroup was similar to those seen for the IBS-1 and IBS-2 subgroups compared to the healthy controls (Table 17). Like in the comparison to the Healthy group both Blautia and Eggertella have increased in abundance and Prevotella has decreased. Flavonifrator has also increased in abundance across both altered IBS groups when comparing to the normal-like IBS group (IBS-3) which was not the case when comparing to the healthy group.

Discussion

Here it is shown that the IBS subgroups identified in Example 6 have distinct microbiome profiles. A number of differentially abundant genera were identified that are increased or decreased in particular subgroups. This may be informative for future stratification.

### Example 8—Metagenomic Profiling and Differential Abundance Analysis (Species Level) of IBS Subgroups

Materials and Methods

Subjects: The same subjects were studied as in Examples 6 and 7. The number of samples analysed in this Example is shown in Table 15.

Metagenome profiling: Samples were sequenced using Shotgun sequencing as described in Example 1. Quality assessment of reads was carried out using FASTQC and MultiQC. The Humann2 pipeline (which includes metaphlan2) was used to determine abundance measures for taxa at the species level. In brief the output files from the humann2 pipeline showing the relative abundance for each taxonomy were merged into a single table of relative abundance values for each taxonomy across all samples. The number of counts associated with each value of relative abundance can be inferred by multiplying each relative abundance value with the total number of reads in the sample which contains each relative abundance value and taking the integer part of the resulting value. The final output was then a count table for species level taxa across all 142 samples. Again, if taxa were present in 30% or less of samples then they were removed from the table.

Analysis of beta diversity: Principal Coordinate Analysis was performed as described in Example 6.

Differential abundance analysis: Statistical analysis was carried out as described in Example 7. Differentially abundant metabolites at the species level were identified for the same six pairwise comparisons.

Results

Principal Coordinate Analysis of Beta Diversity of Metagenomics Data

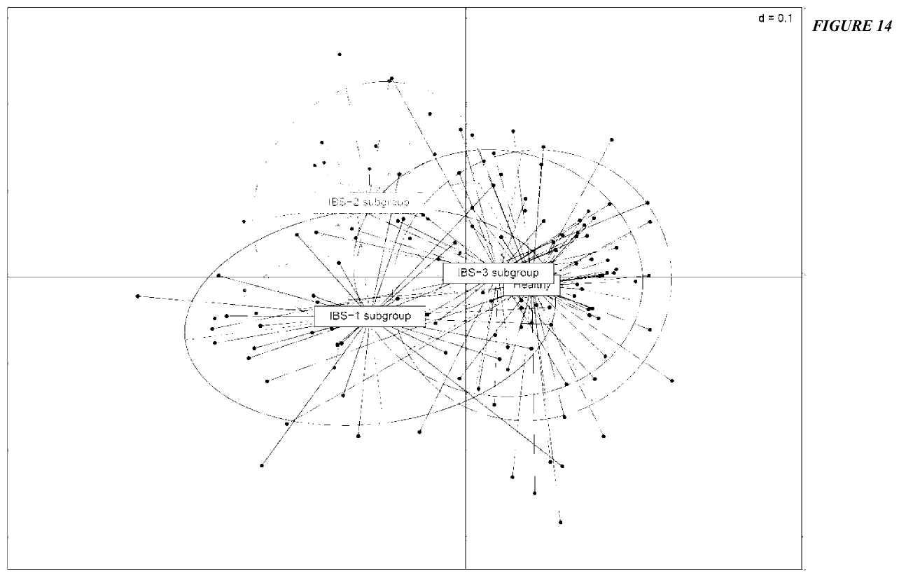

As shown in FIG. 14, the clustering from Example 6 is retained for the metagenomics dataset. Permutational MANOVA tests performed on the same pairwise comparisons as in the microbiome analysis (Example 7) showed the metagenomic beta diversity of the stratified samples to be the same in terms of significance to that of the microbiome beta diversity (Table 16).

Differential Abundance Analysis—Species Level

As in Example 7, an intersection matrix was used to portray the taxa between groups that had increased or decreased in abundance (Table 19). The matrix easily captured the difference between all the IBS groups showing the dissimilarities and similarities between each IBS group compared to the Healthy group relative to significance in species abundance. The fact that the normal-like IBS group is essentially the same as the healthy group in terms of species abundance is reflected in the absence of any species within the normal-like column of the intersection matrix (Table 19). For the altered IBS groups, Ruminoccus gnavus was increased in abundance in both IBS-1 and IBS-2 subgroups. Three different species of Clostridium have also increased across both altered IBS groups when compared to the Healthy group.

Using the same intersection matrix methodology, it was also invenstigated what species were significantly differentially abundant across the altered IBS groups (IBS-2 and IBS-3) when compared to the normal-like IBS group (IBS-3). The results are shown in Table 20. Notable differences were observed. Firstly, no species was found significantly differentially abundant between the IBS-1 subgroup group and the IBS-3 subgroup group. Secondly, in the IBS-2 subgroup group compared to the IBS-3 subgroup group there were only 4 species which were significantly differentially abundant. Amongst these, Ruminoccus gnavus and a Clostridium species showed significant increases in abundance. The comparison between both altered IBS groups also revealed a low number of significantly differentially abundant species.

Discussion

Notably, the separation of altered IBS groups (IBS-1 and IBS-2) to the normal-like (IBS-3) and healthy subjects that was seen here (FIG. 14) was extremely similar to that observed for the microbiome analysis (Example 7, FIG. 13).

This study also revealed that a number of species are significantly differentially abundant across the IBS subgroups, but not between the IBS-3 group and healthy subjects.

In summary, this study demonstrated that the IBS subgroups identified in Example 6 have distinct metagenomic profiles, which may be informative for future stratification.

### Example 9—Metabolomics Profiling and Differential Abundance Analysis of Ibs Subgroups

Materials and Methods

Subjects: The same subjects were studied as in Examples 6-8. The number of samples analysed in this Example is shown in Table 15.

Metabolome profiling: LC/GC-MS was used to measure the quantity of metabolomes for urine and fecal metabolites in each sample, as described in Examples 2 and 3, respectively, except SFCA analysis was not performed. The output measurement is a laser intensity and can be viewed in signal form as a peak on a spectrograph. Results from all samples are collated into a matrix of peak values for each metabolite detected across all 142 samples. Urine peak values were normalised to creatinine values. Faecal peak values were normalised to either dry weight of sample (LC) or wet weight of sample (GC).

Analysis of beta diversity: Principal Coordinate Analysis was performed as described in Example 6.

Results

Principal Coordinate Analysis of Beta Diversity of Fecal and Urine Metabolomics Data

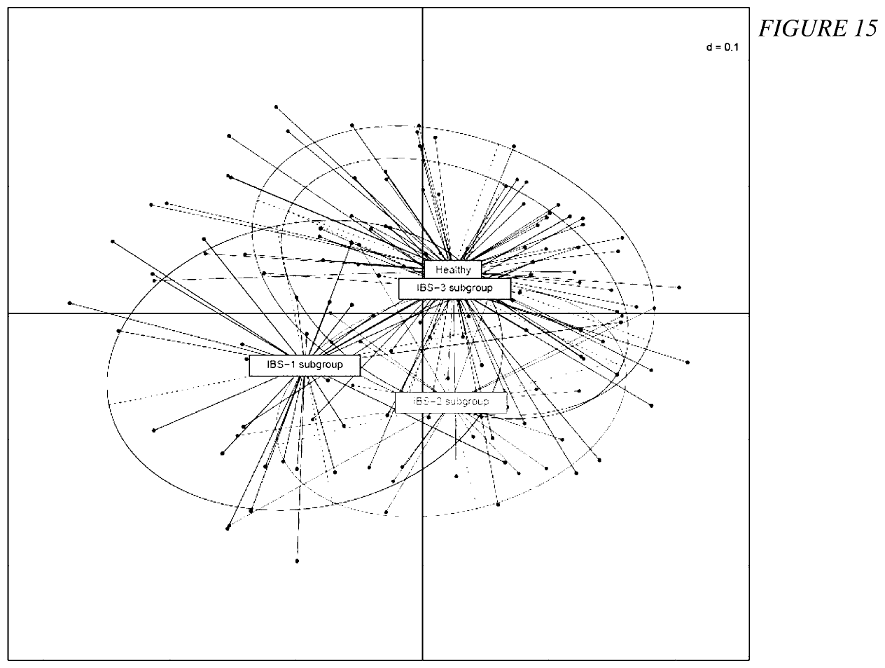

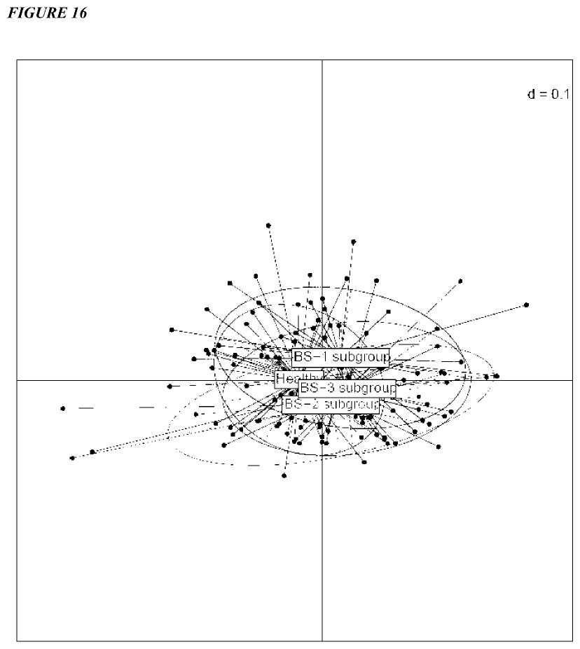

Using the normalised peak value data from the metabolomic results and the stratification from Examples 6-8, the beta diversity between the altered IBS groups, the normal-like IBS group and the Healthy group was determined. The results of Principal Coordinate Analysis for fecal and urine metabolomics data are shown in FIGS. 15 and 16, respectively. With respect to the fecal metabolomics samples, Permutational MANOVA tests of all six pairwise comparisons revealed the separation between groups in terms of significance to be exactly the same as that found previously for both the microbiome samples and the metagenome samples (Table 16). However, with respect to the urine metabolomic samples, the beta diversity analysis displayed different separation between groups in terms of significance, in contrast to other profiles. The Permutational MANOVA results for the separation of groups in the urine metabolomics for pairwise comparisons showed that only the 3 pairwise comparisons of the IBS groups (IBS-1, IBS-2 and IBS-3) to the Healthy were significant in terms of separation (Table 16). Notably, in the urine metabolomic dataset there is a significant separation between the normal-like IBS-3 group and the Healthy group (FIG. 16), whereas the converse result of IBS-3 subgroup and the Healthy subjects not being significantly separated was a characteristic of the microbiome, metagenome (Examples 7 and 8) and faecal metabolomics (FIG. 15) datasets.

Discussion

Here it is shown that the IBS subgroups identified in Example 6 have distinct fecal metabolomic profiles. The results obtained for the urine metabolomics data differed from those obtained for the microbiome, metagenomics and fecal metabolomics data. This may be informative for future stratification.

### Example 10—Urine Metabolome Profiling of Ibs Patients and Controls with an Alternative Machine Learning Pipeline

Materials and Methods

Subject recruitment and sample collection were carried out as described in Example 1.

Urine FAIMS: FAIMS analysis was performed using a protocol modified from that of Arasaradnam et al. (37) and described below. Any other appropriate method known in the art for detecting metabolites may be used in the methods of the invention. Frozen (−80° C.) urine samples were thawed overnight at 4° C., 5 mL of each urine sample was aliquoted into a 20 mL glass vial and placed into an ATLAS sampler (Owlstone, UK) attached to the Lonestar FAIMS instrument (Owlstone, UK). The sample was heated to 40° C. and sequentially run three times.

Each sample run had a flow rate over the sample of 500 mL/min of clean dry air.

Further make-up air was added to create a total flow rate of 2.5 L/min. The FAIMS was scanned from 0 to 99% dispersion field in 51 steps, ′+6 V to −6 V compensation voltage in 512 steps and both positive and negative ions were detected to produce an untargeted volatile organic compound (VOC) profile for each sample. The signals for each sample at each DF were smoothed using the Savitzky-Golay filter (window size=9, degree=3). The signals were trimmed based on an optimized cut-off of 0.007 for positive mode and −0.007 for negative mode outputs, to obtain the region of interest, and reduce the baseline noise. Signals were aligned to the trimmed signals at each DF, using crosscorrelation, using the mean signal as reference to make them comparable. Since the initial DFs of the FAIMS signal, and higher DFs were non-informative, signals corresponding to 17th DF till 42nd DF of both, positive, and negative modes were considered. These pre-processing steps were performed using customized programs developed in Python, v. 2.7.11, with relevant packages (Scipy v-1.1, and Numpy v-1.15.2). To further reduce the complexity, and to retain informative data, kurtosis normality tests were performed on each feature vector and features with raw p-value >0.1, were considered, and final profile was generated for various statistical analyses.

Bioinformatics analysis of urine metabolome data (FAIMS): Each urine sample analysed using FAIMS yielded a profile with ca. 52,224 data points. A pooled profile containing these data points for each sample was generated for pre-processing, to reduce the noise, size, and complexity of the data.

Urine GC/LC MS: 5 mL samples of frozen urine were sent on dry ice to Metabolomic Discoveries (now Metabolon), Potsdam, Germany. Untargeted metabolomics analysis was performed using liquid chromatography (LC) and Solid Phase Microextraction (SPME) gas chromatography (GC) and metabolites were identified using electrospray ionization mass spectrometry (ESI-MS). Short chain fatty acids (SCFA) analysis was also performed by LC-tandem mass spectrometry.

For urine metabolomics, the values of metabolites were normalized with reference to urine creatinine levels in each sample.

Bioinformatics analysis of urine metabolome data (MS): Urine MS metabolomics data was returned for all IBS subjects (n=80) and all but 2 controls (n=63) as these did not pass QC or no sample was available. A total of 2,887 metabolites were returned from untargeted urine metabolomics analysis, of which 594 were identified. Only the identified features with peak values normalized by creatinine levels in urine (mg/dl) were considered for further analysis.

Machine learning: An in-house machine learning pipeline was applied to the urine metabolomic data. The machine learning pipeline used in this example is similar to the machine learning pipeline used in Examples 1 to 3, but comprised additional optimization and validation steps, using a two step approach within a ten-fold cross-validation. Within each validation fold Least Absolute Shrinkage and Selection Operator (LASSO) feature selection was carried out followed by Random Forest (RF) modelling and an optimised model was validated against the cross validation test data which is external to the cross-validation training subset.

The classified urine metabolome sample profiles were log 10 transformed before they were analysed in the machine learning pipeline. The transformed profiles were then used to classify the samples as IBS (80 samples) or Control (63 samples). The classified samples were then analysed in the machine learning pipeline.

FIG. 9 shows the machine learning pipeline used in this example. The classified fecal metabolome sample profiles were first split into a training set and a test set. The training set was then used to generate an optimal lambda (λ) range for use by the LASSO algorithm. The optimal lambda (λ) range was generated using the previously described cross-validated LASSO and using the glmnet package (version 2.0-18). Pre-determination of an optimal lambda (λ) range reduces the computational time to run the pipeline and removes the need for a user to specify the ranges manually.

After determination of the lambda (λ) range, the samples were assigned weights based on their class probabilities. The weights assigned to the training samples in this step were used in all subsequent applicable steps.

A LASSO algorithm substantially as described in Examples 1 to 3 was then applied to the weighted training samples. In this example, the LASSO algorithm used the previously calculated optimal lambda (λ) range, and used the Caret (version 6.0-84 in this example) and glmnet (version 2.0-18 in this example) packages, The ROC AUC (receiver operating characteristic, area under curve) metric was calculated using 10-fold internal cross validation, repeated 10 times. The feature coefficients identified by the optimized LASSO algorithm were extracted and features with non-zero coefficients were selected for further analysis. In FIG. 9, N refers to the number of features returned by the LASSO algorithm. If the number of features selected by LASSO was fewer than 5, then all of the features (pre-LASSO) were used to generate the random forest, i.e. the LASSO filtering was ignored by the random forest generator. If the number of features selected by LASSO was greater than or equal to 5, then only those features selected by LASSO were used for generation of the random forest (downstream classifier generation); otherwise all the features are considered for the classifier generation step.

Following feature selection using LASSO, an optimized random forest classifier (with 1500 trees) was generated using the selected features, or all of the features, as determined by N. This optimised random forest classifier can be used to predict the external test fold. Random forest generation was performed using Caret (version 6.0-84) and internal cross validation, by tuning the ‘mtry’ parameter to maximise the ROC AUC metric. For tuning, if the number of selected features is greater than or equal to 5, mtry ranges from 1 to the square root of the number of selected features or else the range is from 1 to 6. The optimized random forest classifier was then applied to the test set and the performance of the classifier was calculated via the AUC, sensitivity, and specificity metrics.

Both LASSO feature selection and RF modelling were performed within a 10-fold cross validation (CV), which generated an internal 10-fold prediction model that predicts the IBS or control classification of samples. This 10-fold cross-validation procedure was repeated ten times and the average AUC, sensitivity and specificity are reported. The optimized model is then used to predict the cross-validation test subset, and final classifier performance metrics are calculated from across the ten folds of the cross-validation (AUC, Sensitivity and Specificity).

Results

Metabolomic analysis was extended its application to all subjects, focusing initially on urine as a non-invasive test sample. Two methods were compared: FAIMS analysis for volatile organics, and combined GC-/LC-MS. The FAIMS technique did not identify discriminatory metabolites directly, but separated samples/subjects by characteristic plumes of ionized metabolites. In unsupervised analysis, FAIMS readily identified urine samples from controls and IBS (FIG. 4a), but could not distinguish among IBS clinical subtypes (FIG. 5). GC/LC-MS analysis of the urine metabolome also separated IBS patients from controls (FIG. 4b) and with greater accuracy than FAIMS (FIGS. 6a and 6b).

Machine learning identified urine metabolomics features that are predictive of IBS (AUC 1.000; sensitivity: 1.000, specificity: 0.97, see Table 21a and 21b). Features that were highly predictive included dietary components such as epicatechin sulfate and medicagenic acid 3-O-b-Dglucuronide but also an acylgylcine (N-undecanoylglycine) and an acylcarnitine (decanoylcarnitine) (Table 21a and 21b). Pairwise comparison of control and IBS urine metabolomes identified 127 differentially abundant features (Table 6). Eighty nine urine metabolites were significantly less abundant in IBS subjects including a number of amino acids such as L-arginine, a precursor for the biosynthesis of nitric oxide which is associated both with mucosal defence and perhaps IBS pathophysiology. Another 38 metabolites were present at significantly higher levels in IBS including an acylgylcine (N-undecanoylglycine) and an acylcarnitine (decanoylcarnitine). Elevated levels of metabolites from these groups are associated with altered fatty acid oxidation/metabolism and disease.

Discussion

Although urine metabolomics was highly discriminatory for IBS, the machine learning analysis showed that the compounds identified were predominantly diet- or medication-associated. This observation is consistent with the results obtained using a different machine learning pipeline, as described in Example 2.

### CONCLUSION

The findings of the current study have clinical implications. First, the microbiome and fecal metabolome, and the urine metabolome, offer objective biomarkers for IBS.

Second, the traditional Rome subtyping of IBS is not supported by differences in microbiome and metabolome and it may be time to look for an alternative basis for disease classification.

Third, while the results in no way detract from the concept of an altered brain-gut axis in IBS, they point toward disturbances of the diet-microbiome-metabolome axis which are consistent with the complaints of many patients and should inform the design of future therapeutic interventions in IBS.

The taxa, pathways and metabolites that distinguish IBS subjects from controls identified here may be targeted by a range of microbiota-directed therapies such as fecal transplants, antibiotics, probiotics or live biotherapeutics.

Fourth, hierarchical clustering can be used to identify distinct IBS subtypes with differing microbiomes and fecal metabolomes. Some subgroups have an altered microbiome and fecal metabolome, whilst one subgroup had a normal-like microbiome and fecal metabolome. The identification and characterisation of these subgroups as described herein may be informative for future stratification and treatment.

Current stratification into clinical subtypes of IBS should not form the basis for therapeutic decisions, because the altered microbiota (compared to control subjects) is similar in the subtypes, consistent with alternating between constipation and diarrheal forms in many patients. A more informative stratification would be achieved by fecal microbiota and metabolome profiling. The metagenomic and metabolomic signatures that distinguish IBS subjects from controls identified here may be targeted by these microbiota-directed therapies.

### REFERENCES

1. Enck, P. et al Irritable bowel syndrome—dissection of a disease. A 13-steps polemic. Z Gastroenterol. 2017 July; 55(7):679-684.
2. Enck P, Aziz Q, Barbara G, et al. Irritable bowel syndrome. Nat. Rev. Dis. Primers 2016; 2:16014.
3. Soares R L. Irritable bowel syndrome: a clinical review. World J. Gastroenterol. 2014; 20:12144-60.
4. Van Oudenhove L, Aziz Q. The role of psychosocial factors and psychiatric disorders in functional dyspepsia. Nat. Rev. Gastroenterol. Hepatol. 2013; 10:158-67.
5. Koloski N A, Jones M, Kalantar J, et al. The brain-gut pathway in functional gastrointestinal disorders is bidirectional: a 12-year prospective population-based study. Gut 2012; 61:1284-90.
6. Schwille-Kiuntke J, Mazurak N, Enck P. Systematic review with meta-analysis: post-infectious irritable bowel syndrome after travellers' diarrhoea. Aliment. Pharmacol. Ther. 2015; 41:1029-37.
7. Quigley E M M. The Gut-Brain Axis and the Microbiome: Clues to Pathophysiology and Opportunities for Novel Management Strategies in Irritable Bowel Syndrome (IBS). J. Clin. Med. 2018; 7.
8. Lacy, B. E. and Patel, N. K. Rome Criteria and a Diagnostic Approach to Irritable Bowel Syndrome. J Clin Med. 2017 Oct. 26; 6(11).
9. Carroll I M, Ringel-Kulka T, Keku T O, et al. Molecular analysis of the luminal- and mucosalassociated intestinal microbiota in diarrhea-predominant irritable bowel syndrome. Am. J. Physiol. Gastrointest. Liver Physiol. 2011; 301:G799-807.
10. Rajilic-Stojanovic M, Biagi E, Heilig H G, et al. Global and Deep Molecular Analysis of Microbiota Signatures in Fecal Samples From Patients With Irritable Bowel Syndrome. Gastroenterology 2011; 141:1792-1801.
11. Jeffery I B, O'Toole P W, Ohman L, et al. An irritable bowel syndrome subtype defined by species-specific alterations in faecal microbiota. Gut 2012; 61:997-1006.
12. Tap J, Derrien M, Tornblom H, et al. Identification of an Intestinal Microbiota Signature Associated With Severity of Irritable Bowel Syndrome. Gastroenterology 2017; 152:111-123.
13. Collins S M. A role for the gut microbiota in IBS. Nat. Rev. Gastroenterol. Hepatol. 2014; 11:497-505.
14. Ohman L, Simren M. Intestinal microbiota and its role in irritable bowel syndrome (IBS). Curr. Gastroenterol. Rep. 2013; 15:323.
15. Tao Bai, Jing Xia, Yudong Jiang, Huan Cao, Yong Zhao, Lei Zhang, Huan Wang, Jun Song, and Xiaohua Hou. Comparison of the Rome IV and Rome III criteria for IBS diagnosis: A cross-sectional survey. Journal of gastroenterology and hepatology 32.5 (2017), pp. 1018-1025.
16. Magda Guilera, Agustin Balboa, and Fermin Mearin. Bowel habit subtypes and temporal patterns in irritable bowel syndrome: systematic review. The American journal of gastroenterology 100.5 (2005), p. 1174.
17. Drossman D A, Morris C B, Schneck S, et al. International survey of patients with IBS: symptom features and their severity, health status, treatments, and risk taking to achieve clinical benefit. J. Clin. Gastroenterol. 2009; 43:541-50.
18. Marcus J Claesson, Ian B Jeffery, Susana Conde, Susan E Power, Eibhlis M O'connor, Siobhan Cusack, Hugh M B Harris, Mairead Coakley, Bhuvaneswari Lakshminarayanan, Orla O'sullivan, et al. Gut microbiota composition correlates with diet and health in the elderly. Nature 488.7410 (2012), p. 178.
19. Lacy B E, Everhart K K, Weiser K T, et al. IBS patients' willingness to take risks with medications. Am. J. Gastroenterol. 2012; 107:804-9.
20. R. Guevremont, High-field asymmetric waveform ion mobility spectrometry: a new tool for mass spectrometry. J. Chromatogr. A, November 2004: 1058 (1-2): 3-19.
21. Savitzky A., Golay M J E. “Smoothing and Differentiation of Data by Simplified Least Squares Procedures”, Anal. Chem., 36(8), 1964, pages 1627-1639.
22. R. Tibshirani, “Regression Shrinkage and Selection via the Lasso”, Journal of the Royal Statistical Society, Series B, 58(1), 1996, pages 267-288.
23. Jeffery I B, O'Toole P W, Ohman L, Claesson M J, Deane J, Quigley E M, Simren M. 2012. “An irritable bowel syndrome subtype defined by species-specific alterations in fecal microbiota.” Gut 61:997-1006.
24. Zigmond, A. S. and R. P. Snaith, The hospital anxiety and depression scale. Acta Psychiatr. Scand., 1983. 67(6): p. 361-70.
25. Power, S. E., et al., Food and nutrient intake of Irish community-dwelling elderly subjects: who is at nutritional risk? J. Nutr. Health Aging., 2014. 18(6): p. 561-72.
26. Brown J R, Flemer B, Joyce S A, et al. Changes in microbiota composition, bile and fatty acid metabolism, in successful faecal microbiota transplantation for Clostridioides difficile infection. BMC Gastroenterol. 2018; 18:131.
27. Magoc T, Salzberg S L. FLASH: fast length adjustment of short reads to improve genome assemblies. Bioinformatics 2011; 27:2957-63.
28. Edgar R C. Search and clustering orders of magnitude faster than BLAST. Bioinformatics 2010; 26:2460-1.
29. Edgar R C. UPARSE: highly accurate OTU sequences from microbial amplicon reads. Nat. Methods 2013; 10:996-8.
30. Edgar R C, Haas B J, Clemente J C, et al. UCHIME improves sensitivity and speed of chimera detection. Bioinformatics 2011; 27:2194-200.
31. Consortium H M P. The Human Microbiome Project Consortium. Structure, function and diversity of the healthy human microbiome. Nature 2012; 486:207-14.
32. Franzosa E A, McIver L J, Rahnavard G, et al. Species-level functional profiling of metagenomes and metatranscriptomes. Nat. Methods 2018; 15:962-968.
33. Flemer B, Warren R D, Barrett M P, et al. The oral microbiota in colorectal cancer is distinctive and predictive. Gut 2018; 67:1454-1463.
34. Core Team R. R: A language and environment for statistical computing. 2017. R Foundation for Statistical Computing, Vienna, Austria.; https://www.R-project.org/.
35. Shankar V, Homer D, Rigsbee L, et al. The networks of human gut microbe-metabolite associations are different between health and irritable bowel syndrome. ISME J. 2015; 9:1899-903.
36. Vich Vila A, Imhann F, Collij V, et al. Gut microbiota composition and functional changes in inflammatory bowel disease and irritable bowel syndrome. Sci. Transl. Med. 2018; 10.
37. Arasaradnam R P, Westenbrink E, McFarlane M J, et al. Differentiating coeliac disease from irritable bowel syndrome by urinary volatile organic compound analysis—a pilot study. PLoSOne 2014; 9:e107312.
38. Flemer B, Warren R D, Barrett M P, et al. The oral microbiota in colorectal cancer is distinctive and predictive. Gut 2018; 67:1454-1463.
39. Neis, E. P., Dejong, C. H. & Rensen, S. S. The role of microbial amino acid metabolism in host metabolism. Nutrients 7, 2930-2946 (2015). Pedregosa F, et al. Scikit-learn: Machine Learning in Python. Journal of Machine Learning Research 12, 825-2830 (2011).
40. Wallace J L. Nitric oxide in the gastrointestinal tract: opportunities for drug development. Br. J. Pharmacol. 2019; 176:147-154.
41. Hoppel C. The role of carnitine in normal and altered fatty acid metabolism. Am. J. Kidney Dis. 2003; 41:54-12.
42. Liu X, Liu Y, Cheng M, et al. Metabolomic Responses of Human Hepatocytes to Emodin, Aristolochic Acid, and Triptolide: Chemicals Purified from Traditional Chinese Medicines. J. Biochem. Mol. Toxicol. 2015; 29:533-43.
43. Vishwanath V A. Fatty Acid Beta-Oxidation Disorders: A Brief Review. Ann. Neurosci. 2016; 23:51-5.
44. Korman S H, Waterham H R, Gutman A, et al. Novel metabolic and molecular findings in hepatic carnitine palmitoyltransferase I deficiency. Mol. Genet. Metab. 2005; 86:337.
45. Riemsma R, Al M, Corro Ramos I, et al. SeHCAT [tauroselcholic (selenium-75) acid] for the investigation of bile acid malabsorption and measurement of bile acid pool loss: a systematic review and cost-effectiveness analysis. Health Technol. Assess. 2013; 17:1-236.
46. Dior M, Delagreverie H, Duboc H, et al. Interplay between bile acid metabolism and microbiota in irritable bowel syndrome. Neurogastroenterol. Motil. 2016; 28:1330-40.
47. Yu L M, Zhao K J, Wang S S, Wang X and Lu B. Gas chromatography/mass spectrometry based metabolomic study in a murine model of irritable bowel syndrome. World J Gastroenterol. 2018. 24(8):894-904. doi: 10.3748/wjg.v24.i8.894.
48. Nielsen, H. B., Almeida, M., Juncker, A. S., Rasmussen, S., Li, J., Sunagawa, S., . . . MetaHIT Consortium. (2014). Identification and assembly of genomes and genetic elements in complex metagenomic samples without using reference genomes. Nature Biotechnology. hops://doi. org/10. 1038/nbt.2939.
49. Blaxter, M.; Mann, J.; Chapman, T.; Thomas, F.; Whitton, C.; Floyd, R.; Abebe, E. (October 2005). “Defining operational taxonomic units using DNA barcode data”. Philos Trans R Soc Lond B Biol Sci. 360 (1462): 1935-43.

### TABLES

TABLE 1
Genus level (16S) Machine learning LASSO and Random
Forest (RF) statistics of genera predictive of IBS

| | |
|---|---|
| LASSO | RF |

| lambda | AUC | Sens | Spec | mtry | AUC | Sens | Spec |
|---|---|---|---|---|---|---|---|
| 0.074 | 0.780 | 0.824 | 0.501 | 1 | 0.835 | 0.815 | 0.704 |

| | |
|---|---|
| 10-fold Cross Validation | 10-fold Cross Validation |

| Reference | | | Reference | | |
|---|---|---|---|---|---|
| Prediction | Control | IBS | Prediction | Control | IBS |
| Control | 30.3 | 14.2 | Control | 41.7 | 14.7 |
| IBS | 28.7 | 65.8 | IBS | 17.3 | 65.3 |
| Accuracy | (average) | 0.687 | Accuracy | (average) | 0.770 |

| Rank # | Ranking | Genus | Rank # | Ranking | Genus |
|---|---|---|---|---|---|
| 1 | 100.00 | Actinomyces | 1 | 100 | Lachnospiraceae_noname |
| 2 | 12.71 | Oscillibacter | 2 | 99.02 | Oscillibacter |
| 3 | 3.41 | Paraprevotella | 3 | 67.51 | Coprococcus |
| 4 | 3.11 | Lachnospiraceae_noname | 4 | 35.29 | Erysipelotrichaceae_noname |
| 5 | 1.49 | Erysipelotrichaceae_noname | 5 | 25.79 | Paraprevotella |
| 6 | 0.53 | Coprococcus | 6 | 0 | Actinomyces |

Analysis had 2 classes: Control and IBS and included 139 samples (IBS: n = 80 and Control: n = 59)
Metrics reported are the average values from 10 repeats of 10-fold Cross Validation.
Taxonomy classified using the RDP classfier, database version 2.10.1.

TABLE 2
Identification of predictive features of IBS by Shotgun species Machine learning LASSO and Random Forest (RF) statistics

| LASSO | | | | RF | | | |
|---|---|---|---|---|---|---|---|
| lambda | AUC | Sens | Spec | mtry | AUC | Sens | Spec |
| 0.04 | 0.662 | 0.675 | 0.516 | 1 | 0.878 | 0.894 | 0.687 |

| 10-fold Cross Validation | | | 10-fold Cross Validation | | |
|---|---|---|---|---|---|
| Reference | | | Reference | | |
| Prediction | Control | IBS | Prediction | Control | IBS |
| Control | 30.5 | 26 | Control | 40.5 | 8.5 |
| IBS | 28.5 | 54 | IBS | 18.5 | 71.5 |
| Accuracy | (average) | 0.608 | Accuracy | (average) | 0.806 |
| Rank # | Ranking | Taxon | Rank # | Ranking | Taxon |
| 1 | 100 | Prevotella_buccalis | 1 | 100 | Ruminococcus_gnavus |
| 2 | 25.43 | Butyricicoccus_pullicaecorum | 2 | 89.92 | Lachnospiraceae_bacterium_3_1_46FAA |
| 3 | 9.96 | Granulicatella_elegans | 3 | 82.31 | Coprococcus_catus |
| 4 | 2.8 | Pseudoflavonifractor_capillosus | 4 | 78.74 | Lachnospiraceae_bacterium_7_1_58FAA |
| 5 | 2.5 | Clostridium_ramosum | 5 | 77.9 | Barnesiella_intestinihominis |
| 6 | 2.17 | Streptococcus_sanguinis | 6 | 74.39 | Anaerotruncus_colihominis |
| 7 | 1.47 | Clostridium_citroniae | 7 | 71.53 | Eubacterium_eligens |
| 8 | 1.13 | Desulfovibrio_desulfuricans | 8 | 69.19 | Lachnospiraceae_bacterium_1_4_56FAA |
| 9 | 0.76 | Haemophilus_pittmaniae | 9 | 64.93 | Clostridium_symbiosum |
| 10 | 0.72 | Paraprevotella_clara | 10 | 59.37 | Roseburia_inulinivorans |
| 11 | 0.48 | Lachnospiraceae_bacterium_7_1_58FAA | 11 | 54.02 | Paraprevotella_clara |
| 12 | 0.45 | Streptococcus_anginosus | 12 | 53.32 | Ruminococcus_lactaris |
| 13 | 0.35 | Anaerotruncus_colihominis | 13 | 51.1 | Clostridium_citroniae |
| 14 | 0.29 | Lachnospiraceae_bacterium_1_4_56FAA | 14 | 50.26 | Lachnospiraceae_bacterium_2_1_58FAA |
| 15 | 0.24 | Clostridium_symbiosum | 15 | 50.2 | Clostridium_leptum |
| 16 | 0.23 | Mitsuokella_multacida | 16 | 49.57 | Ruminococcus_bromii |
| 17 | 0.21 | Clostridium_nexile | 17 | 47.96 | Bacteroides_thetaiotaomicron |
| 18 | 0.14 | Lachnospiraceae_bacterium_3_1_46FAA | 18 | 47.14 | Eubacterium_biforme |
| 19 | 0.13 | Lactobacillus_fermentum | 19 | 46.17 | Bifidobacterium_adolescentis |
| 20 | 0.12 | Eubacterium_biforme | 20 | 44.94 | Parabacteroides_distasonis |
| 21 | 0.12 | Clostridium_leptum | 21 | 42.72 | Coprococcus_sp_ART55_1 |
| 22 | 0.11 | Bacteroides_pectinophilus | 22 | 37.99 | Dialister_invisus |
| 23 | 0.087 | Coprococcus_catus | 23 | 36.52 | Bacteroides_faecis |
| 24 | 0.047 | Alistipes_sp_AP11 | 24 | 33.42 | Butyrivibrio_crossotus |
| 25 | 0.04 | Eubacterium_eligens | 25 | 33 | Clostridium_nexile |
| 26 | 0.037 | Roseburia_inulinivorans | 26 | 31.09 | Bacteroides_cellulosilyticus |
| 27 | 0.036 | Bacteroides_faecis | 27 | 27.59 | Pseudoflavonifractor_capillosus |
| 28 | 0.034 | Barnesiella_intestinihominis | 28 | 27.43 | Streptococcus_anginosus |
| 29 | 0.025 | Lachnospiraceae_bacterium_2_1_58FAA | 29 | 25.94 | Streptococcus_sanguinis |
| 30 | 0.024 | Bacteroides_thetaiotaomicron | 30 | 21.48 | Desulfovibrio_desulfuricans |
| 31 | 0.0075 | Ruminococcus_bromii | 31 | 21.3 | Clostridium_ramosum |
| 32 | 0.0048 | Ruminococcus_gnavus | 32 | 20.91 | Alistipes_sp_AP11 |
| 33 | 0.0037 | Ruminococcus_lactaris | 33 | 16.77 | Lactobacillus_fermentum |
| 34 | 0.0029 | Parabacteroides_distasonis | 34 | 9.17 | Mitsuokella_multacida |
| 35 | 0.0026 | Butyrivibrio_crossotus | 35 | 7.55 | Haemophilus_pittmaniae |
| 36 | 0.0022 | Bacteroides_cellulosilyticus | 36 | 5.71 | Bacteroides_pectinophilus |
| 37 | 0.00096 | Bifidobacterium_adolescentis | 37 | 3.29 | Prevotella_buccalis |
| 38 | 0.00056 | Bacteroides_sp_1_1_6 | 38 | 1.15 | Bacteroides_sp_1_1_6 |
| 39 | 0.00049 | Dialister_invisus | 39 | 1.04 | Granulicatella_elegans |
| 40 | 0.00048 | Coprococcus_sp_ART55_1 | 40 | 0 | Butyricicoccus_pullicaecorum |

Analysis had 2 classes: Control and IBS and included 139 samples (IBS: n = 80 and Control: n = 59)
LASSO feature selection 288 variables

TABLE 3
Shotgun species differentially abundant between the IBS and Control groups

| | | | | | Wilcoxon | | |
|---|---|---|---|---|---|---|---|
| Species | IBS (IQR) | | Control (IQR) | | Statistic | p-value | q-value |
| Ruminococcus_{—}gnavus | 0.0136 | (0-0.187) | 0 | (0-0) | 1209 | <0.001 | <0.001 |
| Clostridium_{—}bolteae | 0.016 | (0-0.0873) | 0 | (0-0.00248) | 1189 | <0.001 | <0.001 |
| Clostridiales_{—}bacterium_1_7_47FAA | 0 | (0-0.0122) | 0 | (0-0) | 1401 | <0.001 | <0.001 |
| Anaerotruncus_{—}colihominis | 0 | (0-0.0266) | 0 | (0-0) | 1457 | <0.001 | 0.00029 |
| Lachnospiraceae_{—}bacterium_1_4_56FAA | 0.000465 | (0-0.0453) | 0 | (0-0) | 1433 | <0.001 | 0.00029 |
| Flavonifractor_{—}plautii | 0.000835 | (0-0.0266) | 0 | (0-0) | 1480.5 | <0.001 | 0.00087 |
| Clostridium_{—}clostridioforme | 0 | (0-0.0209) | 0 | (0-0) | 1612 | 0.0001 | 0.00087 |
| Clostridium_{—}hathewayi | 0.00177 | (0-0.0316) | 0 | (0-0) | 1468 | 0.000106 | 0.00087 |
| Clostridium_{—}symbiosum | 0.00164 | (0-0.0882) | 0 | (0-0) | 1515 | 0.000201 | 0.00147 |
| Ruminococcus_{—}torques | 0.557 | (0.266-1.33) | 0.249 | (0.107-0.568) | 1428 | 0.000245 | 0.00161 |
| Alistipes_{—}senegalensis | 0 | (0-0.016) | 0.0155 | (0-0.0885) | 3027 | 0.000365 | 0.00218 |
| Prevotella_{—}copri | 0 | (0-0) | 0 | (0-0.596) | 2835 | 0.000607 | 0.00309 |
| Eggerthella_{—}lenta | 0 | (0-0.00447) | 0 | (0-0) | 1645.5 | 0.000612 | 0.00309 |
| Lachnospiraceae_{—}bacterium_5_1_57FAA | 0 | (0-0) | 0 | (0-0) | 1885 | 0.00116 | 0.00546 |
| Lachnospiraceae_{—}bacterium_3_1_46FAA | 0.0729 | (0.0207-0.2) | 0.0212 | (0.00171-0.0787) | 1534.5 | 0.00135 | 0.0059 |
| Clostridium_{—}asparagiforme | 0 | (0-0.0113) | 0 | (0-0) | 1651 | 0.00177 | 0.00705 |
| Barnesiella_{—}intestinihominis | 0.558 | (0-1.75) | 1.41 | (0.587-2.35) | 2968.5 | 0.00182 | 0.00705 |
| Clostridium_{—}citroniae | 0.00289 | (0-0.0237) | 0 | (0-0.00399) | 1630 | 0.00194 | 0.00709 |
| Eubacterium_{—}eligens | 0.669 | (0.0405-1.27) | 1.18 | (0.395-2.12) | 2947 | 0.00258 | 0.00874 |
| Lachnospiraceae_{—}bacterium_7_1_58FAA | 0.0273 | (0.0102-0.0683) | 0.0121 | (0.00511-0.0273) | 1579.5 | 0.00266 | 0.00874 |
| Coprococcus_sp_ART_551 | 0 | (0-0) | 0 | (0-4.25) | 2817.5 | 0.00376 | 0.0118 |
| Lachnospiraceae_{—}bacterium_3_1_57FAA_CT1 | 0.000675 | (0-0.0517) | 0 | (0-0.000522) | 1675 | 0.004 | 0.0119 |
| Clostridium_{—}ramosum | 0 | (0-0) | 0 | (0-0) | 1927.5 | 0.00532 | 0.0152 |
| Coprococcus_{—}catus | 0.238 | (0.0985-0.426) | 0.338 | (0.239-0.512) | 2877 | 0.0068 | 0.0186 |
| Eubacterium_{—}biforme | 0 | (0-0.37) | 0.222 | (0-0.86) | 2815 | 0.00721 | 0.0189 |
| Ruminococcus_{—}lactaris | 0 | (0-0.488) | 0.41 | (0-0.99) | 2814.5 | 0.00986 | 0.0249 |
| Bacteroides_{—}massiliensis | 0 | (0-0) | 0 | (0-1.19) | 2729 | 0.0108 | 0.0253 |
| Lachnospiraceae_{—}bacterium_2_1_58FAA | 0.00245 | (0-0.0446) | 0 | (0-0.0101) | 1735 | 0.0111 | 0.0253 |
| Haemophilus_{—}parainfluenzae | 0 | (0-0.0112) | 0.00638 | (0-0.0493) | 2788.5 | 0.0115 | 0.0253 |
| Clostridium_{—}nexile | 0 | (0-0.00897) | 0 | (0-0) | 1846.5 | 0.0119 | 0.0253 |
| Clostridium_{—}innocuum | 0 | (0-0.00333) | 0 | (0-0) | 1869.5 | 0.012 | 0.0253 |
| Bacteroides_{—}xylanisolvens | 0.00587 | (0-0.103) | 0.0561 | (0.00379-0.163) | 2807 | 0.0144 | 0.0296 |
| Oxalobacter_{—}formigenes | 0 | (0-0) | 0 | (0-0) | 2575 | 0.0167 | 0.0332 |
| Alistipes_{—}putredinis | 1.29 | (0-3.26) | 3.05 | (0.483-4.23) | 2796.5 | 0.0177 | 0.0342 |
| Paraprevotella_{—}clara | 0 | (0-0.014) | 0 | (0-0.179) | 2714 | 0.0192 | 0.036 |
| Odoribacter_{—}splanchnicus | 0.357 | (0-0.687) | 0.573 | (0.0488-0.883) | 2772 | 0.0217 | 0.0395 |
| Eubacterium_sp_3_1_31 | 0 | (0-0) | 0 | (0-0) | 1951 | 0.0266 | 0.0472 |

Shotgun compositional analysis performed on 139 samples (IBS: n = 78 and Control: n = 58)
Median abundance % represented as inter-quartile range (IQR)

TABLE 4
Genes associated with pathways differentially abundant between IBS and the Control groups

| | Pathway | IBS | Control | Wilcoxon | p- | q- |
|---|---|---|---|---|---|---|
| Pathway_Species | names | (IQR) | (IQR) | Statistic | value | value |
| PWY_6700_unclassified | queuosine biosynthesis | 0.00641 | 0.0102 | 3496 | 0 | 0 |
| | | (0.00467-0.0083) | (0.0082-0.0155) | | | |
| NONMEVIPP_PWY_unclassified | methylerythritol phosphate | 0.0124 | 0.017 | 3421 | 0 | 0.000142 |
| | pathway I | (0.00846-0.015) | (0.0138-0.0199) | | | |
| PWY_5667_unclassified | CDP-diacylglycerol | 0.00867 | 0.0129 | 3395 | 0 | 0.000142 |
| | biosynthesis I | (0.00609-0.0115) | (0.00984-0.0159) | | | |
| PWY_6737_unclassified | starch degradation V | 0.0158 | 0.0221 | 3398 | 0 | 0.000142 |
| | | (0.00983-0.02) | (0.0166-0.0268) | | | |
| PWY0_1319_unclassified | CDP-diacylglycerol | 0.00867 | 0.0129 | 3395 | 0 | 0.000142 |
| | biosynthesis II | (0.00609-0.0115) | (0.00984-0.0159) | | | |
| PWY_2942_unclassified | L-lysine biosynthesis III | 0.00753 | 0.0113 | 3374 | 0 | 0.000159 |
| | | (0.00574-0.00975) | (0.00881-0.014) | | | |
| PWY_6387_unclassified | UDP-N-acetylmuramoyl- | 0.0155 | 0.022 | 3376 | 0 | 0.000159 |
| | pentapeptide biosynthesis I | (0.0115-0.0191) | (0.0168-0.0277) | | | |
| | (meso-diaminopimelate | | | | | |
| | containing) | | | | | |
| PWY_724_unclassified | superpathway of L-lysine, L- | 0.00666 | 0.00967 | 3369 | 0 | 0.000159 |
| | threonine and L-methionine | (0.00519-0.00858) | (0.00778-0.0117) | | | |
| | biosynthesis II | | | | | |
| PWY_6386_unclassified | UDP-N-acetylmuramoyl- | 0.016 | 0.0227 | 3360 | 0 | 0.000166 |
| | pentapeptide biosynthesis II | (0.0119-0.0197) | (0.0174-0.0286) | | | |
| | (lysine-containing) | | | | | |
| PWY_6703_unclassified | preQ0 biosynthesis | 0.00304 | 0.00503 | 3357 | 0 | 0.000166 |
| | | (0.00198-0.00418) | (0.00351-0.00691) | | | |
| PWY_5097_unclassified | L-lysine biosynthesis VI | 0.00973 | 0.014 | 3340 | 0 | 0.000219 |
| | | (0.00701-0.0127) | (0.0107-0.0175) | | | |
| PWY0_1296_Clostridium_bolteae | purine ribonucleosides | 0 | 0 | 1467 | 0 | 0.00024 |
| | degradation | (0-0.0000507) | (0-0) | | | |
| UNINTEGRATED_unclassified | UNINTEGRATED | 8.68 | 10.6 | 3328 | 0 | 0.00024 |
| | | (6.59-9.76) | (9.11-11.8) | | | |
| PWY_7187_unclassified | pyrimidine | 0.00302 | 0.00453 | 3323 | 0 | 0.000249 |
| | deoxyribonucleotides de novo | (0.00241-0.00404) | (0.0035-0.00555) | | | |
| | biosynthesis II | | | | | |
| PWY_6124_unclassified | inosine-5′-phosphate | 0.00091 | 0.00153 | 3318 | 0 | 0.000258 |
| | biosynthesis II | (0.000715-0.0013) | (0.00111-0.00211) | | | |
| PEPTIDOGLYCANSYN_PWY_unclassified | peptidoglycan biosynthesis I | 0.0132 | 0.0179 | 3305 | 0 | 0.000305 |
| | (meso-diaminopimelate | (0.00956-0.0165) | (0.014-0.0252) | | | |
| | containing) | | | | | |
| PWY_5686_unclassified | UMP biosynthesis | 0.0146 | 0.0198 | 3302 | 0 | 0.000305 |
| | | (0.0108-0.0187) | (0.0161-0.0242) | | | |
| PWY_6151_unclassified | S-adenosyl-L-methionine | 0.0121 | 0.0159 | 3299 | 0 | 0.000305 |
| | cycle I | (0.00814-0.0148) | (0.0129-0.0189) | | | |
| PWY_7219_unclassified | adenosine ribonucleotides de | 0.0197 | 0.0308 | 3300 | 0 | 0.000305 |
| | novo biosynthesis | (0.0155-0.0268) | (0.021-0.0373) | | | |
| UNINTEGRATED_Ruminococcus_gnavus | UNINTEGRATED | 0 | 0 | 1431.5 | 0 | 0.000326 |
| | | (0-0.21) | (0-0) | | | |
| ANAGLYCOLYSIS_PWY_unclassified | glycolysis III (from glucose) | 0.00221 | 0.00375 | 3285.5 | 0 | 0.000365 |
| | | (0.00115-0.00326) | (0.00288-0.00472) | | | |
| COA_PWY_1_unclassified | coenzyme A biosynthesis I | 0.0129 | 0.0179 | 3273 | 0 | 0.000431 |
| | | (0.00896-0.016) | (0.0131-0.0211) | | | |
| PWY_6123_unclassified | inosine-5′-phosphate | 0.00117 | 0.00198 | 3274 | 0 | 0.000431 |
| | biosynthesis I | (0.000849-0.00167) | (0.0014-0.00266) | | | |
| PWY_5686_Lachnospiraceae_bacterium_7_1_58FAA | UMP biosynthesis | 0 | 0 | 1490 | 0 | 0.000471 |
| | | (0-0.0000914) | (0-0) | | | |
| ASPASN_PWY_unclassified | superpathway of L-aspartate | 0.000953 | 0.00134 | 3245 | 0 | 0.000492 |
| | and L-asparagine biosynthesis | (0.000508-0.0012) | (0.000972-0.00189) | | | |
| COA_PWY_1_Lachnospiraceae_bacterium_7_1_58FAA | coenzyme A biosynthesis I | 0 | 0 | 1511 | 0 | 0.000492 |
| | | (0-0.000138) | (0-0) | | | |
| HISDEG_PWY_unclassified | L-histidine degradation I | 0.00142 | 0.00363 | 3239 | 0 | 0.000492 |
| | | (0.000609-0.00296) | (0.00176-0.00524) | | | |
| PWY_6121_unclassified | 5-aminoimidazole | 0.0133 | 0.0182 | 3248 | 0 | 0.000492 |
| | ribonucleotide biosynthesis I | (0.00989-0.0166) | (0.0137-0.0221) | | | |
| PWY_6122_unclassified | 5-aminoimidazole | 0.0137 | 0.0189 | 3240 | 0 | 0.000492 |
| | ribonucleotide biosynthesis II | (0.0103-0.0177) | (0.0139-0.0227) | | | |
| PWY_6277_unclassified | superpathway of 5- | 0.0137 | 0.0189 | 3240 | 0 | 0.000492 |
| | aminoimidazole ribonucleotide | (0.0103-0.0177) | (0.0139-0.0227) | | | |
| | biosynthesis | | | | | |
| PWY_6737_Ruminococcus_gnavus | starch degradation V | 0 | 0 | 1571 | 0 | 0.000492 |
| | | (0-0.0000431) | (0-0) | | | |
| PWY_7111_Lachnospiraceae_bacterium_7_1_58FAA | pyruvate fermentation to | 0.0000524 | 0 | 1336 | 0 | 0.000492 |
| | isobutanol (engineered) | (0-0.000133) | (0-0.000026) | | | |
| PWY_7219_Lachnospiraceae_bacterium_7_1_58FAA | adenosine ribonucleotides de | 0.0000374 | 0 | 1372 | 0 | 0.000492 |
| | novo biosynthesis | (0-0.000151) | (0-0) | | | |
| PWY_7219_Ruminococcus_gnavus | adenosine ribonucleotides de | 0 | 0 | 1529.5 | 0 | 0.000492 |
| | novo biosynthesis | (0-0.0000285) | (0-0) | | | |
| PWY_7221_unclassified | guanosine ribonucleotides de | 0.0135 | 0.0179 | 3256 | 0 | 0.000492 |
| | novo biosynthesis | (0.00938-0.0172) | (0.0143-0.0237) | | | |
| PWY0_1296_Ruminococcus_gnavus | purine ribonucleosides | 0 | 0 | 1600 | 0 | 0.000492 |
| | degradation | (0-0.0000432) | (0-0) | | | |
| THRESYN_PWY_unclassified | superpathway of L-threonine | 0.00446 | 0.00613 | 3257 | 0 | 0.000492 |
| | biosynthesis | (0.00339-0.00534) | (0.00445-0.00756) | | | |
| TRNA_CHARGING_PWY_unclassified | tRNA charging | 0.0138 | 0.0199 | 3239 | 0 | 0.000492 |
| | | (0.0111-0.0192) | (0.0143-0.0263) | | | |
| UNINTEGRATED_Clostridium_bolteae | UNINTEGRATED | 0 | 0 | 1435 | 0 | 0.000492 |
| | | (0-0.359) | (0-0) | | | |
| VALSYN_PWY_Lachnospiraceae_bacterium_7_1_58FAA | L-valine biosynthesis | 0.0000524 | 0 | 1336 | 0 | 0.000492 |
| | | (0-0.000133) | (0-0.000026) | | | |
| PWY_841_unclassified | superpathway of purine | 0.0018 | 0.00305 | 3237 | 0 | 0.000499 |
| | nucleotides de novo | (0.00142-0.00242) | (0.00178-0.00397) | | | |
| | biosynthesis I | | | | | |
| PWY_2942_Lachnospiraceae_bacterium_7_1_58FAA | L-lysine biosynthesis III | 0 | 0 | 1524 | 0 | 0.000521 |
| | | (0-0.0000645) | (0-0) | | | |
| PWY_5973_unclassified | cis-vaccenate biosynthesis | 0.00195 | 0.00338 | 3231.5 | 0 | 0.000521 |
| | | (0.000762-0.00314) | (0.00249-0.00421) | | | |
| PWY_7221_Lachnospiraceae_bacterium_7_1_58FAA | guanosine ribonucleotides de | 0 | 0 | 1475 | 0 | 0.000521 |
| | novo biosynthesis | (0-0.0000568) | (0-0) | | | |
| DENOVOPURINE2_PWY_unclassified | superpathway of purine | 0.00181 | 0.00318 | 3225 | 0 | 0.000576 |
| | nucleotides de novo | (0.00153-0.0026) | (0.00192-0.00433) | | | |
| | biosynthesis II | | | | | |
| PWY_6545_unclassified | pyrimidine | 0.00126 | 0.00221 | 3222 | 0 | 0.000596 |
| | deoxyribonucleotides de novo | (0.000884-0.00184) | (0.00158-0.00312) | | | |
| | biosynthesis III | | | | | |
| PWY0_1296_unclassified | purine ribonucleosides | 0.0113 | 0.0152 | 3220 | 0 | 0.000596 |
| | degradation | (0.00861-0.0148) | (0.0117-0.0209) | | | |
| PWY0_166_unclassified | superpathway of pyrimidine | 0.00237 | 0.00396 | 3221 | 0 | 0.000596 |
| | deoxyribonucleotides de novo | (0.00171-0.00372) | (0.00313-0.0053) | | | |
| | biosynthesis (E. coli) | | | | | |
| PWY_7663_unclassified | gondoate biosynthesis | 0.00177 | 0.00284 | 3202 | 0 | 0.000826 |
| | (anaerobic) | (0.000653-0.00263) | (0.00208-0.00353) | | | |
| PWY_5695_unclassified | urate biosynthesis/inosine 5′- | 0.00353 | 0.00623 | 3199 | 0 | 0.000857 |
| | phosphate degradation | (0.00208-0.00572) | (0.00364-0.00997) | | | |
| NONMEVIPP_PWY_Ruminococcus_torques | methylerythritol phosphate | 0.000113 | 0 | 1405.5 | 0 | 0.00113 |
| | pathway I | (0-0.00033) | (0-0.000079) | | | |
| PWY_3001_unclassified | superpathway of L-isoleucine | 0.00515 | 0.00742 | 3180 | 0 | 0.00118 |
| | biosynthesis I | (0.00409-0.0064) | (0.00549-0.0085) | | | |
| PANTOSYN_PWY_unclassified | pantothenate and coenzyme A | 0.0028 | 0.00417 | 3169 | 0 | 0.00142 |
| | biosynthesis I | (0.00173-0.00405) | (0.00311-0.00545) | | | |
| PWY_6386_Lachnospiraceae_bacterium_7_1_58FAA | UDP-N-acetylmuramoyl- | 0 | 0 | 1582 | 0 | 0.00145 |
| | pentapeptide biosynthesis II | (0-0.0000798) | (0-0) | | | |
| | (lysine-containing) | | | | | |
| PWY_6387_Lachnospiraceae_bacterium_7_1_58FAA | UDP-N-acetylmuramoyl- | 0 | 0 | 1582 | 0 | 0.00145 |
| | pentapeptide biosynthesis I | (0-0.0000712) | (0-0) | | | |
| | (meso-diaminopimelate | | | | | |
| | containing) | | | | | |
| PWY_7219_Clostridium_bolteae | adenosine ribonucleotides de | 0 | 0 | 1568 | 0 | 0.00145 |
| | novo biosynthesis | (0-0.0000501) | (0-0) | | | |
| PWY_6122_Ruminococcus_gnavus | 5-aminoimidazole | 0 | 0 | 1687.5 | 0 | 0.00154 |
| | ribonucleotide biosynthesis II | (0-0.0000227) | (0-0) | | | |
| PWY_6277_Ruminococcus_gnavus | superpathway of 5- | 0 | 0 | 1687.5 | 0 | 0.00154 |
| | aminoimidazole ribonucleotide | (0-0.0000227) | (0-0) | | | |
| | biosynthesis | | | | | |
| PWY_6737_Clostridium_clostridioforme | starch degradation V | 0 | 0 | 1685.5 | 0 | 0.00154 |
| | | (0-0.000027) | (0-0) | | | |
| UNINTEGRATED_Lachnospiraceae_bacterium_1_4_56FAA | UNINTEGRATED | 0 | 0 | 1566.5 | 0 | 0.00154 |
| | | (0-0.0687) | (0-0) | | | |
| PWY_5188_Lachnospiraceae_bacterium_7_1_58FAA | tetrapyrrole biosynthesis I | 0 | 0 | 1554 | 0 | 0.00155 |
| | (from glutamate) | (0-0.0000684) | (0-0) | | | |
| CALVIN_PWY_unclassified | Calvin-Benson-Bassham cycle | 0.00666 | 0.00836 | 3152 | 0 | 0.00166 |
| | | (0.00482-0.00804) | (0.00703-0.0101) | | | |
| PWY_4984_Lachnospiraceae_bacterium_7_1_58FAA | urea cycle | 0 | 0 | 1510 | 0 | 0.00172 |
| | | (0-0.0000923) | (0-0) | | | |
| PWY_7184_unclassified | pyrimidine | 0.00137 | 0.00233 | 3149 | 0 | 0.00172 |
| | deoxyribonucleotides de novo | (0.000978-0.00222) | (0.00168-0.00388) | | | |
| | biosynthesis I | | | | | |
| PWY_7199_unclassified | pyrimidine | 0.00137 | 0.00215 | 3147 | 0 | 0.00174 |
| | deoxyribonucleosides salvage | (0.000839-0.0022) | (0.00147-0.00285) | | | |
| UNINTEGRATED_Lachnospiraceae_bacterium_2_1_58FAA | UNINTEGRATED | 0 | 0 | 1667 | 0.00011 | 0.00185 |
| | | (0-0.0361) | (0-0) | | | |
| PANTO_PWY_Ruminococcus_gnavus | phosphopantothenate | 0 | 0 | 1642.5 | 0.00012 | 0.00193 |
| | biosynthesis I | (0-0.0000557) | (0-0) | | | |
| PWY_6737_Clostridium_bolteae | starch degradation V | 0 | 0 | 1651 | 0.00012 | 0.00193 |
| | | (0-0.0000318) | (0-0) | | | |
| PWY_6151_Ruminococcus_gnavus | S-adenosyl-L-methionine | 0 | 0 | 1712 | 0.00012 | 0.00198 |
| | cycle I | (0-0.0000345) | (0-0) | | | |
| PWY_6609_Ruminococcus_gnavus | adenine and adenosine salvage | 0 | 0 | 1718 | 0.00014 | 0.00232 |
| | III | (0-0.0000122) | (0-0) | | | |
| PEPTIDOGLYCANSYN_PWY_Lachnospiraceae_bacterium_7_1_58FAA | peptidoglycan biosynthesis I | 0 | 0 | 1629 | 0.00015 | 0.00243 |
| | (meso-diaminopimelate | (0-0.0000774) | (0-0) | | | |
| | containing) | | | | | |
| PWY_7219_Clostridium_symbiosum | adenosine ribonucleotides de | 0 | 0 | 1608.5 | 0.00016 | 0.00248 |
| | novo biosynthesis | (0-0.0000445) | (0-0) | | | |
| PWY_6122_Clostridium_clostridioforme | 5-aminoimidazole | 0 | 0 | 1769 | 0.00016 | 0.00249 |
| | ribonucleotide biosynthesis II | (0-0) | (0-0) | | | |
| PWY_6277_Clostridium_clostridioforme | superpathway of 5- | 0 | 0 | 1769 | 0.00016 | 0.00249 |
| | aminoimidazole ribonucleotide | (0-0) | (0-0) | | | |
| | biosynthesis | | | | | |
| UNINTEGRATED_Lachnospiraceae_bacterium_3_1_46FAA | UNINTEGRATED | 0.062 | 0 | 1481 | 0.00019 | 0.00284 |
| | | (0-0.202) | (0-0.0573) | | | |
| PWY_7219_Lachnospiraceae_bacterium_3_1_46FAA | adenosine ribonucleotides de | 0.0000183 | 0 | 1515.5 | 0.0002 | 0.00304 |
| | novo biosynthesis | (0-0.000202) | (0-0) | | | |
| PWY_6125_unclassified | superpathway of guanosine | 0.0022 | 0.00331 | 3105 | 0.00021 | 0.00309 |
| | nucleotides de novo | (0.00171-0.00324) | (0.00255-0.00535) | | | |
| | biosynthesis II | | | | | |
| PWY_5667_Lachnospiraceae_bacterium_7_1_58FAA | CDP-diacylglycerol | 0 | 0 | 1598.5 | 0.00023 | 0.00325 |
| | biosynthesis I | (0-0.0000801) | (0-0) | | | |
| PWY0_1319_Lachnospiraceae_bacterium_7_1_58FAA | CDP-diacylglycerol | 0 | 0 | 1598.5 | 0.00023 | 0.00325 |
| | biosynthesis II | (0-0.0000801) | (0-0) | | | |
| CENTFERM_PWY_unclassified | pyruvate fermentation to | 0 | 0.000128 | 3033 | 0.00026 | 0.00361 |
| | butanoate | (0-0.000114) | (0-0.000282) | | | |
| NONMEVIPP_PWY_Lachnospiraceae_bacterium_3_1_46FAA | methylerythritol phosphate | 0 | 0 | 1587 | 0.00026 | 0.00361 |
| | pathway I | (0-0.000137) | (0-0) | | | |
| PWY_6590_unclassified | superpathway of Clostridium | 0 | 0.000161 | 3034 | 0.00026 | 0.00361 |
| | acetobutylicum acidogenic | (0-0.000145) | (0-0.000354) | | | |
| | fermentation | | | | | |
| PWY_7220_Clostridiales_bacterium_1_7_47FAA | adenosine | 0 | 0 | 1798 | 0.00027 | 0.00361 |
| | deoxyribonucleotides de novo | (0-0) | (0-0) | | | |
| | biosynthesis II | | | | | |
| PWY_7222_Clostridiales_bacterium_1_7_47FAA | guanosine | 0 | 0 | 1798 | 0.00027 | 0.00361 |
| | deoxyribonucleotides de novo | (0-0) | (0-0) | | | |
| | biosynthesis II | | | | | |
| PWY_7219_Clostridium_clostridioforme | adenosine ribonucleotides de | 0 | 0 | 1723 | 0.00029 | 0.0038 |
| | novo biosynthesis | (0-0.00002) | (0-0) | | | |
| UNINTEGRATED_Clostridium_clostridioforme | UNINTEGRATED | 0 | 0 | 1702.5 | 0.00034 | 0.00441 |
| | | (0-0.238) | (0-0) | | | |
| UNINTEGRATED_Prevotella_copri | UNINTEGRATED | 0 | 0 | 2854 | 0.00034 | 0.00441 |
| | | (0-0) | (0-0.318) | | | |
| PWY_7221_Ruminococcus_gnavus | guanosine ribonucleotides de | 0 | 0 | 1771 | 0.00034 | 0.00442 |
| | novo biosynthesis | (0-0) | (0-0) | | | |
| PWY_6386_Ruminococcus_gnavus | UDP-N-acetylmuramoyl- | 0 | 0 | 1772 | 0.00035 | 0.00447 |
| | pentapeptide biosynthesis II | (0-0) | (0-0) | | | |
| | (lysine-containing) | | | | | |
| PWY_6387_Ruminococcus_gnavus | UDP-N-acetylmuramoyl- | 0 | 0 | 1773 | 0.00036 | 0.00447 |
| | pentapeptide biosynthesis I | (0-0) | (0-0) | | | |
| | (meso-diaminopimelate | | | | | |
| | containing) | | | | | |
| PWY_6737_Lachnospiraceae_bacterium_3_1_46FAA | starch degradation V | 0 | 0 | 1569.5 | 0.00036 | 0.00447 |
| | | (0-0.000114) | (0-0) | | | |
| PWY0_1296_Clostridium_clostridioforme | purine ribonucleosides | 0 | 0 | 1773 | 0.00036 | 0.00447 |
| | degradation | (0-0) | (0-0) | | | |
| PWY_7219_Prevotella_copri | adenosine ribonucleotides de | 0 | 0 | 2850 | 0.00037 | 0.00448 |
| | novo biosynthesis | (0-0) | (0-0.00058) | | | |
| PWY_5667_Ruminococcus_gnavus | CDP-diacylglycerol | 0 | 0 | 1744 | 0.00038 | 0.00453 |
| | biosynthesis I | (0-0.0000191) | (0-0) | | | |
| PWY_7111_Clostridium_bolteae | pyruvate fermentation to | 0 | 0 | 1660.5 | 0.00038 | 0.00453 |
| | isobutanol (engineered) | (0-0.0000345) | (0-0) | | | |
| PWY0_1319_Ruminococcus_gnavus | CDP-diacylglycerol | 0 | 0 | 1744 | 0.00038 | 0.00453 |
| | biosynthesis II | (0-0.0000191) | (0-0) | | | |
| NONOXIPENT_PWY_Clostridium_bolteae | pentose phosphate pathway | 0 | 0 | 1717 | 0.00039 | 0.00456 |
| | (non-oxidative branch) | (0-0.0000225) | (0-0) | | | |
| PWY_7229_unclassified | superpathway of adenosine | 0.00617 | 0.00829 | 3063 | 0.00043 | 0.00492 |
| | nucleotides de novo | (0.00471-0.00799) | (0.00609-0.0109) | | | |
| | biosynthesis I | | | | | |
| UNINTEGRATED_Lachnospiraceae_bacterium_7_1_58FAA | UNINTEGRATED | 0.157 | 0 | 1502 | 0.00043 | 0.00492 |
| | | (0-0.239) | (0-0.155) | | | |
| PWY_6737_Lachnospiraceae_bacterium_2_1_58FAA | starch degradation V | 0 | 0 | 1827 | 0.00044 | 0.00498 |
| | | (0-0) | (0-0) | | | |
| PWY_6122_Clostridium_bolteae | 5-aminoimidazole | 0 | 0 | 1751 | 0.00046 | 0.00511 |
| | ribonucleotide biosynthesis II | (0-0.0000142) | (0-0) | | | |
| PWY_6277_Clostridium_bolteae | superpathway of 5- | 0 | 0 | 1751 | 0.00046 | 0.00511 |
| | aminoimidazole ribonucleotide | (0-0.0000142) | (0-0) | | | |
| | biosynthesis | | | | | |
| PANTO_PWY_unclassified | phosphopantothenate | 0.00787 | 0.00981 | 3058 | 0.00047 | 0.00512 |
| | biosynthesis I | (0.00518-0.0101) | (0.00729-0.0139) | | | |
| THISYNARA_PWY_unclassified | superpathway of thiamin | 0.000524 | 0.000835 | 3053 | 0.0005 | 0.00551 |
| | diphosphate biosynthesis III | (0.000233-0.000888) | (0.000431-0.00134) | | | |
| | (eukaryotes) | | | | | |
| PWY0_1296_Lachnospiraceae_bacterium_3_1_46FAA | purine ribonucleosides | 0 | 0 | 1601.5 | 0.00057 | 0.00615 |
| | degradation | (0-0.000154) | (0-0) | | | |
| PWY_6121_Ruminococcus_gnavus | 5-aminoimidazole | 0 | 0 | 1802.5 | 0.00061 | 0.00642 |
| | ribonucleotide biosynthesis I | (0-0) | (0-0) | | | |
| PWY_7221_Clostridium_clostridioforme | guanosine ribonucleotides de | 0 | 0 | 1802.5 | 0.00061 | 0.00642 |
| | novo biosynthesis | (0-0) | (0-0) | | | |
| PWY_2942_Ruminococcus_gnavus | L-lysine biosynthesis III | 0 | 0 | 1772 | 0.00061 | 0.00645 |
| | | (0-0) | (0-0) | | | |
| PWY_6897_Escherichia_coli | thiamin salvage II | 0 | 0 | 1598.5 | 0.00063 | 0.00655 |
| | | (0-0.000205) | (0-0) | | | |
| UNINTEGRATED_Clostridiales_bacterium_1_7_47FAA | UNINTEGRATED | 0 | 0 | 1773 | 0.00063 | 0.00655 |
| | | (0-0) | (0-0) | | | |
| NONMEVIPP_PWY_Lachnospiraceae_bacterium_7_1_58FAA | methylerythritol phosphate | 0 | 0 | 1682 | 0.0007 | 0.00689 |
| | pathway I | (0-0.0000615) | (0-0) | | | |
| PEPTIDOGLYCANSYN_PWY_Lachnospiraceae_bacterium_3_1_46FAA | peptidoglycan biosynthesis I | 0 | 0 | 1635 | 0.00069 | 0.00689 |
| | (meso-diaminopimelate | (0-0.000136) | (0-0) | | | |
| | containing) | | | | | |
| PWY_6121_Clostridium_bolteae | 5-aminoimidazole | 0 | 0 | 1806.5 | 0.00068 | 0.00689 |
| | ribonucleotide biosynthesis I | (0-0) | (0-0) | | | |
| PWY_6163_Lachnospiraceae_bacterium_3_1_46FAA | chorismate biosynthesis from | 0 | 0 | 1636 | 0.0007 | 0.00689 |
| | 3-dehydroquinate | (0-0.000137) | (0-0) | | | |
| PWY_6700_Prevotella_copri | queuosine biosynthesis | 0 | 0 | 2815 | 0.00069 | 0.00689 |
| | | (0-0) | (0-0.000255) | | | |
| PWY_7221_Prevotella_copri | guanosine ribonucleotides de | 0 | 0 | 2814 | 0.0007 | 0.00689 |
| | novo biosynthesis | (0-0) | (0-0.000402) | | | |
| PWY_5097_Prevotella_copri | L-lysine biosynthesis VI | 0 | 0 | 2813 | 0.00072 | 0.00692 |
| | | (0-0) | (0-0.000589) | | | |
| PWY_6121_Clostridium_clostridioforme | 5-aminoimidazole | 0 | 0 | 1856 | 0.00072 | 0.00692 |
| | ribonucleotide biosynthesis I | (0-0) | (0-0) | | | |
| ARO_PWY_unclassified | chorismate biosynthesis I | 0.0121 | 0.0144 | 3030 | 0.00073 | 0.00696 |
| | | (0.00844-0.0155) | (0.0125-0.018) | | | |
| PWY_2942_Prevotella_copri | L-lysine biosynthesis III | 0 | 0 | 2812 | 0.00074 | 0.00696 |
| | | (0-0) | (0-0.000514) | | | |
| ANAEROFRUCAT_PWY_unclassified | homolactic fermentation | 0.000931 | 0.00178 | 3026 | 0.00077 | 0.00703 |
| | | (0.000346-0.00226) | (0.00105-0.00325) | | | |
| PWY_6122_Ruminococcus_torques | 5-aminoimidazole | 0.0000587 | 0 | 1531.5 | 0.00078 | 0.00703 |
| | ribonucleotide biosynthesis II | (0-0.000253) | (0-0.0000671) | | | |
| PWY_6277_Ruminococcus_torques | superpathway of 5- | 0.0000587 | 0 | 1531.5 | 0.00078 | 0.00703 |
| | aminoimidazole ribonucleotide | (0-0.000253) | (0-0.0000671) | | | |
| | biosynthesis | | | | | |
| PWY_7111_Clostridium_symbiosum | pyruvate fermentation to | 0 | 0 | 1715 | 0.00079 | 0.00703 |
| | isobutanol (engineered) | (0-0.0000329) | (0-0) | | | |
| PWY_7111_Lachnospiraceae_bacterium_1_4_56FAA | pyruvate fermentation to | 0 | 0 | 1754.5 | 0.00079 | 0.00703 |
| | isobutanol (engineered) | (0-0.0000173) | (0-0) | | | |
| VALSYN_PWY_Clostridium_symbiosum | L-valine biosynthesis | 0 | 0 | 1715 | 0.00079 | 0.00703 |
| | | (0-0.0000329) | (0-0) | | | |
| VALSYN_PWY_Lachnospiraceae_bacterium_1_4_56FAA | L-valine biosynthesis | 0 | 0 | 1754.5 | 0.00079 | 0.00703 |
| | | (0-0.0000173) | (0-0) | | | |
| PANTO_PWY_Lachnospiraceae_bacterium_2_1_58FAA | phosphopantothenate | 0 | 0 | 1783 | 0.00081 | 0.00721 |
| | biosynthesis I | (0-0) | (0-0) | | | |
| PANTO_PWY_Lachnospiraceae_bacterium_3_1_46FAA | phosphopantothenate | 0 | 0 | 1603 | 0.00086 | 0.00757 |
| | biosynthesis I | (0-0.000224) | (0-0) | | | |
| PWY_1042_Alistipes_senegalensis | glycolysis IV (plant cytosol) | 0 | 0 | 2807.5 | 0.00095 | 0.00776 |
| | | (0-0) | (0-0.0000773) | | | |
| PWY_1042_unclassified | glycolysis IV (plant cytosol) | 0.00674 | 0.00932 | 3015 | 0.00093 | 0.00776 |
| | | (0.00521-0.0103) | (0.00716-0.0114) | | | |
| PWY_5686_Ruminococcus_gnavus | UMP biosynthesis | 0 | 0 | 1829 | 0.00093 | 0.00776 |
| | | (0-0) | (0-0) | | | |
| PWY_6386_Lachnospiraceae_bacterium_3_1_46FAA | UDP-N-acetylmuramoyl- | 0 | 0 | 1641 | 0.00093 | 0.00776 |
| | pentapeptide biosynthesis II | (0-0.000134) | (0-0) | | | |
| | (lysine-containing) | | | | | |
| PWY_6387_Lachnospiraceae_bacterium_3_1_46FAA | UDP-N-acetylmuramoyl- | 0 | 0 | 1642 | 0.00095 | 0.00776 |
| | pentapeptide biosynthesis I | (0-0.000125) | (0-0) | | | |
| | (meso-diaminopimelate | | | | | |
| | containing) | | | | | |
| PWY_6608_unclassified | guanosine nucleotides | 0.00112 | 0.0016 | 3015 | 0.00093 | 0.00776 |
| | degradation III | (0.000637-0.00162) | (0.00113-0.00218) | | | |
| PWY_6897_unclassified | thiamin salvage II | 0.000728 | 0.00191 | 3015 | 0.00091 | 0.00776 |
| | | (0.000211-0.00193) | (0.000663-0.00348) | | | |
| PWY_7219_Lachnospiraceae_bacterium_2_1_58FAA | adenosine ribonucleotides de | 0 | 0 | 1830 | 0.00095 | 0.00776 |
| | novo biosynthesis | (0-0) | (0-0) | | | |
| PWY0_1296_Clostridiales_bacterium_1_7_47FAA | purine ribonucleosides | 0 | 0 | 1830 | 0.00095 | 0.00776 |
| | degradation | (0-0) | (0-0) | | | |
| PYRIDNUCSYN_PWY_Alistipes_senegalensis | NAD biosynthesis I (from | 0 | 0 | 2822 | 0.00093 | 0.00776 |
| | aspartate) | (0-0) | (0-0.0000477) | | | |
| PEPTIDOGLYCANSYN_PWY_Ruminococcus_gnavus | peptidoglycan biosynthesis I | 0 | 0 | 1831 | 0.00098 | 0.00788 |
| | (meso-diaminopimelate | (0-0) | (0-0) | | | |
| | containing) | | | | | |
| PWY_5097_Ruminococcus_gnavus | L-lysine biosynthesis VI | 0 | 0 | 1800 | 0.00098 | 0.00788 |
| | | (0-0) | (0-0) | | | |
| HISDEG_PWY_Clostridium_symbiosum | L-histidine degradation I | 0 | 0 | 1727 | 0.00102 | 0.00819 |
| | | (0-0.0000378) | (0-0) | | | |
| PWY_6737_Clostridium_nexile | starch degradation V | 0 | 0 | 1833 | 0.00103 | 0.00819 |
| | | (0-0) | (0-0) | | | |
| PWY_7111_Lachnospiraceae_bacterium_3_1_46FAA | pyruvate fermentation to | 0 | 0 | 1630 | 0.00106 | 0.00821 |
| | isobutanol (engineered) | (0-0.000163) | (0-0) | | | |
| UNINTEGRATED_Anaerotruncuscoli_hominis | UNINTEGRATED | 0 | 0 | 1735 | 0.00104 | 0.00821 |
| | | (0-0.0845) | (0-0) | | | |
| VALSYN_PWY_Lachnospiraceae_bacterium_3_1_46FAA | L-valine biosynthesis | 0 | 0 | 1630 | 0.00106 | 0.00821 |
| | | (0-0.000163) | (0-0) | | | |
| COMPLETE_ARO_PWY_unclassified | superpathway of aromatic | 0.0113 | 0.014 | 3006 | 0.00107 | 0.00826 |
| | amino acid biosynthesis | (0.00793-0.0146) | (0.0121-0.0171) | | | |
| PWY_6126_unclassified | superpathway of adenosine | 0.00376 | 0.0063 | 3005 | 0.00109 | 0.00833 |
| | nucleotides de novo | (0.00249-0.00626) | (0.00407-0.00855) | | | |
| | biosynthesis II | | | | | |
| PWY_7221_Clostridium_symbiosum | guanosine ribonucleotides de | 0 | 0 | 1805 | 0.00111 | 0.00847 |
| | novo biosynthesis | (0-0) | (0-0) | | | |
| SULFATE_CYS_PWY_unclassified | superpathway of sulfate | 0 | 0.000348 | 2955 | 0.00112 | 0.00851 |
| | assimilation and cysteine | (0-0.000316) | (0-0.000681) | | | |
| | biosynthesis | | | | | |
| COA_PWY_1_Ruminococcus_gnavus | coenzyme A biosynthesis I | 0 | 0 | 1885 | 0.00116 | 0.00869 |
| | | (0-0) | (0-0) | | | |
| PWY_7221_Lachnospiraceae_bacterium_2_1_58FAA | guanosine ribonucleotides de | 0 | 0 | 1885 | 0.00116 | 0.00869 |
| | novo biosynthesis | (0-0) | (0-0) | | | |
| UNINTEGRATED_Flavonifractor_plautii | UNINTEGRATED | 0 | 0 | 1708 | 0.0012 | 0.00892 |
| | | (0-0.0595) | (0-0) | | | |
| GLUCONEO_PWY_unclassified | gluconeogenesis I | 0 | 0 | 2878 | 0.00121 | 0.00894 |
| | | (0-0) | (0-0.000509) | | | |
| PEPTIDOGLYCANSYN_PWY_Dorea_formicigenerans | peptidoglycan biosynthesis I | 0.000118 | 0.0000523 | 1538.5 | 0.00127 | 0.00919 |
| | (meso-diaminopimelate | (0.0000391-0.000188) | (0-0.0000868) | | | |
| | containing) | | | | | |
| PWY_5345_unclassified | superpathway of L-methionine | 0 | 0.000278 | 2907 | 0.00125 | 0.00919 |
| | biosynthesis (by | (0-0.000269) | (0-0.000587) | | | |
| | sulfhydrylation) | | | | | |
| PWY_6151_Prevotella_copri | S-adenosyl-L-methionine | 0 | 0 | 2780 | 0.00127 | 0.00919 |
| | cycle I | (0-0) | (0-0.000419) | | | |
| COA_PWY_unclassified | coenzyme A biosynthesis I | 0.00195 | 0.00274 | 2993 | 0.00131 | 0.00939 |
| | | (0.00102-0.00281) | (0.00193-0.00357) | | | |
| PWY_5676_unclassified | acetyl-CoA fermentation to | 0 | 0.000349 | 2955 | 0.00132 | 0.00942 |
| | butanoate II | (0-0.000384) | (0-0.000738) | | | |
| PWY_6163_unclassified | chorismate biosynthesis from | 0.0125 | 0.0148 | 2992 | 0.00133 | 0.00942 |
| | 3-dehydroquinate | (0.00817-0.0161) | (0.0126-0.0183) | | | |
| PWY_6121_Ruminococcus_torques | 5-aminoimidazole | 0.0000675 | 0 | 1569.5 | 0.00137 | 0.00968 |
| | ribonucleotide biosynthesis I | (0-0.000275) | (0-0.0000799) | | | |
| PWY_1042_Lachnospiraceae_bacterium_3_1_46FAA | glycolysis IV (plant cytosol) | 0 | 0 | 1692 | 0.00139 | 0.00972 |
| | | (0-0.000148) | (0-0) | | | |
| PWY_1269_Alistipes_senegalensis | CMP-3-deoxy-D-manno- | 0 | 0 | 2813.5 | 0.00143 | 0.00996 |
| | octulosonate biosynthesis I | (0-0) | (0-0.0000637) | | | |
| ARO_PWY_Lachnospiraceae_bacterium_3_1_46FAA | chorismate biosynthesis I | 0 | 0 | 1694 | 0.00144 | 0.00998 |
| | | (0-0.000143) | (0-0) | | | |
| PWY_6386_Dorea_formicigenerans | UDP-N-acetylmuramoyl- | 0.000127 | 0.0000649 | 1544.5 | 0.00147 | 0.0101 |
| | pentapeptide biosynthesis II | (0.0000421-0.0002) | (0-0.0001) | | | |
| | (lysine-containing) | | | | | |
| PWY6163_Clostridium_symbiosum | chorismate biosynthesis from | 0 | 0 | 1858.5 | 0.00153 | 0.0105 |
| | 3-dehydroquinate | (0-0) | (0-0) | | | |
| COA_PWY_1_Lachnospiraceae_bacterium_3_1_46FAA | coenzyme A biosynthesis I | 0 | 0 | 1729 | 0.00163 | 0.011 |
| | | (0-0.000126) | (0-0) | | | |
| PWY_4242_unclassified | pantothenate and coenzyme A | 0.00153 | 0.00235 | 2979 | 0.00162 | 0.011 |
| | biosynthesis III | (0.000733-0.00238) | (0.00155-0.003) | | | |
| PWY_5690_unclassified | TCA cycle II (plants and | 0.000228 | 0.000308 | 2976 | 0.00166 | 0.011 |
| | fungi) | (0.0000986-0.000334) | (0.000183-0.000568) | | | |
| PWY_7219_Lachnospiraceae_bacterium_1_4_56FAA | adenosine ribonucleotides de | 0 | 0 | 1861.5 | 0.00166 | 0.011 |
| | novo biosynthesis | (0-0) | (0-0) | | | |
| PWY0_1296_Eubacterium_eligens | purine ribonucleosides | 0.000272 | 0.00049 | 2976.5 | 0.00163 | 0.011 |
| | degradation | (0-0.000601) | (0.000165-0.00121) | | | |
| DTDPRHAMSYN_PWY_Coprococcus_catus | dTDP-L-rhamnose | 0 | 0.0000813 | 2941 | 0.0017 | 0.0112 |
| | biosynthesis I | (0-0.0000999) | (0-0.000126) | | | |
| PWY_7219_Alistipes_senegalensis | adenosine ribonucleotides de | 0 | 0 | 2841 | 0.00172 | 0.0113 |
| | novo biosynthesis | (0-0) | (0-0.0000724) | | | |
| PWY_6122_Flavonifractor_plautii | 5-aminoimidazole | 0 | 0 | 1745.5 | 0.00175 | 0.0114 |
| | ribonucleotide biosynthesis II | (0-0.0000315) | (0-0) | | | |
| | superpathway of 5- | | | | | |
| PWY_6277_Flavonifractor_plautii | aminoimidazole ribonucleotide | 0 | 0 | 1745.5 | 0.00175 | 0.0114 |
| | biosynthesis | (0-0.0000315) | (0-0) | | | |
| PWY_6703_Barnesiella_intestinihominis | preQ0 biosynthesis | 0.0000794 | 0.000364 | 2960 | 0.00177 | 0.0114 |
| | thiamin formation from | (0-0.000487) | (0.000113-0.000656) | | | |
| PWY_7357_Escherichia_coli | pyrithiamine and oxythiamine | 0.0000172 | 0 | 1634.5 | 0.0018 | 0.0115 |
| | (yeast) | (0-0.000274) | (0-0.00002) | | | |
| | pyrimidine | | | | | |
| PWY_7197_unclassified | deoxyribonucleotide | 0.00157 | 0.0021 | 2971 | 0.00182 | 0.0116 |
| | phosphorylation | (0.00108-0.00214) | (0.00148-0.00331) | | | |
| PWY_7221_Lachnospiraceae_bacterium_3_1_46FAA | guanosine ribonucleotides de | 0 | 0 | 1678 | 0.00185 | 0.0117 |
| | novo biosynthesis | (0-0.000128) | (0-0) | | | |
| NONOXIPENT_PWY_Ruminococcus_gnavus | pentose phosphate pathway | 0 | 0 | 1836 | 0.00189 | 0.0118 |
| | (non-oxidative branch) | (0-0) | (0-0) | | | |
| PWY_6122_Lachnospiraceae_bacterium_3_1_57FAA_CT1 | 5-aminoimidazole | 0 | 0 | 1756 | 0.0019 | 0.0118 |
| | ribonucleotide biosynthesis II | (0-0.0000249) | (0-0) | | | |
| | superpathway of 5- | | | | | |
| PWY_6277_Lachnospiraceae_bacterium_3_1_57FAA_CT1 | aminoimidazole ribonucleotide | 0 | 0 | 1756 | 0.0019 | 0.0118 |
| | biosynthesis | (0-0.0000249) | (0-0) | | | |
| PWY_6527_unclassified | stachyose degradation | 0.00523 | 0.00717 | 2969 | 0.00188 | 0.0118 |
| | | (0.00393-0.0076) | (0.00534-0.0099) | | | |
| COBALSYN_PWY_unclassified | adenosylcobalamin salvage | 0.0009 | 0.00144 | 2967 | 0.00193 | 0.0119 |
| | from cobinamide I | (0.000478-0.00157) | (0.000936-0.00204) | | | |
| PWY_7111_Clostridiales_bacterium_1_7_47FAA | pyruvate fermentation to | 0 | 0 | 1838 | 0.00199 | 0.0121 |
| | isobutanol (engineered) | (0-0) | (0-0) | | | |
| UNINTEGRATED_Clostridium_symbiosum | UNINTEGRATED | 0 | 0 | 1705 | 0.00197 | 0.0121 |
| | | (0-0.174) | (0-0) | | | |
| PWY_5667_Ruminococcus_torques | CDP-diacylglycerol | 0.000305 | 0.000163 | 1562 | 0.00207 | 0.0125 |
| | biosynthesis I | (0.00014-0.000741) | (0.0000809-0.000322) | | | |
| PWY0_1319_Ruminococcus_torques | CDP-diacylglycerol | 0.000305 | 0.000163 | 1562 | 0.00207 | 0.0125 |
| | biosynthesis II | (0.00014-0.000741) | (0.0000809-0.000322) | | | |
| PWY_6121_Lachnospiraceae_bacterium_3_1_46FAA | 5-aminoimidazole | 0 | 0 | 1709.5 | 0.00214 | 0.0128 |
| | ribonucleotide biosynthesis I | (0-0.000138) | (0-0) | | | |
| COMPLETE_ARO_PWY_Lachnospiraceae_bacterium_3_1_46FAA | superpathway of aromatic | 0 | 0 | 1721 | 0.00218 | 0.0129 |
| | amino acid biosynthesis | (0-0.000136) | (0-0) | | | |
| PWY_7228_unclassified | superpathway of guanosine | 0.00256 | 0.00372 | 2959 | 0.00218 | 0.0129 |
| | nucleotides de novo | (0.00191-0.00381) | (0.00261-0.00549) | | | |
| | biosynthesis I | | | | | |
| PWY_7383_unclassified | anaerobic energy metabolism | 0.00124 | 0.00178 | 2959 | 0.00218 | 0.0129 |
| | (invertebrates, cytosol) | (0.000886-0.00209) | (0.00137-0.0024) | | | |
| BRANCHED_CHAIN_AA_SYN_PWY_unclassified | superpathway of branched | 0.0073 | 0.0096 | 2957 | 0.00224 | 0.0131 |
| | amino acid biosynthesis | (0.00558-0.0099) | (0.00716-0.0114) | | | |
| PWY_7111_Clostridium_hathewayi | pyruvate fermentation to | 0 | 0 | 1808 | 0.00224 | 0.0131 |
| | isobutanol (engineered) | (0-0.000012) | (0-0) | | | |
| SO4ASSIM_PWY_unclassified | sulfate reduction I | 0 | 0.000167 | 2911 | 0.00228 | 0.0133 |
| | (assimilatory) | (0-0.000189) | (0-0.000471) | | | |
| PWY_5667_Lachnospiraceae_bacterium_3_1_46FAA | CDP-diacylglycerol | 0 | 0 | 1691 | 0.00234 | 0.0134 |
| | biosynthesis I | (0-0.000123) | (0-0) | | | |
| PWY_6121_Flavonifractor_plautii | 5-aminoimidazole | 0 | 0 | 1787 | 0.00235 | 0.0134 |
| | ribonucleotide biosynthesis I | (0-0.0000305) | (0-0) | | | |
| PWY0_1319_Lachnospiraceae_bacterium_3_1_46FAA | CDP-diacylglycerol | 0 | 0 | 1691 | 0.00234 | 0.0134 |
| | biosynthesis II | (0-0.000123) | (0-0) | | | |
| UNINTEGRATED_Clostridium_asparagiforme | UNINTEGRATED | 0 | 0 | 1772.5 | 0.00232 | 0.0134 |
| | | (0-0.0534) | (0-0) | | | |
| PWY_6387_Dorea_formicigenerans | UDP-N-acetylmuramoyl- | 0.000117 | 0.0000518 | 1577.5 | 0.00242 | 0.0137 |
| | pentapeptide biosynthesis I | (0.0000381-0.000188) | (0-0.000099) | | | |
| | (meso-diaminopimelate | | | | | |
| | containing) | | | | | |
| HISTSYN_PWY_Bifidobacterium_longum | L-histidine biosynthesis | 0 | 0 | 1666.5 | 0.00245 | 0.0139 |
| | | (0-0.000348) | (0-0) | | | |
| PWY_5686_Prevotella_copri | UMP biosynthesis | 0 | 0 | 2741 | 0.00251 | 0.014 |
| | | (0-0) | (0-0.000261) | | | |
| PWY_6163_Clostridium_bolteae | chorismate biosynthesis from | 0 | 0 | 1888 | 0.00251 | 0.014 |
| | 3-dehydroquinate | (0-0) | (0-0) | | | |
| PWY_7219_Clostridiales_bacterium_1_7_47FAA | adenosine ribonucleotides de | 0 | 0 | 1888 | 0.00251 | 0.014 |
| | novo biosynthesis | (0-0) | (0-0) | | | |
| PWY0_1296_Lachnospiraceae_bacterium_2_1_58FAA | purine ribonucleosides | 0 | 0 | 1889 | 0.00258 | 0.0143 |
| | degradation | (0-0) | (0-0) | | | |
| ANAGLYCOLYSIS_PWY_Alistipessenega_lensis | glycolysis III (from glucose) | 0 | 0 | 2699 | 0.00274 | 0.0144 |
| | | (0-0) | (0-0.0000535) | | | |
| P162_PWY_unclassified | L-glutamate degradation V | 0 | 0.000101 | 2878 | 0.00276 | 0.0144 |
| | (via hydroxyglutarate) | (0-0.000097) | (0-0.000248) | | | |
| PWY_5667_Clostridium_symbiosum | CDP-diacylglycerol | 0 | 0 | 1892 | 0.00279 | 0.0144 |
| | biosynthesis I | (0-0) | (0-0) | | | |
| PWY_6122_Lachnospiraceae_bacterium_3_1_46FAA | 5-aminoimidazole | 0 | 0 | 1685 | 0.00279 | 0.0144 |
| | ribonucleotide biosynthesis II | (0-0.000152) | (0-0) | | | |
| PWY_6151_Coprococcus_sp_ART55_1 | S-adenosyl-L-methionine | 0 | 0 | 2831 | 0.0028 | 0.0144 |
| | cycle I | (0-0) | (0-0.00132) | | | |
| PWY_6163_Clostridium_clostridioforme | chorismate biosynthesis from | 0 | 0 | 1892 | 0.00279 | 0.0144 |
| | 3-dehydroquinate | (0-0) | (0-0) | | | |
| PWY_6277_Lachnospiraceae_bacterium_3_1_46FAA | superpathway of 5- | 0 | 0 | 1685 | 0.00279 | 0.0144 |
| | aminoimidazole ribonucleotide | (0-0.000152) | (0-0) | | | |
| | biosynthesis | | | | | |
| PWY_6703_Ruminococcus_lactaris | preQ0 biosynthesis | 0 | 0.000313 | 2896 | 0.0027 | 0.0144 |
| | | (0-0.000472) | (0-0.00112) | | | |
| PWY_7111_Eubacterium_eligens | pyruvate fermentation to | 0.000291 | 0.0006 | 2944 | 0.00266 | 0.0144 |
| | isobutanol (engineered) | (0.0000056-0.000699) | (0.000222-0.00138) | | | |
| PWY_7220_unclassified | adenosine | 0.00178 | 0.00306 | 2943 | 0.00275 | 0.0144 |
| | deoxyribonucleotides de novo | (0.00117-0.00316) | (0.00187-0.00451) | | | |
| | biosynthesis II | | | | | |
| PWY_7222_unclassified | guanosine | 0.00178 | 0.00306 | 2943 | 0.00275 | 0.0144 |
| | deoxyribonucleotides de novo | (0.00117-0.00316) | (0.00187-0.00451) | | | |
| | biosynthesis II | | | | | |
| PWY0_1297_Ruminococcus_gnavus | superpathway of purine | 0 | 0 | 1890 | 0.00265 | 0.0144 |
| | deoxyribonucleosides | (0-0) | (0-0) | | | |
| | degradation | | | | | |
| PWY0_1319_Clostridium_symbiosum | CDP-diacylglycerol | 0 | 0 | 1892 | 0.00279 | 0.0144 |
| | biosynthesis II | (0-0) | (0-0) | | | |
| UNINTEGRATED_Clostridium_hathewayi | UNINTEGRATED | 0 | 0 | 1755 | 0.00272 | 0.0144 |
| | | (0-0.135) | (0-0) | | | |
| VALSYN_PWY_Eubacterium_eligens | L-valine biosynthesis | 0.000291 | 0.0006 | 2944 | 0.00266 | 0.0144 |
| | | (0.0000056-0.000699) | (0.000222-0.00138) | | | |
| PWY_6163_Ruminococcus_gnavus | chorismate biosynthesis from | 0 | 0 | 1894 | 0.00294 | 0.0151 |
| | 3-dehydroquinate | (0-0) | (0-0) | | | |
| PWY_7237_Clostridium_symbiosum | myo-, chiro- and scillo-inositol | 0 | 0 | 1805 | 0.00294 | 0.0151 |
| | degradation | (0-0.0000168) | (0-0) | | | |
| PWY_7221_Eubacterium_eligens | guanosine ribonucleotides de | 0.000313 | 0.00064 | 2934 | 0.003 | 0.0153 |
| | novo biosynthesis | (0-0.000718) | (0.000165-0.00139) | | | |
| PWY_7111_Ruminococcus_gnavus | pyruvate fermentation to | 0 | 0 | 1807 | 0.00307 | 0.0155 |
| | isobutanol (engineered) | (0-0.000012) | (0-0) | | | |
| PWY_7219_Eubacterium_eligens | adenosine ribonucleotides de | 0.000359 | 0.000821 | 2933.5 | 0.00307 | 0.0155 |
| | novo biosynthesis | (0-0.000874) | (0.000196-0.00177) | | | |
| UNINTEGRATED_Alistipes_senegalensis | UNINTEGRATED | 0 | 0 | 2823 | 0.00321 | 0.0161 |
| | | (0-0) | (0-0.0557) | | | |
| PWY_7456_Coprococcus_sp_ART55_1 | mannan degradation | 0 | 0 | 2822 | 0.00327 | 0.0163 |
| | | (0-0) | (0-0.0017) | | | |
| PWY_5667_Clostridium_bolteae | CDP-diacylglycerol | 0 | 0 | 1842 | 0.00333 | 0.0165 |
| | biosynthesis I | (0-0) | (0-0) | | | |
| PWY_6609_Alistipes_senegalensis | adenine and adenosine salvage | 0 | 0 | 2720 | 0.00335 | 0.0165 |
| | III | (0-0) | (0-0.0000493) | | | |
| PWY01319_Clostridium_bolteae | CDP-diacylglycerol | 0 | 0 | 1842 | 0.00333 | 0.0165 |
| | biosynthesis II | (0-0) | (0-0) | | | |
| DTDPRHAMSYN_PWY_Eggerthella_lenta | dTDP-L-rhamnose | 0 | 0 | 1901 | 0.00354 | 0.0168 |
| | biosynthesis I | (0-0) | (0-0) | | | |
| GALACTUROCAT_PWY_unclassified | D-galacturonate degradation I | 0.000718 | 0.000953 | 2926 | 0.00351 | 0.0168 |
| | | (0.000477-0.000989) | (0.000665-0.00121) | | | |
| PANTO_PWY_Coprococcus_sp_ART55_1 | phosphopantothenate | 0 | 0 | 2818 | 0.00349 | 0.0168 |
| | biosynthesis I | (0-0) | (0-0.00103) | | | |
| PWY_5659_Coprococcus_sp_ART55_1 | GDP-mannose biosynthesis | 0 | 0 | 2820.5 | 0.00357 | 0.0168 |
| | | (0-0) | (0-0.00163) | | | |
| PWY_6151_Barnesiella_intestinihominis | S-adenosyl-L-methionine | 0.000125 | 0.000331 | 2915 | 0.00349 | 0.0168 |
| | cycle I | (0-0.000411) | (0.000128-0.0006) | | | |
| PWY_6151_Eubacterium_eligens | S-adenosyl-L-methionine | 0.000375 | 0.000622 | 2921 | 0.00356 | 0.0168 |
| | cycle I | (0-0.000799) | (0.000203-0.00149) | | | |
| PWY_6305_unclassified | putrescine biosynthesis IV | 0.00134 | 0.00181 | 2927 | 0.00346 | 0.0168 |
| | | (0.000913-0.00206) | (0.00136-0.00253) | | | |
| PWY_7219_Coprococcus_sp_ART55_1 | adenosine ribonucleotides de | 0 | 0 | 2821.5 | 0.00352 | 0.0168 |
| | novo biosynthesis | (0-0) | (0-0.00158) | | | |
| PWY_7219_Flavonifractor_plautii | adenosine ribonucleotides de | 0 | 0 | 1791.5 | 0.00342 | 0.0168 |
| | novo biosynthesis | (0-0.0000552) | (0-0) | | | |
| PWY_7221_Ruminococcus_torques | guanosine ribonucleotides de | 0.0000312 | 0 | 1648 | 0.00344 | 0.0168 |
| | novo biosynthesis | (0-0.000251) | (0-0.0000344) | | | |
| TRPSYN_PWY_Coprococcus_sp_ART55_1 | L-tryptophan biosynthesis | 0 | 0 | 2822.5 | 0.00346 | 0.0168 |
| | | (0-0) | (0-0.00129) | | | |
| HSERMETANA_PWY_unclassified | L-methionine biosynthesis III | 0.000828 | 0.00133 | 2923 | 0.00366 | 0.017 |
| | | (0.000622-0.00151) | (0.000792-0.00222) | | | |
| NONMEVIPP_PWY_Lachnospiraceae_bacterium_1_1_57FAA | methylerythritol phosphate | 0 | 0 | 1837.5 | 0.00361 | 0.017 |
| | pathway I | (0-0) | (0-0) | | | |
| PWY_5484_unclassified | glycolysis II (from fructose 6- | 0.000523 | 0.00113 | 2923 | 0.00363 | 0.017 |
| | phosphate) | (0.000135-0.00178) | (0.000569-0.00222) | | | |
| PWY_621_Coprococcus_sp_ART55_1 | sucrose degradation III | 0 | 0 | 2818.5 | 0.0037 | 0.0171 |
| | (sucrose invertase) | (0-0) | (0-0.00264) | | | |
| UNINTEGRATED_Coprococcus_sp_ART55_1 | UNINTEGRATED | 0 | 0 | 2816.5 | 0.00382 | 0.0176 |
| | | (0-0) | (0-0.923) | | | |
| PWY_2942_Coprococcus_sp_ART55_1 | L-lysine biosynthesis III | 0 | 0 | 2814.5 | 0.00395 | 0.0182 |
| | | (0-0) | (0-0.00118) | | | |
| GLYCOGENSYNTH_PWY_Coprococcus_sp_ART55_1 | glycogen biosynthesis I (from | 0 | 0 | 2813.5 | 0.00402 | 0.0183 |
| | ADP-D-Glucose) | (0-0) | (0-0.00155) | | | |
| THRESYN_PWY_Coprococcus_sp_ART55_1 | superpathway of L-threonine | 0 | 0 | 2813.5 | 0.00402 | 0.0183 |
| | biosynthesis | (0-0) | (0-0.00116) | | | |
| COA_PWY_1_Clostridiales_bacterium_1_7_47FAA | coenzyme A biosynthesis I | 0 | 0 | 1917.5 | 0.0041 | 0.0184 |
| | | (0-0) | (0-0) | | | |
| COA_PWY_Clostridiales_bacterium_1_7_47FAA | coenzyme A biosynthesis I | 0 | 0 | 1918.5 | 0.00421 | 0.0184 |
| | | (0-0) | (0-0) | | | |
| GALACT_GLUCUROCAT_PWY_unclassified | superpathway of hexuronide | 0.000628 | 0.000997 | 2912.5 | 0.00423 | 0.0184 |
| | and hexuronate degradation | (0.000439-0.00109) | (0.000653-0.00132) | | | |
| PWY_3001_Coprococcus_sp_ART55_1 | superpathway of L-isoleucine | 0 | 0 | 2811.5 | 0.00415 | 0.0184 |
| | biosynthesis I | (0-0) | (0-0.00107) | | | |
| PWY_5667_Clostridium_clostridioforme | CDP-diacylglycerol | 0 | 0 | 1918.5 | 0.00421 | 0.0184 |
| | biosynthesis I | (0-0) | (0-0) | | | |
| PWY_6123_Coprococcus_sp_ART55_1 | inosine-5′-phosphate | 0 | 0 | 2811.5 | 0.00415 | 0.0184 |
| | biosynthesis I | (0-0) | (0-0.0012) | | | |
| PWY_7111_Coprococcus_sp_ART55_1 | pyruvate fermentation to | 0 | 0 | 2810.5 | 0.00422 | 0.0184 |
| | isobutanol (engineered) | (0-0) | (0-0.00099) | | | |
| PWY_7111_Lachnospiraceae_bacterium_1_1_57FAA | pyruvate fermentation to | 0 | 0 | 1789 | 0.00417 | 0.0184 |
| | isobutanol (engineered) | (0-0.0000348) | (0-0) | | | |
| PWY_7208_Coprococcus_sp_ART55_1 | superpathway of pyrimidine | 0 | 0 | 2811.5 | 0.00415 | 0.0184 |
| | nucleobases salvage | (0-0) | (0-0.00111) | | | |
| PWY01319_Clostridium_clostridioforme | CDP-diacylglycerol | 0 | 0 | 1918.5 | 0.00421 | 0.0184 |
| | biosynthesis II | (0-0) | (0-0) | | | |
| UDPNAGSYN_PWY_Coprococcus_sp_ART55_1 | UDP-N-acetyl-D-glucosamine | 0 | 0 | 2810.5 | 0.00422 | 0.0184 |
| | biosynthesis I | (0-0) | (0-0.00137) | | | |
| VALSYN_PWY_Lachnospiraceae_bacterium_1_1_57FAA | L-valine biosynthesis | 0 | 0 | 1789 | 0.00417 | 0.0184 |
| | | (0-0.0000348) | (0-0) | | | |
| PWY_5097_Coprococcus_sp_ART55_1 | L-lysine biosynthesis VI | 0 | 0 | 2809.5 | 0.00429 | 0.0185 |
| | | (0-0) | (0-0.0012) | | | |
| PWY_6700_Coprococcus_sp_ART55_1 | queuosine biosynthesis | 0 | 0 | 2809.5 | 0.00429 | 0.0185 |
| | | (0-0) | (0-0.00117) | | | |
| PWY_6527_Coprococcus_sp_ART55_1 | stachyose degradation | 0 | 0 | 2808.5 | 0.00436 | 0.0186 |
| | | (0-0) | (0-0.00172) | | | |
| PWY_6737_Clostridium_hathewayi | starch degradation V | 0 | 0 | 1880 | 0.00437 | 0.0186 |
| | | (0-0) | (0-0) | | | |
| PWY0_1296_Clostridium_asparagiforme | purine ribonucleosides | 0 | 0 | 1919.5 | 0.00432 | 0.0186 |
| | degradation | (0-0) | (0-0) | | | |
| PWY_5104_Coprococcus_sp_ART55_1 | L-isoleucine biosynthesis IV | 0 | 0 | 2807.5 | 0.00443 | 0.0187 |
| | | (0-0) | (0-0.0012) | | | |
| PWY_6122_Lachnospiraceae_bacterium_2_1_58FAA | 5-aminoimidazole | 0 | 0 | 1920.5 | 0.00444 | 0.0187 |
| | ribonucleotide biosynthesis II | (0-0) | (0-0) | | | |
| PWY_6277_Lachnospiraceae_bacterium_2_1_58FAA | superpathway of 5- | 0 | 0 | 1920.5 | 0.00444 | 0.0187 |
| | aminoimidazole ribonucleotide | (0-0) | (0-0) | | | |
| | biosynthesis | | | | | |
| BRANCHED_CHAIN_AA_SYN_PWY_Coprococcus_sp_ART55_1 | superpathway of branched | 0 | 0 | 2806.5 | 0.00451 | 0.0189 |
| | amino acid biosynthesis | (0-0) | (0-0.00108) | | | |
| PWY_5103_Coprococcus_sp_ART55_1 | L-isoleucine biosynthesis III | 0 | 0 | 2806.5 | 0.00451 | 0.0189 |
| | | (0-0) | (0-0.000978) | | | |
| ILEUSYN_PWY_Coprococcus_sp_ART55_1 | L-isoleucine biosynthesis I | 0 | 0 | 2804.5 | 0.00466 | 0.0193 |
| | (from threonine) | (0-0) | (0-0.00114) | | | |
| PYRIDNUCSYN_PWY_Coprococcus_sp_ART55_1 | NAD biosynthesis I (from | 0 | 0 | 2804.5 | 0.00466 | 0.0193 |
| | aspartate) | (0-0) | (0-0.00108) | | | |
| VALSYN_PWY_Coprococcus_sp_ART55_1 | L-valine biosynthesis | 0 | 0 | 2804.5 | 0.00466 | 0.0193 |
| | | (0-0) | (0-0.00114) | | | |
| PWY_6124_Coprococcus_sp_ART55_1 | inosine-5′-phosphate | 0 | 0 | 2803.5 | 0.00473 | 0.0195 |
| | biosynthesis II | (0-0) | (0-0.00113) | | | |
| PWY0_1297_Clostridiales_bacterium_1_7_47FAA | superpathway of purine | 0 | 0 | 1923.5 | 0.0048 | 0.0197 |
| | deoxyribonucleosides | (0-0) | (0-0) | | | |
| | degradation | | | | | |
| NONOXIPENT_PWY_Eubacterium_eligens | pentose phosphate pathway | 0.000394 | 0.000617 | 2899 | 0.00486 | 0.0199 |
| | (non-oxidative branch) | (0-0.000819) | (0.000177-0.00142) | | | |
| GLYCOGENSYNTH_PWY_Eubacterium_biforme | glycogen biosynthesis I (from | 0 | 0.000125 | 2840 | 0.00497 | 0.0202 |
| | ADP-D-Glucose) | (0-0.000268) | (0-0.000507) | | | |
| PWY_6737_Clostridium_symbiosum | starch degradation V | 0 | 0 | 1845 | 0.00501 | 0.0202 |
| | | (0-0) | (0-0) | | | |
| UNINTEGRATED_Eubacterium_eligens | UNINTEGRATED | 0.192 | 0.324 | 2900 | 0.00496 | 0.0202 |
| | | (0.0259-0.367) | (0.12-0.706) | | | |
| VALSYN_PWY_Clostridium_bolteae | L-valine biosynthesis | 0 | 0 | 1823.5 | 0.00498 | 0.0202 |
| | | (0-0.0000142) | (0-0) | | | |
| PWY_7111_unclassified | pyruvate fermentation to | 0.0115 | 0.0136 | 2899 | 0.0051 | 0.0205 |
| | isobutanol (engineered) | (0.00851-0.0145) | (0.0108-0.0164) | | | |
| PWY_5667_Ruminococcus_obeum | CDP-diacylglycerol | 0.0000317 | 0.0000943 | 2881 | 0.00517 | 0.0206 |
| | biosynthesis I | (0-0.000122) | (0.0000231-0.00024) | | | |
| PWY_7219_Barnesiella_intestinihominis | adenosine ribonucleotides de | 0.000173 | 0.000512 | 2894 | 0.00513 | 0.0206 |
| | novo biosynthesis | (0-0.000674) | (0.000192-0.000716) | | | |
| PWY0_1319_Ruminococcus_obeum | CDP-diacylglycerol | 0.0000317 | 0.0000943 | 2881 | 0.00517 | 0.0206 |
| | biosynthesis II | (0-0.000122) | (0.0000231-0.00024) | | | |
| ILEUSYN_PWY_unclassified | L-isoleucine biosynthesis I | 0.0117 | 0.0138 | 2897 | 0.00524 | 0.0207 |
| | (from threonine) | (0.00851-0.0145) | (0.0115-0.0164) | | | |
| VALSYN_PWY_unclassified | L-valine biosynthesis | 0.0117 | 0.0138 | 2897 | 0.00524 | 0.0207 |
| | | (0.00851-0.0145) | (0.0115-0.0164) | | | |
| PWY_1042_Ruminococcus_obeum | glycolysis IV (plant cytosol) | 0 | 0 | 2796.5 | 0.0053 | 0.0209 |
| | | (0-0) | (0-0.000149) | | | |
| PWY_7221_Lachnospiraceae_bacterium_1_4_56FAA | guanosine ribonucleotides de | 0 | 0 | 1927.5 | 0.00532 | 0.0209 |
| | novo biosynthesis | (0-0) | (0-0) | | | |
| PWY0_1298_unclassified | superpathway of pyrimidine | 0.000285 | 0.000382 | 2895.5 | 0.00535 | 0.0209 |
| | deoxyribonucleosides | (0.000138-0.00042) | (0.000236-0.000607) | | | |
| | degradation | | | | | |
| PWY_4984_Flavonifractor_plautii | urea cycle | 0 | 0 | 1823 | 0.00561 | 0.0219 |
| | | (0-0.0000361) | (0-0) | | | |
| X1CMET2_PWY_Bacteroides_massiliensis | N10-formyl-tetrahydrofolate | 0 | 0 | 2741 | 0.00562 | 0.0219 |
| | biosynthesis | (0-0) | (0-0.00036) | | | |
| PWY_7111_Barnesiella_intestinihominis | pyruvate fermentation to | 0.000125 | 0.00033 | 2886 | 0.00584 | 0.0225 |
| | isobutanol (engineered) | (0-0.000436) | (0.000123-0.000561) | | | |
| VALSYN_PWY_Barnesiella_intestinihominis | L-valine biosynthesis | 0.000125 | 0.00033 | 2886 | 0.00584 | 0.0225 |
| | | (0-0.000436) | (0.000123-0.000561) | | | |
| PWY_5103_unclassified | L-isoleucine biosynthesis III | 0.00655 | 0.00852 | 2888 | 0.00592 | 0.0228 |
| | | (0.00495-0.00942) | (0.00635-0.0104) | | | |
| PWY_6471_unclassified | peptidoglycan biosynthesis IV | 0 | 0 | 1903 | 0.00604 | 0.0231 |
| | (Enterococcus faecium) | (0-0) | (0-0) | | | |
| PWY0_1296_Eubacterium_biforme | purine ribonucleosides | 0 | 0.000114 | 2824.5 | 0.00604 | 0.0231 |
| | degradation | (0-0.000317) | (0-0.000641) | | | |
| SALVADEHYPOX_PWY_unclassified | adenosine nucleotides | 0.00109 | 0.00141 | 2886 | 0.00608 | 0.0232 |
| | degradation II | (0.000787-0.00145) | (0.00112-0.00192) | | | |
| PWY_6737_Dorea_formicigenerans | starch degradation V | 0.000204 | 0.000116 | 1640 | 0.00619 | 0.0235 |
| | | (0.0000983-0.000285) | (0.0000639-0.000195) | | | |
| PWY_6121_Lachnospiraceae_bacterium_1_1_57FAA | 5-aminoimidazole | 0 | 0 | 1829 | 0.00629 | 0.0238 |
| | ribonucleotide biosynthesis I | (0-0.0000328) | (0-0) | | | |
| PWY7111_Flavonifractor_plautii | pyruvate fermentation to | 0 | 0 | 1817 | 0.00632 | 0.0238 |
| | isobutanol (engineered) | (0-0.0000355) | (0-0) | | | |
| VALSYN_PWY_Flavonifractor_plautii | L-valine biosynthesis | 0 | 0 | 1817 | 0.00632 | 0.0238 |
| | | (0-0.0000355) | (0-0) | | | |
| PWY_7357_Ruminococcus_obeum | thiamin formation from | 0.0000515 | 0.000128 | 2869.5 | 0.00645 | 0.0242 |
| | pyrithiamine and oxythiamine | (0-0.000208) | (0.0000665-0.000336) | | | |
| | (yeast) | | | | | |
| PANTO_PWY_Ruminococcus_torques | phosphopantothenate | 0.000375 | 0.000285 | 1644 | 0.00658 | 0.0245 |
| | biosynthesis I | (0.000178-0.0007) | (0.000106-0.000385) | | | |
| PWY_1042_Barnesiella_intestinihominis | glycolysis IV (plant cytosol) | 0.000203 | 0.000417 | 2875 | 0.00656 | 0.0245 |
| | | (0-0.000564) | (0.000167-0.000662) | | | |
| ARO_PWY_Clostridium_bolteae | chorismate biosynthesis I | 0 | 0 | 1947 | 0.00667 | 0.0246 |
| | | (0-0) | (0-0) | | | |
| COMPLETE_ARO_PWY_Clostridium_bolteae | superpathway of aromatic | 0 | 0 | 1947 | 0.00667 | 0.0246 |
| | amino acid biosynthesis | (0-0) | (0-0) | | | |
| PWY_6121_Eubacterium_biforme | 5-aminoimidazole | 0 | 0.000154 | 2820 | 0.0067 | 0.0246 |
| | ribonucleotide biosynthesis I | (0-0.000322) | (0-0.000682) | | | |
| PWY_7219_Anaerotruncus_colihominis | adenosine ribonucleotides de | 0 | 0 | 1907 | 0.00663 | 0.0246 |
| | novo biosynthesis | (0-0) | (0-0) | | | |
| PWY_5686_Barnesiella_intestinihominis | UMP biosynthesis | 0.000113 | 0.000344 | 2871 | 0.00674 | 0.0247 |
| | | (0-0.000461) | (0.00011-0.000565) | | | |
| PWY_6527_Faecalibacterium_prausnitzii | stachyose degradation | 0 | 0 | 2765 | 0.0069 | 0.0252 |
| | | (0-0) | (0-0.000995) | | | |
| PWY_1042_Lachnospiraceae_bacterium_1_1_57FAA | glycolysis IV (plant cytosol) | 0 | 0 | 1910 | 0.0071 | 0.0258 |
| | | (0-0) | (0-0) | | | |
| PWY_7111_Clostridium_clostridioforme | pyruvate fermentation to | 0 | 0 | 1920 | 0.00724 | 0.0261 |
| | isobutanol (engineered) | (0-0) | (0-0) | | | |
| PWY66_422_Eubacterium_biforme | D-galactose degradation V | 0 | 0.000163 | 2815 | 0.00721 | 0.0261 |
| | (Leloir pathway) | (0-0.000342) | (0-0.000623) | | | |
| VALSYN_PWY_Clostridium_clostridioforme | L-valine biosynthesis | 0 | 0 | 1920 | 0.00724 | 0.0261 |
| | UDP-N-acetylmuramoyl- | (0-0) | (0-0) | | | |
| PWY_6386_Lachnospiraceae_bacterium_1_1_57FAA | pentapeptide biosynthesis II | 0 | 0 | 1864 | 0.00739 | 0.0264 |
| | (lysine-containing) | (0-0) | (0-0)0.0000949 | | | |
| PWY0_1296_Coprococcus_catus | purine ribonucleosides | 0.0000464 | | 2866 | 0.0074 | 0.0264 |
| | degradation | (0-0.000124) | (0.0000532-0.000137) | | | |
| PWY66_422_unclassified | D-galactose degradation V | 0.00592 | 0.0069 | 2871 | 0.00742 | 0.0264 |
| | (Leloir pathway) | (0.00417-0.00827) | (0.0057-0.00939) | | | |
| UNINTEGRATED_Barnesiella_intestinihominis | UNINTEGRATED | 0.162 | 0.368 | 2868.5 | 0.0074 | 0.0264 |
| | | (0-0.485) | (0.163-0.537) | | | |
| PWY_241_unclassified | C4 photosynthetic carbon | 0 | 0 | 2742.5 | 0.00759 | 0.0266 |
| | assimilation cycle, NADP-ME | (0-0) | (0-0.000143) | | | |
| | type | | | | | |
| PWY_6317_unclassified | galactose degradation I (Leloir | 0.00592 | 0.0069 | 2870 | 0.00752 | 0.0266 |
| | pathway) | (0.00417-0.00827) | (0.0057-0.00939) | | | |
| PWY_6387_Lachnospiraceae_bacterium_1_1_57FAA | UDP-N-acetylmuramoyl- | 0 | 0 | 1865 | 0.00754 | 0.0266 |
| | pentapeptide biosynthesis I | (0-0) | (0-0) | | | |
| | (meso-diaminopimelate | | | | | |
| | containing) | | | | | |
| PWY_7111_Lachnospiraceae_bacterium_2_1_58FAA | pyruvate fermentation to | 0 | 0 | 1952 | 0.00759 | 0.0266 |
| | isobutanol (engineered) | (0-0) | (0-0) | | | |
| VALSYN_PWY_Clostridium_hathewayi | L-valine biosynthesis | 0 | 0 | 1887.5 | 0.00755 | 0.0266 |
| | | (0-0) | (0-0) | | | |
| PWY_4981_unclassified | L-proline biosynthesis II (from | 0.00162 | 0.00283 | 2867 | 0.00782 | 0.0273 |
| | arginine) | (0.000595-0.00345) | (0.00117-0.0045) | | | |
| PWY_7111_Ruminococcus_lactaris | pyruvate fermentation to | 0 | 0.000125 | 2826.5 | 0.00798 | 0.0278 |
| | isobutanol (engineered) | (0-0.000202) | (0-0.000338) | | | |
| PEPTIDOGLYCANSYN_PWY_Lachnospiraceae_bacterium_1_1_57FAA | peptidoglycan biosynthesis I | 0 | 0 | 1891.5 | 0.00822 | 0.0284 |
| | (meso-diaminopimelate | (0-0) | (0-0) | | | |
| | containing) | | | | | |
| PWY_6122_Eubacterium_biforme | 5-aminoimidazole | 0 | 0.000167 | 2805 | 0.00833 | 0.0284 |
| | ribonucleotide biosynthesis II | (0-0.000389) | (0-0.000642) | | | |
| PWY_6277_Eubacterium_biforme | superpathway of 5- | 0 | 0.000167 | 2805 | 0.00833 | 0.0284 |
| | aminoimidazole ribonucleotide | (0-0.000389) | (0-0.000642) | | | |
| | biosynthesis | | | | | |
| PWY_6608_Odoribacter_splanchnicus | guanosine nucleotides | 0.0000571 | 0.000171 | 2845 | 0.00823 | 0.0284 |
| | degradation III | (0-0.000193) | (0-0.000303) | | | |
| PWY_6609_Lachnospiraceae_bacterium_2_1_58FAA | adenine and adenosine salvage | 0 | 0 | 1926 | 0.00833 | 0.0284 |
| | III | (0-0) | (0-0) | | | |
| PWY_7219_Bacteroides_massiliensis | adenosine ribonucleotides de | 0 | 0 | 2742 | 0.00821 | 0.0284 |
| | novo biosynthesis | (0-0) | (0-0.00056) | | | |
| PWY_7221_Barnesiella_intestinihominis | guanosine ribonucleotides de | 0.0000759 | 0.000342 | 2856 | 0.00834 | 0.0284 |
| | novo biosynthesis | (0-0.000478) | (0.000114-0.000576) | | | |
| PWY_7221_Flavonifractor_plautii | guanosine ribonucleotides de | 0 | 0 | 1851.5 | 0.00856 | 0.0291 |
| | novo biosynthesis | (0-0.0000288) | (0-0) | | | |
| NONOXIPENT_PWY_Ruminococcus_lactaris | pentose phosphate pathway | 0 | 0 | 2742 | 0.00878 | 0.0292 |
| | (non-oxidative branch) | (0-0) | (0-0.000296) | | | |
| PWY_3841_Bacteroides_massiliensis | folate transformations II | 0 | 0 | 2730 | 0.00867 | 0.0292 |
| | | (0-0) | (0-0.000393) | | | |
| PWY_5667_Barnesiella_intestinihominis | CDP-diacylglycerol | 0.00013 | 0.000316 | 2852 | 0.00878 | 0.0292 |
| | biosynthesis I | (0-0.000513) | (0.000143-0.000605) | | | |
| PWY_6609_Eubacterium_biforme | adenine and adenosine salvage | 0 | 0.000121 | 2799.5 | 0.00871 | 0.0292 |
| | III | (0-0.000335) | (0-0.00067) | | | |
| PWY_7219_Clostridium_hathewayi | adenosine ribonucleotides de | 0 | 0 | 1872 | 0.00867 | 0.0292 |
| | novo biosynthesis | (0-0) | (0-0) | | | |
| | thiamin formation from | | | | | |
| PWY_7357_Eubacterium_biforme | pyrithiamine and oxythiamine | 0 | 0 | 2706 | 0.00867 | 0.0292 |
| | (yeast) | (0-0) | (0-0.000193) | | | |
| PWY0_1319_Barnesiella_intestinihominis | CDP-diacylglycerol | 0.00013 | 0.000316 | 2852 | 0.00878 | 0.0292 |
| | biosynthesis II | (0-0.000513) | (0.000143-0.000605) | | | |
| | superpathway of thiamin | | | | | |
| THISYNARA_PWY_Ruminococcus_obeum | diphosphate biosynthesis III | 0 | 0.000078 | 2833 | 0.00881 | 0.0292 |
| | (eukaryotes) | (0-0.0000872) | (0-0.000176) | | | |
| PWY_7219_Lachnospiraceae_bacterium_1_1_57FAA | adenosine ribonucleotides de | 0 | 0 | 1814 | 0.00884 | 0.0293 |
| | novo biosynthesis | (0-0.0000818) | (0-0) | | | |
| PANTO_PWY_Eggerthella_lenta | phosphopantothenate | 0 | 0 | 1881 | 0.00902 | 0.0297 |
| | biosynthesis I | (0-0) | (0-0) | | | |
| PWY_5121_unclassified | superpathway of | 0 | 0 | 2657 | 0.00899 | 0.0297 |
| | geranylgeranyl diphosphate | (0-0) | (0-0.000128) | | | |
| | biosynthesis II (via MEP) | | | | | |
| PWY_7219_Ruminococcus_torques | adenosine ribonucleotides de | 0.000353 | 0.000225 | 1669 | 0.00912 | 0.0299 |
| | novo biosynthesis | (0.000193-0.000936) | (0.000127-0.000465) | | | |
| PWY_7219_Paraprevotella_clara | adenosine ribonucleotides de | 0 | 0 | 2758 | 0.00918 | 0.03 |
| | novo biosynthesis | (0-0.0000432) | (0-0.000387) | | | |
| VALSYN_PWY_Dorea_formicigenerans | L-valine biosynthesis | 0.000131 | 0.0000863 | 1671 | 0.00929 | 0.0303 |
| | | (0.0000661-0.0002) | (0.0000473-0.000133) | | | |
| PWY_6122_Lachnospiraceae_bacterium_1_1_57FAA | 5-aminoimidazole | 0 | 0 | 1828 | 0.00943 | 0.0306 |
| | ribonucleotide biosynthesis II | (0-0.0000438) | (0-0) | | | |
| PWY_6277_Lachnospiraceae_bacterium_1_1_57FAA | superpathway of 5- | 0 | 0 | 1828 | 0.00943 | 0.0306 |
| | aminoimidazole ribonucleotide | (0-0.0000438) | (0-0) | | | |
| | biosynthesis | | | | | |
| PWY_5097_Barnesiella_intestinihominis | L-lysine biosynthesis VI | 0.000202 | 0.000436 | 2846 | 0.00961 | 0.0311 |
| | | (0-0.000581) | (0.000171-0.000668) | | | |
| PWY_2723_Escherichia_coli | trehalose degradation V | 0 | 0 | 1781.5 | 0.00986 | 0.0317 |
| | | (0-0.0000739) | (0-0) | | | |
| PYRIDNUCSYN_PWY_unclassified | NAD biosynthesis I (from | 0.00198 | 0.00236 | 2849 | 0.00986 | 0.0317 |
| | aspartate) | (0.00123-0.00265) | (0.0017-0.00321) | | | |
| PWY_7219_Eubacterium_biforme | adenosine ribonucleotides de | 0 | 0.000158 | 2792 | 0.01 | 0.0321 |
| | novo biosynthesis | (0-0.000456) | (0-0.000928) | | | |
| PWY_6151_Eubacterium_biforme | S-adenosyl-L-methionine | 0 | 0.000196 | 2790 | 0.0103 | 0.0329 |
| | cycle I | (0-0.000483) | (0-0.0007) | | | |
| UNINTEGRATED_Eubacterium_biforme | UNINTEGRATED | 0 | 0.11 | 2790 | 0.0103 | 0.0329 |
| | | (0-0.192) | (0-0.235) | | | |
| PWY_7357_unclassified | thiamin formation from | 0.00307 | 0.00454 | 2845 | 0.0104 | 0.033 |
| | pyrithiamine and oxythiamine | (0.00178-0.00543) | (0.00291-0.00637) | | | |
| | (yeast) | | | | | |
| PWY_5188_Coprococcus_catus | tetrapyrrole biosynthesis I | 0 | 0.000028 | 2794.5 | 0.0106 | 0.0336 |
| | (from glutamate) | (0-0.0000326) | (0-0.0000766) | | | |
| PWY_5686_Ruminococcus_obeum | UMP biosynthesis | 0.0000767 | 0.000124 | 2838 | 0.0108 | 0.034 |
| | | (0-0.000177) | (0.0000616-0.000249) | | | |
| PWY_7111_Clostridium_asparagiforme | pyruvate fermentation to | 0 | 0 | 1890 | 0.0108 | 0.034 |
| | isobutanol (engineered) | (0-0) | (0-0) | | | |
| DTDPRHAMSYN_PWY_Eubacterium_biforme | dTDP-L-rhamnose | 0 | 0.0000106 | 2759 | 0.011 | 0.0346 |
| | biosynthesis I | (0-0.0000352) | (0-0.000117) | | | |
| PWY_1042_Eggerthella_lenta | glycolysis IV (plant cytosol) | 0 | 0 | 1939 | 0.0112 | 0.0351 |
| | | (0-0) | (0-0) | | | |
| PWY_6700_Paraprevotella_clara | queuosine biosynthesis | 0 | 0 | 2726.5 | 0.0112 | 0.0351 |
| | | (0-0) | (0-0.000342) | | | |
| UNINTEGRATED_Clostridium_citroniae | UNINTEGRATED | 0 | 0 | 1795 | 0.0115 | 0.0358 |
| | | (0-0.0845) | (0-0) | | | |
| ARO_PWY_Dorea_formicigenerans | chorismate biosynthesis I | 0.0001 | 0.0000586 | 1701 | 0.0115 | 0.0359 |
| | | (0-0.000171) | (0-0.0000962) | | | |
| PWY_6122_Eubacterium_eligens | 5-aminoimidazole | 0.000314 | 0.000572 | 2833 | 0.0117 | 0.0362 |
| | ribonucleotide biosynthesis II | (0-0.000728) | (0.00017-0.0012) | | | |
| PWY_6277_Eubacterium_eligens | superpathway of 5- | 0.000314 | 0.000572 | 2833 | 0.0117 | 0.0362 |
| | aminoimidazole ribonucleotide | (0-0.000728) | (0.00017-0.0012) | | | |
| | biosynthesis | | | | | |
| PWY_6737_Roseburia_inulinivorans | starch degradation V | 0.000516 | 0.000276 | 1690 | 0.0117 | 0.0362 |
| | | (0.0000853-0.00175) | (0.0000219-0.000721) | | | |
| PANTO_PWY_Bacteroides_xylanisolvens | phosphopantothenate | 0 | 0.0000913 | 2797.5 | 0.0118 | 0.0365 |
| | biosynthesis I | (0-0.00015) | (0-0.000371) | | | |
| PWY_2942_Eggerthella_lenta | L-lysine biosynthesis III | 0 | 0 | 1917 | 0.0119 | 0.0365 |
| | | (0-0) | (0-0) | | | |
| PWY_6151_Bacteroides_massiliensis | S-adenosyl-L-methionine | 0 | 0 | 2714.5 | 0.0119 | 0.0365 |
| | cycle I | (0-0) | (0-0.0004) | | | |
| COA_PWY_1_Ruminococcus_torques | coenzyme A biosynthesis I | 0.000285 | 0.000164 | 1693 | 0.0121 | 0.0366 |
| | | (0.000109-0.000644) | (0.0000489-0.000362) | | | |
| PWY_5686_Eubacterium_biforme | UMP biosynthesis | 0 | 0.000118 | 2779 | 0.012 | 0.0366 |
| | | (0-0.000297) | (0-0.000531) | | | |
| PWY_6386_Ruminococcus_torques | UDP-N-acetylmuramoyl- | 0.000366 | 0.00023 | 1691 | 0.012 | 0.0366 |
| | pentapeptide biosynthesis II | (0.000152-0.000861) | (0.0000872-0.000405) | | | |
| | (lysine-containing) | | | | | |
| GLYCOLYSIS_unclassified | glycolysis I (from glucose 6- | 0.000572 | 0.00128 | 2832 | 0.0122 | 0.0368 |
| | phosphate) | (0.000142-0.00214) | (0.000583-0.00265) | | | |
| ARO_PWY_Lachnospiraceae_bacterium_1_1_57FAA | chorismate biosynthesis I | 0 | 0 | 1920 | 0.0127 | 0.0379 |
| | | (0-0) | (0-0) | | | |
| NONMEVIPP_PWY_Paraprevotella_clara | methylerythritol phosphate | 0 | 0 | 2718.5 | 0.0127 | 0.0379 |
| | pathway I | (0-0) | (0-0.000313) | | | |
| PWY_2942_Flavonifractor_plautii | L-lysine biosynthesis III | 0 | 0 | 1898 | 0.0126 | 0.0379 |
| | | (0-0) | (0-0) | | | |
| PWY_6163_Lachnospiraceae_bacterium_1_1_57FAA | chorismate biosynthesis from | 0 | 0 | 1898 | 0.0126 | 0.0379 |
| | 3-dehydroquinate | (0-0) | (0-0) | | | |
| PWY_7219_Eggerthella_lenta | adenosine ribonucleotides de | 0 | 0 | 1898 | 0.0126 | 0.0379 |
| | novo biosynthesis | (0-0) | (0-0) | | | |
| PWY_6121_Eubacterium_eligens | 5-aminoimidazole | 0.000339 | 0.000526 | 2825.5 | 0.0128 | 0.0382 |
| | ribonucleotide biosynthesis I | (0-0.00074) | (0.000181-0.00124) | | | |
| TCA_unclassified | TCA cycle I (prokaryotic) | 0.0000488 | 0.000173 | 2802 | 0.0128 | 0.0382 |
| | | (0-0.000226) | (0-0.000483) | | | |
| PWY_5695_Lachnospiraceae_bacterium_3_1_57FAA_CT1 | urate biosynthesis/inosine 5′- | 0 | 0 | 1946 | 0.0131 | 0.0385 |
| | phosphate degradation | (0-0) | (0-0) | | | |
| PWY_6387_Barnesiella_intestinihominis | UDP-N-acetylmuramoyl- | 0.000121 | 0.000347 | 2818 | 0.0131 | 0.0385 |
| | pentapeptide biosynthesis I | (0-0.000453) | (0.000104-0.000553) | | | |
| | (meso-diaminopimelate | | | | | |
| | containing) | | | | | |
| PWY_6936_Eubacterium_biforme | seleno-amino acid biosynthesis | 0 | 0.0000634 | 2761 | 0.0131 | 0.0385 |
| | | (0-0.00025) | (0-0.000465) | | | |
| UNINTEGRATED_Roseburia_hominis | UNINTEGRATED | 0.23 | 0.303 | 2825.5 | 0.013 | 0.0385 |
| | | (0.155-0.33) | (0.227-0.417) | | | |
| PWY_2942_Bacteroides_massiliensis | L-lysine biosynthesis III | 0 | 0 | 2707.5 | 0.0133 | 0.0391 |
| | | (0-0) | (0-0.000345) | | | |
| ARGININE_SYN4_PWY_unclassified | L-ornithine de novo | 0 | 0.0000637 | 2772.5 | 0.0135 | 0.0395 |
| | biosynthesis | (0-0.000111) | (0-0.000168) | | | |
| PWY_5100_Eubacterium_biforme | pyruvate fermentation to | 0 | 0.000138 | 2768 | 0.0135 | 0.0395 |
| | acetate and lactate II | (0-0.000376) | (0-0.000697) | | | |
| PWY_6527_Lachnospiraceae_bacterium_3_1_57FAA_CT1 | stachyose degradation | 0 | 0 | 1902 | 0.0136 | 0.0396 |
| | superpathway of &beta;-D- | (0-0) | (0-0) | | | |
| GLUCUROCAT_PWY_unclassified | glucuronide and D-glucuronate | 0.000751 | 0.00107 | 2822.5 | 0.0137 | 0.0397 |
| | degradation | (0.000425-0.0012) | (0.000643-0.00137) | | | |
| PWY_6609_Coprococcus_catus | adenine and adenosine salvage | 0.0000683 | 0.000126 | 2816.5 | 0.0137 | 0.0397 |
| | III | (0-0.000172) | (0.0000688-0.000197) | | | |
| PWY_6737_Clostridium_asparagiforme | starch degradation V | 0 | 0 | 1931.5 | 0.0137 | 0.0397 |
| | | (0-0) | (0-0) | | | |
| UNINTEGRATED_Bacteroides_massiliensis | UNINTEGRATED | 0 | 0 | 2712 | 0.014 | 0.0404 |
| | peptidoglycan biosynthesis I | (0-0) | (0-0.428) | | | |
| PEPTIDOGLYCANSYN_PWY_Barnesiella_intestinihominis | (meso-diaminopimelate | 0.000123 | 0.000352 | 2811 | 0.0143 | 0.0405 |
| | containing) | (0-0.000469) | (0.0000938-0.000593) | | | |
| PWY_5667_Ruminococcus_lactaris | CDP-diacylglycerol | 0 | 0.0000533 | 2768.5 | 0.0143 | 0.0405 |
| | biosynthesis I | (0-0.000104) | (0-0.00028) | | | |
| | UDP-N-acetylmuramoyl- | | | | | |
| PWY_6386_Barnesiella_intestinihominis | pentapeptide biosynthesis II | 0.000133 | 0.000362 | 2811 | 0.0143 | 0.0405 |
| | (lysine-containing) | (0-0.000464) | (0.000117-0.000573) | | | |
| PWY_6700_Barnesiella_intestinihominis | queuosine biosynthesis | 0.000178 | 0.000401 | 2813.5 | 0.0142 | 0.0405 |
| | | (0-0.000563) | (0.000155-0.00058) | | | |
| PWY_7282_Bacteroides_fragilis | 4-amino-2-methyl-5- | 0 | 0 | 1852 | 0.0142 | 0.0405 |
| | phosphomethylpyrimidine | (0-0.000108) | (0-0) | | | |
| | biosynthesis (yeast) | | | | | |
| PWY0_1319_Ruminococcus_lactaris | CDP-diacylglycerol | 0 | 0.0000533 | 2768.5 | 0.0143 | 0.0405 |
| | biosynthesis II | (0-0.000104) | (0-0.00028) | | | |
| PWY66_399_unclassified | gluconeogenesis III | 0.000175 | 0.000336 | 2803 | 0.0142 | 0.0405 |
| | | (0-0.00044) | (0-0.000942) | | | |
| PANTO_PWY_Paraprevotella_clara | phosphopantothenate | 0 | 0 | 2728 | 0.0144 | 0.0406 |
| | biosynthesis I | (0-0.0000509) | (0-0.000372) | | | |
| PANTO_PWY_Lachnospiraceae_bacterium_1_1_57FAA | phosphopantothenate | 0 | 0 | 1830 | 0.0144 | 0.0407 |
| | biosynthesis I | (0-0.0000863) | (0-0) | | | |
| PWY_5667_Clostridium_nexile | CDP-diacylglycerol | 0 | 0 | 1960 | 0.0147 | 0.0412 |
| | biosynthesis I | (0-0) | (0-0) | | | |
| PWY0_1319_Clostridium_nexile | CDP-diacylglycerol | 0 | 0 | 1960 | 0.0147 | 0.0412 |
| | biosynthesis II | (0-0) | (0-0) | | | |
| PWY_2942_Barnesiella_intestinihominis | L-lysine biosynthesis III | 0.00013 | 0.000425 | 2809 | 0.0149 | 0.0414 |
| | | (0-0.000585) | (0.000138-0.000612) | | | |
| PWY_5855_Escherichia_coli | ubiquinol-7 biosynthesis | 0 | 0 | 1802.5 | 0.0151 | 0.0414 |
| | (prokaryotic) | (0-0.000103) | (0-0) | | | |
| PWY_5856_Escherichia_coli | ubiquinol-9 biosynthesis | 0 | 0 | 1802.5 | 0.0151 | 0.0414 |
| | (prokaryotic) | (0-0.000103) | (0-0) | | | |
| PWY_5857_Escherichia_coli | ubiquinol-10 biosynthesis | 0 | 0 | 1802.5 | 0.0151 | 0.0414 |
| | (prokaryotic) | (0-0.000103) | (0-0) | | | |
| PWY_6708_Escherichia_coli | ubiquinol-8 biosynthesis | 0 | 0 | 1802.5 | 0.0151 | 0.0414 |
| | (prokaryotic) | (0-0.000103) | (0-0) | | | |
| PWY_6737_Lachnospiraceae_bacterium_7_1_58FAA | starch degradation V | 0 | 0 | 1893.5 | 0.0148 | 0.0414 |
| | | (0-0) | (0-0) | | | |
| PWY_7111_Clostridium_citroniae | pyruvate fermentation to | 0 | 0 | 1961 | 0.015 | 0.0414 |
| | isobutanol (engineered) | (0-0) | (0-0) | | | |
| VALSYN_PWY_Clostridium_citroniae | L-valine biosynthesis | 0 | 0 | 1961 | 0.015 | 0.0414 |
| | | (0-0) | (0-0) | | | |
| HISTSYN_PWY_Lachnospiraceae_bacterium_7_1_58FAA | L-histidine biosynthesis | 0 | 0 | 1936.5 | 0.0152 | 0.0416 |
| | | (0-0) | (0-0) | | | |
| ARGSYN_PWY_Escherichia_coli | L-arginine biosynthesis I (via | 0 | 0 | 1839 | 0.0155 | 0.0425 |
| | L-ornithine) | (0-0.000139) | (0-0) | | | |
| CALVIN_PWY_Ruminococcus_torques | Calvin-Benson-Bassham cycle | 0.0000569 | 0 | 1755 | 0.0157 | 0.0428 |
| | | (0-0.000357) | (0-0.0000924) | | | |
| PANTO_PWY_Barnesiella_intestinihominis | phosphopantothenate | 0.000134 | 0.000375 | 2805 | 0.0157 | 0.0428 |
| | biosynthesis I | (0-0.000584) | (0.0000885-0.000625) | | | |
| PWY_7221_Bacteroides_massiliensis | guanosine ribonucleotides de | 0 | 0 | 2696.5 | 0.0158 | 0.0429 |
| | novo biosynthesis | (0-0) | (0-0.000451) | | | |
| UBISYN_PWY_Escherichia_coli | superpathway of ubiquinol-8 | 0 | 0 | 1813 | 0.0159 | 0.0431 |
| | biosynthesis (prokaryotic) | (0-0.000111) | (0-0) | | | |
| PWY_6125_Eggerthella_lenta | superpathway of guanosine | 0 | 0 | 1964 | 0.0161 | 0.0434 |
| | nucleotides de novo | (0-0) | (0-0) | | | |
| | biosynthesis II | | | | | |
| PWY_6737_Lachnospiraceae_bacterium_1_1_57FAA | starch degradation V | 0 | 0 | 1845.5 | 0.0161 | 0.0434 |
| | | (0-0.0000376) | (0-0) | | | |
| PWY0_1296_Roseburia_inulinivorans | purine ribonucleosides | 0.000235 | 0.000104 | 1720 | 0.0163 | 0.0439 |
| | degradation | (0.0000364-0.000771) | (0-0.000394) | | | |
| PWY0_162_unclassified | superpathway of pyrimidine | 0.00204 | 0.00306 | 2808 | 0.0164 | 0.044 |
| | ribonucleotides de novo | (0.00166-0.00355) | (0.00191-0.00456) | | | |
| | biosynthesis | | | | | |
| PWY_5188_Flavonifractor_plautii | tetrapyrrole biosynthesis I | 0 | 0 | 1900.5 | 0.0168 | 0.0449 |
| | (from glutamate) | (0-0) | (0-0) | | | |
| PWY_6609_Eggerthella_lenta | adenine and adenosine salvage | 0 | 0 | 1941.5 | 0.0168 | 0.0449 |
| | III | (0-0) | (0-0) | | | |
| UNINTEGRATED_Lachnospiraceae_bacterium_3_1_57FAA_CT1 | UNINTEGRATED | 0 | 0 | 1840.5 | 0.017 | 0.0453 |
| | | (0-0.164) | (0-0) | | | |
| PWY_4981_Eggerthella_lenta | L-proline biosynthesis II (from | 0 | 0 | 1921 | 0.0172 | 0.0456 |
| | arginine) | (0-0) | (0-0) | | | |
| PWY0_1586_Eggerthella_lenta | peptidoglycan maturation | 0 | 0 | 1921 | 0.0172 | 0.0456 |
| | (meso-diaminopimelate | (0-0) | (0-0) | | | |
| | containing) | | | | | |
| PWY_5667_Eubacterium_eligens | CDP-diacylglycerol | 0.000143 | 0.000286 | 2797 | 0.0173 | 0.0457 |
| | biosynthesis I | (0-0.00054) | (0.0000566-0.000829) | | | |
| PWY0_1319_Eubacterium_eligens | CDP-diacylglycerol | 0.000143 | 0.000286 | 2797 | 0.0173 | 0.0457 |
| | biosynthesis II | (0-0.00054) | (0.0000566-0.000829) | | | |
| PWY_5097_Paraprevotella_clara | L-lysine biosynthesis VI | 0 | 0 | 2708 | 0.0175 | 0.0459 |
| | | (0-0) | (0-0.000309) | | | |
| PWY_6163_Flavonifractor_plautii | chorismate biosynthesis from | 0 | 0 | 1943.5 | 0.0175 | 0.0459 |
| | 3-dehydroquinate | (0-0) | (0-0) | | | |
| PWY0_1296_Clostridium_hathewayi | purine ribonucleosides | 0 | 0 | 1922 | 0.0175 | 0.0459 |
| | degradation | (0-0) | (0-0) | | | |
| COA_PWY_1_Roseburia_inulinivorans | coenzyme A biosynthesis I | 0.00024 | 0.0000936 | 1732.5 | 0.0176 | 0.046 |
| | | (0-0.000831) | (0-0.000258) | | | |
| PEPTIDOGLYCANSYN_PWY_Flavonifractor_plautii | peptidoglycan biosynthesis I | 0 | 0 | 1969 | 0.0179 | 0.0466 |
| | (meso-diaminopimelate | (0-0) | (0-0) | | | |
| | containing) | | | | | |
| PWY_5097_Roseburia_hominis | L-lysine biosynthesis VI | 0 | 0.0000777 | 2767 | 0.018 | 0.0466 |
| | | (0-0.000114) | (0-0.000217) | | | |
| PWY_6386_Flavonifractor_plautii | UDP-N-acetylmuramoyl- | 0 | 0 | 1969 | 0.0179 | 0.0466 |
| | pentapeptide biosynthesis II | (0-0) | (0-0) | | | |
| | (lysine-containing) | | | | | |
| PWY_6387_Flavonifractor_plautii | UDP-N-acetylmuramoyl- | 0 | 0 | 1969 | 0.0179 | 0.0466 |
| | pentapeptide biosynthesis I | (0-0) | (0-0) | | | |
| | (meso-diaminopimelate | | | | | |
| | containing) | | | | | |
| UNINTEGRATED_Eggerthella_lenta | UNINTEGRATED | 0 | 0 | 1904.5 | 0.0181 | 0.0467 |
| | | (0-0) | (0-0) | | | |
| PWY_6700_Bacteroides_massiliensis | queuosine biosynthesis | 0 | 0 | 2683 | 0.0182 | 0.0471 |
| | | (0-0) | (0-0.000379) | | | |
| ENTBACSYN_PWY_Escherichia_coli | enterobactin biosynthesis | 0 | 0 | 1816.5 | 0.0185 | 0.0472 |
| | | (0-0.000363) | (0-0) | | | |
| PWY_5667_Bacteroides_xylanisolvens | CDP-diacylglycerol | 0 | 0.0000409 | 2747 | 0.0185 | 0.0472 |
| | biosynthesis I | (0-0.00008) | (0-0.000238) | | | |
| PWY_6936_Eubacterium_ventriosum | seleno-amino acid biosynthesis | 0 | 0 | 1796 | 0.0185 | 0.0472 |
| | | (0-0.000117) | (0-0.0000282) | | | |
| PWY0_1319_Bacteroides_xylanisolvens | CDP-diacylglycerol | 0 | 0.0000409 | 2747 | 0.0185 | 0.0472 |
| | biosynthesis II | (0-0.00008) | (0-0.000238) | | | |
| ILEUSYN_PWY_Dorea_formicigenerans | L-isoleucine biosynthesis I | 0.000123 | 0.0000638 | 1737 | 0.0186 | 0.0474 |
| | (from threonine) | (0-0.0002) | (0-0.000127) | | | |
| PWY_6151_Ruminococcus_lactaris | S-adenosyl-L-methionine | 0 | 0.000105 | 2756 | 0.0186 | 0.0474 |
| | cycle I | (0-0.000141) | (0-0.000277) | | | |
| COMPLETE_ARO_PWY_Lachnospiraceae_bacterium_1_1_57FAA | superpathway of aromatic | 0 | 0 | 1947.5 | 0.019 | 0.0482 |
| | amino acid biosynthesis | (0-0) | (0-0) | | | |
| PWY_6703_Bacteroides_massiliensis | preQ0 biosynthesis | 0 | 0 | 2671 | 0.0193 | 0.0488 |
| | | (0-0) | (0-0.000264) | | | |
| PWY_7111_Bacteroides_massiliensis | pyruvate fermentation to | 0 | 0 | 2679 | 0.0194 | 0.0488 |
| | isobutanol (engineered) | (0-0) | (0-0.00032) | | | |
| VALSYN_PWY_Bacteroides_massiliensis | L-valine biosynthesis | 0 | 0 | 2679 | 0.0194 | 0.0488 |
| | | (0-0) | (0-0.00032) | | | |
| VALSYN_PWY_Ruminococcus_lactaris | L-valine biosynthesis | 0 | 0.0000852 | 2755 | 0.0193 | 0.0488 |
| | | (0-0.000131) | (0-0.000222) | | | |
| PWY_5505_unclassified | L-glutamate and L-glutamine | 0 | 0 | 2614.5 | 0.0198 | 0.0493 |
| | biosynthesis | (0-0) | (0-0.0000582) | | | |
| PWY_5667_Paraprevotella_clara | CDP-diacylglycerol | 0 | 0 | 2703 | 0.0197 | 0.0493 |
| | biosynthesis I | (0-0.0000281) | (0-0.000278) | | | |
| PWY_5695_Roseburia_inulinivorans | urate biosynthesis/inosine 5′- | 0.00028 | 0.000136 | 1737.5 | 0.0199 | 0.0493 |
| | phosphate degradation | (0.00005-0.000871) | (0-0.000359) | | | |
| PWY_6122_Eggerthella_lenta | 5-aminoimidazole | 0 | 0 | 1916 | 0.0199 | 0.0493 |
| | ribonucleotide biosynthesis II | (0-0) | (0-0) | | | |
| PWY_6277_Eggerthella_lenta | superpathway of 5- | 0 | 0 | 1916 | 0.0199 | 0.0493 |
| | aminoimidazole ribonucleotide | (0-0) | (0-0) | | | |
| | biosynthesis | | | | | |
| PWY_7221_Lachnospiraceae_bacterium_3_1_57FAA_CT1 | guanosine ribonucleotides de | 0 | 0 | 1929 | 0.02 | 0.0493 |
| | novo biosynthesis | (0-0) | (0-0) | | | |
| PWY0_1319_Paraprevotella_clara | CDP-diacylglycerol | 0 | 0 | 2703 | 0.0197 | 0.0493 |
| | biosynthesis II | (0-0.0000281) | (0-0.000278) | | | |
| UNINTEGRATED_Clostridium_nexile | UNINTEGRATED | 0 | 0 | 1898 | 0.0198 | 0.0493 |
| | | (0-0.14) | (0-0) | | | |
| UNINTEGRATED_Paraprevotella_clara | UNINTEGRATED | 0 | 0 | 2705 | 0.02 | 0.0493 |
| | | (0-0.0495) | (0-0.292) | | | |
| PANTO_PWY_Ruminococcus_lactaris | phosphopantothenate | 0 | 0.000092 | 2738 | 0.0202 | 0.0496 |
| | biosynthesis I | (0-0.000108) | (0-0.000242) | | | |
| PWY_5097_Bacteroides_massiliensis | L-lysine biosynthesis VI | 0 | 0 | 2684 | 0.0202 | 0.0496 |
| | | (0-0) | (0-0.000376) | | | |

Shotgun functional analysis performed on 139 samples (IBS: n = 78 and Control: n = 58)
Median abundance % represented as inter-quartile range (IQR)

TABLE 5
Urine MS metabolomic Machine learning LASSO and Random Forest
(RF) statistics of urine metabolites predictive of IBS

| | |
|---|---|
| LASSO | RF |

| lambda | AUC | Sens | Spec | mtry | AUC | Sens | Spec |
|---|---|---|---|---|---|---|---|
| 0.050 | 1 | 0.978 | 1 | 1 | 0.999 | 0.988 | 1.000 |

| | |
|---|---|
| 10-fold Cross Validation | 10-fold Cross Validation |

| Reference | | | Reference | | |
|---|---|---|---|---|---|
| Prediction | Control | IBS | Prediction | Control | IBS |
| Control | 64 | 1.8 | Control | 64 | 1 |
| IBS | 0 | 78.2 | IBS | 0 | 79 |
| Accuracy | (average) | 0.9875 | Accuracy | (average) | 0.9931 |

| Rank # | Ranking | Metabolite | Rank # | Ranking | Metabolite |
|---|---|---|---|---|---|
| 1 | 100.00 | A 80987 | 1 | 100 | A 80987 |
| 2 | 60.15 | Ala-Leu-Trp-Gly | 2 | 89.74 | Ala-Leu-Trp-Gly |
| 3 | 38.02 | Medicagenic acid 3-O-b-D-glucuronide | 3 | 86.81 | Medicagenic acid 3-O-b-D-glucuronide |
| 4 | 1.95 | (−)-Epigallocatechin sulfate | 4 | 0.00 | (−)-Epigallocatechin sulfate |

Analysis had 2 classes: Control and IBS and included 144 samples (IBS: n = 80 and Control: n = 64)
Metrics reported are the average values from 10 repeats of 10-fold Cross Validation.

TABLE 6
urine metabolites significantly differentially abundant between IBS patients and non-IBS patients

| | IBS | Control | | Wilcoxon | | |
|---|---|---|---|---|---|---|
| Metabolite | (A.U.) | (A.U.) | Log2FC | Statistic | p-value | q-value |
| N-Undecanoylglycine | 212.2 | 16.5 | 3.686 | 28 | <0.001 | <0.001 |
| Gamma-glutamyl-Cysteine | 614.2 | 84.8 | 2.856 | 410 | <0.001 | <0.001 |
| Alloathyriol | 1.5 | 453.1 | −8.265 | 4101 | <0.001 | <0.001 |
| Trp-Ala-Pro | 6.1 | 0.2 | 4.646 | 763.5 | <0.001 | <0.001 |
| A 80987 | 730.8 | 0.1 | 12.885 | 0 | <0.001 | <0.001 |
| Medicagenic acid 3-O-b-D- | 475.4 | 12.8 | 5.212 | 0 | <0.001 | <0.001 |
| glucuronide | | | | | | |
| Ala-Leu-Trp-Gly | 420.3 | 120.5 | 1.802 | 83 | <0.001 | <0.001 |
| Butoctamide hydrogen succinate | 319 | 3.1 | 6.677 | 423 | <0.001 | <0.001 |
| (−)-Epicatechin sulfate | 274 | 209.8 | 0.385 | 506 | <0.001 | <0.001 |
| 1,4,5-Trimethyl-naphtalene | 15.2 | 0 | 8.739 | 658.5 | <0.001 | <0.001 |
| Tricetin 3′-methyl ether 7,5′- | 0.6 | 22.5 | −5.156 | 4094 | <0.001 | <0.001 |
| diglucuronide | | | | | | |
| Torasemide | 0.5 | 38.2 | −6.289 | 4023 | <0.001 | <0.001 |
| (−)-Epigallocatechin sulfate | 129.1 | 165.2 | −0.356 | 3826 | <0.001 | <0.001 |
| Dodecanedioylcarnitine | 61.9 | 9.7 | 2.679 | 1054 | <0.001 | <0.001 |
| 1,6,7-Trimethylnaphthalene | 17.2 | 0.1 | 7.234 | 1082.5 | <0.001 | <0.001 |
| Tetrahydrodipicolinate | 1.8 | 71.6 | −5.324 | 3671 | <0.001 | <0.001 |
| Sumiki's acid | 84667.1 | 58728.8 | 0.528 | 1181 | <0.001 | <0.001 |
| Silicic acid | 4 | 734.3 | −7.527 | 3556 | <0.001 | <0.001 |
| Delphinidin 3-(6″-O-4-malyl- | 0.2 | 16.2 | −6.341 | 3548 | <0.001 | <0.001 |
| glucosyl)-5-glucoside | | | | | | |
| L-Arginine | 0 | 13.7 | −8.547 | 3540 | <0.001 | <0.001 |
| Leucyl-Methionine | 9.5 | 60.7 | −2.682 | 3526 | <0.001 | <0.001 |
| Phe-Gly-Gly-Ser | 420 | 359.6 | 0.224 | 1250 | <0.001 | <0.001 |
| Gln-Met-Pro-Ser | 179.8 | 272.8 | −0.601 | 3507 | <0.001 | <0.001 |
| Creatinine | 729604.9 | 752607.5 | −0.045 | 3500 | <0.001 | <0.001 |
| Ala-Asn-Cys-Gly | 177.5 | 229.7 | −0.372 | 3431 | <0.001 | <0.001 |
| 2-hydroxy-2-(hydroxymethyl)-2H- | 508.8 | 256 | 0.991 | 1329 | <0.001 | <0.001 |
| pyran-3(6H)-one | | | | | | |
| Thiethylperazine | 38 | 9.7 | 1.974 | 1365 | <0.001 | <0.001 |
| 5-((2-iodoacetamido)ethyl)-1- | 627.5 | 257.7 | 1.284 | 1366.5 | <0.001 | <0.001 |
| aminonapthalene sulfate | | | | | | |
| dCTP | 379 | 323.1 | 0.231 | 1390 | <0.001 | <0.001 |
| Isoleucyl-Proline | 10391.2 | 12988.5 | −0.322 | 3362 | <0.001 | <0.001 |
| 3,4-Methylenesebacic acid | 452826.1 | 482052 | −0.09 | 3344 | <0.001 | <0.001 |
| Dimethylallylpyrophosphate/Isopentenyl | 15680 | 9743.9 | 0.686 | 1425 | <0.001 | <0.001 |
| pyrophosphate | | | | | | |
| (4-Hydroxybenzoyl)choline | 68.6 | 112.2 | −0.711 | 3329 | <0.001 | <0.001 |
| Diazoxide | 145.7 | 212 | −0.541 | 3318 | <0.001 | <0.001 |
| 3,5-Di-O-galloyl-1,4- | 638.5 | 539.3 | 0.243 | 1458 | <0.001 | <0.001 |
| galactarolactone | | | | | | |
| 2-Hydroxypyridine | 37.8 | 164.2 | −2.121 | 3300 | <0.001 | <0.001 |
| Decanoylcamitine | 152.9 | 46.7 | 1.71 | 1463 | <0.001 | <0.001 |
| Asp-Met-Asp-Pro | 894.5 | 744.1 | 0.266 | 1473 | <0.001 | <0.001 |
| 3-Methyldioxyindole | 203 | 326 | −0.683 | 3250 | 0.00022 | 0.00161 |
| (1S,3R,4S)-3,4- | 749.3 | 1010.2 | −0.431 | 3244 | 0.000243 | 0.00173 |
| Dihydroxy cyclohexane-1- | | | | | | |
| carboxylate | | | | | | |
| Ala-Lys-Phe-Cys | 47.3 | 107.7 | −1.186 | 3238 | 0.000269 | 0.00187 |
| 3-Indolehydracrylic acid | 972.6 | 1898.6 | −0.965 | 3216 | 0.000385 | 0.00261 |
| [FA (18:0)] N-(9Z-octadecenoyl)- | 197 | 178.2 | 0.145 | 1545 | 0.000404 | 0.00267 |
| taurine | | | | | | |
| Ferulic acid 4-sulfate | 1569 | 3452.2 | −1.138 | 3174 | 0.000746 | 0.00482 |
| Urea | 188415 | 198969.2 | −0.079 | 3172 | 0.000769 | 0.00487 |
| N-Carboxyacetyl-D-phenylalanine | 307.4 | 438.4 | −0.512 | 3166 | 0.000843 | 0.00522 |
| 4-Methoxyphenylethanol sulfate | 476.3 | 889.1 | −0.9 | 3155 | 0.000996 | 0.00604 |
| UDP-4-dehydro-6-deoxy-D-glucose | 192.4 | 171.7 | 0.164 | 1606 | 0.00104 | 0.00606 |
| Linalyl formate | 20.8 | 30.6 | −0.555 | 3153 | 0.00103 | 0.00606 |
| Demethyloleuropein | 9.1 | 21.5 | −1.233 | 3148 | 0.00111 | 0.0063 |
| 5′-Guanosyl-methylene-triphosphate | 337.4 | 428.7 | −0.346 | 3140 | 0.00125 | 0.00683 |
| Allyl nonanoate | 18.4 | 24 | −0.385 | 3140 | 0.00125 | 0.00683 |
| 2-Phenylethyl octanoate | 67.9 | 184.7 | −1.444 | 3132 | 0.0014 | 0.00754 |
| beta-Cellobiose | 163.4 | 117.1 | 0.48 | 1628 | 0.00145 | 0.00762 |
| D-Galactopyranosyl-(1−>3)-D- | 271.6 | 756.8 | −1.479 | 3125 | 0.00156 | 0.00805 |
| galactopyranosyl-(1−>3)-L-arabinose | | | | | | |
| Cys-Phe-Phe-Gln | 41.1 | 62 | −0.593 | 3114 | 0.00182 | 0.00927 |
| Hippuric acid | 89463.1 | 125800 | −0.492 | 3108 | 0.00199 | 0.00993 |
| Cys-Pro-Pro-Tyr | 51.1 | 73.6 | −0.527 | 3098 | 0.00229 | 0.0112 |
| Met-Met-Thr-Trp | 112 | 151.5 | −0.436 | 3085 | 0.00275 | 0.0132 |
| methylphosphonate | 476.1 | 515.7 | −0.115 | 3084 | 0.00279 | 0.0132 |
| 3′-Sialyllactosamine | 84.8 | 129.1 | −0.606 | 3082 | 0.00287 | 0.0134 |
| 2,4,6-Octatriynoic acid | 1438.5 | 1703.3 | −0.244 | 3079 | 0.00299 | 0.0137 |
| Delphinidin 3-O-3″,6″-O- | 229.7 | 164.6 | 0.481 | 1681 | 0.00307 | 0.0139 |
| dimalonylglucoside | | | | | | |
| L-Valine | 8240.3 | 7936.7 | 0.054 | 1685 | 0.00325 | 0.0142 |
| Met-Met-Cys | 192.1 | 163.5 | 0.233 | 1685 | 0.00325 | 0.0142 |
| Cysteinyl-Cysteine | 14357 | 11017.4 | 0.382 | 1687 | 0.00334 | 0.0144 |
| (all-E)-1,8,10-Heptadecatriene-4,6- | 378 | 788.6 | −1.061 | 3068 | 0.00348 | 0.0145 |
| diyne-3,12-diol | | | | | | |
| L-Lysine | 135.9 | 76.8 | 0.823 | 1689 | 0.00343 | 0.0145 |
| Pivaloylcarnitine | 1262.9 | 1788 | −0.502 | 3059 | 0.00393 | 0.0159 |
| Lenticin | 113 | 217.8 | −0.946 | 3059 | 0.00393 | 0.0159 |
| Phenol glucuronide | 405.7 | 287.8 | 0.495 | 1701 | 0.00403 | 0.0159 |
| Tyrosyl-Cysteine | 957.9 | 802.2 | 0.256 | 1705 | 0.00426 | 0.0159 |
| Osmundalin | 533.8 | 317.1 | 0.751 | 1703 | 0.00414 | 0.0159 |
| Tetrahydroaldosterone-3-glucuronide | 781.6 | 975.4 | −0.32 | 3054 | 0.0042 | 0.0159 |
| N-Methylpyridinium | 3882.3 | 13043 | −1.748 | 3055 | 0.00414 | 0.0159 |
| L-prolyl-L-proline | 3080.2 | 5296.2 | −0.782 | 3056 | 0.00409 | 0.0159 |
| Glutarylcamitine | 698.7 | 864.8 | −0.308 | 3042 | 0.00492 | 0.018 |
| [FA (15:4)] 6,8,10,12- | 2303.5 | 3781 | −0.715 | 3042 | 0.00492 | 0.018 |
| pentadecatetraenal | | | | | | |
| Methyl bisnorbiotinyl ketone | 2259.7 | 1986.7 | 0.186 | 1720 | 0.00519 | 0.0187 |
| Acetoin | 1239.3 | 785.6 | 0.658 | 1726 | 0.00561 | 0.02 |
| LysoPC(18:2(9Z,12Z)) | 0.8 | 48.3 | −5.859 | 3029 | 0.00584 | 0.0205 |
| Hexyl 2-furoate | 17 | 24.7 | −0.537 | 3021 | 0.00647 | 0.0225 |
| N-carbamoyl-L-glutamate | 331.9 | 423.8 | −0.353 | 3018 | 0.00673 | 0.0231 |
| L-Homoserine | 4000.7 | 5333.1 | −0.415 | 3012 | 0.00726 | 0.0246 |
| L-Asparagine | 300 | 384.8 | −0.359 | 3011 | 0.00736 | 0.0246 |
| Tiglylcarnitine | 314.5 | 762.4 | −1.278 | 3008 | 0.00764 | 0.025 |
| Thymine | 110 | 76.8 | 0.519 | 1751 | 0.00774 | 0.025 |
| 3-hydroxypyridine | 271.4 | 556.5 | −1.036 | 3007 | 0.00774 | 0.025 |
| Menadiol disuccinate | 793.6 | 2024.6 | −1.351 | 3005 | 0.00793 | 0.0254 |
| 9-Decenoylcamitine | 1951.1 | 2609.8 | −0.42 | 2996 | 0.00888 | 0.0275 |
| Pyrocatechol sulfate | 27377.5 | 40427.9 | −0.562 | 2996 | 0.00888 | 0.0275 |
| sedoheptulose anhydride | 4159 | 10851.9 | −1.384 | 2995 | 0.00899 | 0.0275 |
| (+)-gamma-Hydroxy-L- | 272.2 | 398.3 | −0.549 | 2997 | 0.00877 | 0.0275 |
| homoarginine | | | | | | |
| Thioridazine | 884.1 | 1048.3 | −0.246 | 2984 | 0.0103 | 0.0312 |
| Cys-Glu-Glu-Glu | 37.3 | 56.8 | −0.609 | 2977 | 0.0112 | 0.0329 |
| Marmesin rutinoside | 17.8 | 36.1 | −1.025 | 2977 | 0.0112 | 0.0329 |
| L-Serine | 991.6 | 1146.2 | −0.209 | 2978 | 0.0111 | 0.0329 |
| L-Urobilinogen | 8.5 | 139.7 | −4.035 | 2976 | 0.0113 | 0.033 |
| Isobutyrylglycine | 2274.1 | 2694.4 | −0.245 | 2974 | 0.0116 | 0.0334 |
| S-Adenosylhomocysteine | 135.5 | 454.5 | −1.746 | 2968 | 0.0125 | 0.0356 |
| 2,3-dioctanoylglyceramide | 887.1 | 1277.1 | −0.526 | 2966 | 0.0128 | 0.0357 |
| 3-Methoxy-4-hydroxyphenylglycol | 0.5 | 10.7 | −4.335 | 2966.5 | 0.0127 | 0.0357 |
| glucuronide | | | | | | |
| sulfoethylcysteine | 5602.3 | 8425.3 | −0.589 | 2965 | 0.013 | 0.0358 |
| Hydroxyphenylacetylglycine | 460.1 | 568.5 | −0.305 | 2962 | 0.0134 | 0.0367 |
| Pyrroline hydroxycarboxylic acid | 13972.6 | 16170.5 | −0.211 | 2961 | 0.0136 | 0.0368 |
| 1-(alpha-Methyl-4-(2- | 131.6 | 259.6 | −0.98 | 2956 | 0.0144 | 0.0383 |
| methylpropyl)benzeneacetate)-beta- | | | | | | |
| D-Glucopyranuronic acid | | | | | | |
| 2-Methylbutylacetate | 1958.5 | 2726.3 | −0.477 | 2956 | 0.0144 | 0.0383 |
| N1-Methyl-4-pyridone-3- | 6162.4 | 9041.6 | −0.553 | 2955 | 0.0146 | 0.0384 |
| carboxamide | | | | | | |
| Cortolone-3-glucuronide | 520.3 | 620.8 | −0.255 | 2953 | 0.0149 | 0.039 |
| Asn-Cys-Gly | 255.4 | 231 | 0.145 | 1813 | 0.0164 | 0.0413 |
| N6,N6,N6-Trimethyl-L-lysine | 2282.7 | 2591.3 | −0.183 | 2946 | 0.0162 | 0.0413 |
| Benzylamine | 66.3 | 218.7 | −1.722 | 2947 | 0.016 | 0.0413 |
| 5-Hydroxy-L-tryptophan | 177.9 | 218.1 | −0.294 | 2945 | 0.0164 | 0.0413 |
| Armillaric acid | 25 | 44.5 | −0.833 | 2941 | 0.0172 | 0.0429 |
| Leucine/Isoleucine | 979.3 | 1135.6 | −0.214 | 2939 | 0.0176 | 0.0435 |
| 2-Butylbenzothiazole | 441.4 | 381.3 | 0.211 | 1821 | 0.018 | 0.0441 |
| D-Sedoheptulose 7-phosphate | 297.5 | 497.5 | −0.742 | 2936 | 0.0182 | 0.0442 |
| [Fv Dimethoxy,methyl(9:1)] (2S)- | 651.1 | 1201.2 | −0.883 | 2935 | 0.0184 | 0.0444 |
| 5,7-Dimethoxy-3′,4′- | | | | | | |
| methylenedioxyflavanone | | | | | | |
| Oxoadipic acid | 487.5 | 617.2 | −0.341 | 2934 | 0.0186 | 0.0445 |
| Thr-Cys-Cys | 2325.9 | 2798.8 | −0.267 | 2933 | 0.0188 | 0.0446 |
| Creatine | 4511 | 15140.7 | −1.747 | 2930 | 0.0195 | 0.0458 |
| Hydroxybutyrylcarnitine | 156.7 | 259.7 | −0.729 | 2929 | 0.0197 | 0.0459 |
| 5′-Dehydroadenosine | 168.5 | 106.9 | 0.656 | 1833 | 0.0206 | 0.0462 |
| Phe-Thr-Val | 47.6 | 82.5 | −0.793 | 2925 | 0.0206 | 0.0462 |
| dUDP | 149.3 | 319.2 | −1.096 | 2925 | 0.0206 | 0.0462 |
| L-Glutamine | 616.2 | 706.6 | −0.197 | 2926 | 0.0204 | 0.0462 |
| Kaempferol 3-(2″,3″-diacetyl-4″-p- | 32.8 | 113.1 | −1.788 | 2927 | 0.0201 | 0.0462 |
| coumaroylrhamnoside) | | | | | | |

Metabolomic analysis performed on 139 samples (IBS: n = 78 and Control: n = 61)
Median concentration represented as arbitary unit (A.U.)
Log2FC, log2 fold change between the groups

TABLE 7
Fecal MS metabolomic Machine learning LASSO and Random Forest (RF) statistics for diagnosing IBS

| | |
|---|---|
| LASSO | RF |

| lambda | AUC | Sens | Spec | mtry | AUC | Sens | Spec |
|---|---|---|---|---|---|---|---|
| 0.051 | 1 | 0.700 | 0.475 | 1 | 0.862 | 0.821 | 0.647 |

| 10-fold Cross Validation for Training Set | | | 10-fold Cross Validation for Training Set | | |
|---|---|---|---|---|---|
| Reference | | | Reference | | |
| Prediction | Control | IBS | Prediction | Control | IBS |
| Control | 29.9 | 24 | Control | 40.5 | 14.4 |
| IBS | 33.1 | 56 | IBS | 22.5 | 65.6 |
| Accuracy | (average) | 0.601 | Accuracy | (average) | 0.742 |

| Rank # | Ranking | Metabolite | Rank # | Ranking | Metabolite |
|---|---|---|---|---|---|
| 1 | 100.00 | 3-deoxy-D-galactose | 1 | 100 | 3-deoxy-D-galactose |
| 2 | 97.93 | Tyrosine | 2 | 86.3 | Tyrosine |
| 3 | 51.16 | I-Urobilin | 3 | 80.8 | I-Urobilin |
| 4 | 0.13 | Adenosine | 4 | 80.0 | Adenosine |
| 5 | 0.09 | Glu-Ile-Ile-Phe | 5 | 78.9 | Glu-Ile-Ile-Phe |
| 6 | 0.06 | 3,6-Dimethoxy-19-norpregna- | 6 | 77.1 | 3,6-Dimethoxy-19-norpregna- |
| | | l,3,5,7,9-pentaen-20-one | | | l,3,5,7,9-pentaen-20-one |
| 7 | 0.04 | 2-Phenylpropionate | 7 | 62.9 | 2-Phenylpropionate |
| 8 | 0.04 | MG(20:3(8Z,11Z,14Z)/0:0/0:0) | 8 | 61.9 | MG(20:3(8Z,11Z,14Z)/0:0/0:0) |
| 9 | 0.03 | 1,2,3-Tris(1-ethoxyethoxy)propane | 9 | 60.4 | 1,2,3-Tris(1-ethoxyethoxy)propane |
| 10 | 0.03 | Staphyloxanthin | 10 | 60.3 | Staphyloxanthin |
| 11 | 0.02 | Hexoses | 11 | 59.0 | Hexoses |
| 12 | 0.02 | 20-hydroxy-E4-neuroprostane | 12 | 58.2 | 20-hydroxy-E4-neuroprostane |
| 13 | 0.02 | Nonyl acetate | 13 | 56.7 | Nonyl acetate |
| 14 | 0.01 | 3-Feruloyl-1,5-quinolactone | 14 | 56.2 | 3-Feruloyl-1,5-quinolactone |
| 15 | 0.01 | trans-2-Heptenal | 15 | 53.0 | trans-2-Heptenal |
| 16 | 0.01 | Pyridoxamine | 16 | 48.9 | Pyridoxamine |
| 17 | 0.01 | L-Arginine | 17 | 46.3 | L-Arginine |
| 18 | 0.01 | Dodecanedioic acid | 18 | 44.9 | Dodecanedioic acid |
| 19 | 0.01 | Ursodeoxycholic acid | 19 | 43.5 | Ursodeoxycholic acid |
| 20 | 0.003 | 1-(Malonylamino)cyclopropanecarboxylic acid | 20 | 43.5 | 1-(Malonylamino)cyclopropanecarboxylic acid |
| 21 | 0.002 | Cortisone | 21 | 42.5 | Cortisone |
| 22 | 0.002 | 9,10,13-Trihydroxystearic acid | 22 | 42.4 | 9,10,13-Trihydroxystearic acid |
| 23 | 0.002 | Glu-Ala-Gln-Ser | 23 | 36.6 | Glu-Ala-Gln-Ser |
| 24 | 0.002 | Quasiprotopanaxatriol | 24 | 36.3 | Quasiprotopanaxatriol |
| 25 | 0.001 | N-Methylindolo[3,2-b]-5alpha-cholest-2-ene | 25 | 35.3 | N-Methylindolo[3,2-b]-5alpha-cholest-2-ene |
| 26 | 0.001 | PG(20:0/22:1(11Z)) | 26 | 34.4 | PG(20:0/22:1(11Z)) |
| 27 | 0.001 | (−)-Epigallocatechin | 27 | 34.3 | (−)-Epigallocatechin |
| 28 | 0.001 | 2-Methyl-3-ketovaleric acid | 28 | 30.8 | 2-Methyl-3-ketovaleric acid |
| 29 | 0.001 | Secoeremopetasitolide B | 29 | 30.4 | Secoeremopetasitolide B |
| 30 | 0.001 | PC(20:1(11Z)/P-16:0) | 30 | 28.7 | PC(20:1(11Z)/P-16:0) |
| 31 | 0.001 | Glu-Asp-Asp | 31 | 26.3 | Glu-Asp-Asp |
| 32 | 0.001 | N5-acetyl-N5-hydroxy-L-ornithine acid | 32 | 23.9 | N5-acetyl-N5-hydroxy-L-ornithine acid |
| 33 | 0.001 | Silicic acid | 33 | 22.7 | Silicic acid |
| 34 | 0.0005 | (1xi,3xi)-1,2,3,4-Tetrahydro-1-methyl-beta- | 34 | 22.2 | (1xi,3xi)-1,2,3,4-Tetrahydro-1-methyl-beta- |
| | | carboline-3-carboxylic acid | | | carboline-3-carboxylic acid |
| 35 | 0.0004 | PS(36:5) | 35 | 21.9 | PS(36:5) |
| 36 | 0.0002 | Chorismate | 36 | 17.6 | Chorismate |
| 37 | 0.0002 | Isoamyl isovalerate | 37 | 17.5 | Isoamyl isovalerate |
| 38 | 0.0002 | PA(O-36:4) | 38 | 12.5 | PA(O-36:4) |
| 39 | 0.0001 | PE(P-28:0) | 39 | 8.0 | PE(P-28:0) |
| 40 | 0.00001 | gamma-Glutamyl-S-methylcysteinyl-beta- | 40 | 0 | gamma-Glutamyl-S-methylcysteinyl-beta- |
| | | alanine | | | alanine |

Analysis had 2 classes: Control and IBS and included 143 samples (IBS: n = 80 and Control: n = 63)
753 predictors were used in the model
No test set

TABLE 8
Fecal metabolites differentially abundant between the IBS and Control groups

| | IBS | Control | | Wilcoxon | | |
|---|---|---|---|---|---|---|
| Metabolite | (A.U.) | (A.U.) | Log2FC | Statistic | p-value | q-value |
| 2-Phenylpropionate | 1323182.1 | 3247921.9 | −1.296 | 3374 | 0 | 0.00505 |
| 3-Buten-1-amine | 280286.1 | 167168.2 | 0.746 | 1388 | 0 | 0.00505 |
| Adenosine | 125862.8 | 222491.1 | −0.822 | 3340 | 0 | 0.00505 |
| I-Urobilin | 2129046.3 | 508459.2 | 2.066 | 1444 | 0 | 0.00505 |
| 2,3-Epoxymenaquinone | 245989.6 | 547357.5 | −1.154 | 3313 | 0 | 0.00505 |
| [FA (22:5)] 4,7,10,13,16-Docosapentaynoic | 516717.6 | 1051721 | −1.025 | 3309 | 0 | 0.00505 |
| acid | | | | | | |
| 3,6-Dimethoxy-19-norpregna-1,3,5,7,9- | 961706.2 | 2013326.2 | −1.066 | 3298 | 0 | 0.00505 |
| pentaen-20-one | | | | | | |
| Cucurbitacin S | 1617422.3 | 812194.6 | 0.994 | 1462 | 0.0001 | 0.00505 |
| N-Heptanoylglycine | 581244.7 | 1189914.8 | −1.034 | 3296 | 0.0001 | 0.00505 |
| 11-Deoxocucurbitacin I | 1509367.4 | 1026985.6 | 0.556 | 1478 | 0.000132 | 0.00599 |
| Staphyloxanthin | 125908.3 | 208397.6 | −0.727 | 3264 | 0.000174 | 0.00716 |
| Piperidine | 536820.9 | 366827.8 | 0.549 | 1501 | 0.000196 | 0.00722 |
| Leu-Ser-Ser-Tyr | 194085.5 | 88714.9 | 1.129 | 1509 | 0.000224 | 0.00722 |
| L-Urobilin | 31844915.4 | 58134193.3 | −0.868 | 3249 | 0.000224 | 0.00722 |
| L-Phenylalanine | 2052003.6 | 1343878.1 | 0.611 | 1513 | 0.000239 | 0.00722 |
| Ala-Leu-Trp-Pro | 323939 | 638393.1 | −0.979 | 3238 | 0.000269 | 0.0074 |
| 3-Feruloyl-1,5-quinolactone | 524541.4 | 876281.8 | −0.74 | 3236 | 0.000278 | 0.0074 |
| PG(P-16:0/14:0) | 426308.9 | 798780.6 | −0.906 | 3223 | 0.000343 | 0.00832 |
| 3-deoxy-D-galactose | 226693.6 | 145983.2 | 0.635 | 1536 | 0.000349 | 0.00832 |
| MG(20:3(8Z,11Z,14Z)/0:0/0:0) | 89430.2 | 214373.1 | −1.261 | 3215 | 0.000391 | 0.00857 |
| Mesobilirubinogen | 696662.3 | 251218.8 | 1.472 | 1544 | 0.000397 | 0.00857 |
| L-Alanine | 1429957.1 | 1081997.7 | 0.402 | 1548 | 0.000424 | 0.00872 |
| Tyrosine | 533603.6 | 368180.1 | 0.535 | 1564 | 0.000546 | 0.0106 |
| PG(O-30:1) | 140723.9 | 291063.6 | −1.048 | 3192 | 0.000564 | 0.0106 |
| beta-Pinene | 171.8 | 276.9 | −0.689 | 3187 | 0.00061 | 0.011 |
| 2,4,8-Eicosatrienoic acid isobutylamide | 53648.9 | 167764.9 | −1.645 | 3170.5 | 0.000787 | 0.0135 |
| Glutarylglycine | 1561150.4 | 2367236.8 | −0.601 | 3169 | 0.000805 | 0.0135 |
| [PR] gamma-Carotene/beta,psi-Carotene | 39594.5 | 55014.4 | −0.475 | 3155 | 0.000996 | 0.0161 |
| Neuromedin B (1-3) | 1195664.8 | 414438.1 | 1.529 | 1610 | 0.00111 | 0.0173 |
| Heptane-1-thiol | 435910.8 | 336879.8 | 0.372 | 1613 | 0.00116 | 0.0174 |
| Violaxanthin | 688839.8 | 991237.9 | −0.525 | 3143 | 0.00119 | 0.0174 |
| Isolimonene | 6.8 | 19 | −1.492 | 3138 | 0.00128 | 0.0182 |
| Ile-Lys-Cys-Gly | 422439.2 | 241750.4 | 0.805 | 1625 | 0.00138 | 0.0187 |
| His-Met-Val-Val | 377162.4 | 223544.7 | 0.755 | 1626 | 0.0014 | 0.0187 |
| Allyl caprylate | 9.6 | 7.7 | 0.326 | 1632 | 0.00153 | 0.0196 |
| Hydroxyprolyl-Tryptophan | 323127 | 123183.6 | 1.391 | 1633 | 0.00156 | 0.0196 |
| Dodecanedioic acid | 671845.4 | 956268.6 | −0.509 | 3122 | 0.00162 | 0.0199 |
| 2-O-Benzoyl-D-glucose | 220717.8 | 469968.1 | −1.09 | 3119 | 0.0017 | 0.0199 |
| 2-Ethylsuberic acid | 384419 | 749840 | −0.964 | 3118 | 0.00172 | 0.0199 |
| D-Urobilin | 1792754.5 | 301418.7 | 2.572 | 1641.5 | 0.00176 | 0.0199 |
| 20-hydroxy-E4-neuroprostane | 125388 | 208519 | −0.734 | 3113 | 0.00185 | 0.02 |
| PG(O-31:1) | 525453.8 | 924227.6 | −0.815 | 3113 | 0.00185 | 0.02 |
| Anigorufone | 754382 | 1783246.9 | −1.241 | 3110 | 0.00193 | 0.0203 |
| Nonyl acetate | 13.1 | 8.2 | 0.677 | 1658 | 0.00223 | 0.0229 |
| L-Arginine | 32851.3 | 72856.2 | −1.149 | 3095 | 0.00239 | 0.0239 |
| PG(P-32:1) | 164475.2 | 226435.7 | −0.461 | 3094 | 0.00242 | 0.0239 |
| Glu-Ala-Gln-Ser | 375851.9 | 273805.4 | 0.457 | 1668 | 0.00256 | 0.0247 |
| PG(31:0) | 160964.6 | 277244.2 | −0.784 | 3087 | 0.00267 | 0.0252 |
| Cucurbitacin I | 793831.5 | 470668 | 0.754 | 1683 | 0.00316 | 0.0275 |
| Arg-Lys-Phe-Val | 479994 | 2477823.7 | −2.368 | 3075 | 0.00316 | 0.0275 |
| Genipinic acid | 269618.2 | 535154 | −0.989 | 3072.5 | 0.00327 | 0.0275 |
| Hexoses | 63587.8 | 102387.4 | −0.687 | 3072.5 | 0.00327 | 0.0275 |
| Lys-Phe-Phe-Phe | 144955.5 | 76014.5 | 0.931 | 1686 | 0.00329 | 0.0275 |
| PI(41:2) | 523352.3 | 289816 | 0.853 | 1686 | 0.00329 | 0.0275 |
| D-galactal | 236791 | 433511.6 | −0.872 | 3071 | 0.00334 | 0.0275 |
| Traumatic acid | 235655 | 352893.3 | −0.583 | 3066 | 0.00357 | 0.0287 |
| Adenine | 312165.1 | 445818.4 | −0.514 | 3065 | 0.00362 | 0.0287 |
| PC(22:2(13Z,16Z)/15:0) | 249100.9 | 131882.9 | 0.917 | 1695 | 0.00372 | 0.0287 |
| 2-Phenylethyl beta-D-glucopyranoside | 330200 | 576025.7 | −0.803 | 3061 | 0.00382 | 0.0287 |
| PG(37:2) | 208672.4 | 309558.5 | −0.569 | 3060 | 0.00387 | 0.0287 |
| Glycerol tributanoate | 1818865.6 | 790191.7 | 1.203 | 1699 | 0.00393 | 0.0287 |
| Arg-Leu-Pro-Arg | 1113239.6 | 805486.6 | 0.467 | 1699 | 0.00393 | 0.0287 |
| 2-O-p-Coumaroyl-D-glucose | 177559.4 | 309984.6 | −0.804 | 3057 | 0.00403 | 0.029 |
| 3,4-Dihydroxyphenyllactic acid methyl ester | 172842.1 | 321573.9 | −0.896 | 3055 | 0.00414 | 0.0293 |
| PG(P-28:0) | 70315.5 | 138650.1 | −0.98 | 3054 | 0.0042 | 0.0293 |
| PG(34:0) | 80115.9 | 135649.9 | −0.76 | 3050 | 0.00443 | 0.0298 |
| L-Lysine | 391680.5 | 290959.3 | 0.429 | 1710 | 0.00455 | 0.0298 |
| Ribitol | 139100.6 | 308432.9 | −1.149 | 3048 | 0.00455 | 0.0298 |
| LysoPE(18:2(9Z,12Z)/0:0) | 41861 | 70972.6 | −0.762 | 3048 | 0.00455 | 0.0298 |
| PA(20:4(5Z,8Z,11Z,14Z)e/2:0) | 117279.2 | 179176 | −0.611 | 3046 | 0.00467 | 0.0298 |
| 5-Dehydroshikimate | 270282 | 486194.1 | −0.847 | 3046 | 0.00467 | 0.0298 |
| Threoninyl-Isoleucine | 302458.5 | 194748 | 0.635 | 1715 | 0.00486 | 0.0301 |
| L-Methionine | 296185.5 | 228939.8 | 0.372 | 1717 | 0.00499 | 0.0301 |
| PS(26:0)) | 3551762.1 | 1704565.8 | 1.059 | 1717 | 0.00499 | 0.0301 |
| alpha-Pinene | 92.1 | 215.6 | −1.227 | 3041 | 0.00499 | 0.0301 |
| Fenchene | 12.1 | 26.4 | −1.124 | 3039 | 0.00512 | 0.0305 |
| Glu-Ile-Ile-Phe | 171216.3 | 125559 | 0.447 | 1721 | 0.00526 | 0.0305 |
| Gln-Phe-Phe-Phe | 367594.4 | 170906.7 | 1.105 | 1721 | 0.00526 | 0.0305 |
| Ursodeoxycholic acid | 12666176 | 6449124.1 | 0.974 | 1726 | 0.00561 | 0.0318 |
| PC(34:2) | 112528.4 | 208697.3 | −0.891 | 3032 | 0.00561 | 0.0318 |
| 3,17-Androstanediol glucuronide | 469180.9 | 755540.9 | −0.687 | 3031 | 0.00569 | 0.0318 |
| Pyridoxamine | 56652.2 | 41022 | 0.466 | 1730.5 | 0.00595 | 0.0324 |
| [ST hydrox] (25R)-3alpha,7alpha-dihydroxy- | 319975.1 | 229268.9 | 0.481 | 1732 | 0.00607 | 0.0324 |
| 5beta-cholestan-27-oyl taurine | | | | | | |
| PA(42:2) | 1782124.8 | 686161.8 | 1.377 | 1732 | 0.00607 | 0.0324 |
| [FA (16:0)] 2-bromo-hexadecanal | 515055.9 | 256899.2 | 1.004 | 1733 | 0.00615 | 0.0324 |
| 3,6-Dihydro-4-(4-methyl-3-pentenyl)-1,2- | 479922.9 | 701686.5 | −0.548 | 3025 | 0.00615 | 0.0324 |
| dithiin | | | | | | |
| 3-Methylcrotonylglycine | 161596.9 | 287502.4 | −0.831 | 3024 | 0.00623 | 0.0324 |
| xi-7-Hydroxyhexadecanedioic acid | 48647.5 | 70410.5 | −0.533 | 3020 | 0.00656 | 0.0337 |
| Camphene | 7.7 | 17.7 | −1.192 | 3017 | 0.00681 | 0.0345 |
| 2-Hydroxy-3-carboxy-6-oxo-7-methylocta- | 375469 | 560318.7 | −0.578 | 3014 | 0.00708 | 0.0345 |
| 2,4-dienoate | | | | | | |
| 7C-aglycone | 1658154.1 | 2581551 | −0.639 | 3014 | 0.00708 | 0.0345 |
| 1-(3-Aminopropyl)-4-aminobutanal | 1007823.6 | 194401.6 | 2.374 | 1744 | 0.00708 | 0.0345 |
| Benzyl isobutyrate | 79.6 | 152.6 | −0.938 | 3014 | 0.00708 | 0.0345 |
| (S)-(E)-8-(3,6-Dimethyl-2-heptenyl)-4′,5,7- | 213117.3 | 551116.6 | −1.371 | 3010 | 0.00745 | 0.0346 |
| trihydroxyflavanone | | | | | | |
| 1,3-di-(5Z,8Z,11Z,14Z,17Z- | 100264.5 | 212921.2 | −1.087 | 3010 | 0.00745 | 0.0346 |
| eicosapentaenoyl)-2-hydroxy-glycerol (d5) | | | | | | |
| SM(d18:0/18:0) | 80949.8 | 49417.7 | 0.712 | 1748 | 0.00745 | 0.0346 |
| L-Homoserine | 292067.7 | 226802.4 | 0.365 | 1749 | 0.00754 | 0.0346 |
| 17beta-(Acetylthio)estra-1,3,5 (10)-trien-3-ol | 630945.8 | 1067932 | −0.759 | 3009 | 0.00754 | 0.0346 |
| acetate | | | | | | |
| [ST (2:0)] 5beta-Chola-3,11-dien-24-oic | 695679.3 | 320859.8 | 1.116 | 1750 | 0.00764 | 0.0346 |
| Acid | | | | | | |
| PG(33:2) | 75974.9 | 50361.9 | 0.593 | 1750 | 0.00764 | 0.0346 |
| PE(22:4(7Z,10Z,13Z,16Z)/P-16:0) | 81995.8 | 100782 | −0.298 | 3006 | 0.00783 | 0.0351 |
| Protoporphyrinogen IX | 255656.7 | 187102.1 | 0.45 | 1756 | 0.00824 | 0.0366 |
| alpha-Tocopherol succinate | 47245.3 | 108160.8 | −1.195 | 3001 | 0.00834 | 0.0367 |
| Methyl (9Z)-6′-oxo-6,5′-diapo-6-carotenoate | 899831 | 218922.6 | 2.039 | 1760 | 0.00866 | 0.037 |
| PG(16:1(9Z)/16:1(9Z)) | 103173.2 | 74458 | 0.471 | 1760 | 0.00866 | 0.037 |
| PC(o-22:1(13Z)/20:4(8Z,11Z,14Z,17Z)) | 52447 | 106165.5 | −1.017 | 2998 | 0.00866 | 0.037 |
| PG(31:2) | 136942.9 | 86564.2 | 0.662 | 1761 | 0.00877 | 0.0371 |
| alpha-phellandrene | 61.7 | 199.1 | −1.69 | 2992 | 0.00933 | 0.0391 |
| [PS (12:0/13:0)] 1-dodecanoyl-2-tridecanoyl- | 7612945.4 | 10637361.3 | −0.483 | 2991 | 0.00945 | 0.0393 |
| sn-glycero-3-phosphoserine (ammonium salt) | | | | | | |
| Glu-Asp-Asp | 340635.1 | 461045.6 | −0.437 | 2989 | 0.00968 | 0.0399 |
| PG(33:1) | 215737.5 | 257078.3 | −0.253 | 2984 | 0.0103 | 0.0416 |
| PA(O-20:0/22:6(4Z,7Z,10Z,13Z,16Z,19Z)) | 235161.1 | 348850.6 | −0.569 | 2984 | 0.0103 | 0.0416 |
| [FA oxo(19:0)] 18-oxo-nonadecanoic acid | 191012.9 | 264717.7 | −0.471 | 2979 | 0.0109 | 0.0438 |
| PG(16:1(9Z)/18:0) | 714625.3 | 987020.3 | −0.466 | 2978 | 0.0111 | 0.0438 |
| Leu-Val | 450170.2 | 278770.9 | 0.691 | 1781 | 0.0112 | 0.0438 |
| demethylmenaquinone-6 | 576044.2 | 696566.9 | −0.274 | 2977 | 0.0112 | 0.0438 |
| PC(o-16:1(9Z)/14:1(9Z)) | 400429.2 | 269886.5 | 0.569 | 1782 | 0.0113 | 0.0439 |
| PG(P-32:0) | 306573.5 | 512926.2 | −0.743 | 2974 | 0.0116 | 0.0444 |
| (24E)-3beta,15alpha,22S-Triacetoxylanosta- | 390549.4 | 641541.7 | −0.716 | 2973 | 0.0118 | 0.0444 |
| 7,9(11),24-trien-26-oic acid | | | | | | |
| PA(33:5) | 2066319.8 | 1269332.8 | 0.703 | 1785 | 0.0118 | 0.0444 |
| LysoPC(0:0/18:0) | 210818 | 418649.1 | −0.99 | 2970 | 0.0122 | 0.0457 |
| Ile-Arg-Ile | 56540.6 | 70311.6 | −0.314 | 2968 | 0.0125 | 0.0464 |
| Lauryl acetate | 3.3 | 4.8 | −0.525 | 2967 | 0.0126 | 0.0466 |
| Glu-Glu-Gly-Tyr | 292531 | 208064.8 | 0.492 | 1793 | 0.013 | 0.0473 |
| 3-(Methylthio)-1-propanol | 215.9 | 162 | 0.414 | 1794 | 0.0131 | 0.0475 |
| (−)-(E)-1-(4-Hydroxyphenyl)-7-phenyl-6- | 1827847.9 | 2763203.9 | −0.596 | 2962 | 0.0134 | 0.0479 |
| hepten-3-ol | | | | | | |
| Dimethyl benzyl carbinyl butyrate | 5.4 | 12.7 | −1.232 | 2962 | 0.0134 | 0.0479 |
| Methyl 2,3-dihydro-3,5-dihydroxy-2-oxo-3- | 1058511.5 | 1884600.2 | −0.832 | 2960 | 0.0137 | 0.0486 |
| indoleacetic acid | | | | | | |

Metabolomic analysis performed on 139 samples (IBS: n = 78 and Control: n = 61)
Median concentration represented as arbitary unit (A.U.)
Log2FC, log2 fold change between the groups

TABLE 9a
Wilcox Rank Sum Statistical analysis is bile acids (BAs) between the subgroups of IBS, as defined by the Rome Criteria

| | | | Primary | | Secondary | | Sulfated | | | | Conjugated | | | |
|---|---|---|---|---|---|---|---|---|---|---|---|---|---|---|
| Subgroup | Total BAs | | BAs | | BAs | | BAs | | UDCA | | BAs | | Tauro/glyco | |
| Control | 7.11 | (0.285) | 5.038 | (4.446) | 94.962 | (4.446) | 8.336 | (6.14) | 47.186 | (22.212) | 13.374 | (9.323) | 1.932 | (1.521) |
| IBS-C | 7.22 | (0.322) | 5.216 | (4.271) | 94.784 | (4.271) | 9.028 | (9.923) | 55.094 | (21.022) | 14.244 | (12.293) | 2.247 | (2.568) |
| IBS-D | 7.37 | (0.31) * | 3.593 | (4.117) | 96.407 | (4.117) | 4.126 | (3.507) * | 67.022 | (15.419) ** | 7.719 | (6.03) * | 1.77 | (1.649) |
| IBS-M | 7.18 | (0.345) | 4.127 | (3.121) | 95.873 | (3.121) | 9.603 | (10.878) | 51.007 | (22.764) | 13.73 | (11.995) | 2.624 | (3.051) |

Statistical analysis was performed on 139 samples (IBS: n = 78 and Control: n = 61)
Significance after p value adjustment (Benjamini-Hochberg), was observed only in Control vs IBS-D.
* p-adj < 0.05,
** p-adj < 0.01.
Total bile acids are represented as mean of log10 values.
Others bile acid categories are presented as a percentage of the total bile acids.
Taur/Glyco ratio was calculated as ratio of taurine- and glyco-conjugated BAs (without log10 transformation).

TABLE 9b
Spearman correlation analysis between bile acids (Bas) and secondary BA synthesis pathways

| Pathway | Total BAs | Primary BAs | Secondary BAs | Sulfated BAs | UDCA | Conjugated BAs |
|---|---|---|---|---|---|---|
| ursodeoxycholate biosynthesis (PWY_7588) | 0.258* | 0.007 | 0.26* | −0.113 | 0.298** | −0.133 |
| glycocholate metabolism (PWY_6518) | 0.362** | −0.045 | 0.37** | −0.125 | 0.42** | −0.156 |

Statistical analysis was performed on 135 samples (IBS: n = 78 and Control: n = 57)
Significance after p value adjustment (Benjamini-Hochberg), was observed only in Control vs IBS-D.
* p-adj < 0.05,
** p-adj < 0.01.
Total bile acids are represented as mean of log10 values.
Others bile acid categories are presented as a percentage of the total bile acids (without Log10 transformation).

TABLE 10
Descriptive statistics of control and IBS subjects studied

| | |
|---|---|
| Control (n = 65) | IBS (n = 80) |

| | | | | |
|---|---|---|---|---|
| Age range, years \|mean) | 19-65 | (45) | 17-66 | \|39) |

| | | |
|---|---|---|
| Sex \|male/female) | 16/49 | 15/65 |
| BMI Class, n \|%) | | |

| | | | | |
|---|---|---|---|---|
| Normal | 25 | (58) | 31 | \|39) |
| Obese Class I | 11 | (17) | 14 | \|18) |
| Obese Class II | 3 | (5) | 5 | (6) |
| Obese Class III | 1 | (2) | 3 | (4) |
| Overweight | 21 | (22) | 22 | \|22) |
| Underweight | 3 | (3) | 3 | (4) |

HADS: Anxiety, n |%)

| | | | | |
|---|---|---|---|---|
| Normal \|0-10) | 59 | (91) | 58 | \|73) |
| Abnormal \|11-21) | 6 | (9) | 22 | \|28) |

HADS: Depression, n (%)

| | | | | |
|---|---|---|---|---|
| Normal \|0-10) | 64 | (98) | 70 | \|88) |
| Abnormal \|11-21) | 1 | (2) | 10 | \|13) |

Bristol Stool Score, n (%)

| | | | | |
|---|---|---|---|---|
| Normal | 54 | (83) | 18 | \|23) |
| Constipated | 8 | (12) | 22 | \|28) |
| Diarrhoea | 3 | (5) | 42 | \|50) |

| IBS subtype, n \|%) | | | |
|---|---|---|---|
| IBS-C | | 30 | \|38) |
| IBS-D | N/A | 21 | \|36) |
| IBS-M | | 29 | \|36) |

| | | | | |
|---|---|---|---|---|
| SeHCAT assayed, n (%) | 9 | (14) | 46 | \|56) |

Dietary group |FFQ), n |%)

| | | | | |
|---|---|---|---|---|
| Omnivore | 63 | (97) | 74 | \|93) |
| Vegetarian | 1 | (2) | 2 | (3) |
| Pescatarian | 1 | (2) | 1 | (1) |
| Gluten-free | 0 | (0) | 4 | (5) |

Drinks alcohol, n |%)

| | | | | |
|---|---|---|---|---|
| Current | 54 | (83) | 57 | \|71) |
| Previous | 0 | (0) | 1 | (1) |
| Never | 10 | (15) | 22 | \|28) |

Smoker, n (%)

| | | | | |
|---|---|---|---|---|
| Current | 10* | (15) | 14* | \|18) |
| Previous | 13 | (20) | 18 | \|23) |
| Never | 42 | (65) | 48 | \|60) |

*1 subject in each group smoked e-cigarettes
N/A, not applicable

TABLE 11
16S OTU Machine learning LASSO and Random Forest (RF) statistics

| | |
|---|---|
| LASSO | RF |

| lambda | AUC | Sens | Spec | mtry | AUC | Sens | Spec |
|---|---|---|---|---|---|---|---|
| 0.1 | 0.757 | 0.883 | 0.469 | 1 | 0.851 | 0.924 | 0.542 |

| | |
|---|---|
| Ten-fold cross-validation | Ten-fold cross-validation |

| Reference | | | Reference | | |
|---|---|---|---|---|---|
| Prediction | IBS | Healthy | Prediction | IBS | Healthy |
| IBS | 68.8 | 34.0 | IBS | 72.1 | 29.3 |
| Healthy | 9.2 | 30.0 | Healthy | 5.9 | 34.7 |
| Accuracy | (average) | 0.6958 | Accuracy | (average) | 0.7521 |

| RF Ranking | | | | | | |
|---|---|---|---|---|---|---|
| Rank # | Ranking | Phylum | Class | Order | Family | Genus |
| 1 | 100 | Firmicutes | Clostridia | Clostridiales | Lachnospiraceae | |
| 2 | 87.5 | Firmicutes | | | | |
| 3 | 82.1 | Firmicutes | Clostridia | Clostridiales | Ruminococcaceae | Butyricicoccus |
| 4 | 66.3 | Firmicutes | Clostridia | Clostridiales | Lachnospiraceae | |
| 5 | 62.4 | Firmicutes | Clostridia | Clostridiales | | |
| 6 | 57.2 | Firmicutes | Clostridia | Clostridiales | Ruminococcaceae | |
| 7 | 43.7 | Firmicutes | Clostridia | Clostridiales | Ruminococcaceae | |
| 8 | 30.8 | Firmicutes | | | | |
| 9 | 15.1 | Firmicutes | Clostridia | Clostridiales | Ruminococcaceae | |
| 10 | 0 | Firmicutes | Clostridia | Clostridiales | Lachnospiraceae | |

Analysis had 2 classes: Control and IBS and included 139 samples (IBS: n = 80 and Control: n = 59) Coriobacteriaceae
Metrics reported are the average values from 10 repeats of 10-fold Cross Validation.
Taxonomy classified using the RDP classfier, database version 2.10.1.

TABLE 12
16S OTU Machine learning LASSO and Random Forest (RF) statistics sequence information

| Rank | | | | | | | |
|---|---|---|---|---|---|---|---|
| # | Ranking | Phylym | Class | Order | Family | Genus | Sequence |
| 1 | 100 | Firmicutes | Clostridia | Clostridiales | Lachno- | | CCTACGGGGGGCAGCAGTGGGGAATATTG |
| | | | | | spiraceae | | CACAATGGGGGAAACCCTGATGCAGCGAC |
| | | | | | | | GCCGCGTGAGTGAAGAAGTATTTCGGTAT |
| | | | | | | | GTAAAGCTCTATCAGCAGGGAAGAAAATG |
| | | | | | | | ACGGTACCTGACTAAGAAGCCCCGGCTAA |
| | | | | | | | CTACGTGGCCAGCAGCCGCGGTAATACGT |
| | | | | | | | AGGGGGCAAGCGTTATCCGGATTTACTGG |
| | | | | | | | GTGTAAAGGGAGCGTAGGTGGTATGGCAA |
| | | | | | | | GTCAGAGGTGAAAACCCAGGGCTTAACCT |
| | | | | | | | TGGGATTGCCTTTGAAACTGTCAGACTAG |
| | | | | | | | AGTGCAGGAGGGGTAAGTGGAATTCCTAG |
| | | | | | | | TGTAGCGGTGAAATGCGTAGATATTAGGA |
| | | | | | | | GGAACACCAGTGGCGAAGGCGGCTTACTG |
| | | | | | | | GACTGTAACTGACACTGAGGCTCGAAAGC |
| | | | | | | | GTGGGGAGCAAACAGGATTAGATACCCGA |
| | | | | | | | GTAGTC (SEQ ID No: 1) |
| 2 | 87.5 | Firmicutes | | | | | CCTACGGGGGGCTGCAGTGGGGAATATTG |
| | | | | | | | GGCAATGGAGGAAACTCTGACCCAGCAAC |
| | | | | | | | GCCGCGTGGAGGAAGAAGTTTTCGGATCG |
| | | | | | | | TAAACTCCTGTCCTTGGAGACGAGTAGAA |
| | | | | | | | GACGGTATCCAAGGAGGAAGCCCCGGCTA |
| | | | | | | | ACTACGTGCCAGCAGCCGCGGTAATACGT |
| | | | | | | | AGGGGGCAAGCGTTGTCCGGAATAATTGG |
| | | | | | | | GCGTAAAGGGCGCGTAGGCGGCTCGGTAA |
| | | | | | | | GTCTGGAGTGAAAGTCCTGCTTTTAAGGT |
| | | | | | | | GGGAATTGCTTTGGATACTGTCGGGCTTG |
| | | | | | | | AGTGCAGGAGAGGTTAGTGGAATTCCCAG |
| | | | | | | | TGTAGCGGTGAAATGCGTAGAGATTGGGA |
| | | | | | | | GGAACACCAGTGGCGAAGGCGACTAACTG |
| | | | | | | | GACTGTAACTGACGCTGAGGCGCGAAAGT |
| | | | | | | | GTGGGGAGCAAACAGGATTAGATACCCCA |
| | | | | | | | GTAGTC (SEQ ID No: 2) |
| 3 | 82.1 | Firmicutes | Clostridia | Clostridiales | Ruminoco- | Buty- | CCTACGGGGGGCTGCAGTGGGGAATATTG |
| | | | | | ccaceae | ricico- | CGCAATGGGGGAAACCCTGACGCAGCAAC |
| | | | | | | ccus | GCCGCGTGATTGAAGAAGGCCTTCGGGTT |
| | | | | | | | GTAAAGATCTTTAATCAGGGACGAAACAT |
| | | | | | | | GACGGTACCTGAAGAATAAGCTCCGGCTA |
| | | | | | | | ACTACGTGCCAGCAGCCGCGGTAATACGT |
| | | | | | | | AGGGAGCAAGCGTTATCCGGATTTACTGG |
| | | | | | | | GTGTAAAGGGCGCGCAGGCGGGCCGGCAA |
| | | | | | | | GTTGGAAGTGAAATCCGGGGGCTTAACCC |
| | | | | | | | CCGAACTGCTTTCAAAACTGCTGGTCTTG |
| | | | | | | | AGTGATGGAGAGGCAGGCGGAATTCCGTG |
| | | | | | | | TGTAGCGGTGAAATGCGTAGATATACGGA |
| | | | | | | | GGAACACCAGTGGCGAAGGCGGCCTGCTG |
| | | | | | | | GACATTAACTGACGCTGAGGCGCGAAAGC |
| | | | | | | | GTGGGGAGCAAACAGGATTAGATACCCCT |
| | | | | | | | GTAGTC (SEQ ID No: 3) |
| 4 | 66.3 | Firmicutes | Clostridia | Clostridiales | Lachno- | | CCTACGGGTGGCTGCAGTGGGGAATATTG |
| | | | | | spiraceae | | CACAATGGGGGAAACCCTGATGCAGCAAC |
| | | | | | | | GCCGCGTGAGTGAAGAAGTATTTCGGTAT |
| | | | | | | | GTAAAGCTCTATCAGCAGGAAAGAAAATG |
| | | | | | | | ACGGTACCTGACTAAGAAGCCCCGGCTAA |
| | | | | | | | CTACGTGCCAGCAGCCGCGGTAATACGTA |
| | | | | | | | GGGGGCAAGCGTTATCCGGATTTACTGGG |
| | | | | | | | TGTAAAGGGAGCGTAGACGGTGAGGCAAG |
| | | | | | | | TCTGAAGTGAAATGCCGGGGCTCAACCCC |
| | | | | | | | GGAACTGCTTTGGAAACTGTCGTACTAGA |
| | | | | | | | GTGTCGGAGGGGTAAGCGGAATTCCTAGT |
| | | | | | | | GTAGCGGTGAAATGCGTAGATATTAGGAG |
| | | | | | | | GAACACCAGTGGCGAAGGCGGCTTGCTGG |
| | | | | | | | ACTGTAACTGACACTGAGGCTCGAAAGCG |
| | | | | | | | TGGGGAGCAAACAGGATTAGATACCCTTG |
| | | | | | | | TAGTC (SEQ ID No: 4) |
| 5 | 62.4 | Firmicutes | Clostridia | Clostridiales | | | CCTACGGGGGGCAGCAGTCGGGAATATTG |
| | | | | | | | CGCAATGGAGGAAACTCTGACGCAGTGAC |
| | | | | | | | GCCGCGTATAGGAAGAAGGTTTTCGGATT |
| | | | | | | | GTAAACTATTGTCGTTAGGGAAGATACAA |
| | | | | | | | GACAGTACCTAAGGAGGAAGCTCCGGCTA |
| | | | | | | | ACTACGTGCCAGCAGCCGCGGTAATACGT |
| | | | | | | | AGGGAGCAAGCGTTATCCGGATTTATTGG |
| | | | | | | | GTGTAAAGGGTGCGTAGACGGGACAACAA |
| | | | | | | | GTTAGTTGTGAAATCCCTCGGCTTAACTG |
| | | | | | | | AGGAACTGCAACTAAAACTATTGTTCTTG |
| | | | | | | | AGTGTTGGAGAGGAAAGTGGAATTCCTAG |
| | | | | | | | TGTAGCGGTGAAATGCGTAGATATTAGGA |
| | | | | | | | GGAACACCGGTGGCGAAGGCGACTTTCTG |
| | | | | | | | GACAATAACTGACGTTGAGGCACGAAAGT |
| | | | | | | | GTGGGGAGCAAACAGGATTAGATACCCCA |
| | | | | | | | GTAGTC (SEQ ID No: 5) |
| 6 | 57.2 | Firmicutes | Clostridia | Clostridiales | Ruminoco- | | CCTACGGGGGGCTGCAGTGGGGAATATTG |
| | | | | | ccaceae | | GGCAATGGGCGAAAGCCTGACCCAGCAAC |
| | | | | | | | GCCGCGTGAAGGAAGAAGGTCTTCGGATT |
| | | | | | | | GTAAACTTCTTTTATGAGGGACGAAGGAA |
| | | | | | | | GTGACGGTACCTCATGAATAAGCCACGGC |
| | | | | | | | TAACTACGTGCCAGCAGCCGCGGTAATAC |
| | | | | | | | GTAGGTGGCAAGCGTTGTCCGGATTTACT |
| | | | | | | | GGGTGTAAAGGGCGCGTAGGCGGGATGGC |
| | | | | | | | AAGTCAGATGTGAAATCCATGGGCTCAAC |
| | | | | | | | CCATGAACTGCATTTGAAACTGTCGTTCT |
| | | | | | | | TGAGTATCGGAGAGGCAAGCGGAATTCCT |
| | | | | | | | AGTGTAGCGGTGAAATGCGTAGATATTAG |
| | | | | | | | GAGGAACACCAGTGGCGAAGGCGGCTTGC |
| | | | | | | | TGGACGACAACTGACGCTGAGGCGCGAAA |
| | | | | | | | GCGTGGGGAGCAAACAGGATTAGATACCC |
| | | | | | | | CTGTAGTC (SEQ ID No: 6) |
| 7 | 43.7 | Firmicutes | Clostridia | Clostridiales | Ruminoco- | | CCTACGGGGGGCTGCAGTGGGGGATATTG |
| | | | | | ccaceae | | CACAATGGGGGAAACCCTGATGCAGCAAC |
| | | | | | | | GCCGCGTGAGGGAAGAAGGTTTTCGGATT |
| | | | | | | | GTAAACCTCTGTCCTCAGGGAAGATAATG |
| | | | | | | | ACGGTACCTGAGGAGGAAGCTCCGGCTAA |
| | | | | | | | CTACGTGCCAGCAGCCGCGGTAATACGTA |
| | | | | | | | GGGAGCAAGCGTTGTCCGGATTTACTGGG |
| | | | | | | | TGTAAAGGGTGCGTAGGCGGGATATCAAG |
| | | | | | | | TCAGACGTGAAATCCATCGGCTTAACTGA |
| | | | | | | | TGAACTGCGTTTGAAACTGGTATTCTTGA |
| | | | | | | | GTGAGTCAGAGGCAGGCGGAATTCCCGGT |
| | | | | | | | GTAGCGGTGAAATGCGTAGAGATCGGGAG |
| | | | | | | | GAACACCAGTGGCGAAGGCGGCCTGCTGG |
| | | | | | | | GGCTTAACTGACGCTGAGGCACGAAAGCG |
| | | | | | | | TGGGGAGCAAACAGGATTAGATACCCGAG |
| | | | | | | | TAGTC (SEQ ID No: 7) |
| 8 | 30.8 | Firmicutes | | | | | CCTACGGGGGGCTGCAGTGGGGAATATTG |
| | | | | | | | GGCAATGGAGGGAACTCTGACCCAGCAAT |
| | | | | | | | GCCGCGTGAGTGAAGAAGGTTTTCGGATT |
| | | | | | | | GTAAAACTCTTTAAGCAGGGACGAAGAAA |
| | | | | | | | GTGACGGTACCTGCAGAATAAGCATCGGC |
| | | | | | | | TAACTACGTGCCAGCAGCCGCGGTAATAC |
| | | | | | | | GTAGGATGCAAGCGTTATCCGGAATGACT |
| | | | | | | | GGGCGTAAAGGGTGCGTAGGCGGTAAATC |
| | | | | | | | AAGTTGGCAGCGTAATTCCGGGGCTTAAC |
| | | | | | | | TCCGGAACTACTGCCAAAACTGGTGAACT |
| | | | | | | | AGAGTGTGTCAGGGGTAAGTGGAATTCCT |
| | | | | | | | AGTGTAGCGGTGGAATGCGTAGATATTAG |
| | | | | | | | GAGGAACACCGGAGGCGAAAGCGACTTAC |
| | | | | | | | TGGGGCACAACTGACGCTGAGGCACGAAA |
| | | | | | | | GCGTGGGGAGCAAACAGGATTAGATACCC |
| | | | | | | | CGGTAGTC (SEQ ID No: 8) |
| 9 | 15.1 | Firmicutes | Clostridia | Clostridiales | Ruminoco- | | CCTACGGGAGGCAGCAGTGGGGGATATTG |
| | | | | | ccaceae | | CACAATGGAGGAAACTCTGATGCAGCAAC |
| | | | | | | | GCCGCGTGAGGGAAGAAGGATTTCGGTTT |
| | | | | | | | GTAAACCTCTGTCTTCGGTGACGAAATGA |
| | | | | | | | CGGTAGCCGAGGAGGAAGCTCCGGCTAAC |
| | | | | | | | TACGTGCCAGCAGCCGCGGTAATACGTAG |
| | | | | | | | GGAGCAAGCGTTGTCCGGAATTACTGGGT |
| | | | | | | | GTAAAGGGTGCGTAGGTGGGACTGCAAGT |
| | | | | | | | CAGGTGTGAAAACGGTCGGCTCAACCGAT |
| | | | | | | | CGCCTGCACTTGAAACTGTGGTTCTTGAG |
| | | | | | | | TGAAGTAGAGGTAGGCGGAATTCCCGGTG |
| | | | | | | | TAGCGGTGAAATGCGTAGAGATCGGGAGG |
| | | | | | | | AACACCAGTGGCGAAGGCGGCCTACTGGG |
| | | | | | | | CTTTAACTGACGCTGAGGCACGAAAGCAT |
| | | | | | | | GGGTAGCAAACAGGATTAGATACCCCGGT |
| | | | | | | | AGTC (SEQ ID No: 9) |
| 10 | 0 | Firmicutes | Clostridia | Clostridiales | Lachno- | | CCTACGGGGGGCTGCAGTGGGGAATATTG |
| | | | | | spiraceae | | CACAATGGGGGAAACCCTGATGCAGCGAC |
| | | | | | | | GCCGCGTGAGCGAAGAAGTATTTCGGTAT |
| | | | | | | | GTAAAGCTCTATCAGCAGGGAAGATAATG |
| | | | | | | | ACGGTACCTGACTAAGAAGCCCCGGCTAA |
| | | | | | | | ATACGTGCCAGCAGCCGCGGTAATACGTA |
| | | | | | | | GGGAGCAAGCGTTATCCGGATTTATTGGG |
| | | | | | | | TGTAAAGGGTGCGTAGACGGGACAACAAG |
| | | | | | | | TTAGTTGTGAAATCCCTCGGCTTAACTGA |
| | | | | | | | GGAACTGCAACTAAAACTATTGTTCTTGA |
| | | | | | | | GTGTTGGAGAGGAAAGTGGAATTCCTAGT |
| | | | | | | | GTAGCGGTGAAATGCGTAGATATTAGGAG |
| | | | | | | | GAACACCGGTGGCGAAGGCGGCCTACTGG |
| | | | | | | | GCACCAACTGACGCTGAGGCTCGAAAGTG |
| | | | | | | | TGGGTAGCAAACAGGATTAGATACCCTAG |
| | | | | | | | TAGTC (SEQ ID No: 10) |

TABLE 13
Fecal Metabolomics Machine learning with alternative pipeline is predictive of IBS versus Control

| | LASSO Optimisation | Random Forest Optimisation | Model Performance |
|---|---|---|---|
| AUC | 0.683 (0.139) | 0.909 (0.084) | 0.686 (0.132) |
| Sensitivity | 0.624 (0.177) | 0.903 (0.108) | 0.737 (0.181) |
| Specificity | 0.608 (0.202) | 0.706 (0.188) | 0.476 (0.122) |

10-fold Cross Validation

| | Predicted IBS | Predicted Control |
|---|---|---|
| IBS | 59 | 21 |
| Control | 33 | 30 |

| Rank | | | Random Forest |
|---|---|---|---|
| # | Metabolite ID | LASSO coefficients | feature importance |
| 1 | L-Phenylalanine | −0.788 | 88.34 |
| 2 | Adenosine | 0.345 | 78.31 |
| 3 | MG(20:3(8Z, 11Z, 14Z)/0:0/0:0) | 0.33 | 64.62 |
| 4 | L-Alanine | −0.752 | 56.24 |
| 5 | 3,6-Dimethoxy-19-norpregna-1,3,5,7,9-pentaen-20-one | 0.292 | 53.14 |
| 6 | Glu-Ile-Ile-Phe | −0.569 | 49.57 |
| 7 | Glu-Ala-Gln-Ser | −0.948 | 48.99 |
| 8 | 2,4,8-Eicosatrienoic acid isobutylamide | 0.179 | 43.67 |
| 9 | Piperidine | −0.161 | 38.43 |
| 10 | Staphyloxanthin | 0.251 | 37.03 |
| 11 | beta-Carotinal | 0.368 | 35.35 |
| 12 | Hexoses | 0.107 | 35.21 |
| 13 | Ile-Arg-Ile | 0.663 | 35.06 |
| 14 | 11-Deoxocucurbitacin I | −0.141 | 34.94 |
| 15 | 1-(Malonylamino)cyclopropanecarboxylic acid | 0.353 | 31.96 |
| 16 | PG(37:2) | 0.908 | 31.75 |
| 17 | [PR] gamma-Carotene/beta.psi-Carotene | 0.122 | 31.31 |
| 18 | 20-hydroxy-E4-neuroprostane | 0.126 | 29.99 |
| 19 | Ethylphenyl acetate | 0.185 | 29.86 |
| 20 | Dodecanedioic acid | 0.089 | 28.24 |
| 21 | Ile-Lys-Cys-Gly | −0.12 | 27.87 |
| 22 | Tuberoside | 0.873 | 27.39 |
| 23 | D-galactal | 0.223 | 26.84 |
| 24 | 3,6-Dihydro-4-(4-methyl-3-pentenyl)-1,2-dithiin | 0.146 | 21.83 |
| 25 | demethylmenaquinone-6 | 0.079 | 20.51 |
| 26 | L-Arginine | 0.071 | 20.33 |
| 27 | PC(o-16:1(9Z)/14:1(9Z)) | −0.09 | 19.9 |
| 28 | Mesobilirubinogen | −0.155 | 19.84 |
| 29 | Traumatic acid | 0.172 | 19.82 |
| 30 | alpha-Tocopherol succinate | 0.123 | 18.74 |
| 31 | 3-Methylcrotonylglycine | 0.182 | 18.39 |
| 32 | (S)-(E)-8-(3,6-Dimethyl-2-heptenyl)-4′,5,7-trihydroxyflavanone | 0.072 | 18.03 |
| 33 | xi-7-Hydroxyhexadecanedioic acid | 0.031 | 17.96 |
| 34 | beta-Pinene | 0.025 | 16.94 |
| 35 | Leu-Ser-Ser-Tyr | −0.041 | 16.69 |
| 36 | Orotic acid | −0.143 | 16.59 |
| 37 | Heptane-1-thiol | −0.047 | 15.82 |
| 38 | Glu-Asp-Asp | 0.038 | 15.43 |
| 39 | LysoPE(18:2(9Z,12Z)/0:0) | 0.02 | 15.28 |
| 40 | LysoPE(22:0/0:0) | 0.282 | 15.14 |
| 41 | Creatine | 0.209 | 15.03 |
| 42 | Inosine | 0.027 | 13.46 |
| 43 | SM(d32:2) | −0.077 | 13.19 |
| 44 | Arg-Leu-Val-Cys | 0.043 | 12.52 |
| 45 | PS(O-18:0/15:0) | −0.229 | 12.45 |
| 46 | Pyridoxamine | −0.105 | 11.89 |
| 47 | N-Heptanoylglycine | 0.045 | 11.53 |
| 48 | Hematoporphyrin IX | −0.161 | 11.4 |
| 49 | 3beta,5beta-Ketotriol | −0.096 | 10.59 |
| 50 | 2-Phenylpropionate | 0.026 | 10 |
| 51 | trans-2-Heptenal | 0.014 | 9.63 |
| 52 | LysoPC(0:0/18:0) | 0.028 | 9.08 |
| 53 | Linoleoyl ethanolamide | −0.025 | 8.93 |
| 54 | LysoPE(24:0/0:0) | 0.044 | 8.8 |
| 55 | 2-Methyl-3-hydroxyvaleric acid | −0.119 | 8.58 |
| 56 | Quasiprotopanaxatriol | 0.162 | 8.56 |
| 57 | N-oleoyl isoleucine | 0.059 | 8.49 |
| 58 | (-)-(E)-1-(4-Hydroxyphenyl)-7-phenyl-6-hepten-3-ol | 0.028 | 8.44 |
| 59 | [FA hydroxy(4:0)] N-(3S-hydroxy-butanoyl)-homoserine lactone | 0.024 | 8.43 |
| 60 | Riboflavin cyclic-4′,5′-phosphate | 0.092 | 8 |
| 61 | Arg-Lys-Trp-Val | −0.626 | 7.86 |
| 62 | PC(20:1(11Z)/P-16:0) | 0.033 | 7.8 |
| 63 | 3,5-Dihydroxybenzoic acid | 0.083 | 7.67 |
| 64 | Tyrosine | −0.012 | 7.43 |
| 65 | 2,3-Epoxymenaquinone | 0.005 | 7.02 |
| 66 | His-Met-Val-Val | −0.018 | 6.86 |
| 67 | PI(41:2) | −0.021 | 6.84 |
| 68 | Phenol | −0.018 | 6.74 |
| 69 | 3,3′-Dithiobis[2-methylfuran] | −0.053 | 6.73 |
| 70 | Ala-Leu-Trp-Pro | 0 | 6.7 |
| 71 | 1,2,3-Tris(1-ethoxyethoxy)propane | −0.051 | 6.48 |
| 72 | Vanilpyruvic acid | −0.052 | 6.43 |
| 73 | 2-Hydroxy-3-carboxy-6-oxo-7-methylocta-2,4-dienoate | 0.035 | 6.2 |
| 74 | Secoeremopetasitolide B | 0.023 | 5.77 |
| 75 | 2-O-Benzoyl-D-glucose | 0.033 | 5.65 |
| 76 | Ile-Leu-Phe-Trp | 0.094 | 5.49 |
| 77 | (R)-lipoic acid | 0.036 | 5.18 |
| 78 | PA(20:4(5Z,8Z,11Z,14Z)e/2:0) | 0.013 | 5.15 |
| 79 | PE(P-16:0e/0:0) | 0.003 | 5.15 |
| 80 | Benzyl isobutyrate | 0.001 | 5.04 |
| 81 | Hexyl 2-furoate | −0.099 | 5.04 |
| 82 | Trp-Ala-Ser | 0.012 | 4.95 |
| 83 | LysoPC(15:0) | −0.093 | 4.72 |
| 84 | 4-Hydroxycrotonic acid | −0.007 | 4.72 |
| 85 | 3-Feruloyl-1,5-quinolactone | 0.05 | 4.6 |
| 86 | Furfuryl octanoate | 0.178 | 4.44 |
| 87 | PC(22:2(13Z,16Z)/15:0) | −0.006 | 4.26 |
| 88 | (-)-1-Methylpropyl 1-propenyl disulfide | −0.021 | 4.07 |
| 89 | PC (36:6) | 0.073 | 4.05 |
| 90 | Leucyl-Glycine | −0.096 | 3.96 |
| 91 | CE(16:2) | 0.041 | 3.81 |
| 92 | Triterpenoid | 0 | 3.79 |
| 93 | Violaxanthin | 0.002 | 3.79 |
| 94 | [FA hydroxy(17:0)] heptadecanoic acid | −0.059 | 3.6 |
| 95 | 2-Hydroxyundecanoate | 0.077 | 3.6 |
| 96 | Chorismate | −0.003 | 3.52 |
| 97 | delta-Dodecalactone | 0.161 | 3.34 |
| 98 | 3-O-Protocatechuoylceanothic acid | 0.058 | 3.31 |
| 99 | PG(16:1(9Z)/16:1(9Z)) | −0.004 | 3.17 |
| 100 | p-Cresol sulfate | −0.003 | 3.15 |
| 101 | Quercetin 3′-sulfate | 0.02 | 3.03 |
| 102 | PS(26:0)) | −0.02 | 2.94 |
| 103 | Ala-Leu-Phe-Trp | 0.016 | 2.93 |
| 104 | L-Glutamic acid 5-phosphate | −0.003 | 2.87 |
| 105 | N,2,3-Trimethyl-2-(1-methylethyl)butanamide | −0.058 | 2.86 |
| 106 | Isoamyl isovalerate | −0.06 | 2.85 |
| 107 | n-Dodecane | −0.029 | 2.81 |
| 108 | PC(14:1(9Z)/14:1(9Z)) | −0.089 | 2.8 |
| 109 | Lucyoside Q | 0.007 | 2.76 |
| 110 | Endomorphin-1 | −0.017 | 2.51 |
| 111 | 3-Hydroxy-10′-apo-b,y-carotenal | 0.013 | 2.5 |
| 112 | Pyrroline hydroxycarboxylic acid | 0.014 | 2.39 |
| 113 | S-Propyl 1-propanesulfinothioate | 0.019 | 2.38 |
| 114 | N-Methylindolo[3,2-b]-5alpha-cholest-2-ene | −0.007 | 2.31 |
| 115 | Tocopheronic acid | 0.05 | 2.26 |
| 116 | 1-(2,4,6-Trimethoxyphenyl)-1,3-butanedione | 0.018 | 2.24 |
| 117 | Homogentisic acid | 0.011 | 2.22 |
| 118 | LysoPE(18:1(9Z)/0:0) | 0.008 | 2.19 |
| 119 | N-stearoyl valine | 0.009 | 2.17 |
| 120 | trans-Carvone oxide | 0.07 | 2.14 |
| 121 | 1,1′-Thiobis-1-propanethiol | 0.002 | 2.14 |
| 122 | 2-(Ethylsulfonylmethyl)phenyl methylcarbamate | 0.076 | 2.05 |
| 123 | menaquinone-4 | 0.004 | 2.04 |
| 124 | Benzeneacetamide-4-O-sulphate | 0.01 | 2 |
| 125 | N5-acetyl-N5-hydroxy-L-ornithine | 0.001 | 1.98 |
| 126 | Succinic acid | 0 | 1.97 |
| 127 | Asn-Lys-Val-Pro | 0.083 | 1.92 |
| 128 | LysoPC(14:1(9Z)) | 0.003 | 1.88 |
| 129 | Phenol glucuronide | −0.015 | 1.71 |
| 130 | 2-methyl-Butanoic acid, 2-methylbutyl ester | 0.01 | 1.67 |
| 131 | 3-O-Caffeoyl-1-O-methylquinic acid | 0.004 | 1.66 |
| 132 | [FA hydroxy(24:0)] 3-hydroxy-tetracosanoic acid | 0.01 | 1.63 |
| 133 | N-(2-hydroxyhexadecanoyl)-sphinganine-1-phospho-(1'-myo-inositol) | 0.146 | 1.56 |
| 134 | gamma-Dodecalactone | 0.117 | 1.54 |
| 135 | PA(22:1(11Z)/0:0) | −0.074 | 1.49 |
| 136 | Butyl butyrate | 0.025 | 1.44 |
| 137 | TG(20:5(5Z,8Z,11Z,14Z,17Z)/18:1(9Z)/22:5(7Z,10Z,13Z,16Z,19Z))[iso6] | −0.035 | 1.38 |
| 138 | Clausarinol | 0.03 | 1.36 |
| 139 | 4-Methyl-2-pentanone | 0.006 | 1.31 |
| 140 | Trigonelline | 0.02 | 1.18 |
| 141 | Arg-Val-Pro-Tyr | 0.008 | 1.17 |
| 142 | 2,3-Methylenesuccinic acid | 0.016 | 1.04 |
| 143 | Serinyl-Threonine | 0.005 | 1.04 |
| 144 | Lycoperoside D | −0.009 | 1.03 |
| 145 | Geraniol | 0.012 | 1 |
| 146 | 1-18:2-lysophosphatidylglycerol | 0.098 | 0.89 |
| 147 | omega-6-Hexadecalactone, Ambrettolide | 0.031 | 0.83 |
| 148 | gamma-Glutamyl-S-methylcysteinyl-beta-alanine | 0.008 | 0.79 |
| 149 | FA oxo(22:0) | 0.005 | 0.53 |
| 150 | D-Ribose | −0.021 | 0.53 |
| 151 | LysoPC(17:0) | 0.036 | 0.47 |
| 152 | PA(O-36:4) | 0.02 | 0.38 |
| 153 | C19 Sphingosine-1-phosphate | −0.018 | 0.34 |
| 154 | 4-Hydroxy-5-(dihydroxyphenyl)-valeric acid-O-methyl-O-sulphate | 0.016 | 0.29 |
| 155 | PE(14:1(9Z)/14:0) | 0.015 | 0.28 |
| 156 | Citronellyl tiglate | 0.052 | 0.27 |
| 157 | Ethyl methylphenylglycidate (isomer 1) | −0.038 | 0.24 |
| 158 | N-Acetyl-leu-leu-tyr | 0.003 | 0 |
| 158 | PS(O-34:3) | −0.002 | 0 |

LASSO and Random Forest (RF) statistics of metabolites predictive of IBS versus Control; Analysis had 2 classes: Control and IBS and included 143 samples (IBS: n = 80 and Control: n = 63); Metrics reported are the mean and the standard deviation of values from Cross Validation.

TABLE 14
Strain level (CAG) Machine learning is predictive of IBS versus Control

| | LASSO | Random Forest | Model |
|---|---|---|---|
| | Optimisation | Optimisation | Performance |
| AUC | 0.754 (0.146) | 0.897 (0.09) | 0.814 (0.134) |
| Sensitivity | 0.814 (0.162) | 0.95 (0.074) | 0.875 (0.102) |
| Specificity | 0.525 (0.241) | 0.57 (0.205) | 0.497 (0.217) |

10-fold Cross Validation

| | Predicted IBS | Predicted Control |
|---|---|---|
| IBS | 70 | 10 |
| Control | 30 | 29 |

| | | LASSO | Random Forest |
|---|---|---|---|
| Rank # | CAG ID | coefficients | feature importance |
| 1 | unclassified_00060 | 0.001381 | 60.04 |
| 2 | unclassified_13382 | 0.068289 | 57.19 |
| 3 | Ambiguous_02465 | 0.010803 | 55.91 |
| 4 | unclassified_10544 | 0.030574 | 43.01 |
| 5 | unclassified_01797 | 0.020433 | 42.42 |
| 6 | unclassified_01214 | 0.001162 | 40.69 |
| 7 | unclassified_04033 | 0.008943 | 40.54 |
| 8 | Ambiguous_00664 | 0.001472 | 39.75 |
| 9 | unclassified_07453 | 0.027742 | 39.38 |
| 10 | unclassified_09604 | 0.025831 | 38.55 |
| 11 | unclassified_04421 | 0.018453 | 37.31 |
| 12 | unclassified_02178 | 0.014262 | 33.23 |
| 13 | unclassified_04275 | 0.022114 | 32.93 |
| 14 | unclassified_00992 | 0.003028 | 32.52 |
| 15 | unclassified_08180 | 0.03671 | 32.5 |
| 16 | unclassified_02378 | 0.00303 | 30.66 |
| 17 | unclassified_14410 | 0.028182 | 28.63 |
| 18 | unclassified_14848 | 0.00442 | 28.44 |
| 19 | Escherichia_coli_08281 | 0.007697 | 26.73 |
| 20 | unclassified_01723 | 0.002795 | 25.28 |
| 21 | unclassified_01973 | 0.003755 | 23.46 |
| 22 | unclassified_07490 | 0.017293 | 23.05 |
| 23 | unclassified_04642 | 0.010974 | 22.99 |
| 24 | unclassified_12490 | 0.041094 | 22.65 |
| 25 | unclassified_04705 | 0.004598 | 22.01 |
| 26 | unclassified_01929 | 0.013678 | 21.88 |
| 27 | unclassified_04761 | 0.025652 | 21.43 |
| 28 | unclassified_13688 | 0.010278 | 20.66 |
| 29 | Clostridium_spp_04742 | 0.005228 | 19.73 |
| 30 | Streptococcus_spp_01624 | 0.001426 | 19.23 |
| 31 | unclassified_12615 | 0.036959 | 18.59 |
| 32 | unclassified_10766 | 0.05376 | 17.8 |
| 33 | unclassified_11165 | 0.035285 | 17.52 |
| 34 | unclassified_00496 | 0.001305 | 17.34 |
| 35 | unclassified_07581 | 0.007595 | 15.91 |
| 36 | unclassified_10074 | 0.012338 | 15.41 |
| 37 | unclassified_01227 | 0.000621 | 13.73 |
| 38 | unclassified_01850 | 0.004519 | 13.48 |
| 39 | unclassified_01534 | 0.001799 | 12.87 |
| 40 | unclassified_00657 | 0.001686 | 12.77 |
| 41 | unclassified_03784 | 0.012933 | 12.67 |
| 42 | Streptococcus_anginosus_14524 | 0.01304 | 12.16 |
| 43 | unclassified_04216 | 0.003356 | 12.02 |
| 44 | Parabacteroides_johnsonii_04505 | 0.007269 | 11.48 |
| 45 | unclassified_02737 | 0.0006 | 10.34 |
| 46 | Streptococcus_gordonii_00694 | 0.00061 | 10.11 |
| 48 | Ambiguous_00350 | 0.011386 | 10 |
| 49 | Ambiguous_01019 | 0.008179 | 10 |
| 50 | unclassified_00612 | 0.004216 | 10 |
| 51 | Clostridium_spp_00680 | 0.003678 | 10 |
| 52 | Ambiguous_00176 | 0.002303 | 10 |
| 53 | Ambiguous_00008 | 0.000835 | 10 |
| 47 | Ambiguous_01504 | 0.000006 | 10 |
| 54 | unclassified_07058 | 0.001504 | 9.82 |
| 55 | Clostridium_spp_11230 | 0.002081 | 9.47 |
| 56 | Ambiguous_01105 | 0.002674 | 9.4 |
| 57 | unclassified_02000 | 0.003605 | 9.28 |
| 58 | unclassified_01034 | 0.005573 | 9.27 |
| 59 | unclassified_06517 | 0.041237 | 8.95 |
| 60 | Clostridium_bolteae_00697 | 0.001039 | 8.78 |
| 61 | Turicibacter_sanguinis_07698 | 0.041323 | 8.57 |
| 62 | unclassified_04716 | 0.004963 | 8.29 |
| 63 | unclassified_06120 | 0.023365 | 8.22 |
| 64 | Clostridiales_bacterium_1_7_47FAA_00444 | 0.000169 | 8.15 |
| 65 | unclassified_00404 | 0.004334 | 8.15 |
| 66 | Ambiguous_06054 | 0.000061 | 8.14 |
| 67 | Clostridium_spp_09935 | 0.008296 | 8.09 |
| 68 | unclassified_03271 | 0.001025 | 8 |
| 69 | Ambiguous_03591 | 0.007581 | 7.86 |
| 70 | unclassified_11816 | 0.030684 | 7.6 |
| 71 | Ambiguous_03760 | 0.004159 | 7.52 |
| 72 | Clostridiales_bacterium_1_7_47FAA_00369 | 0.000864 | 7.46 |
| 73 | unclassified_04974 | 0.003624 | 7.35 |
| 74 | Streptococcus_anginosus_02750 | 0.000721 | 6.82 |
| 75 | unclassified_08690 | 0.003226 | 6.72 |
| 76 | unclassified_06706 | 0.00206 | 6.56 |
| 77 | Paraprevotella_xylaniphila_07441 | 0.00209 | 6.41 |
| 78 | unclassified_04992 | 0.005196 | 6.09 |
| 79 | unclassified_08989 | 0.011704 | 6.08 |
| 80 | unclassified_02911 | 0.002799 | 6 |
| 81 | unclassified_02952 | 0.006054 | 5.87 |
| 82 | unclassified_00342 | 0.000084 | 5.49 |
| 83 | Eubacterium_sp_3_1_31_00679 | 0.001407 | 5.12 |
| 84 | Lachnospiraceae_bacterium_5_1_57FAA_01560 | 0.000291 | 5 |
| 85 | Escherichia_coli_01241 | 0.000114 | 4.84 |
| 86 | unclassified_02624 | 0.002928 | 4.72 |
| 87 | Clostridiaceae_bacterium_JC118_03657 | 0.005134 | 4.58 |
| 88 | unclassified_09127 | 0.001119 | 4.5 |
| 89 | unclassified_05532 | 0.000001 | 4.48 |
| 90 | unclassified_09184 | 0.005517 | 4.45 |
| 91 | Bacteroides_spp_03730 | 0.000523 | 4.4 |
| 92 | Paraprevotella_xylaniphila_08998 | 0.002821 | 4.3 |
| 93 | unclassified_03065 | 0.001211 | 4.27 |
| 94 | Ambiguous_01649 | 0.000779 | 4.26 |
| 95 | Streptococcus_mutans_09018 | 0.005574 | 4.26 |
| 96 | Ambiguous_13545 | 0.00493 | 4.22 |
| 97 | unclassified_08505 | 0.004519 | 4.12 |
| 98 | Escherichia_coli_00201 | 0.000672 | 3.9 |
| 99 | unclassified_03041 | 0.004803 | 3.78 |
| 100 | unclassified_05056 | 0.007699 | 3.77 |
| 101 | unclassified_01365 | 0.000379 | 3.38 |
| 102 | Bacteroides_plebeius_08099 | 0.009286 | 3.37 |
| 103 | Ambiguous_05609 | 0.008937 | 3.32 |
| 104 | unclassified_05684 | 0.00422 | 3.25 |
| 105 | unclassified_02242 | 0.002019 | 3.21 |
| 106 | Clostridium_clostridioforme_06211 | 0.061218 | 3.16 |
| 107 | Klebsiella_pneumoniae_01817 | 0.012099 | 2.92 |
| 108 | Clostridium_hathewayi_06002 | 0.000291 | 2.87 |
| 109 | Ambiguous_03727 | 0.000144 | 2.8 |
| 110 | Bacteroides_fragilis_14807 | 0.011963 | 2.71 |
| 111 | unclassified_01340 | 0.001622 | 2.66 |
| 112 | unclassified_08925 | 0.000758 | 2.57 |
| 113 | unclassified_08324 | 0.000257 | 2.48 |
| 114 | Prevotella_disiens_10832 | 0.004206 | 2.48 |
| 115 | Clostridium_leptum_11975 | 0.002101 | 2.35 |
| 116 | unclassified_01283 | 0.004063 | 2.09 |
| 117 | Pseudoflavonifractor_capillosus_03569 | 0.000849 | 2.06 |
| 118 | unclassified_12165 | 0.006268 | 2.02 |
| 119 | unclassified_07203 | 0.000139 | 1.84 |
| 120 | Bacteroides_intestinalis_14747 | 0.001208 | 1.73 |
| 121 | unclassified_08104 | 0.000055 | 1.6 |
| 122 | unclassified_14839 | 0.000932 | 1.54 |
| 123 | Enterococcus_faecalis_01189 | 0.00061 | 1.52 |
| 124 | Streptococcus_infantis_14065 | 0.00542 | 1.24 |
| 125 | Lachnospiraceae_bacterium_1_4_56FAA_13504 | 0.000698 | 1.09 |
| 126 | Alistipes_shahii_15132 | 0.000646 | 1.04 |
| 127 | Clostridium_spp_10114 | 0.000481 | 1.03 |
| 128 | unclassified_13766 | 0.000045 | 0.94 |
| 129 | Ambiguous_06549 | 0.00035 | 0.73 |
| 130 | unclassified_14263 | 0.00382 | 0.7 |
| 131 | Eubacterium_sp_3_1_31_05331 | 0.001123 | 0.55 |
| 132 | Clostridium_asparagiforme_06161 | 0.000488 | 0.4 |
| 133 | Streptococcus_mutans_07592 | 0.000826 | 0.33 |
| 134 | unclassified_12188 | 0.003405 | 0.26 |
| 135 | Clostridium_symbiosum_14754 | 0.002328 | 0.17 |
| 136 | Streptococcus_sanguinis_11557 | 0.001 | 0 |

LASSO and Random Forest (RF) statistics of CAGs predictive of IBS versus Control
Analysis had 2 classes: Control and IBS and included 139 samples (IBS: n = 80 and Control: n = 59)
Metrics reported are the mean and the standard deviation of values from Cross Validation.
Taxonomy is assigned where greater than 60% of the gene families are associated with a genus level.
LASSO coefficients are absolute values for the CAG dataset

TABLE 15
Number of samples used in analysis of IBS subtypes

| | 16S | Shotgun | Fecal | Urine |
|---|---|---|---|---|
| | Genus | Species | Metabolomics | Metabolomics |
| Number of Samples | 138 | 135 | 139 | 138 |

TABLE 16
Permuational MANOVA results for beta diversity analysis

| | 16S | Shotgun | Fecal | Urine |
|---|---|---|---|---|
| | Genus | Species | Metabolomics | Metabolomics |
| IBS-1 subgroup vs | 0.0006 | 0.006 | 0.0012 | 0.906 |
| IBS-2 subgroup | | | | |
| IBS-1 subgroup vs | 0.0006 | 0.006 | 0.006 | 0.006 |
| IBS-3 subgroup | | | | |
| IBS-2 subgroup vs | 0.0006 | 0.006 | 0.002 | 0.774 |
| IBS-3 subgroup | | | | |
| IBS-1 subgroup vs | 0.0006 | 0.006 | 0.001 | 0.006 |
| Healthy | | | | |
| IBS-2 subgroup vs | 0.0006 | 0.006 | 0.012 | 1 |
| Healthy | | | | |
| IBS-3 subgroup vs | 1 | 1 | 0.059 | 0.006 |
| Healthy | | | | |

All values are adjusted p-values using Bonferroni correction

TABLE 21a
Urine metabolomics machine learning with alternative
pipeline is predictive of IBS versus Control: metabolites
present at higher levels in controls

| | LASSO | Random Forest | Model |
|---|---|---|---|
| | Optimisation | Optimisation | Performance |
| AUC | 1 (0) | 0.999 (0.001) | 1 (0) |
| Sensitivity | 0.992 (0.027) | 1 (0) | 1 (0) |
| Specificity | 0.881 (0.142) | 0.976 (0.064) | 0.969 (0.066) |

10-fold Cross Validation

| | Predicted IBS | Predicted Control |
|---|---|---|
| IBS | 80 | 0 |
| Control | 2 | 61 |

| | | AUC (Prediction |
|---|---|---|
| Rank # | Metabolite ID | of/higher in Controls) |
| 1 | Tricetin 3′-methyl ether 7,5′- | 0.86 |
| | diglucuronide | |
| 2 | Alloathyriol | 0.86 |
| 3 | Torasemide | 0.85 |
| 4 | (−)-Epigallocatechin sulfate | 0.8 |
| 5 | Tetrahydrodipicolinate | 0.78 |
| 6 | Silicic acid | 0.75 |
| 7 | Delphinidin 3-(6″-O-4-malyl- | 0.75 |
| | glucosyl)-5-glucoside | |
| 8 | Creatinine | 0.75 |
| 9 | L-Arginine | 0.74 |
| 10 | Leucyl-Methionine | 0.74 |
| 11 | Gln-Met-Pro-Ser | 0.73 |
| 12 | Ala-Asn-Cys-Gly | 0.72 |
| 13 | Isoleucyl-Proline | 0.71 |
| 14 | 3,4-Methylenesebacic acid | 0.71 |
| 15 | (4-Hydroxybenzoyl)choline | 0.71 |
| 16 | Diazoxide | 0.7 |
| 17 | (1S,3R,4S)-3,4-Dihydroxycyclohexane- | 0.69 |
| | 1-carboxylate | |
| 18 | 2-Hydroxypyridine | 0.69 |
| 19 | Ala-Lys-Phe-Cys | 0.69 |
| 20 | 3-Methyldioxyindole | 0.68 |
| 21 | N-Carboxyacetyl-D-phenylalanine | 0.68 |
| 22 | Urea | 0.67 |
| 23 | Ferulic acid 4-sulfate | 0.67 |
| 24 | 3-Indolehydracrylic acid | 0.67 |
| 25 | Demethyloleuropein | 0.67 |
| 26 | 5′-Guanosyl-methylene-triphosphate | 0.67 |
| 27 | Linalyl formate | 0.67 |
| 28 | 4-Methoxyphenylethanol sulfate | 0.67 |
| 29 | Allyl nonanoate | 0.66 |
| 30 | D-Galactopyranosyl-(1−>3)-D- | 0.66 |
| | galactopyranosyl-(1−>3)-L-arabinose | |
| 31 | Met-Met-Thr-Trp | 0.66 |
| 32 | Cys-Pro-Pro-Tyr | 0.66 |
| 33 | methylphosphonate | 0.66 |
| 34 | 2-Phenylethyl octanoate | 0.66 |
| 35 | Hippuric acid | 0.65 |
| 36 | Glutarylcarnitine | 0.65 |
| 37 | Cys-Phe-Phe-Gln | 0.65 |

LASSO and Random Forest (RF) statistics of metabolites predictive of IBS versus Control
Analysis had 2 classes: Control and IBS and included 143 samples (IBS: n = 80 and Control: n = 63)
Metrics reported are the mean and the standard deviation of values from Cross Validation.
Data used was log10 transformed.
For all the external cross validation folds, lasso did not return more than 5 features.
Therefore, all the trained models are based on random forest with all the features.
Metabolites presented are the most predictive as defined by a AUC of greater than 0.65 when tested on the full dataset (applied as a feature selection methodology).

TABLE 21b
Urine metabolomics machine learning with alternative
pipeline is predictive of IBS versus Control:
metabolites present at higher levels in IBS

| | LASSO | Random Forest | Model |
|---|---|---|---|
| | Optimisation | Optimisation | Performance |
| AUC | 1 (0) | 0.999 (0.001) | 1 (0) |
| Sensitivity | 0.992 (0.027) | 1 (0) | 1 (0) |
| Specificity | 0.881 (0.142) | 0.976 (0.064) | 0.969 (0.066) |

10-fold Cross Validation

| | Predicted IBS | Predicted Control |
|---|---|---|
| IBS | 80 | 0 |
| Control | 2 | 61 |

| | | AUC (Prediction |
|---|---|---|
| Rank # | Metabolite ID | of/higher in IBS) |
| 1 | A 80987 | 1 |
| 2 | Medicagenic acid 3-O-b-D- | 1 |
| | glucuronide | |
| 3 | N-Undecanoylglycine | 0.99 |
| 4 | Ala-Leu-Trp-Gly | 0.98 |
| 5 | Gamma-glutamyl-Cysteine | 0.92 |
| 6 | Butoctamide hydrogen succinate | 0.91 |
| 7 | (−)-Epicatechin sulfate | 0.89 |
| 8 | 1,4,5-Trimethyl-naphtalene | 0.86 |
| 9 | Trp-Ala-Pro | 0.83 |
| 10 | Dodecanedioylcarnitine | 0.77 |
| 11 | 1,6,7-Trimethylnaphthalene | 0.76 |
| 12 | Sumiki's acid | 0.76 |
| 13 | Phe-Gly-Gly-Ser | 0.75 |
| 14 | 2-hydroxy-2-(hydroxymethyl)- | 0.73 |
| | 2H-pyran-3(6H)-one | |
| 15 | 5-((2-iodoacetamido)ethyl)-1- | 0.72 |
| | aminonapthalene sulfate | |
| 16 | Thiethylperazine | 0.72 |
| 17 | dCTP | 0.71 |
| 18 | Dimethylallylpyrophosphate/ | 0.71 |
| | Isopentenyl pyrophosphate | |
| 19 | Asp-Met-Asp-Pro | 0.7 |
| 20 | 3,5-Di-O-galloyl-1,4- | 0.7 |
| | galactarolactone | |
| 21 | Decanoylcarnitine | 0.69 |
| 22 | [FA (18:0)] N-(9Z- | 0.67 |
| | octadecenoyl)-taurine | |
| 23 | UDP-4-dehydro-6-deoxy-D-glucose | 0.66 |
| 24 | Delphinidin 3-O-3″,6″- | 0.66 |
| | O-dimalonylglucoside | |
| 25 | Osmundalin | 0.65 |
| 26 | Cysteinyl-Cysteine | 0.65 |

LASSO and Random Forest (RF) statistics of metabolites predictive of IBS versus Control
Analysis had 2 classes: Control and IBS and included 143 samples (IBS: n = 80 and Control: n = 63)
Metrics reported are the mean and the standard deviation of values from Cross Validation.
Data used was log10 transformed.
For all the external cross validation folds, lasso did not return more than 5 features.
Therefore, all the trained models are based on random forest with all the features.
Metabolites presented are the most predictive as defined by a AUC of greater than 0.65 when tested on the full dataset (applied as a feature selection methodology).

1.-40. (canceled)

41. A method comprising:
detecting in a biological sample from a subject the level of at least two of (i), (ii), and (iii):
(i) a bacterial strain of a taxa associated with irritable bowel syndrome (IBS);
(ii) a microbial gene involved in a pathway associated with IBS, wherein the pathway is selected from the group consisting of amino acid biosynthesis, amino acid degradation, starch degradation, galactose degradation, sulfate reduction, sulfate assimilation, and cysteine biosynthesis; or
(iii) a metabolite associated with IBS, a precursor thereof, or a breakdown product thereof, wherein the metabolite is a urine metabolite or a fecal metabolite,
and comparing the detected level of (i), (ii), or (iii) to the corresponding level of (i), (ii), or (iii) in a biological sample from a subject that does not have IBS,
wherein the subject is determined to have IBS when there is an increase in the detected level of (i), (ii), or (iii) compared to the corresponding level of (i), (ii), or (iii) in the biological sample from the subject that does not have IBS.

42. The method of claim 41, wherein the detecting of the bacterial strain comprises 16S amplicon sequencing or shotgun sequencing.

43. The method of claim 41, wherein the detecting of the metabolite comprises performing gas chromatography and liquid chromatography mass spectrometry (GC/LC MS).

44. The method of claim 41, wherein the biological sample comprises a fecal sample, a urine sample, or an oral sample.

45. The method of claim 41, wherein the subject is a human.

46. The method of claim 41, wherein the bacterial strain comprises a 16S rRNA gene sequence having at least 97% sequence identity to any one of SEQ ID NOs:1-10 or is of the group consisting of Lachnospiraceae, Firmicutes, Butyricicoccus, Clostridiales, and Ruminococcaceae.

47. The method of claim 41, wherein the bacterial strain belongs to an operational taxonomic unit (OTU) selected from Table 11.

48. The method of claim 41, wherein the pathway is selected from the group consisting of pathways listed in Table 4.

49. The method of claim 41, wherein the detecting the microbial gene comprises detecting a bacterial species carrying the gene or detecting a nucleic acid sequence encoding the gene.

50. The method of claim 41, wherein the urine metabolite comprises A 80987, Ala-Leu-Trp-Gly, Medicagenic acid 3-O-b-D-glucuronide, or (−)-Epigallocatechin sulfate or is selected from the group consisting of the metabolites listed in Table 6.

51. The method of claim 41, wherein the subject is determined to have a subcategory of IBS based on the comparing.

52. The method of claim 41, wherein the urine metabolite is: A 80987, Medicagenic acid 3-O-b-D-glucuronide, N-Undecanoylglycine, Ala-Leu-Trp-Gly, or Gamma-glutamyl-Cysteine, Tricetin 3′-methyl ether 7,5′-diglucuronide, Alloathyriol, Torasemide, (−)-Epigallocatechin sulfate, or Tetrahydrodipicolinate.

53. The method of claim 41, wherein the urine metabolite is selected from the group consisting of the metabolites listed in Table 21a and Table 21b.

54. The method of claim 41, wherein the fecal metabolite comprises 3-deoxy-D-galactose, Tyrosine, I-Urobilin, Adenosine, Glu-Ile-Ile-Phe, 3,6-Dimethoxy-19-norpregna-1,3,5,7,9-pentaen-20-one, 2-Phenylpropionate, MG(20:3(8Z,11Z,14Z)/0:0/0:0), 1,2,3-Tris(1-ethoxyethoxy)propane, Staphyloxanthin, Hexoses, 20-hydroxy-E4-neuroprostane, Nonyl acetate, 3-Feruloyl-1,5-quinolactone, trans-2-Heptenal, Pyridoxamine, L-Arginine, Dodecanedioic acid, Ursodeoxycholic acid, 1-(Malonylamino)cyclopropanecarboxylic acid, Cortisone, 9,10,13-Trihydroxystearic acid, Glu-Ala-Gln-Ser, Quasiprotopanaxatriol, N-Methylindolo[3,2-b]-5alpha-cholest-2-ene, PG(20:0/22:1(11Z)), (−)-Epigallocatechin, 2-Methyl-3-ketovaleric acid, Secoeremopetasitolide B, PC(20:1(11Z)/P-16:0), Glu-Asp-Asp, N5-acetyl-N5-hydroxy-L-ornithine acid, Silicic acid, (1xi,3xi)-1,2,3,4-Tetrahydro-1-methyl-beta-carboline-3-carboxylic acid, PS(36:5), Chorismate, Isoamyl isovalerate, PA(O-36:4), PE(P-28:0) or gamma-Glutamyl-S-methylcysteinyl-beta-alanine.

55. The method of claim 41, wherein the fecal metabolite is selected from the group consisting of metabolites listed in Table 8.

56. The method of claim 41, wherein the fecal metabolite is selected from the group consisting of metabolites listed in Table 13.

57. The method of claim 41, further comprising detecting two or more bacterial strains of two or more bacterial taxa associated with IBS, two or more microbial genes involved in a pathway associated with IBS, or two or more metabolites associated with IBS.

58. The method of claim 41, wherein the method further comprises treating the subject determined to have IBS.

59. A method of treating irritable bowel syndrome (IBS) in a subject in need thereof comprising administering to the subject a treatment for IBS selected from loperamide, a laxative, an antidepressant, an antibiotic, a probiotic, or a live biotherapeutic after detecting in a biological sample from the subject an elevated level of at least two of (i), (ii), and (iii):
(i) a bacterial strain of a taxa associated with irritable bowel syndrome (IBS), wherein the bacteria strain comprises a 16S rRNA gene sequence having at least 97% sequence identity to any one of SEQ ID NOs:1-10,
(ii) a microbial gene involved in a pathway associated with IBS, wherein the pathway is selected from the group consisting of amino acid biosynthesis, amino acid degradation, starch degradation, galactose degradation, sulfate reduction, sulfate assimilation, and cysteine biosynthesis, or
(iii) a metabolite associated with IBS, a precursor thereof, or a breakdown product thereof, wherein the metabolite is a urine metabolite or a fecal metabolite,
as compared to the corresponding level of (i), (ii), or (iii) in a biological sample from a subject that does not have IBS.

60. A kit comprising reagents for detecting:
a. a bacterial strain of a taxa associated with IBS, wherein the bacteria strain comprises a 16S rRNA gene sequence having at least 97% sequence identity to any one of SEQ ID NOs:1-10;
b. a microbial gene involved in a pathway associated with IBS, wherein the pathway is selected from the group consisting of pathways listed in Table 4; or
c. a metabolite associated with IBS, wherein the metabolite is a urine metabolite or a fecal metabolite.
